(19) United States
(12) Patent Application Publication
(10) Pub. No.: US 2021/0063414 A1
(43) Pub. Date: Mar. 4, 2021

(54) METHODS FOR PREVENTING AND/OR TREATING BONE LOSS CONDITIONS BY MODULATING IRISIN

(71) Applicants: Bruce M. Spiegelman, Waban, MA (US); Hyeonwoo Kim, Boston, MA (US); Clifford Rosen, Freeport, ME (US); Lynda Bonewald, Indianapolis, IN (US); Dana-Farber Cancer Institute, Inc., Boston, MA (US); Indiana University, Bloomington, IN (US); Maine Medical Center Research Institute, Scarborough, ME (US)

(72) Inventors: Bruce M. Spiegelman, Waban, MA (US); Hyeonwoo Kim, Boston, MA (US); Clifford Rosen, Freeport, ME (US); Lynda Bonewald, Indianapolis, IN (US)

(21) Appl. No.: 16/957,534

(22) PCT Filed: Feb. 12, 2019
(86) PCT No.: PCT/US19/17629

§ 371 Date: Jun. 24, 2020

Related U.S. Application Data
(60) Provisional application No. 62/769,125, filed on Nov. 19, 2018.
(60) Provisional application No. 62/629,447, filed on Feb. 12, 2018.

(51) Int. Cl. G01N 33/68; A61K 38/07; G01N 33/574; G01N 33/50

(52) U.S. Cl. CPC G01N 33/6893; A61K 38/07; G01N 33/57407; G01N 2800/52; G01N 33/5044; G01N 2333/70546; G01N 33/57415

(57) ABSTRACT

The present invention relates, in part, to methods of preventing and/or treating a subject afflicted with bone loss conditions comprising administering to the subject a therapeutically effective amount of an agent that decreases the amount and/or activity of irisin.

### CROSS-REFERENCE TO RELATED APPLICATIONS

This application claims the benefit of U.S. Provisional Application No. 62/629,447, filed on Feb. 12, 2018; and U.S. Provisional Application No. 62/769,125, filed on Nov. 19, 2018; the entire contents of each of said applications are incorporated herein in their entirety by this reference.

### STATEMENT OF RIGHTS

This invention was made with government support under grant numbers DK054077, DK092759, and P01 AG039355 awarded by The National Institutes of Health. The government has certain rights in the invention.

### BACKGROUND OF THE INVENTION

The mechanism of bone loss is not well understood, but in practical effect, the disorder arises from an imbalance in the formation of new healthy bone and the resorption of old bone, with the result being a net loss of bone tissue. This bone loss includes a decrease in both mineral content and protein matrix components of the bone, and leads to an increased fracture rate of, predominantly, femoral bones and bones in the forearm and vertebrae. These fractures, in turn, lead to an increase in general morbidity, a marked loss of stature and mobility, and, in many cases, an increase in mortality resulting from complications. Unchecked, bone loss can lead to osteoporosis and/or osteopenia. Osteopenia is reduced bone mass due to a decrease in the rate of osteoid synthesis to a level insufficient to compensate normal bone lysis. Osteoporosis is a major debilitating disease whose prominent feature is the loss of bone mass (decreased density and enlargement of bone spaces) without a reduction in bone volume, producing porosity and fragility.

Physical activity has been shown to benefit several metabolic disorders, including obesity, diabetes and fatty liver disease (Kirwan et al. (2017) Cleve. Clin. J. Med. 84:S15-S21). Older cross-sectional studies indicated exercise can prevent age-related bone loss (Krolner et al. (1983) Clin. Sci. (Lond} 64:541-546; Prince et al. (1991) N. Engl. J. Med. 325:1189-1195). Loss of bone mass with age has significant socio-economic and medical implications due to the heightened susceptibility to fractures. Osteoporosis impairs mobility, increases co-morbidities, reduces quality of life and can shorten lifespan, particularly in the elderly (Li et al. (2017) Bmc. Musculoskelet. Disord. 18:46).

The evidence that an exercise program can prevent bone loss is somewhat conflicted in part because different types of physical activity impact the skeleton at distinct sites in different ways. For example, several studies have shown that resistance training is associated with relative preservation of femoral but not lumbar bone mass in adults (Eatemadololama et al. (2017) Clin. Cases Miner. Bone Metab. 14:157-160; Spindler et al. (1997) Nephrol. Dial. Transplant 12:128-132; Vincent & Braith (2002) Med. Sci. Sports Exerc. 34:17-23). On the other hand, fracture risk reduction has not been established in randomized trials with long term physical activity. Importantly, results from endurance exercise trials, particularly in the elderly, are even less convincing, with some studies showing preservation of bone mass and others showing no effect or even bone loss (Braam et al. (2003) Am. J. Sports Med. 31:889-895; Duckham et al. (2013) Calcif. Tissue Lnt. 92:444-450; Scofield & Hecht (2012) Curr. Sports Med. Rep. 11:328-334). Consistent with the latter effect, brief bouts of endurance training have been shown to increase bone resorption and stimulate sclerostin, an endogenous inhibitor of bone formation (Pickering et al. (2017) Calcif. Tissue. Lnt. 101:170-173; Baron & Kneissel et al. (2013) Nat Med, 19:179-192; Kohrt et al. (2018) J. Bone Miner. Res. 33:1326-1334). Sclerostin is produced almost exclusively by osteocytes, the ‘command and control’ cell of the bone remodeling unit (Van Bezooijen et al. (2004) J. Exp. Med. 199, 805-814; Bonewald et al. (2011) J. Bone Miner. Res. 26:229-238). Osteocytes arise from mature osteoblasts, are imbedded in the cortical matrix, and comprise nearly 90% of the cellular composition of bone (Bonewald et al. (2011) J. Bone Miner. Res. 26:229-238). As such they are thought to be the transducers of mechanical signals arising from physical activity and loading (Bonewald et al. (2011) J. Bone Miner. Res. 26:229-238). In turn, these cells, through an elaborate network of canaliculi, communicate with both osteoblasts and osteoclasts, tightly regulating remodeling (Bonewald et al. (2011) J. Bone Miner. Res. 26:229-238). Emerging evidence indicates that osteocytes can also directly resorb bone during periods of excessive calcium demand (Qing and Bonewald (2009) Lnt. J. Oral. Sci. 1:59-65) or after ovariectomy (Almeida et al. (2017) Physiol. Rev. 97:135-187) and as such these cells have become a prime target for anabolic osteoporotic therapies such as parathyroid hormone and monoclonal anti-sclerostin antibodies (Bellido et al. (2005) Endocrinology 146:4577-4583; Keller and Kneissel (2005) Bone 37:148-158; Ominsky el al. (2010) J. Bone Miner. Res. 25:948-959; Li et al. (2009) J. Bone Miner. Res. 24:578-588). Anti-sclerostin antibodies increase bone mass dramatically in humans but also may have cardiovascular side-effects that could limit their use in practice (Mcclung et al. (2017) Ther. Adv. Musculoskelet. Dis. 9:263-270).

Physical activity targets osteocytes but also stimulates the production of several hormone-like molecules from skeletal muscle termed “myokines” (Pedersen & Febbraio (2012) Nat. Rev. Endocrinol. 8:457-465). These include IL-6, irisin and meteorin-like (Keller et al. (2001) Faseb. J. 15:2748-2750; Bostrom et al. (2012) Nature 481:463-468; Rao et al. (2014) Cell 157:1279-1291). Irisin has been shown to be induced in many (but not all) studies of endurance exercise in both mice and humans (Bostrom et al. (2012) Nature 481:463-468; Jedrychowski et al. (2015) Cell Metab. 22:734-740; Lee et al. (2014) Cell Metab. 19:302-309; Pekkala et al. (2013) J. Physiol. 591:5393-5400). It is a cleaved product from a type I membrane protein, Fibronectin type III domaincontaining protein 5 (FNDC5), and is shed into the extracellular milieu and circulation (Bostrom et al. (2012) Nature 481:463-468). The crystal structure of irisin has been determined and contains an FNIII domain (Schumacher et al. (2013) J. Biol. Chem. 288:33738-33744) that is also contained in fibronectin and many other proteins (Hynes et al. (1973) Proc. Natl. Acad. Sci. U.S.A. 70:3170-3174; Potts & Campbell (1994) Curr. Opin. Cell Biol. 6:648-655; Bork & Doolittle (1992) Natl. Acad. Sci. U.S.A. 89:8990-8994). FNIII domains in polypeptides are quite common, with over 200 polypeptides having these motifs (Potts & Campbell (1994) Curr. Opin. Cell Biol. 6:648-655; Bork & Doolittle (1992) Natl. Acad. Sci. U.S.A. 89:8990-8994). Importantly, they bind to a wide range of different receptors, including fibroblast growth factor receptor and hemojuvelin (Kiselyov et al. (2003) Structure 11:691-701; Yang et al. (2008) Biochemistry 47:4237-4245).

Irisin is a hormone-like molecule secreted from skeletal muscle in response to exercise both in mice and in humans. It is the secreted form of FNDC5 and, in some embodiments, contains 112 amino acids. FNDC5 is a glycosylated type I membrane protein and is released into the circulation after proteolytic cleavage. FNDC5, a PGC-1α-dependent myokine, is cleaved and secreted from muscle during exercise and induces some major metabolic benefits of exercise (Bostrom et al. (2012) Nature 481:463-468). Irisin acts preferentially on the subcutaneous ‘beige’ fat and causes it to ‘brown’ by increasing the expression of UCP-1 and other thermogenic genes (Bostrom et al. (2012) Nature 481:463-468 and Wu et al. (2012) Cell 150:366-376). Clinical studies in humans have confirmed this positive correlation between increased FNDC5 expression and circulating irisin with the level of exercise performance (Huh et al. (2012) Metabolism 61:1725-1738 and Lecker et al. (2012) Circ. Heart Failure 5:812-818). Irisin is found in human blood at concentrations of 3-5 ng/ml (Jedrychowski et al. (2015) Cell Metab. 22:734-740); it has been shown to induce adipose tissue browning when FNDC5 is expressed in the liver through adenoviral vectors, resulting in elevated irisin serum levels (Bostrom et al. (2012) Nature 481:463-468). However, the full range of irisin's effects are just beginning to be explored and, critically, the functioning receptor for irisin has not yet been identified.

Researchers have shown that irisin is involved in bone metabolism by increasing the differentiation of bone marrow stromal cells into mature osteoblasts (Colaianni et al. (2014) Int. J. Endocrinol. 2014:902186). Irisin has also been shown to play a role in the control of bone mass with positive effects on cortical mineral density and geometry in vivo. Recombinant irisin (r-irisin) induced increased cortical BMD, periosteal circumference, and polar moment of inertia in long bones of healthy young mice (Colaianni et al. (2015) Proc. Natl. Acad. Sci. U.S.A. 112:12157-12162). In addition, r-irisin treatment ameliorates disuse-induced osteoporosis and muscle atrophy in hind-limb suspended mice (Colaianni et al. (2017) Sci. Rep. 7:2811). Several recent papers have shown that irisin injections can impact skeletal remodeling. For example, very low dose irisin injections, given intermittently, were shown to improve cortical bone mineral density and strength in mice (Colaianni et al. (2015) Proc. Natl. Acad. Sci. U.S.A. 112:12157-12162; Colaianni et al. (2017) Sci. Rep. 7:2811). These effects were consistent with in vitro studies showing that irisin could enhance osteoblast differentiation (Qiao et al. (2016) Sci. Rep. 6:18732). However, no studies have examined the effects of irisin on the osteocyte, a major regulator of bone structure and function and a cell type critical in the mediation of both mechanical and chemical signals. In addition, the effects of genetic manipulation of FNDC5/irisin on bone have not been reported. The mechanisms underlying irisin-mediated modulation of bone metabolism is not well understood and methods of modulating bone metabolism are currently unknown, such as irisin-based methods for treating loss of bone mass and addressing the increased incidentce of fractures in the elderly or among bedridden patients.

### SUMMARY OF THE INVENTION

The present invention is based, at least in part, on the discovery that irisin activates osteocytes to produce factors that diminish bone mineral content, and loss of irisin/FNDC5 inhibits osteocyte degradative function and pretects bone loss.

In one aspect, a method of preventing and/or treating a subject afflicted with bone loss conditions, comprising administering to the subject a therapeutically effective amount of an agent that decreases the amount and/or activity of irisin, is provided.

Numerous embodiments are further provided that can be applied to any aspect of the present invention described herein. For example, in one embodiment, the agent binds to irisin, or to an irisin receptor in osteocytes, and blocks the binding of irisin to the irisin receptor. In another embodiment, the irisin receptor is an integrin which comprises alpha V subunit, optionally wherein the irisin receptor is alpha V beta 5 (αVβ5)-integrin or αVβ1-integrin. In still another embodiment, the agent is a small molecule inhibitor, peptide or peptidomimetic inhibitor, aptamer, antibody, or intrabody. In yet another embodiment, the agent comprises an antibody and/or intrabody, or an antigen binding fragment thereof, which specifically binds to irisin or the irisin receptor in osteocytes. In another embodiment, the agent comprises an antibody and/or intrabody, or an antigen binding fragment thereof, which specifically binds to irisin, such as an antibody and/or intrabody, or antigen binding fragment thereof, that is murine, chimeric, humanized, composite, or human; and/or an antibody and/or intrabody, or antigen binding fragment thereof, that comprises an effector domain, comprises an Fc domain, and/or is selected from the group consisting of Fv, Fav, F(ab′)2, Fab′, dsFv, scFv, sc(Fv)2, and diabodies fragments. In another embodiment, the agent binds to amino acids 60-76 and/or 101-118 of irisin, or to amino acids 162-174, 196-202, 208-227, and/or 340-346 of integrin β5. In still another embodiment, the agent is a RGD inhibitory peptide, such as RGDS peptide. In yet another embodiment, the agent is a specific inhibitor for integrin αV. Representative specific inhibitors for integrin αV include, for example, echistatin, cyclo RGDyK and SB273005. In another embodiment, the agent decreases the copy number and/or amount of FNDC5, the precursor of irisin, or irisin. In still another embodiment, the agent is a small molecule inhibitor, CRISPR guide RNA (gRNA), RNA interfering agent, antisense oligonucleotide, peptide or peptidomimetic inhibitor, aptamer, antibody, or intrabody. In yet the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA). In another embodiment, the agent comprises an antibody and/or intrabody, or an antigen binding fragment thereof, which specifically binds to FNDC5. In still another embodiment, the antibody and/or intrabody, or antigen binding fragment thereof, is murine, chimeric, humanized, composite, or human. In yet another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments. In another embodiment, the agent inhibits the cleavage of FNDC5 into irisin. In still another embodiment, the agent decreases the copy number, amount and/or activity of the protease that cleaves FNDC5. In yet another embodiment, the agent is a small molecule inhibitor, CRISPR guide RNA (gRNA), RNA interfering agent, antisense oligonucleotide, peptide or peptidomimetic inhibitor, aptamer, antibody, or intrabody. In another embodiment, the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA). In still another embodiment, the agent is a protease inhibitor, such as a DPP4 inhibitor. In yet another embodiment, the agent comprises an antibody and/or intrabody, or an antigen binding fragment thereof, which specifically binds to the protease that cleaves FNDC5, such as an antibody and/or intrabody, or antigen binding fragment thereof, that is murine, chimeric, humanized, composite, or human; and/or an antibody and/or intrabody, or antigen binding fragment thereof, that comprises an effector domain, comprises an Fc domain, and/or is selected from the group consisting of Fv, Fav, F(ab′)2, Fab′, dsFv, scFv, sc(Fv)2, and diabodies fragments.

In another aspect, a method of preventing and/or treating a subject afflicted with bone loss conditions, comprising administering to the subject a therapeutically effective amount of a biologically inactive or inhibitory irisin mutant that binds to the irisin receptor in osteocytes, is provided.

As described above, certain embodiments are applicable to any method described herein. For example, in one embodiment, the irisin receptor is an integrin which comprises alpha V subunit, optionally wherein the irisin receptor is alpha V beta 5 (αVβ5)-integrin or αVβ1-integrin. In another embodiment, the irisin mutant is recombinant or synthetic. In still another embodiment, the agent reduces the irisin-induced signaling. In yet another embodiment, the agent reduces the phosphorylation of FAK, Zyxin, AKT, and/or CREB. In another embodiment, the agent reduces the level of sclerostin and/or RANKL. In still another embodiment, the agent prevents OVX-induced bone resorption and/or bone loss. In yet another embodiment, the agent prevents OVX-induced decrease in the ratio of bone volume to total bone volume, OVX-induced decrease in travecular number, OVX-induced separation between trabeculae in the lumbar vertebrae, OVX-induced increase in osteoclast number and eroded surfaces, and/or OVX-induced perilacunar enlargement. In another embodiment, the agent reduces osteocyte degradative function. In yet another embodiment, the agent prevents trabecular bone loss, osteoclastic bone resorption, and/or osteocytic osteolysis. In still another embodiment, the method further comprises administering one or more agents that reduce bone mineral density loss. In yet another embodiment, the one or more agents that reduce bone mineral density loss are selected from the group consisting of calcium supplements, estrogen, calcitonin, estradiol, diphosphonates, vitamin D3 and/or metabolites thereof, and parathyroid hormone (PTH) and/or deritaves or fragments thereof.

In still another aspect, a method of assessing the efficacy of an agent for treating bone loss conditions in a subject, comprising a) detecting in a subject sample at a first point in time the amount and/or acvitity of irisin; b) repeating step a) during at least one subsequent point in time after administration of the agent; and c) comparing the amount detected in steps a) and b), wherein the absence of, or a significant decrease in amount and/or activity of irisin in the subsequent sample as compared to the amount and/or activity of irisin in the sample at the first point in time, indicates that the agent treats bone loss in the subject, is provided.

As described above, certain embodiments are applicable to any method described herein. For example, in one embodiment, the subject has undergone treatment, completed treatment, and/or is in remission for the bone loss conditions in between the first point in time and the subsequent point in time. In another embodiment, the first and/or at least one subsequent sample is selected from the group consisting of ex vivo and in vivo samples. In still another embodiment, the first and/or at least one subsequent sample is obtained from an animal model of the bone loss condition. In yet another embodiment, the first and/or at least one subsequent sample is a portion of a single sample or pooled samples obtained from the subject. In another embodiment, the sample comprises cells, serum, and/or bone tissue obtained from the subject. In still another embodiment, the method further comprises determining osteocyte function, level of sclerostin and/or RANKL, activation of targets of the irisin receptor, bone mineral volume/total volume, trabecular thickness, trabecular number, eroded bone surface, osteoclast surface, osteoclast number, the separation between trabeculae in the lumbar vertebrae, osteocytic osteolysis, lacunae enlargement, and/or lacunae area. In yet another embodiment, the agent is administered in a pharmaceutically acceptable formulation. In another embodiment, the subject is an animal model of bone loss conditions, such as a mouse model. In still another embodiment, the subject is a mammal, such as a mouse or a human. In yet another embodiment, the bone loss condition is selected from the group consisting of osteopenia, osteoporosis, and cancer, such as multiple myeloma or breast cancer.

In yet another aspect, a cell-based assay for screening for a biologically inactive or inhibitory irisin mutant that binds to the irisin receptor in osteocytes, comprising a) contacting osteocytes with an irisin mutant; b) detecting binding of the test irisin mutant to the isrin receptor; and c) determining the effect of the test irisin mutant on (1) activitation of downstream targets of the irisin receptor; (2) expression level of scleostin and/or RANKL; and/or (3) H₂O₂-induced osteocyte cell death, is provided.

As described above, certain embodiments are applicable to any method described herein. For example, in one embodiment, the step of contacting occurs in vivo, ex vivo, or in vitro. In another embodiment, the irisin receptor is an integrin which comprises alpha V subunit, optionally wherein the irisin receptor is alpha V beta 5 (αVβ5)-integrin or αVβ1-integrin. In still another embodiment, the downstream targets of the irisin receptor comprise pFAK, pZyxin, pAKT, and/or pCREB. In still another embodiment, the method further comprises determining a reduction in the degradative function of the osteocyte cells.

### BRIEF DESCRIPTION OF THE DRAWINGS

FIG. 1A-FIG. 1B show that irisin blocks osteocyte cell death and stimulates sclerostin expression at the mRNA level. FIG. 1A shows the percentage of cell death of MLO-Y4 cells pre-treated with indicated concentration of irisin for 24 hours followed by treatment of 0.3 mM H2O2 with indicated concentration of irisin for 4 more hours. Cells were stained with Hoechst 33342 and Eth-D1, and analyzed to determine the percentage of cell death. *: p<0.05, ***: p<0.001 vs 0.3 mM H2O2 treated condition. FIG. 1B shows sclerostin mRNA level. MLO-Y4 cells were seeded and incubated until 60% cell density. The cells were incubated with Freestyle293 medium for 4 hours and were treated with indicated concentrations of irisin for 16 hours. Sclerostin mRNA level was analyzed by qRT-PCR. Cyclophilin was used as a control house-keeping gene.

FIG. 2A-FIG. 2D show that irisin stimulated a very potent pathway of “integrin-like” signaling including pFAK, pZyxin and pCREB. FIG. 2A shows the scheme of crosslinking/co-immunoprecipitation/mass spectrometry experiments to identify irisin receptors. FIG. 2B shows the model of canonical integrin signaling. Integrin heterodimer binds to its ligand. The interaction results in phosphorylation of FAK and Zyxin, followed by phosphorylation of AKT (at T308) and CREB. PM is plasma membrane. FIG. 2C and FIG. 2D show the immunoblots. MLO-Y4 cells were seeded and incubated until 60% cell density. The cells were incubated with serum free medium (FreeStyle™ 293 medium) for 4 hours and were treated for indicated time with 10 nM norepinephrine or irisin (FIG. 2C) or indicated concentrations of irisin for 10 minutes (FIG. 2D). Cells were lysed to detect the indicated protein level using immunoblot analysis.

FIG. 3 shows that irisin stimulated “integrin-like” signaling in adipose cells. Differentiated 3T3 F442A adipose cells were incubated in serum free medium for 4 hours followed by treatment of indicated concentrations of irisin for 10 minutes. Cells were lysed to detect the indicated protein level using immunoblot analysis.

FIG. 4 shows that irisin bound in vitro to integrins and the binding was blocked by RGDS integrin inhibitor. 100 nM flag-tag irisin was incubated with 5 nM of indicated integrins with his-tag in the presence of RGDS peptide or its control peptide (10 uM). This was followed by immunoprecipitation using anti-his-tag agarose. Co-precipitated irisin was analyzed by immunoblot analysis with antibody against flag tag.

FIG. 5A-FIG. 5E show that irisin-induced signaling and gene expression in osteocytes was reduced by integrin inhibitors such as RGD peptide and echistatin. FIGS. 5A and 5B show the immunoblots. MLO-Y4 were treated and analyzed as FIG. 2D with addition of pre-treatment of integrin inhibitors, 100 nM RGDS (FIG. 5A) or echistatin (FIG. 5B). Cells were lysed to detect the indicated protein level using immunoblot analysis. FIG. 5C shows the mRNA level of sclerostin. MLO-Y4 cells were treated as described in FIG. 1B except with addition of the pretreatment of integrin inhibitors for 10 minutes. FIGS. 1D and 1E show mRNA and protein levels of sclerostin. Mice were treated as described in FIGS. 1C-1D except co-injection of 1 mg/kg cyclo RGDyK (cRGDyK). Data are represented as mean±SEM. For FIG. 5C and FIG. 5D, n=9-12 animals/group. *: p<0.05.

FIG. 6A-FIG. 6B show that irisin treatment stimulated sclerostin expression at the mRNA in MLO-Y4 cells and integrin inhibitors prevent the stimulation. MLO-Y4 cells were incubated in serum free medium for 3 hours followed by treatment of indicated concentrations of irisin for indicated time (FIG. 6A), or followed by treatment of 0 or 10 nM irisin in the presence of indicated integrin inhibitors or their control peptide for 16 hours (FIG. 6B). Sclerostin mRNA level was analyzed by qPCR.

FIG. 7A-FIG. 7B show that low dose irisin injections in vivo stimulated sclerostin expression at the mRNA (FIG. 7A) and circulating protein (FIG. 7B) level. 8 weeks old mice were injected daily with indicated dose of irisin for 6 days. Femurs were collected and treated with collagenase to yield osteocyte-enriched bones. Sclerostin mRNA level from osteocyte-enriched tibia was analyzed by qPCR (FIG. 7A). Cyclophilin was used as a house-keeping gene. Plasma was collected to analyze the circulating sclerostin protein level by ELISA assay (FIG. 7B). Data are represented as mean±SEM. For FIG. 7A and FIG. 7B, n=5 animals/group. * indicates p<0.05, and *** indicates p<0.001.

FIG. 8 shows that low dose irisin injections stimulated the classical adipose thermogenic pathway and genes of the futile creatine cycle. Eight week old mice were injected with indicated dose of irisin or 1 mg/kg CL316243 for 6 days. Epididymal fats (eWAT) were collected and indicated genes mRNA level was analyzed by qPCR. GATM is the first and rate-limiting step of creatine synthesis (Sandell et al. (2003) Proc. Natl. Acad. Sci. U.S.A. 100:4622-4627).

FIG. 9A-FIG. 9J show that irisin/FNDC5 global KO mice were resistant to OVX-induced trabecular bone loss at 9-months of age. Ovariectomy (OVX) was performed on 9 months old wild-type mice (WT) and global FNDC5 knockout mice (FNDC KO) followed by collection of lumbar vertebra and tibia after 3 weeks. FIGS. 9A-9D show the representative figures of Von Kossa stained lumbar vertebra from wild-type mice or FNDC5/irisin knockout mice after OVX. Mineralized bone was stained black. Arrow indicates mineralized bone. FIGS. 9E-9J show the bone histomorphometric analysis was performed in the lumbar vertebra. Data are represented as mean±SEM. N=4-7 animals/group. See also Tables 8 and 9. *: p<0.05.

FIG. 10A-FIG. 10E show that deletion of irisin/FNDC5 prevented OVX-induced osteoclastic bone resorption and osteocytic osteolysis at 9-months of age. Mice were treated and analyzed as FIG. 9A-FIG. 9J. Tibia samples from FIGS. 9A-9J were analyzed to measure lacunae area using backscatter scanning electron microscopy. FIGS. 3A-3D show the representative figures. Arrow indicates lacunae. FIG. 10E shows the analyzed lacunae area. The osteocyte lacunae analysis was performed in tibia. Data are represented as mean±SEM. n=4-7 animals/group. See also Tables 10 and 11. *: p<0.05.

FIG. 11 shows that the half-life of recombinant his-tag irisin in vivo is less than an hour. C57BL/6 mice were injected with irisin (1 mg/kg, I.P.) or sterilized PBS and blood was collected at indicated time point. Irisin in plasma was detected using immunoblot analysis against his-tag.

FIG. 12A-FIG. 12G show gene expression analysis and quantification of irisin in plasma after OVX. FIGS. 12A-12C show mRNA levels of sclerostin, RANKL and OPG. (OVX) was performed on 5 months old wild-type mice (WT) and global FNDC5/irisin knockout mice (FNDC KO). RNA was extracted from whole bone tibia including bone marrow. Indicated mRNA levels were analyzed by qRT-PCR. Cyclophilin was used for house-keeping gene. FIGS. 12D-12F show mRNA levels of sclerostin, RANKL and OPG. RNA was extracted from whole bone tibia without bone marrow. Indicated mRNA levels were analyzed by qRT-PCR. Cyclophilin was used as a control house-keeping gene. FIG. 12G shows irisin level in plasma. (OVX) was performed on 8 weeks old wild-type C57BL/6 mice and plasma was collected and irisin was quantified by quantitative proteomics. 4 mice per group.

FIG. 13A-FIG. 13D show the Von Kossa staining of vertebrae demonstrates deletion of FNDC5 prevented ovariectomy-induced trabecular bone loss. Figures of Von Kossa staining of vertebrae from mice in FIG. 9. FIG. 13A shows the Von Kossa staining of vertebrae from sham operated wild-type group. FIG. 13B shows the Von Kossa staining of vertebrae from OVX'd wild-type group. FIG. 13C shows the Von Kossa staining of vertebrae from sham operated FNDC5 KO group. FIG. 13D shows the Von Kossa staining of vertebrae from OVX'd FNDC5 KO group.

FIG. 14A-FIG. 14E show that irisin directly interacts with integrin complexes and mapping of binding motifs. FIG. 14A shows the immunoblot data. 100 nM irisin was incubated with 5 nM indicated his-tag integrins followed by immunoprecipitation using Ni-NTA agaroses. Precipitated integrins and co-precipitated irisin were analyzed by immunoblot analysis. FIG. 14B shows the immunoblot data. HEK293T cells were seeded and incubated until 50% cell density. The cells were transfected with 0.1 μg plasmids of indicated integrins. After 48 hours, the cells were incubated with Freestyle293 medium for 3 hours and were treated with indicated concentration of irisin for 5 minutes. Cells were lysed to detect the indicated protein level using immunoblot analysis. FIG. 14C shows the immunoblot data. MLO-Y4 cells were treated as described in FIG. 2D with addition of pretreatment of indicated antagonistic antibodies for 10 minutes. Cells were lysed to detect the indicated protein level using immunoblot analysis. FIG. 14D shows the mRNA level of sclerostin. MLO-Y4 cells were treated as described in FIG. 1B except with addition of the pretreatment of indicated antagonistic antibodies for 10 minutes. Sclerostin mRNA level was analyzed by qRT-PCR. Cyclophilin was used for house-keeping gene. FIG. 14E shows the docking model of interaction between irisin and integrin αV/β5 (see Example 1). The ribbon diagram is colored by HDX stabilization/destabilization. Percentages of deuterium differences are color-coded according to the smooth color gradient key at the bottom. Crystal structure of irisin dimer is from Protein Data Bank (PDB) (4 lsd) and a homology model of integrin β5 was built based on integrin β3 structure from PDB (4MMX).

FIG. 15A-FIG. 15D show that irisin binds via integrin αV. FIG. 15A shows the immunoblot data. 100 nM irisin was incubated with 5 nM indicated his-tag integrins followed by immunoprecipitation using Ni-NTA agaroses. Precipitated integrins and co-precipitated irisin were analyzed by immunoblot analysis. FIG. 15B shows the immunoblot data. HEK293T cells were seeded and incubated until 50% cell density. The cells were transfected with 0.1 μg plasmids of indicated integrins. After 48 hours, the cells were re-split to indicated dose of vitronectin-coated plates. Cells were incubated with culture medium for 3 hours. Cells were lysed to detect the indicated protein level using immunoblot analysis. FIG. 15C and FIG. 15D show the immunoblot data. Cells were treated and analyzed as FIG. 14B except using plasmids encoding integrin α5/β1 or integrin α11/β1 (FIG. 15C) integrin αV/β1(FIG. 15D).

FIG. 16A-FIG. 16E show that single amino acid consolidated differential HDX map of integrin αV/β5: irisin complex. FIGS. 16A and 16B show the differential HDX map of integrin β5 (FIG. 16A) and irisin (FIG. 16B). The amino acid sequences are colored by HDX stabilization/destabilization. Percentages of deuterium differences are colorcoded according to the smooth color gradient key at the bottom. FIGS. 16C-16E show the average percent change of deuteration of the indicated peptides in irisin. Red line is apo-form and blue line is integrin bound form. *: p<0.05; **: p<0.01; ***: p<0.001.

FIG. 17A-FIG. 17C show that integrin αV specific inhibitors block irisin-induced signaling and gene expression. FIG. 17A shows the immunoblot data. MLO-Y4 cells were treated and analyzed as described in FIG. 5A with addition of pretreatment of control RGD peptide, cyclo RGDyK (cRGDyK), or SB273005. FIG. 17B shows the immunoblot data. HEK293 cells were treated and analyzed as described in FIG. 14B except the treatment of different dose of cyclo RGDyK. FIG. 17C shows the mRNA level of sclerostin. 8 weeks old male mice were treated and analyzed as described in FIG. 5D except the additional group with co-injection of 1 mg/kg SB273005.

FIG. 18A-FIG. 18E show that integrin mediates irisin-induced thermogenesis. FIG. 18A-FIG. 18B show mRNA and protein levels of indicated genes. 1 mg/kg irisin was injected to 8-week old mice every other day for 6 days. mRNA levels of indicated genes in inguinal fat were analyzed by qRT-PCR. Cyclophilin was used for housekeeping gene (FIG. 18A). Inguinal fats were also lysed to detect the indicated protein level using immunoblot analysis (FIG. 18B). FIGS. 18C and 18D show mRNA and protein levels of indicated genes. Mice were treated and analyzed as (A-B) with addition of coinjection of 1 m/kg control RGD peptide or cyclo RGDyK (cRGDyK). mRNA levels of indicated genes in inguinal fat were analyzed by qRT-PCR. Cyclophilin was used for house-keeping gene (FIG. 18C). Inguinal fats were also lysed to detect the indicated protein level using immunoblot analysis (FIG. 18D). FIG. 18E shows mRNA level of Ucp1. Primary inguinal fat cells were treated with indicated concentration of irisin with 10 μM control peptide or cyclo RGDyK (cRGDyK) every other day during 6 days differentiation. Ucp1 mRNA level was analyzed by qRT-PCR. Cyclophilin was used as a control house-keeping gene. Data are represented as mean±SEM. For FIG. 18A and FIG. 18B, n=12-13 animals/group. For FIG. 18C and FIG. 18D, n=11-13 animals/group. *: p<0.05; **: p<0.01.

For any figure showing a bar histogram, curve, or other data associated with a legend, the bars, curve, or other data presented from left to right for each indication correspond directly and in order to the boxes from top to bottom, or from left to right, of the legend.

### DETAILED DESCRIPTION OF THE INVENTION

It has been determined herein that irisin activates osteocytes to produce factors that diminish bone mineral content, and loss of irisin/FNDC5 inhibits osteocyte degradative function and protects bone loss. For example, osteocytes stimulated by irisin were determined to survive and secrete bone mobilizing hormones, especially sclerostin. Mice engineered to knockout FNDC5 were determined to completely resist osteoporosis as a consequence of ovariectomy, the most common model of experimental osteoporosis. Accordingly, the present invention relates, in part, to methods for preventing and/or treating a subject afflicted with bone loss conditions, such as by administering to the subject a therapeutically effective amount of an agent that decreases the amount and/or activity of irisin, or by administering to the subject a therapeutically effective amount of a biologically inactive or inhibitory irisin mutant that binds to the irisin receptor in osteocytes.

In order that the present invention may be more readily understood, certain terms are first defined. Additional definitions are set forth throughout the detailed description.

### I. Definitions

The articles “a” and “an” are used herein to refer to one or to more than one (i.e. to at least one) of the grammatical object of the article. By way of example, “an element” means one element or more than one element.

The term “altered amount” or “altered level” refers to increased or decreased copy number (e.g., germline and/or somatic) of a biomarker nucleic acid, e.g., increased or decreased expression level in a bone loss condition sample, as compared to the expression level or copy number of the biomarker nucleic acid in a control sample. The term “altered amount” of a biomarker also includes an increased or decreased protein level of a biomarker protein in a sample, e.g., a bone loss condition sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from bone loss conditions, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a bone loss condition sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies of the present invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the present invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

The term “assigned score” refers to the numerical value designated for each of the biomarkers after being measured in a patient sample. The assigned score correlates to the absence, presence or inferred amount of the biomarker in the sample. The assigned score can be generated manually (e.g., by visual inspection) or with the aid of instrumentation for image acquisition and analysis. In certain embodiments, the assigned score is determined by a qualitative assessment, for example, detection of a fluorescent readout on a graded scale, or quantitative assessment. In one embodiment, an “aggregate score,” which refers to the combination of assigned scores from a plurality of measured biomarkers, is determined. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score.”

The term “biomarker” refers to a measurable entity of the present invention that has been determined to be predictive of an irisin-based therapy effects on a bone loss condition. Biomarkers can include, without limitation, nucleic acids and proteins, including those shown in the Tables, the Examples, the Figures, and otherwise described herein (e.g., irisin, FNDC5, irisin receptor, or protease that cleaves FNDC5 into irisin). As described herein, any relevant characteristic of a biomarker can be used, such as the copy number, amount, activity, location, modification (e.g., phosphorylation), and the like.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s). Blocking antibodies of FNDC5/irisin, as well as non-activating forms of FNDC5/irisin, are contemplated as agents useful in inhibiting FNDC5/irisin.

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The three major types of bone cells are osteocytes, osteoblasts and osteoclasts. Osteocytes are the most abundant cell type in bone (Nijweide et al. (1996) Principles of Bone Biology (Bilezikian, Riasz and Rodan, eds.), Academic Press, New York, N.Y., pp. 115-126), with approximately ten times more osteocytes than osteoblasts (Parfitt et al. (1977) Clin. Orthop. Rel. Res. 127:236-247), and with osteoblasts far more abundant than osteoclasts. Each of these different types of bone cell has a different phenotype, morphology and function. Osteocytes are localized within the mineral matrix at regular intervals, and arise from osteoblasts. During their transition from osteoblasts, osteocytes maintain certain osteoblastic features, but acquire several osteocyte-specific characteristics. Mature osteocytes are stellate shaped or dendritic cells enclosed within the lacuno-canalicular network of bone. Long, slender cytoplasmic processes radiate from the central cell body, with most of the processes perpendicular to the bone surface. The processes connect the osteocyte to neighboring osteocytes and to the cells lining the bone surface. The functions of osteocytes include: to respond to mechanical strain and to send signals of bone formation or bone resorption to the bone surface, to modify their microenvironment, and to regulate both local and systemic mineral homeostasis. Increasing evidence indicates that osteocytes may regulate physiological local bone remodeling, in part through their cell death and apoptosis that trigger osteoclasts formation and bone resorption, and in part by secreting sclerostin, a molecule specifically produced by osteocytes that acts as an inhibitor of bone formation (Giuliani et al. (2015) in Bone Cancer (Second Edition), Chapter 42, pp 491-500). Osteoblasts are the skeletal cells responsible for bone formation, and thus synthesize and regulate the deposition and mineralization of the extracellular matrix of bone (Aubin and Liu, (1996) Principles of Bone Biology (Bilezikian, Riasz and Rodan, eds.), Academic Press, New York, N.Y., pp. 51-67). Osteoclasts are multinucleated giant cells with resorbing activity of mineralized bone (Suda et al., (1996) Principles of Bone Biology (Bilezikian, Riasz and Rodan, eds.), Academic Press, New York, N.Y., pp. 87-102).

The term “bone loss condition” refers to a condition that occurs when the body doesn't make new bone as quickly as it reabsorbs old bone. In one embodiment, “bone loss conditions” include bone diseases, such as osteopenia, osteoporosis, osteoplasia (osteomalacia), and Paget's disease of bone. In another embodiment, “bone loss conditions” include other diseases, such as diabetes, chronic renal failure, hyperparathyroidism, and cancer (e.g., multiple myeloma and breast cancer), which result in abnormal or excessive bone loss. The present invention is directed to methods of treating and/or preventing bone loss conditions, such as osteoporosis and osteopenia and other diseases where inhibiting bone loss may be beneficial, including Paget's disease, malignant hypercalcemia, periodontal disease, joint loosening and metastatic bone disease, as well as reducing the risk of fractures, both vertebral and nonvertebral.

Osteopenia refers to bone density that is lower than normal density but not low enough to be classified as osteoporosis. Osteopenia is reduced bone mass due to a decrease in the rate of osteoid synthesis to a level insufficient to compensate normal bone lysis. Osteopenia is commonly seen in people over age 50 that have lower than average bone density but do not have osteoporosis.

Osteoporosis is a structural deterioration of the skeleton caused by loss of bone mass resulting from an imbalance in bone formation, bone resorption, or both, such that the resorption dominates the bone formation phase, thereby reducing the weight-bearing capacity of the affected bone. In a healthy adult, the rate at which bone is formed and resorbed is tightly coordinated so as to maintain the renewal of skeletal bone. However, in osteoporotic individuals an imbalance in these bone remodeling cycles develops which results in both loss of bone mass and in formation of microarchitectural defects in the continuity of the skeleton. These skeletal defects, created by perturbation in the remodeling sequence, accumulate and finally reach a point at which the structural integrity of the skeleton is severely compromised and bone fracture is likely. Although this imbalance occurs gradually in most individuals as they age (“senile osteoporosis”), it is much more severe and occurs at a rapid rate in postmenopausal women. In addition, osteoporosis also may result from nutritional and endocrine imbalances, hereditary disorders and a number of malignant transformations.

Bone loss is also an important consideration for treatment among cancers, particularly among multiple myeloma and breast cancer.

Current treatments for osteoporosis or osteopenia are based on inhibiting further bone resorption, e.g., by 1) inhibiting the differentiation of hemopoietic mononuclear cells into mature osteoclasts, 2) by directly preventing osteoclast-mediated bone resorption, or 3) by affecting the hormonal control of bone resorption. Drug regimens used for the treatment of osteoporosis include calcium supplements, estrogen, calcitonin, estradiol, and diphosphonates. Vitamin D3 and its metabolites, known to enhance calcium and phosphate absorption, can also be used. Similarly, parathyroid hormone (PTH, such as the 84-amino acid PTH peptide or fragments thereof, such as the teriparatide first 1-34 amino acids of human PTH, can also be used (see, for example, U.S. Pat. Publ. 2018/0028622 and U.S. Pat. No. 8,110,547, each of which is incorporated in their entirety herein by this reference).

Osteoplasia, also known as osteomalacia (“soft bones”), is a defect in bone mineralization (e.g., incomplete mineralization), and classically is related to vitamin D deficiency (1,25-dihydroxy vitamin D3). The defect can cause compression fractures in bone, and a decrease in bone mass, as well as extended zones of hypertrophy and proliferative cartilage in place of bone tissue. The deficiency may result from a nutritional deficiency (e.g., rickets in children), malabsorption of vitamin D or calcium, and/or impaired metabolism of the vitamin.

Paget's disease (osteitis deformans) is a disorder currently thought to have a viral etiology and is characterized by excessive bone resorption at localized sites which flare and heal but which ultimately are chronic and progressive, and may lead to malignant transformation. The disease typically affects adults over the age of twenty five years old.

Patients suffering from chronic renal (kidney) failure almost universally suffer loss of skeletal bone mass (renal osteodystrophy). While it is known that kidney malfunction causes a calcium and phosphate imbalance in the blood, to date replenishment of calcium and phosphate by dialysis does not significantly inhibit osteodystrophy in patients suffering from chronic renal failure. In adults, osteodystrophic symptoms often are a significant cause of morbidity. In children, renal failure often results in a failure to grow, due to the failure to maintain and/or to increase bone mass.

Hyperparathyroidism (overproduction of the parathyroid hormone) is known to cause malabsorption of calcium, leading to abnormal bone loss. In children, hyperparathyroidism can inhibit growth, in adults the skeleton integrity is compromised and fracture of the ribs and vertebrae are characteristic. The parathyroid hormone imbalance typically may result from thyroid adenomas or gland hyperplasia, or may result from prolonged pharmacological use of a steroid. Secondary hyperparathyroidism also may result from renal osteodystrophy. In the early stages of the disease osteoclasts are stimulated to resorb bone in response to the excess hormone present. As the disease progresses, the trabecular bone ultimately is resorbed and marrow is replaced with fibrosis, macrophages and areas of hemorrhage as a consequence of microfractures. This condition is referred to clinically as osteitis fibrosa.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. Unless otherwise stated, the terms include metaplasias. Cancer is a major risk factor for both generalized and local bone loss, with bone loss in cancer patients substantially greater than in the general population. Cancer-associated bone loss is due to the direct effects of cancer cells and the effects of therapies used in cancer treatment, including chemotherapeutics, corticosteroids, aromatase inhibitors and androgen deprivation therapy (ADT).

In one embodiment, the cancer is multiple myeloma. Multiple myeloma is the second most common hematologic cancer, accounting for 10 percent of all hematologic cancers. Patients have both generalized bone loss and focal osteolytic lesions. Nearly two-thirds of patients with multiple myeloma have bone pain at presentation, and fracture rates are increased 16-fold relative to the general population in the year preceding diagnosis. Even with disease remission, skeletal lesions rarely heal. Both pamidronate and zoledronate are approved by the Food and Drug Administration for the treatment of multiple myeloma-related bone disease and have been shown in placebo-controlled trials to reduce hypercalcemia, bone pain and fracture incidence. In another embodiment, the cancer is breast cancer.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control bone loss condition patient (can be stored sample or previous sample measurement) with a known outcome; normal tissue or cells isolated from a subject, such as a normal patient or the bone loss condition patient, cultured primary cells/tissues isolated from a subject such as a normal subject or the bone loss condition patient, adjacent normal cells/tissues obtained from the same organ or body location of the bone loss condition patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care bone loss condition therapy). It will be understood by those of skill in the art that such control samples and reference standard expression product levels can be used in combination as controls in the methods of the present invention.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a biomarker nucleic acid or “normal” level of expression of a biomarker nucleic acid or protein is the activity/level of expression or copy number in a biological sample from a subject, e.g., a human, not afflicted with bone loss conditions, or from a corresponding non-bone tissue in the same subject who has bone loss conditions.

The term “determining a suitable treatment regimen for the subject” is taken to mean the determination of a treatment regimen (i.e., a single therapy or a combination of different therapies that are used for the prevention and/or treatment of the bone loss in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

The term “expression signature” or “signature” refers to a group of one or more coordinately expressed biomarkers related to a measured phenotype. For example, the genes, proteins, metabolites, and the like making up this signature may be expressed in a specific cell lineage, stage of differentiation, or during a particular biological response. Expression data and gene expression levels can be stored on computer readable media, e.g., the computer readable medium used in conjunction with a microarray or chip reading device. Such expression data can be manipulated to generate expression signatures.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the terms “Fndc5” and “Frcp2” refer to fibronectin type III domain containing 5 protein and are intended to include fragments, variants (e.g., allelic variants) and derivatives thereof. Representative, non-limiting examples of Fndc5 sequences, and variants and fragments thereof, are shown in Table 1. For example. the nucleotide and amino acid sequences of mouse Fndc5, which correspond to Genbank Accession number NM_027402.4 and NP_081678.1 respectively, are set forth in SEQ ID NOs: 1 and 2. At least three splice variants encoding distinct human Fndc5 isoforms exist (isoform 1, NM_001171941.2 and NP_001165412.1; isoform 2, NM_153756.2 and NP_715637.2; and isoform 3, NM_001171940.1 and NP_001165411.2). The nucleic acid and polypeptide sequences for each isoform is provided herein as SEQ ID NOs: 3-8, respectively. Nucleic acid and polypeptide sequences of FNDC5 orthologs in organisms other than mice and human are well known and include, for example, monkey FNDC5 (XM_015134578.1 and XP_014990064.1; XM_015134578.1 and XP_014990064.1; XM_015134578.1 and XP_014990064.1), dog FNDC5 (XM_022411872.1 and XP_022267580.1; XM_014109741.2 and XP_013965216.1; XM_014109742.1 and XP_013965217.1), rat FNDC5 (NM_001270981.1 and NP_001257910.1), chicken FNDC5 (NM_001318986.1 and NP_001305915.1), zebrafish FNDC5b (NM_001044337.1 and NP_001037802.1), and zebrafish FNDC5a (XM_021480899.1 and XP_021336574.1). In addition, numerous anti-FNDC5 antibodies having a variety of characterized specificities and suitabilities for various immunochemical assays are commercially available and well known in the art, including antibody LS-C166197 from Lifespan Biosciences, antibodies AG-25B-0027 and -0027B from Adipogen, antibody HPA051290 from Atlas Antibodies, antibodies PAN576Hu71 and Hu01 and Hu02 and Mu01 from Uscn Lifesciences, antibody AP18024PU-N from Acris Antibodies, antibody OAAB05345 from Aviva Systems Biology, antibody CPBT-33932RH from Creative Biomart, antibody orb39441 from Biorbyt, antibody ab93373 from Abcam, antibody NBP2-14024 from Novus Biologicals, antibody F4216-25 from United States Biological, antibody AP8746b from Abgent, and the like.

In some embodiments, fragments of Fndc5 having one or more biological activities of the full-length Fndc5 protein are described and employed. Such fragments can comprise or consist of at least one fibronectin domain of an Fndc5 protein without containing the full-length Fndc5 protein sequence. In some embodiments, Fndc5 fragments can comprise or consist of a signal peptide, extracellular, fibronectin, hydrophobic, and/or C-terminal domains of an Fndc5 protein without containing the full-length Fndc5 protein sequence. As further indicated in the Examples, Fndc5 orthologs are highly homologous and retain common structural domains well-known in the art.

Irisin is a secreted form of FNDC5, which is generated by proteolytic cleavage and released into the circulation (Bostrom et al. (2012) Nature 481:463-468). Irisin has been crystallized and its structure has been solved (Schumacher et al. (2013) J Bio. Chem. 288: 33738-33744). Subsequent biochemical experiments confirmed the existence of irisin (bacterial recombinant) as a homodimer. Irisin induces trans-differentiation of the white adipocytes into brown (Hu et al. (2012) Metabolism 61:1725-1738). FNDC5 or irisin also potently increases energy expenditure, reduces body weight and alleviates diabetes. Irisin is induced with exercise in both mouse and man, and increased irisin blood levels cause an increase in energy expenditure, which results in improvement in metabolic disorders (e.g., obesity, insulin resistance, and glucose homeostasis; see, for example, U.S. Pat. Appl. No. 20130074199). Other studies revealed the role of FNDC5 or irisin in the nervous system (Wrann et al. (2015) Brain Plast. 1:55-61). For example, cerebellar purkinje cells of rat and mouse express irisin, whose function would be to induce the neuronal differentiation of embryonic stem cells of mouse. Irisin is also activated by exercise in the hippocampus in mice and induces a neuroprotective gene program, including Bdnf. It is also known that the energetic depletion, peculiar to myocardial infarction, negatively affects the circulating concentration of irisin, indicating a negative association of this myokine with infarction. Other known uses are, for example, the use of irisin in inducing the oxidation of fatty acids and mitochondrial biogenesis, as well as its use to prevent the damage by post-ischemic reperfusion after infarction. Further, irisin exerts an anabolic action on bone tissue, e.g., it induces differentiation of bone marrow stromal cells into mature osteoblasts (Colaianni et al. (2014) Int. J. Endocrinol. 2014:902186), plays a role in the control of bone mass with positive effects on cortical mineral density and geometry in vivo (Colaianni et al. (2015) Proc. Natl. Acad. Sci. U.S.A. 112:12157-12162), and ameliorates disuse-induced osteoporosis and muscle atrophy in hind-limb suspended mice (Colaianni et al. (2017) Sci. Rep. 7:2811). Additional examples of uses of irisin are described in PCT Publication No. WO 2016081603, US Publication No. 2016/0256522, US Publication No. 2017/0028018, US Publication No. 2016/0213753, which are incorporated herein by reference.

In some embodiments, the term “irisin” refers to the fragment representing residues 29 to 140, 30 to 140, or 73-140 of SEQ ID NO: 2 or the corresponding residues in an FNDC5 ortholog thereof. In other embodiments, irisin or an FNDC5 molecule useful herein is encoded by an isolated nucleic acid molecule, such as one selected from the group consisting of: a) an isolated nucleic acid molecule which encodes at least one fibronectin domain of an Fndc5 protein and which does not encode full-length Fndc5; b) an isolated nucleic acid molecule which encodes at least one fibronectin domain of an Fndc5 protein and which does not encode one or more functional domain(s) of an Fndc5 protein selected from the group consisting of signal peptide, hydrophobic, and C-terminal domains; c) an isolated nucleic acid molecule which encodes a polypeptide comprising an amino acid sequence having at least 70% identity to the amino acid sequence of residues 73-140 of SEQ ID NO:2, 30-140 of SEQ ID NO:2 or 29-140 of SEQ ID NO:2 and which does not encode one or more functional domain(s) of an Fndc5 protein selected from the group consisting of signal peptide, hydrophobic, and C-terminal domains; d) an isolated nucleic acid molecule which encodes a polypeptide comprising an amino acid sequence having at least 70% identity to the amino acid sequence of residues 73-140 of SEQ ID NO:2, 30-140 of SEQ ID NO:2 or 29-140 of SEQ ID NO:2 and which is less than 630 nucleotides in length; e) an isolated nucleic acid molecule which encodes a polypeptide consisting essentially of an amino acid sequence having at least 70% identity to the amino acid sequence of residues 73-140 of SEQ ID NO:2, 30-140 of SEQ ID NO:2 or 29-140 of SEQ ID NO:2; f) an isolated nucleic acid molecule which encodes a polypeptide comprising an amino acid sequence having at least 70% identity to the amino acid sequence of residues 73-140 of SEQ ID NO:2, 30-140 of SEQ ID NO:2 or 29-140 of SEQ ID NO:2 and which does not encode the full-length amino acid sequence of SEQ ID NO:2; g) an isolated nucleic acid molecule which encodes a polypeptide comprising the amino acid sequence of residues 73-140 of SEQ ID NO:2, 30-140 of SEQ ID NO:2 or 29-140 of SEQ ID NO:2 and which does not encode the full-length amino acid sequence of SEQ ID NO:2; h) an isolated nucleic acid molecule which encodes a polypeptide consisting essentially of the amino acid sequence of residues 73-140 of SEQ ID NO:2, 30-140 of SEQ ID NO:2 or 29-140 of SEQ ID NO:2; i) an isolated nucleic acid molecule comprising a nucleotide sequence which is at least 70% identical to the nucleotide sequence of nucleotides 217-420 of SEQ ID NO:1, 88-420 of SEQ ID NO:1 or 85-420 of SEQ ID NO:1 and which does not encode one or more functional domain(s) of an Fndc5 protein selected from the group consisting of signal peptide, hydrophobic, and C-terminal domains; and an isolated nucleic acid molecule consisting essentially of a nucleotide sequence which is at least 70% identical to the nucleotide sequence of nucleotides 217-420 of SEQ ID NO:1, 88-420 of SEQ ID NO:1 or 85-420 of SEQ ID NO:1. In some embodiments, an isolated nucleic acid molecule comprising a nucleotide sequence is provided which is complementary to a nucleic acid sequence described herein. In still other embodiments, isolated nucleic acid molecules described herein further comprise a nucleic acid sequence encoding a heterologous polypeptide (e.g., selected from the group consisting of a signal peptide, a peptide tag, a dimerization domain, an oligomerization domain, an antibody, or an antibody fragment). In addition, it is contemplated that such polypeptides are inclusive of nucleic acid and polypeptide molecules encompassing the corresponding nucleotides and residues in an FNDC5 ortholog of SEQ ID NOs: 1 and 2, such as human FNDC5 nucleic acid and polypeptide sequences (see, for example, sequence provided in Table 1).

Similarly, in some embodiments, irisin or an FNDC5 molecule useful herein also encompasses polypeptides selected from the group consisting of: a) an isolated polypeptide fragment of an Fndc5 protein comprising at least one fibronectin domain and is not full-length Fndc5; b) an isolated polypeptide fragment of an Fndc5 protein comprising at least one fibronectin domain and which lacks one or more functional domain(s) selected from the group consisting of signal peptide, hydrophobic, and C-terminal domains; c) an isolated polypeptide comprising an amino acid sequence that is at least 70% identity to the amino acid sequence comprising residues 73-140 of SEQ ID NO:2, 30-140 of SEQ ID NO:2 or 29-140 of SEQ ID NO:2 and which lacks one or more functional domain(s) of an Fndc5 protein selected from the group consisting of signal peptide, hydrophobic, and C-terminal domains; d) an isolated polypeptide comprising an amino acid sequence that is at least 70% identity to the amino acid sequence comprising residues 73-140 of SEQ ID NO:2, 30-140 of SEQ ID NO:2 or 29-140 of SEQ ID NO:2 and which is less than 195 amino acids in length; e) an isolated polypeptide consisting essentially of an amino acid sequence that is at least 70% identity to the amino acid sequence comprising residues 73-140 of SEQ ID

NO:2, 30-140 of SEQ ID NO:2 or 29-140 of SEQ ID NO:2; f) an isolated polypeptide fragment of SEQ ID NO:2 comprising residues 73-140 of SEQ ID NO:2, 30-140 of SEQ ID NO:2 or 29-140 of SEQ ID NO:2 and which is not full-length; g) an isolated polypeptide fragment of SEQ ID NO:2 consisting essentially of residues 73-140 of SEQ ID NO:2, 30-140 of SEQ ID NO:2 or 29-140 of SEQ ID NO:2; h) an isolated polypeptide which is encoded by a nucleic acid molecule comprising a nucleotide sequence encoding at least one fibronectin domain of an Fndc5 protein and does not encode full-length Fndc5; i) an isolated polypeptide fragment of an Fndc5 protein which is encoded by a nucleic acid molecule comprising a nucleotide sequence encoding at least one fibronectin domain and which does not encode one or more functional domain(s) selected from the group consisting of signal peptide, hydrophobic, and C-terminal domains; j) an isolated polypeptide which is encoded by a nucleic acid molecule comprising a nucleotide sequence encoding an amino acid sequence that is at least 70% identical to the amino acid sequence of residues 73-140 of SEQ ID NO:2, 30-140 of SEQ ID NO:2 or 29-140 of SEQ ID NO:2 and which does not encode one or more functional domain(s) of an Fndc5 protein selected from the group consisting of signal peptide, hydrophobic, and C-terminal domains; k) an isolated polypeptide which is encoded by a nucleic acid molecule comprising a nucleotide sequence encoding an amino acid sequence that is at least 70% identical to the amino acid sequence of residues 73-140 of SEQ ID NO:2, 30-140 of SEQ ID NO:2 or 29-140 of SEQ ID NO:2 and which is less than 630 nucleotides in length; l) an isolated polypeptide which is encoded by a nucleic acid molecule consisting essentially of a nucleotide sequence encoding an amino acid sequence having at least 70% identity to the amino acid sequence of residues 73-140 of SEQ ID NO:2, 30-140 of SEQ ID NO:2 or 29-140 of SEQ ID NO:2; m) an isolated polypeptide which is encoded by a nucleic acid molecule comprising a nucleotide sequence encoding an amino acid sequence that is at least 70% identical to the amino acid sequence of residues 73-140 of SEQ ID NO:2, 30-140 of SEQ ID NO:2 or 29-140 of SEQ ID NO:2 and which does not encode the full-length amino acid sequence of SEQ ID NO:2; n) an isolated polypeptide which is encoded by a nucleic acid molecule comprising a nucleotide sequence encoding the amino acid sequence of residues 73-140 of SEQ ID NO:2, 30-140 of SEQ ID NO:2 or 29-140 of SEQ ID NO:2 and which does not encode the full-length amino acid sequence of SEQ ID NO:2; o) an isolated polypeptide which is encoded by a nucleic acid molecule consisting essentially of a nucleotide sequence encoding the amino acid sequence of residues 73-140 of SEQ ID NO:2, 30-140 of SEQ ID NO:2 or 29-140 of SEQ ID NO:2; p) an isolated polypeptide which is encoded by a nucleic acid molecule comprising a nucleotide sequence which is at least 70% identical to the nucleotide sequence of nucleotides 217-420 of SEQ ID NO:1, residues 88-420 of SEQ ID NO:1, or residues 85-420 of SEQ ID NO:1 and which does not encode one or more functional domain(s) of an Fndc5 protein selected from the group consisting of signal peptide, hydrophobic, and C-terminal domains; and q) an isolated polypeptide which is encoded by a nucleic acid molecule consisting essentially of a nucleotide sequence which is at least 70% identical to the nucleotide sequence of nucleotides 217-420 of SEQ ID NO:1, residues 88-420 of SEQ ID NO:1, or residues 85-420 of SEQ ID NO:1. In some embodiments, the isolated polypeptide maintains the ability to promote one or more biological activities selected from the group consisting of: a) expression of a marker selected from the group consisting of: cidea, adiponectin, adipsin, otopetrin, type II deiodinase, cig30, ppar gamma 2, pgc1α, ucp1, elovl3, cAMP, Prdm16, cytochrome C, cox4i1, coxIII, cox5b, cox7a1, cox8b, glut4, atpase b2, cox II, atp₅₀, ndufb5, ap2, ndufs1, GRP109A, acylCoA-thioesterase 4, EARA1, claudin1, PEPCK, fgf21, acylCoA-thioesterase 3, and dio2; b) thermogenesis in adipose cells; c) differentiation of adipose cells; d) insulin sensitivity of adipose cells; e) basal respiration or uncoupled respiration; f) hepatosteatosis reduction; g) appetite reduction; h) insulin secretion of pancreatic beta cells; i) cardiac function reduction; j) cardiac hypertrophy; and k) muscle hypoplasia reduction. In other embodiments, the polypeptide is less than 195 amino acids in length. In still other embodiments, the polypeptide is between 70 and 125 amino acids in length. In yet other embodiments, the polypeptide does not comprise the amino acid sequence of SEQ ID NO:2. In other embodiments, the polypeptide contains one or more conservative amino acid substitutions. In still other embodiments, at least one amino acid residue is glycosylated or pegylated. In yet other embodiments, at least one glycosylated amino acid residue corresponds to asparagine at position 36 and/or the asparagine at position 81 of SEQ ID NO:2. In other embodiments, the polypeptide is a secreted polypeptide. In still other embodiments, the polypeptide further comprises a heterologous polypeptide (e.g., a signal peptide; peptide tag such as a 6-His, thioredoxin, hemaglutinin, albumin, GST, or OmpA signal sequence tag; a dimerization or oligomerization domain; an agent that promotes plasma solubility; an antibody or fragment thereof such as an Fc domain (e.g., an IgG1 Fc domain, an IgG2 Fc domain, an IgG3 Fc domain or an IgG4 Fc domain)). In yet other embodiments, the polypeptide is immobilized on an object selected from the group consisting of a cell, a metal, a resin, a polymer, aceramic, a glass, a microelectrode, a graphitic particle, a bead, a gel, a plate, an array, and a capillary tube. In addition, it is contemplated that such polypeptides are inclusive of nucleic acid and polypeptide molecules encompassing the corresponding nucleotides and residues in an FNDC5 ortholog of SEQ ID NOs: 1 and 2, such as human FNDC5 nucleic acid and polypeptide sequences (see, for example, sequence provided in Table 1).

Modulators of FNDC5/irisin nucleic acid and polypeptide molecules can inhibit or promote the copy number, expression level and/or activity of one or more FNDC5/irisin nucleic acid and/or polypeptide molecules described herein, such as being specific for a particular FNDC5 and/or irisin form, or modulating a group of FNDC5 and/or irisin forms sharing a common structure.

As used herein, the term “integrin” refers to the extracellular receptors that are expressed in a wide variety of cells and bind to specific ligands in the extracellular matrix. The specific ligands bound by integrins can contain an arginine-glycine-aspartic acid tripeptide (Arg-Gly-Asp; RGD) or a leucine-aspartic acid-valine tripeptide, and include, for example, fibronectin, vitronectin, osteopontin, tenascin, and von Willebrand's factor. The integrins comprise a superfamily of heterodimers composed of an α subunit and a β subunit. Numerous a subunits, designated, for example, αV, α5 and the like, and numerous β subunits, designated, for example, β1, β2, β3, β5 and the like, have been identified, and various combinations of these subunits are represented in the integrin superfamily, including α5β1, αVβ3 and αVβ5. There are at least 18 α and eight β subunits are known in humans, generating 24 heterodimers (Takada et al. (2007) Gen. Biol. 8:215). The superfamily of integrins can be subdivided into families, for example, as αV-containing integrins, including αVβ3 and αVβ5, or the β1-containing integrins, including α5β1 and αVβ1. Integrins are expressed in a wide range of organisms, including C. elegans, Drosophila sp., amphibians, reptiles, birds, and mammals, including humans.

Integrins link the extracellular matrix (ECM) to the cytoskeleton and transmit signals and mechanical forces bi-directionally across the plasma membrane (Hynes et al. (2002) Cell 110:673-687). Integrins are regulated by clustering and conformational changes triggered either “outside in” by binding to their specific ECM ligands, or “inside out” by interaction between the intracellular tails of integrin subunits and cytoplasmic proteins (Margadant et al. (2011) Curr. Opin. Cell Bio. 23:607-614). The β subunit cytoplasmic tails share significant sequence similarity; several cytoplasmic proteins directly bind most β subunits to regulate integrin activation, trafficking and signaling (Moser et al. (2009) Science 324:895-899; Calderwood, et al. (2004) J. Cell Sci. 117:657-666). In contrast, the α integrin subunit tails share only a short, conserved membrane-proximal sequence that interacts directly with the β subunit and with proteins that regulate integrin trafficking (Ivaska and Heino (2011) Annu. Rev. Cell Dev. Biol. 27:291-320), and with Sharpin, a negative regulator of integrin activation (Rantala et al. (2011) Nat Cell Biol. 13:1315-1324).

In some embodiments, irisin binds an integrin that comprises β1 subunit (ITGB1/CD29), βa or β5, including but is not limited to, α1β1, α2β1, α3β1, α4β1, α5β1, α6β1, α7β1, α8β1, α9β1, α10β1, α11β1, αDβ1, αEβ1, αLβ1, αMβ1, α2Bβ1, αXβ1, and αVβ1, as well as such alpha integrins heterodimerized with βa or β5 subunits. In other embodiments, irisin binds an integrin that comprises alpha V subunit (ITGAV), such as including, but not limited to, αVβ1, αVβ3, αVβ5, αVβ6 and αVβ8. In one embodiment, irisin binds alpha V beta 5 (αVβ5)-integrin, α1β1-integrin, αVβ1-integrin, or α5β1-integrin. In some embodiments, irisin binds αVβ5-integrin or αVβ1-integrin.

Integrin subunits are well-known in the art. For example, integrin alpha-V is a type I integral membrane glycoprotein, known as vitronectin receptor a chain, or CD51 (NCBI mouse gene ID 16410 and human gene ID 3685). It forms a heterodimer with integrin β1 (CD29), β3 (CD61), β5, β6, or β8. It contains two disulfide-linked subunits of 125 kDa and 24 kDa, and is expressed on endothelial cells, fibroblasts, macrophages, platelets, osteoclasts, neuroblastoma, melanoma, and hepatoma cells. Many extracellular matrix proteins with RGD-motifs are integrin alpha-V ligands. In association with its β chains, alpha-V integrin binds vitronectin, von Willebrand factor, fibronectin, thrombospondin, osteopontin, fibrinogen, and laminin. As an adhesion molecule, it plays important roles in angiogenesis, leukocyte homing and rolling, and bone absorption.

Integrin β5 is a 95 kDa glycoprotein heterodimer (NCBI mouse gene ID 16419 and NCBI human gene ID 3693) with the αV and α5 subunits and is found on many types of tissue cells, such as epithelial cells, endothelial cells, keratinocytes, and osteoblastic cells. The αV/β5 integrin complex binds to vitronectin. Agents that target integrin β5 are well-known in the art, such as anti-human β5 integrin antibody AST-3T.

Integrin alpha-5 is a type I integral membrane glycoprotein, known as CD49e and VLA-5 α chain (NCBI mouse gene ID 16402 and NCBI human gene ID 3678). It forms a non-covalent heterodimer with integrin β1 (CD29). CD49e contains two disulfide-linked subunits of 135 kDa and 24 kDa, and is mainly expressed on thymocytes, activated lymphocytes, endothelial cells, osteoblasts, melanoma, and some myeloid leukemia cells, and functions in adhesion and regulates cell survival and apoptosis.

Integrin beta-1 is a 130 kDa single chain type I glycoprotein, known as CD29, VLA-β chain, or gpIIa (NCBI mouse gene ID 16412 and human gene ID 3688). It is broadly expressed on a majority of hematopoietic and non-hematopoietic cells, including leukocytes (although at low level on granulocytes), platelets, fibroblasts, endothelial cells, epithelial cells, and mast cells. It is non-covalently associated with integrin α1-α6 chains to form VLA-1 to VLA-6 molecules, respectively. Heterodimers that include integrin beta-1 bind to several cell surfaces (e.g., VCAM-1 and MadCAM-1) and extracellular matrix molecules. It acts as a fibronectin receptor and is involved in a variety of cell-cell and cell-matrix interactions. As each of these subunits is widely expressed, a wide variety of cells can express this heterodimer. αVβ1 is expressed early in differentiation for oligodendrocytes, astrocytes and pancreatic β cells, but down-regulated following their differentiation. αVβ1 has also been implicated as a receptor for certain types of virus, like human metaneumovirus. The heterodimer has a number of functions, including mediating fibrosis (Reed et al. (2015) Sci. Transl. Med. 7:288ra79; Smith and Henderson (2016) Exp. Opin. Drug Disc. 11:749-751; Song et al. (2016) Ann. Transl. Med. 4:411). Agents that target αVβ5-integrin and/or αVβ1-integrin are well-known in the art, such as anti-human αV (CD51) integrin antibody NKI-M9, anti-mouse αV (CD51) integrin antibody RMV-7, anti-human β1 integrin (CD29) antibodies TS2/16 and Poly6004, anti-mouse/rat β1 integrin (CD29) antibody HMβ1-1, and anti-human β5 integrin antibody AST-3T.

The heterodimer α5β1 is an integrin that binds to matrix macromolecules and proteinases and thereby stimulates angiogenesis (Boudreau et al. (2004) J. Biol. Chem. 279:4862-4868). It is composed of α5 (ITGA5/CD49e) and β1 (ITGB1/CD29) subunits. α5β1 integrin is the primary receptor for soluble fibronectin and plays the predominant role in assembling fibronectin into fibrils (Yang et al. (1999) Dev. Biol. 215:264-277). Studies in experimental animal models and in mutant mice indicate that the α5β1 integrin also plays a key role in regulating angiogenesis (Brooks et al. (1994) Science 264:569-571; Brooks et al. (1994) Cell 79:1157-1164; Friedlander et al. (1995) Science 270:1500-1502). Studies have also demonstrated that loss of the gene encoding the integrin α5 subunit is embryonic lethal in mice and is associated with a complete absence of the posterior somites and with some vascular and cardiac defects (Yang et al. (1993) Development 119:1093-1105; Goh et al. (1997) Development 124:4309-4319). The association of α5β1 integrin with tumor angiogenesis is also well-established. Therefore, α5β1 integrin has become a therapeutic target for numerous diseases mediated by angiogenic processes including cancerous tumor growth. Recent studies have also shown that overexpression of α5β1 is associated with a poor prognosis for patients in solid tumors, in particular in colon, breast, ovarian, lung and brain tumors (Schaffner et al. (2013) Cancers 5:27-47). α5β1 integrin antagonists have been developed that block specific binding to fibronectin. These antagonists include, but are not limited to, α5β1 antibodies such as IIA1 (Sawada et al. (2008) Cancer Res. 68:2329-2339), M200/volociximab (PDL BioPharma and Biogen Idec), or PF-04605412 (Pfizer), RGD-like molecules such as SJ749 or JSM6427, and non RGD-like peptides such as ATN-161 (Attenuon LLC). Other agents that target integrin α5β1 are well-known in the art, such as anti-human α5 (CD49e) integrin antibody NKI-SAM-1, anti-mouse α5 (CD49e) integrin antibody 5H10-27 (MRF5), and anti-mouse/rat α5 (CD49e) integrin antibody HMα5-1.

Integrin alpha-1 is a 1179 aa, type I transmembrane glycoprotein, also known as CD49a, VLA-1 α chain, or integrin α1 (NCBI mouse gene ID 109700 and NCBI human gene ID 3672). Integrin alpha-1 is an adhesion molecule and is involved in the regulation of leukocyte migration, T cell proliferation, and cytokine production. Agents that target integrin α5β1 are well-known in the art, such as anti-human α1 (CD49a) integrin antibody TS2/7 and anti-mouse α1 (CD49a) integrin antibody HMα1.

The heterodimer α1β1 is a collagen IV and alminin-1 receptor that is expressed on activated T cells, smooth muscle cells, endothelial cells, neuronal cells, fibroblasts, and mesenchymal cells. It plays a role in fibroblast proliferation, collagen synthesis, matrix metalloproteinase expression, and renal injury response.

The term “protease” refers to a group of enzymes whose catalytic function is to hydrolyze peptide bonds of proteins (e.g., to cleave FNDC5 into irisin). “Protease inhibitors” are molecules that inhibit the function of proteases. Protease inhibitors may be classified either by the type of protease they inhibit, or by their mechanism of action. In 2004 Rawlings and colleagues introduced a classification of protease inhibitors based on similarities detectable at the level of amino acid sequence (Rawlings et al. (2004), Biochem. J. 378: 705-16). In one embodiment, the protease inhibitor is a DPP4 inhibitor. Dipeptidyl peptidase (DPP4) inhibitors, that include sitagliptin, vildagliptin and saxagliptin, are a new class of drugs that inhibit the proteolytic activity of dipeptidyl peptidase-4.

The term “inhibit” includes the decrease, limitation, or blockage, of, for example a particular action, function, or interaction.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g. a package or container) comprising at least one reagent, e.g. a probe or small molecule, for specifically detecting and/or affecting the expression of a marker of the present invention. The kit may be promoted, distributed, or sold as a unit for performing the methods of the present invention. The kit may comprise one or more reagents necessary to express a composition useful in the methods of the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

The “normal” level of expression of a biomarker is the level of expression of the biomarker in cells of a subject, e.g., a human patient, not afflicted with a bone loss condition. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “pre-determined” biomarker amount and/or activity measurement(s) may be a biomarker amount and/or activity measurement(s) used to, by way of example only, evaluate a subject that may be selected for a particular treatment, evaluate a response to a treatment such as irisin-based therapy, and/or evaluate the disease state. A pre-determined biomarker amount and/or activity measurement(s) may be determined in populations of patients with or without bone loss conditions. The pre-determined biomarker amount and/or activity measurement(s) can be a single number, equally applicable to every patient, or the pre-determined biomarker amount and/or activity measurement(s) can vary according to specific subpopulations of patients. Age, weight, height, and other factors of a subject may affect the pre-determined biomarker amount and/or activity measurement(s) of the individual. Furthermore, the pre-determined biomarker amount and/or activity can be determined for each subject individually. In one embodiment, the amounts determined and/or compared in a method described herein are based on absolute measurements. In another embodiment, the amounts determined and/or compared in a method described herein are based on relative measurements, such as ratios (e.g., serum biomarker normalized to the expression of housekeeping or otherwise generally constant biomarker). The pre-determined biomarker amount and/or activity measurement(s) can be any suitable standard. For example, the pre-determined biomarker amount and/or activity measurement(s) can be obtained from the same or a different human for whom a patient selection is being assessed. In one embodiment, the pre-determined biomarker amount and/or activity measurement(s) can be obtained from a previous assessment of the same patient. In such a manner, the progress of the selection of the patient can be monitored over time. In addition, the control can be obtained from an assessment of another human or multiple humans, e.g., selected groups of humans, if the subject is a human. In such a manner, the extent of the selection of the human for whom selection is being assessed can be compared to suitable other humans, e.g., other humans who are in a similar situation to the human of interest, such as those suffering from similar or the same condition(s) and/or of the same ethnic group.

The term “predictive” includes the use of a biomarker nucleic acid and/or protein status, e.g., over- or under-activity, emergence, expression, growth, remission, recurrence or resistance of bone loss conditions before, during or after therapy, for determining the likelihood of response of a bone loss condition to irisin-based therapy (e.g., treatment with an agent that decreases the amount and/or activity of irisin or a biologically inactive or inhibitory irisin mutant that binds to the irisin receptor in osteocytes). Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human bone loss samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with bone loss conditions; (3) its absolute or relatively modulated presence or absence in clinical subset of patients with bone loss conditions (e.g., those responding to a particular irisin-based therapy or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “treatment,” as used herein, is defined as the application or administration of a therapeutic agent to a patient, or application or administration of a therapeutic agent to an isolated tissue or cell line from a patient, who has a disease or disorder, a symptom of a disease or disorder or a predisposition toward a disease or disorder, with the purpose of curing, healing, alleviating, relieving, altering, remedying, ameliorating, improving or affecting the disease or disorder, the symptoms of disease or disorder or the predisposition toward a disease or disorder. A therapeutic agent includes, but is not limited to, polypeptides, small molecules, peptides, peptidomimetics, nucleic acid molecules, antibodies, ribozymes, siRNA molecules, and sense and antisense oligonucleotides described herein

The term “probe” refers to any molecule which is capable of selectively binding to a specifically intended target molecule, for example, a nucleotide transcript or protein encoded by or corresponding to a biomarker nucleic acid. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, indicating that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

As used herein, the term “intracellular immunoglobulin molecule” is a complete immunoglobulin which is the same as a naturally-occurring secreted immunoglobulin, but which remains inside of the cell following synthesis. An “intracellular immunoglobulin fragment” refers to any fragment, including single-chain fragments of an intracellular immunoglobulin molecule. Thus, an intracellular immunoglobulin molecule or fragment thereof is not secreted or expressed on the outer surface of the cell. Single-chain intracellular immunoglobulin fragments are referred to herein as “single-chain immunoglobulins.” As used herein, the term “intracellular immunoglobulin molecule or fragment thereof” is understood to encompass an “intracellular immunoglobulin,” a “single-chain intracellular immunoglobulin” (or fragment thereof), an “intracellular immunoglobulin fragment,” an “intracellular antibody” (or fragment thereof), and an “intrabody” (or fragment thereof). As such, the terms “intracellular immunoglobulin,” “intracellular Ig,” “intracellular antibody,” and “intrabody” may be used interchangeably herein, and are all encompassed by the generic definition of an “intracellular immunoglobulin molecule, or fragment thereof.” An intracellular immunoglobulin molecule, or fragment thereof of the present invention may, in some embodiments, comprise two or more subunit polypeptides, e.g., a “first intracellular immunoglobulin subunit polypeptide” and a “second intracellular immunoglobulin subunit polypeptide.”However, in other embodiments, an intracellular immunoglobulin may be a “single-chain intracellular immunoglobulin” including only a single polypeptide. As used herein, a “single-chain intracellular immunoglobulin” is defined as any unitary fragment that has a desired activity, for example, intracellular binding to an antigen. Thus, single-chain intracellular immunoglobulins encompass those which comprise both heavy and light chain variable regions which act together to bind antigen, as well as single-chain intracellular immunoglobulins which only have a single variable region which binds antigen, for example, a “camelized” heavy chain variable region as described herein. An intracellular immunoglobulin or Ig fragment may be expressed anywhere substantially within the cell, such as in the cytoplasm, on the inner surface of the cell membrane, or in a subcellular compartment (also referred to as cell subcompartment or cell compartment) such as the nucleus, Golgi, endoplasmic reticulum, endosome, mitochondria, etc. Additional cell subcompartments include those that are described herein and well known in the art.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method of the present invention further comprises obtaining the sample from the individual prior to detecting or determining the presence or level of at least one marker in the sample.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having bone loss conditions, to inhibit expression of a biomarker gene which is overexpressed in bone loss conditions and thereby treat, prevent, or inhibit bone loss in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term refering to the ability of an antibody to discriminate the binding of one antigen over another.

The term “subject” refers to any healthy animal, mammal or human, or any animal, mammal or human afflicted with a bone loss condition. The term “subject” is interchangeable with “patient”.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (median effective dose) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

### Genetic Code

Alanine (Ala, A) GCA, GCC, GCG, GCT
Arginine (Arg, R) AGA, ACG, CGA, CGC, CGG, CGT
Asparagine (Asn, N) AAC, AAT
Aspartic acid (Asp, D) GAC, GAT
Cysteine (Cys, C) TGC, TGT
Glutamic acid (Glu, E) GAA, GAG
Glutamine (Gln, Q) CAA, CAG
Glycine (Gly, G) GGA, GGC, GGG, GGT
Histidine (His, H) CAC, CAT
Isoleucine (Ile, I) ATA, ATC, ATT
Leucine (Leu, L) CTA, CTC, CTG, CTT, TTA, TTG
Lysine (Lys, K) AAA, AAG
Methionine (Met, M) ATG
Phenylalanine (Phe, F) TTC, TTT
Proline (Pro, P) CCA, CCC, CCG, CCT
Serine (Ser, S) AGC, AGT, TCA, TCC, TCG, TCT
Threonine (Thr, T) ACA, ACC, ACG, ACT
Tryptophan (Trp, W) TGG
Tyrosine (Tyr, Y) TAC, TAT
Valine (Val, V) GTA, GTC, GTG, GTT
Termination signal (end) TAA, TAG, TGA

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information for the loci and biomarkers of the present invention are well-known in the art and readily available on publicly available databases, such as the National Center for Biotechnology Information (NCBI). For example, exemplary nucleic acid and amino acid sequences of Fndc5 derived from publicly available sequence databases are provided below.

TABLE 1
SEQ ID NO: 1 Mouse Fndc5 cDNA Sequence

| | |
|---|---|
| 1 | atgcccccag ggccgtgcgc ctggccgccc cgcgccgcgc tccgcctgtg gctaggctgc |
| 61 | gtctgcttcg cgctggtgca ggcggacagc ccctcagccc ctgtgaacgt gaccgtccgg |
| 121 | cacctcaagg ccaactctgc cgtggtcagc tgggatgtcc tggaggatga agtggtcatt |
| 181 | ggctttgcca tctctcagca gaagaaggat gtgcggatgc tccggttcat tcaggaggtg |
| 241 | aacaccacca cccggtcctg cgctctctgg gacctggagg aggacacaga atatatcgtc |
| 301 | catgtgcagg ccatctccat ccagggacag agcccagcca gtgagcctgt gctcttcaag |
| 361 | accccacgcg aggctgaaaa gatggcctca aagaacaaag atgaggtgac catgaaggag |
| 421 | atggggagga accagcagct gcgaacgggg gaggtgctga tcattgttgt ggtcctcttc |
| 481 | atgtgggcag gtgttatagc tctcttctgc cgccagtatg atatcatcaa ggacaacgag |
| 541 | cccaataaca acaaggagaa aaccaagagc gcatcagaaa ccagcacacc ggagcatcag |
| 601 | ggtgggggtc tcctccgcag caagatatga |

SEQ ID NO: 2 Mouse Fndc5 Amino Acid Sequence

| | |
|---|---|
| 1 | mppgpcawpp raalrlwlgc vcfalvqads psapvnvtvr hlkansavvs wdvledevvi |
| 61 | gfaisqqkkd vrmlrfiqev ntttrscalw dleedteyiv hvgaisiqgq spasepvlfk |
| 121 | tpreaekmas knkdevtmke mgrnqqlrtg evliivvvlf mwagvialfc rqydiikdne |
| 181 | pnnnkektks asetstpehq gggllrski |

SEQ ID NO: 3 Human Fndc5 (isoform 1) cDNA Sequence

| | |
|---|---|
| 1 | atgctgcgct tcatccagga ggtgaacacc accacccgct catgtgccct ctgggacctg |
| 61 | gaggaggata cggagtacat agtccacgtg caggccatct ccattcaggg ccagagccca |
| 121 | gccagcgagc ctgtgctctt caagaccccg cgtgaggctg agaagatggc ctccaagaac |
| 181 | aaagatgagg taaccatgaa agagatgggg aggaaccaac agctgcggac aggcgaggtg |
| 241 | ctgatcatcg tcgtggtcct gttcatgtgg gcaggtgtca ttgccctctt ctgccgccag |
| 301 | tatgacatca tcaaggacaa tgaacccaat aacaacaagg aaaaaaccaa gagtgcatca |
| 361 | gaaaccagca caccagagca ccagggcggg gggcttctcc gcagcaaggt gagggcaaga |
| 421 | cctgggcctg ggtgggccac cctgtgcctc atgctctggt aa |

SEQ ID NO: 4 Human Fndc5 (isoform 1) Amino Acid Sequence

| | |
|---|---|
| 1 | mlrfiqevnt ttrscalwdl eedteyivhv qaisiqgqsp asepvlfktp reaekmaskn |
| 61 | kdevtmkemg rnqqlrtgev liivvvlfmw agvialfcrq ydiikdnepn nnkektksas |
| 121 | etstpehqgg gllrskvrar pgpgwatlcl mlw |

SEQ ID NO: 5 Human Fndc5 (isoform 2) cDNA Sequence

| | |
|---|---|
| 1 | atacaccccg ggtcgccgag cgcctggccg ccccgcgccc gcgccgcgct ccgcctgtgg |
| 61 | ctgggctgcg tctgcttcgc gctggtgcag gcggacagtc cctcagcccc agtgaacgtc |
| 121 | accgtcaggc acctcaaggc caactctgca gtggtgagct gggatgttct ggaggatgag |
| 181 | gttgtcatcg gatttgccat ctcccagcag aagaaggatg tgcggatgct gcgcttcatc |
| 241 | caggaggtga acaccaccac ccgctcatgt gccctctggg acctggagga ggatacggag |
| 301 | tacatagtcc acgtgcaggc catctccatt cagggccaga gcccagccag cgagcctgtg |
| 361 | ctcttcaaga ccccgcgtga ggctgagaag atggcctcca agaacaaaga tgaggtaacc |
| 421 | atgaaagaga tggggaggaa ccaacagctg cggacaggcg aggtgctgat catcgtcgtg |
| 481 | gtcctgttca tgtgggcagg tgtcattgcc ctcttctgcc gccagtatga catcatcaag |
| 541 | gacaatgaac ccaataacaa caaggaaaaa accaagagtg catcagaaac cagcacacca |
| 601 | gagcaccagg gcggggggct tctccgcagc aagatatga |

SEQ ID NO: 6 Human Fndc5 (isoform 2) Amino Acid Sequence

| | |
|---|---|
| 1 | mhpgspsawp praraalrlw lgcvcfalvq adspsapvnv tvrhlkansa vvswdvlede |
| 61 | vvigfaisqq kkdvrmlrfi qevntttrsc alwdleedte yivhvqaisi qgqspasepv |
| 121 | lfktpreaek masknkdevt mkemgrnqql rtgevliivv vlfmwagvia lfcrqydiik |
| 181 | dnepnnnkek tksasetstp ehqgggllrs ki |

SEQ ID NO: 7 Human Fndc5 (isoform 3) cDNA Sequence

| | |
|---|---|
| 1 | atacaccccg ggtcgccgag cgcctggccg ccccgcgccc gcgccgcgct ccgcctgtgg |
| 61 | ctgggctgcg tctgcttcgc gctggtgcag gcggacagtc cctcagcccc agtgaacgtc |
| 121 | accgtcaggc acctcaaggc caactctgca gtggtgagct gggatgttct ggaggatgag |
| 181 | gttgtcatcg gatttgccat ctcccagcag aagaaggatg tgcggatgct gcgcttcatc |
| 241 | caggaggtga acaccaccac ccgctcatgt gccctctggg acctggagga ggatacggag |
| 301 | tacatagtcc acgtgcaggc catctccatt cagggccaga gcccagccag cgagcctgtg |
| 361 | ctcttcaaga ccccgcgtga ggctgagaag atggcctcca agaacaaaga tgaggtaacc |
| 421 | atgaaagaga tggggaggaa ccaacagctg cggacaggcg aggtgctgat catcgtcgtg |
| 481 | gtcctgttca tgtgggcagg tgtcattgcc ctcttctgcc gccagtatga catcattgaa |
| 541 | gcgtga |

SEQ ID NO: 8 Human Fndc5 (isoform 3) Amino Acid Sequence

| | |
|---|---|
| 1 | mhpgspsawp praraalrlw lgcvcfalvq adspsapvnv tvrhlkansa vvswdvlede |
| 61 | vvigfaisqq kkdvrmlrfi qevntttrsc alwdleedte yivhvqaisi qgqspasepv |
| 121 | lfktpreaek masknkdevt mkemgrnqql rtgevliivv vlfmwagvia lfcrqydiie |
| 181 | a |

SEQ ID NO: 9 Human Fndc5 (isoform 4) cDNA Sequence

| | |
|---|---|
| 1 | atgctgcgct tcatccagga ggtgaacacc accacccgct catgtgccct ctgggacctg |
| 61 | gaggaggata cggagtacat agtccacgtg caggccatct ccattcaggg ccagagccca |
| 121 | gccagcgagc ctgtgctctt caagaccccg cgtgaggctg agaagatggc ctccaagaac |
| 181 | aaagatgagg taaccatgaa agagatgggg aggaaccaac agctgcggac aggcgaggtg |
| 241 | ctgatcatcg tcgtggtcct gttcatgtgg gcaggtgtca ttgccctctt ctgccgccag |
| 301 | tatgacatca tcaaggacaa tgaacccaat aacaacaagg aaaaaaccaa gagtgcatca |
| 361 | gaaaccagca caccagagca ccagggcggg gggcttctcc gcagcaagat atga |

SEQ ID NO: 10 Human Fndc5 (isoform 4) Amino Acid Sequence

| | |
|---|---|
| 1 | mlrfiqevnt ttrscalwdl eedteyivhv qaisiqgqsp asepvlfktp reaekmaskn |
| 61 | kdevtmkemg rnqqlrtgev liivvvlfmw agvialfcrq ydiikdnepn nnkektksas |
| 121 | etstpehqgg gllrski |

SEQ ID NO: 11 Human Fndc5 (isoform 5) cDNA Sequence

| | |
|---|---|
| 1 | atgctgcgct tcatccagga ggtgaacacc accacccgct catgtgccct ctgggacctg |
| 61 | gaggaggata cggagtacat agtccacgtg caggccatct ccattcaggg ccagagccca |
| 121 | gccagcgagc ctgtgctctt caagaccccg cgtgaggctg agaagatggc ctccaagaac |
| 181 | aaagatgagg taaccatgaa agagatgggg aggaaccaac agctgcggac aggcgaggtg |
| 241 | ctgatcatcg tcgtggtcct gttcatgtgg gcaggtgtca ttgccctctt ctgccgccag |
| 301 | tatgacatca ttgaagcgtg a |

SEQ ID NO: 12 Human Fndc5 (isoform 5) Amino Acid Sequence

| | |
|---|---|
| 1 | mlrfiqevnt ttrscalwdl eedteyivhv qaisiqgqsp asepvlfktp reaekmaskn |
| 61 | kdevtmkemg rnqqlrtgev liivvvlfmw agvialfcrq ydiiea |

SEQ ID NO: 13 Chicken Fndc5 (isoform 1) cDNA Sequence

| | |
|---|---|
| 1 | atggagccct tcctgggctg caccggcgcc gcgctcctgc tctgcttcag ctacgccggt |
| 61 | ctgcggccgg tggaggcaga cagcccttcg gctccggtca atgtcacagt caaacacctg |
| 121 | aaggccaact cagctgtagt gacttgggac gttctggagg atgaagttgt cattggattt |
| 181 | gccatttccc agcagaagaa ggacgtgcgg atgctgcgct tcatccagga ggtgaacacc |
| 241 | accacccgct cctgtgccct ctgggaccta gaggaggaca ctgagtacat tgtgcatgtc |
| 301 | caggccatca gcatccaagg ccagagccct gccagtgagc cagtcctctt caagaccccc |
| 361 | agggaagctg agaaactggc ttctaaaaat aaagatgagg tgacaatgaa ggagatggcg |
| 421 | aagaaaaacc aacagctgcg cgcaggggaa atactcatca ttgtggtggt gttgtttatg |
| 481 | tgggcagggg tgatcgccct gttctgcagg cagtacgaca tcatcaaaga caacgagccg |
| 541 | aacaacagca aggagaaagc caagagcgcc tcagagaaca gcaccccyga gcaccagggt |
| 601 | ggggggctgc tccgcagcaa gttcccaaaa aacaaaccct cagtgaacat cattgaggca |
| 661 | taa |

SEQ ID NO: 14 Chicken Fndc5 (isoform 1) Amino Acid Sequence

| | |
|---|---|
| 1 | mepflgctga alllcfsyag lrpveadsps apvnvtvkhl kansavvtwd vledevvigf |
| 61 | aisqqkkdvr mlrfiqevnt ttrscalwdl eedteyivhv qaisiqgqsp asepvlfktp |
| 121 | reaeklaskn kdevtmkema kknqqlrage iliivvvlfm wagvialfcr qydiikdnep |
| 181 | nnskekaksa senstpehqg ggllrskfpk nkpsvniiea |

SEQ ID NO: 15 Chicken Fndc5 (isoform 2) cDNA Sequence

| | |
|---|---|
| 1 | atggagaaga acagggacgg ccgcggcccc cctggtgtcc atctggggat ggagaaggaa |
| 61 | gatgatttag agcccggtga cacgccgggg ctgcgcgaag ccctggtggc gagatgtcac |
| 121 | cgctgccgcg cacccgccgg gggtctcacc gggacgggcc ccgtttgctc cttccggcga |
| 181 | tggggagcgg tccgggccga gggctcccgg tcccgcctgg gggaaactga ggcagacggc |
| 241 | ggggccgggc ggggcggggg ccgagccgcc cccgggccgg gggagggacc ggagcggggc |
| 301 | tgcccagcgc tgcagcgggc ggagccgggg ctcggcgggg ccgcctcccg gccgagccga |
| 361 | gccgaaccga gccgcgctgc cgagggccgc cgagcccgca gccgcccccg gccgaaccgg |
| 421 | gcggccccgc cggttccggg ccccggagct ctccgcggtg ctgaacggcg ccgccgcgcc |
| 481 | cgcgggacgc cggccccgga gcggctcggc cccggcgcgg cgcggcgggc cgcgggggga |
| 541 | tggagccctt cctgggctgc accggcgccg cgctcctgct ctgctttcag ctacgccggt |
| 601 | ctgcggccgg tggaggcaga cagcccttcg gctccggtca atgtcacagt caaacacctg |
| 661 | aaggccaact cagctgtagt gacttgggac gttctggagg atgaagttgt cattggattt |
| 721 | gccatttccc agcagaagaa ggacgtgcgg atgctgcgct tcatccagga ggtgaacacc |
| 781 | accacccgct cctgtgccct ctgggaccta gaggaggaca ctgagtacat tgtgcatgtc |
| 841 | caggccatca gcatccaagg ccagagccct gccagtgagc cagtcctctt caagaccccc |
| 901 | agggaagctg agaaactggc ttctaaaaat aaagatgagg tgacaatgaa ggagatggcg |
| 961 | aagaaaaacc aacagctgcg cgcaggggaa atactcatca ttgtggtggt gttgtttatg |
| 1021 | tgggcagggg tgatcgccct gttctgcagg cagtacgaca tcatcaaaga caacgagccg |
| 1081 | aacaacagca aggagaaagc caagagcgcc tcagagaaca gcacccccga gcaccagggt |
| 1141 | ggggggctgc tccgcagcaa gttcccaaaa aacaaaccct cagtgaacat cattgaggca |
| 1201 | taa |

SEQ ID NO: 16 Chicken Fndc5 (isoform 2) Amino Acid Sequence

| | |
|---|---|
| 1 | meknrdgrgp pgvhlgmeke ddlepgdtpg lrealvarch rcrapagglt gtgpvcsfrr |
| 61 | wgavraegsr srlgeteadg gagrgggraa pgpgegperg cpalqraepg lggaasrpsr |
| 121 | aepsraaegr rarsrprpnr aappvpgpga lrgaerrrra rgtpaperlg pgaarraagg |
| 181 | wspswaapap rscsafsyag lrpveadsps apvnvtvkhl kansavvtwd vledevvigf |
| 241 | aisqqkkdvr mlrfiqevnt ttrscalwdl eedteyivhv qaisiqgqsp asepvlfktp |
| 301 | reaeklaskn kdevtmkema kknqqlrage iliivvvlfm wagvialfcr qydiikdnep |
| 361 | nnskekaksa senstpehqg ggllrskfpk nkpsvniiea |

SEQ ID NO: 17 Zebrafish Fndc5 cDNA Sequence

| | |
|---|---|
| 1 | atgagttctt acagtttggc agctccagtg aatgtgtcca tcagggatct gaagagcagc |
| 61 | tcagccgtgg tgacatggga cacgccagac ggagagccag tcatcggctt cgccatcaca |
| 121 | caacagaaga aagatgtccg catgctgcgc tttattcaag aagtgaacac caccacgcgg |
| 181 | agctgtgcat tgtgggatct ggaagctgat acggattaca ttgtgcacgt tcagtctatc |
| 241 | agcatcagcg gggcgagtcc tgttagtgaa gctgtgcact tcaagacccc gacagaagtt |
| 301 | gaaacacagg cctccaagaa caaagacgag gtgacgatgg aggaggtcgg gccgaacgct |
| 361 | cagctcaggg ccggagagtt catcattatt gtggtggtcc tcatcatgtg ggcaggtgtg |
| 421 | atcgcactat tctgccgtca gtatgacatc attaaagaca acgaaccaaa caataacaag |
| 481 | gataaagcca agaactcgtc tgaatgcagc actccagagc acacgtcagg tggcctgctg |
| 541 | cgcagtaagg tataa |

SEQ ID NO: 18 Zebrafish Fndc5 Amino Acid Sequence

| | |
|---|---|
| 1 | mssyslaapv nvsirdlkss savvtwdtpd gepvigfait qqkkdvrmlr fiqevntttr |
| 61 | scalwdlead tdyivhvqsi sisgaspvse avhfktptev etqasknkde vtmeevgpna |
| 121 | qlragefiii vvvlimwagv ialfcrqydi ikdnepnnnk dkaknssecs tpehtsggll |
| 181 | rskv |

SEQ ID NO: 19 Rat Fndc5 cDNA Sequence

| | |
|---|---|
| 1 | atgcccccag ggccgtgcgc ctggccgccc cgcgccgctc tccggctgtg gctgggctgc |
| 61 | gtgtgcttcg cgctggtgca ggcggacagc ccctcggccc ccgtgaacgt aaccgtcagg |
| 121 | cacctcaagg ccaactcggc agtggtcagc tgggacgtcc tggaggacga ggttgtcatc |
| 181 | ggctttgcca tctctcagca gaagaaggat gtgaggatgc tgcgcttcat tcaggaggtg |
| 241 | aacaccacca cccgatcctg cgctctctgg gacctggagg aggacacaga gtatatcgtc |
| 301 | cacgtgcagg ccatctccat ccagggccag agcccagcca gtgagcccgt gctcttcaag |
| 361 | accccacgtg aggccgagaa gatggcctct aagaacaaag atgaggtgac catgaaggag |
| 421 | atggggagga accagcagct gcggacgggc gaggtgctga tcatcgtcgt ggtcctcttc |
| 481 | atgtgggcag gtgtcatagc tctcttctgc cgccagtatg acatcatcaa ggacaacgag |
| 541 | cccaataaca acaaggaaaa aaccaagagt gcatcagaga ccagcacccc agagcaccag |
| 601 | ggtgggggtc tcctccgaag caagatatga |

SEQ ID NO: 20 Rat Fndc5 Amino Acid Sequence

| | |
|---|---|
| 1 | mppgpcawpp raalrlwlgc vcfalvqads psapvnvtvr hlkansavvs wdvledevvi |
| 61 | gfaisqqkkd vrmlrfiqev ntttrscalw dleedteyiv hvqaisiqgq spasepvlfk |
| 121 | tpreaekmas knkdevtmke mgrnqqlrtg evliivvvlf mwagvialfc rqydiikdne |
| 181 | pnnnkektks asetstpehq gggllrski |

SEQ ID NO: 21 Fragment of Murine Fndc5 Nucleic Acid Sequence that
encodes amino acid residues 29-140 of murine Fndc5

| | |
|---|---|
| 85 | gacagc ccctcagccc ctgtgaacgt gaccgtccgg |
| 121 | cacctcaagg ccaactctgc cgtggtcagc tgggatgtcc tggaggatga agtggtcatt |
| 181 | ggctttgcca tctctcagca gaagaaggat gtgcggatgc tccggttcat tcaggaggtg |
| 241 | aacaccacca cccggtcctg cgctctctgg gacctggagg aggacacaga atatatcgtc |
| 301 | catgtgcagg ccatctccat ccagggacag agcccagcca gtgagcctgt gctcttcaag |
| 361 | accccacgcg aggctgaaaa gatggcctca aagaacaaag atgaggtgac catgaaggag |

SEQ ID NO: 22 Fragment of Murine Fndc5 Amino Acid Sequence
(residues 29-140)
DSPSAPVNVTVRHLKANSAVVSWDVLEDEVVIGFAISQQKKDVRMLRFIQEVNTTTRSCAL
WDLEEDTEYIVHVQAISIQGQSPASEPVLFKTPREAEKMASKNKDEVTMKE
SEQ ID NO: 23 Fragment of Human Fndc5 (isoform 1) Nucleic Acid
Sequence that encodes amino acid residues 1-68 of Human Fndc5

| | |
|---|---|
| 1 | atgctgcgct tcatccagga ggtgaacacc accacccgct catgtgccct ctgggacctg |
| 61 | gaggaggata cggagtacat agtccacgtg caggccatct ccattcaggg ccagagccca |
| 121 | gccagcgagc ctgtgctctt caagaccccg cgtgaggctg agaagatggc ctccaagaac |
| 181 | aaagatgagg taaccatgaa agag |

SEQ ID NO: 24 Fragment of Human Fndc5 (isoform 1) Amino Acid
Sequence (residues 1-68)
MLRFIQEVNTTTRSCALWDLEEDTEYIVHVQAISIQGQSPASEPVLFKTPREAEKMASKNK
DEVTMKE
SEQ ID NO: 25 Fragment of Human Fndc5 (isoform 2) Nucleic Acid
Sequence that encodes amino acid residues 32-143 of Human Fndc5

| | |
|---|---|
| 94 | gacagtc cctcagcccc agtgaacgtc |
| 121 | accgtcaggc acctcaaggc caactctgca gtggtgagct gggatgttct ggaggatgag |
| 181 | gttgtcatcg gatttgccat ctcccagcag aagaaggatg tgcggatgct gcgcttcatc |
| 241 | caggaggtga acaccaccac ccgctcatgt gccctctggg acctggagga ggatacggag |
| 301 | tacatagtcc acgtgcaggc catctccatt cagggccaga gcccagccag cgagcctgtg |
| 361 | ctcttcaaga ccccgcgtga ggctgagaag atggcctcca agaacaaaga tgaggtaacc |
| 421 | atgaaagag |

SEQ ID NO: 26 Fragment of Human Fndc5 (isoform 2) Amino Acid
Sequence (residues 32-143)
DSPSAPVNVTVRHLKANSAVVSWDVLEDEVVIGFAISQQKKDVRMLRFIQEVNTTTRSCAL
WDLEEDTEYIVHVQAISIQGQSPASEPVLFKTPREAEKMASKNKDEVTMKE
SEQ ID NO: 27 Fragment of Human Fndc5 (isoform 3) Nucleic Acid
Sequence that encodes amino acid residues 32-143 of Human Fndc5

| | |
|---|---|
| 94 | gacagtc cctcagcccc agtgaacgtc |
| 121 | accgtcaggc acctcaaggc caactctgca gtggtgagct gggatgttct ggaggatgag |
| 181 | gttgtcatcg gatttgccat ctcccagcag aagaaggatg tgcggatgct gcgcttcatc |
| 241 | caggaggtga acaccaccac ccgctcatgt gccctctggg acctggagga ggatacggag |
| 301 | tacatagtcc acgtgcaggc catctccatt cagggccaga gcccagccag cgagcctgtg |
| 361 | ctcttcaaga ccccgcgtga ggctgagaag atggcctcca agaacaaaga tgaggtaacc |
| 421 | atgaaagag |

SEQ ID NO: 28 Fragment of Human Fndc5 (isoform 3) Amino Acid
Sequence (residues 32-143)
DSPSAPVNVTVRHLKANSAVVSWDVLEDEVVIGFAISQQKKDVRMLRFIQEVNTTTRSCAL
WDLEEDTEYIVHVQAISIQGQSPASEPVLFKTPREAEKMASKNKDEVTMKE
* Fragments of SEQ ID NOs: 21-28 are non-limiting representative embodiments of irisin.
* Included in Table 1 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any SEQ ID NO listed in Table 1, or a portion thereof, such as fragments that are less than about 1000, 950, 900, 850, 800, 750, 700, 650, 600, 550, 500, 450, 400, 350, 300, 250, 210, or less, or any range in between, inclusive, such as 210-585 nucleotides. Such nucleic acid molecules can have a function of the full-length nucleic acid as described further herein.
* Included in Table 1 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1, or a portion thereof, such as fragments that are less than about 195, 190, 185, 180, 175, 170, 165, 160, 155, 150, 145, 140, 135, 130, 125, 120, 115, 110, 105, 100, 95, 90, 85, 80, 75, or 70 amino acids in length, or any range in between, inclusive, such as 70-195 amino acids. Such polypeptides can have a function of the full-length polypeptide as described further herein.

### II. Nucleic Acids, Polypeptides, Antibodies, Vectors, and Host Cells Useful for the Methods Described Herein

Nucleic acids, polypeptides, and antibodies related to Fndc5, irisin, irisin receptor, or protease that cleaves Fndc5 into irisin, or fragments thereof, are useful for carrying out the methods described herein.

In some embodiments, the present invention contemplates the use of antisense nucleic acid molecules, i.e., molecules which are complementary to a sense nucleic acid of the present invention, e.g., complementary to the coding strand of a double-stranded cDNA molecule corresponding to a marker of the present invention (e.g., FNDC5 or protease that cleaves FNDC5) or complementary to an mRNA sequence corresponding to a marker of the present invention (e.g., FNDC5 or protease that cleaves FNDC5). Accordingly, an antisense nucleic acid molecule of the present invention can hydrogen bond to (i.e. anneal with) a sense nucleic acid of the present invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a polypeptide of the present invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the present invention are typically administered to a subject or generated in situ such that they hybridize with or bind to cellular mRNA and/or genomic DNA encoding a polypeptide corresponding to a selected marker of the present invention to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the present invention includes direct injection at a tissue site or infusion of the antisense nucleic acid into a blood- or bone marrow-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the present invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The present invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide corresponding to a marker of the present invention (e.g., FNDC5 or protease that cleaves FNDC5) can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the present invention can be used to select a catalytic RNA having a specific ribonuclease activity from a pool of RNA molecules (see, e.g., Bartel and Szostak, 1993, Science 261:1411-1418).

The present invention also encompasses nucleic acid molecules which form triple helical structures. For example, expression of FNDC5 or protease that cleaves FNDC5 can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNASE H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The present invention also pertains to variants of the polypeptides described herein (e.g., irisin, FNDC5, irisin receptor, or protease that cleaves FNDC5 into irisin). Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein (e.g., irisin, FNDC5, irisin receptor, or protease that cleaves FNDC5 into irisin) which function as either agonists (mimetics) or as antagonists can be identified by screening combinatorial libraries of mutants, e.g., truncation mutants, of the protein of the present invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the present invention from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of fragments of the coding sequence of a polypeptide corresponding to a marker of the present invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with Si nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the present invention (Arkin and Yourvan, 1992, Proc. Natl. Acad. Sci. USA 89:7811-7815; Delgrave et al., 1993, Protein Engineering 6(3):327-331).

An isolated polypeptide or a fragment thereof (or a nucleic acid encoding such a polypeptide) corresponding to one or more markers of the invention (e.g., irisin, FNDC5, irisin receptor, or protease that cleaves FNDC5 into irisin), including the ones listed in Table 1 or fragments thereof, can be used as an immunogen to generate antibodies that bind to said immunogen, using standard techniques for polyclonal and monoclonal antibody preparation according to well-known methods in the art. An antigenic peptide comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody and/or an intrbody, binds substantially specifically to irisin and inhibits or blocks its biological function, such as by interrupting its interaction with an irisin receptor. In another embodiment, an antibody and/or an intrbody, binds substantially specifically to an irisin receptor, such as the irisin receptors described herein, and inhibits or blocks its biological function, such as by interrupting its interaction to irisin. In still another embodiment, an antibody and/or an introbody, binds substantially specifically to FNDC5 and decreases the amount of FNDC5 or inhibits its cleavage into irisin. In yet another embodiment, an antibody and/or an introbody, binds substantially specifically to the protease that cleaves FNDC5 and decreases the amount of the protease that cleaves FNDC5 or inhibits or blocks its biological function in cleaving FNDC5 into irisin.

For example, a polypeptide immunogen typically is used to prepare antibodies by immunizing a suitable subject (e.g., rabbit, goat, mouse or other mammal) with the immunogen. A preferred animal is a mouse deficeint in the desired target antigen. For example, a PD-1 knockout mouse if the desired antibody is an anti-PD-1 antibody, may be used. This results in a wider spectrum of antibody recognition possibilities as antibodies reactive to common mouse and human epitopes are not removed by tolerance mechanisms. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography, to obtain the IgG fraction. At an appropriate time after immunization, e.g., when the antibody titers are highest, antibody-producing cells can be obtained from the subject and used to prepare monoclonal antibodies by standard techniques, such as the hybridoma technique (originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well-known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically. In some embodiments, the immunization is performed in a cell or animal host that has a knockout of a target antigen of interest (e.g., does not produce the antigen prior to immunization).

Any of the many well-known protocols used for fusing lymphocytes and immortalized cell lines can be applied for the purpose of generating a monoclonal antibody against one or more markers of the invention (e.g., irisin, FNDC5, irisin receptor, or protease that cleaves FNDC5 into irisin), including the ones listed in Table 1 or fragments thereof (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Since it is well-known in the art that antibody heavy and light chain CDR3 domains play a particularly important role in the binding specificity/affinity of an antibody for an antigen, the recombinant monoclonal antibodies of the present invention prepared as set forth above preferably comprise the heavy and light chain CDR3s of variable regions of the antibodies described herein and well-known in the art. Similarly, the antibodies can further comprise the CDR2s of variable regions of said antibodies. The antibodies can further comprise the CDR1s of variable regions of said antibodies. In other embodiments, the antibodies can comprise any combinations of the CDRs.

The CDR1, 2, and/or 3 regions of the engineered antibodies described above can comprise the exact amino acid sequence(s) as those of variable regions of the present invention described herein. However, the ordinarily skilled artisan will appreciate that some deviation from the exact CDR sequences may be possible while still retaining the ability of the antibody, especially an introbody, to bind a desired target, such as irisin and/or a binding partner thereof effectively (e.g., conservative sequence modifications). Accordingly, in another embodiment, the engineered antibody may be composed of one or more CDRs that are, for example, 50%, 60%, 70%, 80%, 85%, 90%, 91%, 92%, 93%, 94%, 95%, 96%, 97%, 98%, 99%, or 99.5% identical to one or more CDRs of the present invention described herein or otherwise publicly available.

For example, the structural features of non-human or human antibodies (e.g., a rat anti-mouse/anti-human antibody) can be used to create structurally related human antibodies, especially introbodies, that retain at least one functional property of the antibodies of the present invention, such as binding to irisin, irisin binding partners/substrates. Another functional property includes inhibiting binding of the original known, non-human or human antibodies in a competition ELISA assay.

Antibodies, immunoglobulins, and polypeptides of the invention can be used in an isolated (e.g., purified) form or contained in a vector, such as a membrane or lipid vesicle (e.g. a liposome). Moreover, amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Similarly, modifications and changes may be made in the structure of the antibodies described herein, and in the DNA sequences encoding them, and still obtain a functional molecule that encodes an antibody and polypeptide with desirable characteristics. For example, antibody glycosylation patterns can be modulated to, for example, increase stability. By “altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr et al. (1987) and by Edge et al. (1981). Enzymatic cleavage of carbohydrate moieties on antibodies can be achieved by the use of a variety of endo- and exo-glycosidases as described by Thotakura et al. (1987).

Other modifications can involve the formation of immunoconjugates. For example, in one type of covalent modification, antibodies or proteins are covalently linked to one of a variety of non proteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Conjugation of antibodies or other proteins of the present invention with heterologous agents can be made using a variety of bifunctional protein coupling agents including but not limited to N-succinimidyl (2-pyridyldithio) propionate (SPDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody (WO 94/11026).

In another aspect, the present invention features antibodies conjugated to a therapeutic moiety, such as a cytotoxin, a drug, and/or a radioisotope. When conjugated to a cytotoxin, these antibody conjugates are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Conjugated antibodies, in addition to therapeutic utility, can be useful for diagnostically or prognostically to monitor polypeptide levels in tissue as part of a clinical testing procedure, e.g., to determine the efficacy of a given treatment regimen. Detection can be facilitated by coupling (i e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (PE); an example of a luminescent material includes luminol; examples of bioluminescent materials include luciferase, luciferin, and aequorin, and examples of suitable radioactive material include ¹²⁵I, ¹³¹I, ³⁵S, or ³H. [0134] As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody, as well as indirect labeling of the antibody by reactivity with a detectable sub stance.

The antibody conjugates of the present invention can be used to modify a given biological response. The therapeutic moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, Pseudomonas exotoxin, or diphtheria toxin; a protein such as tumor necrosis factor or interferon y; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other cytokines or growth factors.

In one embodiment, an antibody for use in the instant invention is a bispecific or multispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a polypeptide or a fragment thereof of one or more markers of the invention (e.g., irisin, FNDC5, irisin receptor, or protease that cleaves FNDC5 into irisin), including the ones listed in Table 1, or a fragment thereof. In one embodiment, the bispecific antibody could specifically bind to both a polypeptide or a fragment thereof and its natural binding partner(s) or a fragment(s) thereof.

Techniques for modulating antibodies, such as humanization, conjugation, recombinant techniques, and the like are well-known in the art.

In another aspect of this invention, peptides or peptide mimetics can be used to antagonize the activity of one or more markers of the invention (e.g., irisin, FNDC5, irisin receptor, or protease that cleaves FNDC5 into irisin), including the ones listed in Table 1 or fragments thereof. In one embodiment, variants of one or more markers listed in Table 1 which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with Si nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more one or more markers of the invention (e.g., irisin, FNDC5, irisin receptor, or protease that cleaves FNDC5 into irisin), including the ones listed in Table 1 or fragments thereof. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences described herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides described herein can be used therapeutically to treat disease, e.g., by altering costimulation in a patient.

Peptidomimetics (Fauchere (1986) Adv. Drug Res. 15:29; Veber and Freidinger (1985) TINS p.392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (e.g., inhibit) interactions, e.g., between markers described herein or listed in Table 1 and their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Chimeric or fusion proteins can be prepared for the irisin inhibitors and/or irisin mutants described herein, such as inhibitors to one or more markers of the invention (e.g., irisin, FNDC5, irisin receptor, or protease that cleaves FNDC5 into irisin), including the ones listed in Table 1, or fragments thereof. As used herein, a “chimeric protein” or “fusion protein” comprises one or more markers of the invention (e.g., irisin, FNDC5, irisin receptor, or protease that cleaves FNDC5 into irisin), including the ones listed in Table 1, or fragments thereof, operatively linked to another polypeptide having an amino acid sequence corresponding to a protein which is not substantially homologous to the respective biomarker. In a preferred embodiment, the fusion protein comprises at least one biologically active portion of one or more biomarkers of the invention (e.g., irisin, FNDC5, irisin receptor, or protease that cleaves FNDC5 into irisin), including the ones listed in Table 1, or fragments thereof. Within the fusion protein, the term “operatively linked” is intended to indicate that the biomarker sequences and the non-biomarker sequences are fused in-frame to each other in such a way as to preserve functions exhibited when expressed independently of the fusion. The “another” sequences can be fused to the N-terminus or C-terminus of the biomarker sequences, respectively.

Such a fusion protein can be produced by recombinant expression of a nucleotide sequence encoding the first peptide and a nucleotide sequence encoding the second peptide. The second peptide may optionally correspond to a moiety that alters the solubility, affinity, stability or valency of the first peptide, for example, an immunoglobulin constant region. In another preferred embodiment, the first peptide consists of a portion of a biologically active molecule (e.g. the extracellular portion of the polypeptide or the ligand binding portion). The second peptide can include an immunoglobulin constant region, for example, a human Cγ1 domain or Cγ4 domain (e.g., the hinge, CH2 and CH3 regions of human IgCγ 1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well-known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992).

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between one or more polypeptides or a fragment thereof and its natural binding partner(s) or a fragment(s) thereof.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more biomarkers of the invention (e.g., irisin, FNDC5, irisin receptor, or protease that cleaves FNDC5 into irisin), including one or more biomarkers listed in Table 1, or fragment(s) thereof. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21,20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA. In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

miRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to biomarkers listed in Table 1). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21,20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. W088/09810) or the blood-brain barrier (see, e.g., PCT Publication No. W089/10134), hybridization-triggered cleavage agents. (See, e.g., Krol et al. (1988) BioTech. 6:958-976) or intercalating agents. (See, e.g., Zon (1988) Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells, or piwiRNAs. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nat. Biotechnol. 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The modulatory agents described herein (e.g., antibodies, small molecules, peptides, fusion proteins, or small nucleic acids) can be incorporated into pharmaceutical compositions and administered to a subject in vivo. The compositions may contain a single such molecule or agent or any combination of agents described herein. “Single active agents” described herein can be combined with other pharmacologically active compounds (“second active agents”) known in the art according to the methods and compositions provided herein.

The production and use of biomarker nucleic acid and/or biomarker polypeptide molecules described herein can be facilitated by using standard recombinant techniques. In some embodiments, such techniques use vectors, preferably expression vectors, containing a nucleic acid encoding a biomarker polypeptide or a portion of such a polypeptide. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the present invention is intended to include such other forms of expression vectors, such as viral vectors (e.g., replication defective retroviruses, adenoviruses and adeno-associated viruses), which serve equivalent functions.

The recombinant expression vectors of the present invention comprise a nucleic acid of the present invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the present invention can be introduced into host cells to thereby produce proteins or peptides, including fusion proteins or peptides, encoded by nucleic acids as described herein.

The recombinant expression vectors for use in the present invention can be designed for expression of a polypeptide corresponding to a marker of the present invention in prokaryotic (e.g., E. coli) or eukaryotic cells (e.g., insect cells {using baculovirus expression vectors}, yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., p. 60-89, In Gene Expression Technology: Methods in Enzymology vol. 185, Academic Press, San Diego, Calif., 1991). Target biomarker nucleic acid expression from the pTrc vector relies on host RNA polymerase transcription from a hybrid trp-lac fusion promoter. Target biomarker nucleic acid expression from the pET 11d vector relies on transcription from a T7 gn10-lac fusion promoter mediated by a co-expressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

One strategy to maximize recombinant protein expression in E. coli is to express the protein in a host bacterium with an impaired capacity to proteolytically cleave the recombinant protein (Gottesman, p. 119-128, In Gene Expression Technology: Methods in Enzymology vol. 185, Academic Press, San Diego, Calif., 1990. Another strategy is to alter the nucleic acid sequence of the nucleic acid to be inserted into an expression vector so that the individual codons for each amino acid are those preferentially utilized in E. coli (Wada et al., 1992, Nucleic Acids Res. 20:2111-2118). Such alteration of nucleic acid sequences of the present invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J. 6:229-234), pMFa (Kurjan and Herskowitz, 1982, Cell 30:933-943), pJRY88 (Schultz et al., 1987, Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the present invention is expressed in mammalian cells using a mammalian expression vector. Examples of mammalian expression vectors include pCDM8 (Seed, 1987, Nature 329:840) and pMT2PC (Kaufman et al., 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc. Natl. Acad. Sci. USA 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Camper and Tilghman, 1989, Genes Dev. 3:537-546).

The present invention further provides a recombinant expression vector comprising a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the present invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue-specific or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes (see Weintraub et al., 1986, Trends in Genetics, Vol. 1(1)).

Another aspect of the present invention pertains to host cells into which a recombinant expression vector of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

### III. Uses and Methods of the Present Invention

The compositions described herein can be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

a. Screening Methods

In one embodiment, the present invention relates to assays for screening test agents which bind to, or modulate the biological activity of, at least one biomarker described herein (e.g., irisin, FNDC5, irisin receptor, or protease that cleaves FNDC5 into irisin). In one embodiment, a method for identifying such an agent entails determining the ability of the agent to modulate, e.g. inhibit, the at least one biomarker described herein.

In one embodiment, an assay is a cell-free or cell-based assay, comprising contacting at least one biomarker described herein, with a test agent, and determining the ability of the test agent to modulate (e.g., inhibit) the enzymatic activity of the biomarker, such as by measuring direct binding of substrates or by measuring indirect parameters as described below.

For example, in a direct binding assay, biomarker protein (or their respective target polypeptides or molecules) can be coupled with a radioisotope or enzymatic label such that binding can be determined by detecting the labeled protein or molecule in a complex. For example, the targets can be labeled with ¹²⁵I, ³⁵S, ¹⁴C, or ³H, either directly or indirectly, and the radioisotope detected by direct counting of radioemmission or by scintillation counting. Alternatively, the targets can be enzymatically labeled with, for example, horseradish peroxidase, alkaline phosphatase, or luciferase, and the enzymatic label detected by determination of conversion of an appropriate substrate to product. Determining the interaction between biomarker and substrate can also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target can be accomplished in any vessel suitable for containing the reactants. Non-limiting examples of such vessels include microtiter plates, test tubes, and micro-centrifuge tubes. Immobilized forms of the antibodies described herein can also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In an alternative embodiment, determining the ability of the agent to modulate the interaction between the biomarker and a substrate or a biomarker and its natural binding partner can be accomplished by determining the ability of the test agent to modulate the activity of a polypeptide or other product that functions downstream or upstream of its position within the signaling pathway (e.g., feedback loops). Such feedback loops are well-known in the art (see, for example, Chen and Guillemin (2009) Int. J. Tryptophan Res. 2:1-19).

In another embodiment, the present invention relates to assays for screening for a biologically inactive or inhibitory irisin mutant that binds to the irisin receptor in osteocytes.

In one embodiment, an assay is a cell-based assay in which a cell, such as an osteocyte, is contacted with a test agent, such as an irisin mutant polypeptide, or fragments thereof, and the biological activity of the irsin mutant and its binding to irisn receptor is determined. Determining the biological activity of the irsin mutant can be accomplished by testing its effects on, for example, activitation of substrates (e.g., pFAK, pZyxin, and pCREB) of the irisin receptor, scleostin induction, osteocyte survival, the degradative function of osteocyte, and the like. Determining the binding of the irsin mutant to irsin receptor can be accomplished by, for example, the direct binding assay described above.

The present invention further pertains to novel agents identified by the above-described screening assays. Accordingly, it is within the scope of this invention to further use an agent identified as described herein, such as in an appropriate animal model. For example, an agent identified as described herein can be used in an animal model to determine the efficacy, toxicity, or side effects of treatment with such an agent. Alternatively, an antibody identified as described herein can be used in an animal model to determine the mechanism of action of such an agent.

b. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining the amount and/or activity level of a biomarker described herein (e.g., irisin, FNDC5, irisin receptor, or protease that cleaves FNDC5 into irisin) in the context of a biological sample (e.g., blood, serum, cells, or tissue) to thereby determine whether an individual afflicted with a bone loss condition is likely to respond to an irsin-based therapy. Such assays can be used for prognostic or predictive purpose alone, or can be coupled with a therapeutic intervention to thereby prophylactically treat an individual prior to the onset or after recurrence of a disorder characterized by or associated with biomarker polypeptide, nucleic acid expression or activity. The skilled artisan will appreciate that any method can use one or more (e.g., combinations) of biomarkers described herein, such as those in the tables, figures, examples, and otherwise described in the specification (e.g., irisin, FNDC5, irisin receptor, or protease that cleaves FNDC5 into irisin).

Another aspect of the present invention pertains to monitoring the influence of agents (e.g., drugs, compounds, antibodies, and small nucleic acid-based molecules) on the expression or activity of a biomarker described herein (e.g., irisin, FNDC5, irisin receptor, or protease that cleaves FNDC5 into irisin). These and other agents are described in further detail in other sections.

The skilled artisan will also appreciated that, in certain embodiments, the methods of the present invention implement a computer program and computer system. For example, a computer program can be used to perform the algorithms described herein. A computer system can also store and manipulate data generated by the methods of the present invention which comprises a plurality of biomarker signal changes/profiles which can be used by a computer system in implementing the methods of this invention. In certain embodiments, a computer system receives biomarker expression data; (ii) stores the data; and (iii) compares the data in any number of ways described herein (e.g., analysis relative to appropriate controls) to determine the state of informative biomarkers from disease tissue. In other embodiments, a computer system (i) compares the determined expression biomarker level to a threshold value; and (ii) outputs an indication of whether said biomarker level is significantly modulated (e.g., above or below) the threshold value, or a phenotype based on said indication.

In certain embodiments, such computer systems are also considered part of the present invention. Numerous types of computer systems can be used to implement the analytic methods of this invention according to knowledge possessed by a skilled artisan in the bioinformatics and/or computer arts. Several software components can be loaded into memory during operation of such a computer system. The software components can comprise both software components that are standard in the art and components that are special to the present invention (e.g., dCHIP software described in Lin et al. (2004) Bioinformatics 20, 1233-1240; radial basis machine learning algorithms (RBM) known in the art).

The methods of the present invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.).

In certain embodiments, the computer comprises a database for storage of biomarker data. Such stored profiles can be accessed and used to perform comparisons of interest at a later point in time. For example, biomarker expression profiles of a sample derived from the non-disease tissue of a subject and/or profiles generated from population-based distributions of informative loci of interest in relevant populations of the same species can be stored and later compared to that of a sample derived from the disease tissue of the subject or tissue suspected of being affected of the subject.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

c. Diagnostic Assays

The present invention provides, in part, methods, systems, and code for accurately classifying whether a biological sample is associated with a bone loss condition that is likely to respond to an irisin-based therapy. In some embodiments, the present invention is useful for classifying a sample (e.g., from a subject) as associated with or at risk for responding to or not responding to an irisin-based therapy using a statistical algorithm and/or empirical data (e.g., the amount or activity of a biomarker described herein, such as in the tables, figures, examples, and otherwise described in the specification (e.g., irisin, FNDC5, irisin receptor, or protease that cleaves FNDC5 into irisin)).

An exemplary method for detecting the amount or activity of a biomarker described herein (e.g., irisin, FNDC5, irisin receptor, or protease that cleaves FNDC5 into irisin), and thus useful for classifying whether a sample is likely or unlikely to respond to irisin-based therapy involves obtaining a biological sample from a test subject and contacting the biological sample with an agent, such as a protein-binding agent like an antibody or antigen-binding fragment thereof, or a nucleic acid-binding agent like an oligonucleotide, capable of detecting the amount or activity of the biomarker in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as a based upon a prediction or probability value and the presence or level of the biomarker. The use of a single learning statistical classifier system typically classifies the sample as, for example, a likely immunotherapy responder or progressor sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the sample classification results to a clinician, e.g., an oncologist.

In another embodiment, the diagnosis of a subject is followed by administering to the individual a therapeutically effective amount of a defined treatment based upon the diagnosis.

In one embodiment, the methods further involve obtaining a control biological sample (e.g., biological sample from a subject who does not have a bone loss condition or whose bone loss condition is susceptible to irisin-based therapy), a biological sample from the subject during remission, or a biological sample from the subject during treatment for developing a bone loss condition despite irisin-based therapy.

d. Prognostic Assays

The diagnostic methods described herein can furthermore be utilized to identify subjects having or at risk of developing a bone loss condition that is likely or unlikely to be responsive to an irisin-based therapy. The assays described herein, such as the preceding diagnostic assays or the following assays, can be utilized to identify a subject having or at risk of developing a disorder associated with a misregulation of the amount or activity of at least one biomarker described herein (e.g., irisin, FNDC5, irisin receptor, or protease that cleaves FNDC5 into irisin), such as bone loss conditions. Alternatively, the prognostic assays can be utilized to identify a subject having or at risk for developing a disorder associated with a misregulation of the at least one biomarker described herein (e.g., irisin, FNDC5, irisin receptor, or protease that cleaves FNDC5 into irisin), such as bone loss conditions. Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with the aberrant biomarker expression or activity, such as bone loss conditions.

e. Methods of Treatment

The present invention provides for both prophylactic and therapeutic methods of treating or preventing a bone loss condition in a subject, e.g., a human, at risk of (or susceptible to) bone loss, by administering to said subject a therapeutically effective amount of an agent that decreases the amount and/or activity of irisin, or a biologically inactive or inhibitory irisin mutant that binds to the irisin receptor in osteocytes. In some embodiments, which includes both prophylactic and therapeutic methods, the irisin modulator or mutant is administered by in a pharmaceutically acceptable formulation.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either irisin inhibitors or mutants according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the present invention provides a method for treating or preventing a subject afflicted with bone loss conditions by administering to the subject a therapeutically effective amount of an agent that decreases the amount and/or activity of irisin. The present invention also provides a method for treating or preventing a subject afflicted with bone loss conditions by administering to the subject a therapeutically effective amount of a biologically inactive or inhibitory irisin mutant that binds to the irisin receptor in osteocytes. Subjects at risk for a bone loss condition can be identified by, for example, any or a combination of the diagnostic or prognostic assays described herein. Administration of a prophylactic agent can occur prior to the manifestation of symptoms characteristic of a bone loss condition, such that bone loss condition or symptom thereof, is prevented or, alternatively, delayed in its progression.

2. Therapeutic Methods

The therapeutic compositions described herein, such as the irisin inhibitor or the biologically inactive or inhibitory irisin mutant that binds to the irisin receptor on the osteocyte, can be used in a variety of in vitro and in vivo therapeutic applications using the formulations and/or combinations described herein. In one embodiment, the therapeutic agents can be used to treat bone loss conditions determined to be responsive thereto. For example, single or multiple agents that decrease the amount and/or activity of irisin can be used to treat bone loss conditions in subjects identified as likely responders thereto.

Modulatory methods of the present invention involve contacting a cell, such as an osteocyte with an agent that decreases the amount and/or activity of of irisin, or with a biologically inactive or inhibitory irisin mutant that binds to the irisin receptor on the osteocyte. Exemplary agents useful in such methods are described above. Such agents can be administered in vitro or ex vivo (e.g., by contacting the cell with the agent) or, alternatively, in vivo (e.g., by administering the agent to a subject).

### IV. Clinical Efficacy

The present invention further provides methods for determining the effectiveness of an irisin-based therapy (e.g., an agent that decreases the amount and/or activity of irisin, or a biologically inactive or inhibitory irisin mutant that binds to the irisin receptor) in treating or preventing a bone loss condition or assessing risk of developing a bone loss condition in a subject. For example, the effectiveness of such an irisin-based therapy can be monitored in clinical trials of subjects. In such clinical trials, the amount or activity of irisin, FNDC5, protease that cleaves FNDC5 into irsin, or other genes that have been implicated in, for example, a irisin-activated pathway can be used as a “read out” or marker of the phenotype of a particular cell.

To study the effect of agents which modulate irsin amount and/or activity in subjects suffering from or at risk of developing a bone loss condition, or agents to be used as a prophylactic, for example, in a clinical trial, cells can be isolated and analyzed for the levels of irisin and other genes implicated in irisin activity or amount. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of irisin or other genes, such as the FNDC5. In this way, the gene expression pattern can serve as a marker, indicative of the physiological response of the cells to the agent which modulates irisin level or activity. This response state may be determined before, and at various points during treatment of the individual with the agent which modulates irisin level or activity

In one embodiment, the present invention provides a method of assessing the efficacy of an agent for treating bone loss conditions in a subject including the steps of (a) detecting in a subject sample at a first point in time the amount and/or acvitity of irisin; (b) repeating step (a) during at least one subsequent point in time after administration of the agent; and (c) comparing the amount detected in steps (a) and (b), wherein the absence of, or a significant decrease in amount and/or activity of irisin in the subsequent sample as compared to the amount and/or activity of irisin in the sample at the first point in time, indicates that the agent treats bone loss in the subject. The agent may be an antibody, peptidomimetic, protein, peptide, nucleic acid, siRNA, or small molecule identified by the screening assays described herein which decreases the level and/or activity of irisin. According to such an embodiment, irisin level or activity may be used as an indicator of the effectiveness of an agent, even in the absence of an observable phenotypic response.

### V. Administration of Agents

The agents of the invention are administered to subjects in a biologically compatible form suitable for pharmaceutical administration in vivo, to prevent and/or treat the bone loss conditions. By “biologically compatible form suitable for administration in vivo” is meant a form to be administered in which any toxic effects are outweighed by the therapeutic effects. The term “subject” is intended to include living organisms in which irisin level or activity can be modulated, e.g., mammals. Examples of subjects include humans, dogs, cats, mice, rats, and transgenic species thereof. Administration of an agent as described herein can be in any pharmacological form including a therapeutically active amount of an agent alone or in combination with a pharmaceutically acceptable carrier.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “therapeutically-effective amount” as used herein means that amount of an agent that modulates (e.g., inhibits) irisin level and/or activity, or composition comprising an agent that modulates (e.g., inhibits) irisin level and/or activity, which is effective for producing some desired therapeutic effect, e.g., treatment of bone loss conditions, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically-acceptable salts” refers to the relatively non-toxic, inorganic and organic acid addition salts of the agents that modulates (e.g., inhibits) irisin level and/or activity encompassed by the present invention. These salts can be prepared in situ during the final isolation and purification of the therapeutic agents, or by separately reacting a purified therapeutic agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66:1-19).

In other cases, the agents useful in the methods of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of agents that modulates (e.g., inhibits) irisin level and/or activity. These salts can likewise be prepared in situ during the final isolation and purification of the therapeutic agents, or by separately reacting the purified therapeutic agent in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well-known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent that modulates (e.g., inhibits) irisin level and/or activity, with the carrier and, optionally, one or more accessory ingredients. In general, the formulations are prepared by uniformly and intimately bringing into association a therapeutic agent with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a therapeutic agent as an active ingredient. A compound may also be administered as a bolus, electuary or paste.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well-known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more therapeutic agents with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Formulations which are suitable for vaginal administration also include pessaries, tampons, creams, gels, pastes, foams or spray formulations containing such carriers as are known in the art to be appropriate.

Dosage forms for the topical or transdermal administration of an agent that modulates (e.g., inhibits) irisin level and/or activity include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain, in addition to a therapeutic agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an agent that modulates (e.g., inhibits) irisin level and/or activity, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The agent that modulates (e.g., inhibits) irisin level and/or activity, can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a therapeutic agent to the body. Such dosage forms can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Pharmaceutical compositions of this invention suitable for parenteral administration comprise one or more therapeutic agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of an agent that modulates (e.g., inhibits) irisin level and/or activity, in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

When the therapeutic agents of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

The nucleic acid molecules of the present invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, an agent of the invention is an antibody. As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays.

### VI. Kits

The present invention also encompasses kits for detecting and/or modulating biomarkers (e.g., irisin, FNDC5, irisin receptor, or protease that cleaves FNDC5 into irisin) described herein. A kit of the present invention may also include instructional materials disclosing or describing the use of the kit or an antibody of the disclosed invention in a method of the disclosed invention as provided herein. A kit may also include additional components to facilitate the particular application for which the kit is designed. For example, a kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents recognized for use in a method of the disclosed invention. Non-limiting examples include agents to reduce non-specific binding, such as a carrier protein or a detergent.

### EXEMPLIFICATION

This invention is further illustrated by the following examples, which should not be construed as limiting.

### Example 1

### Materials and Methods for Examples 2-8

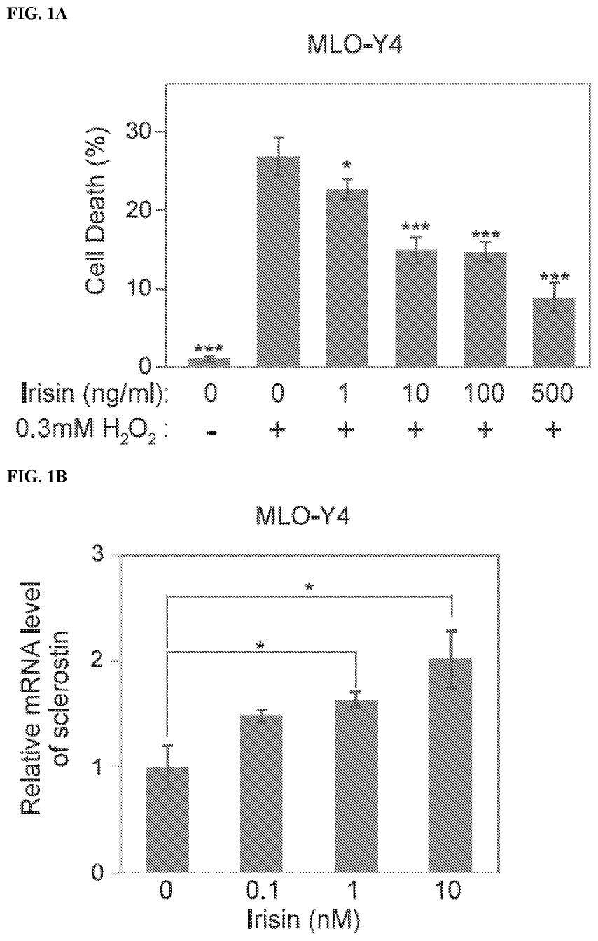

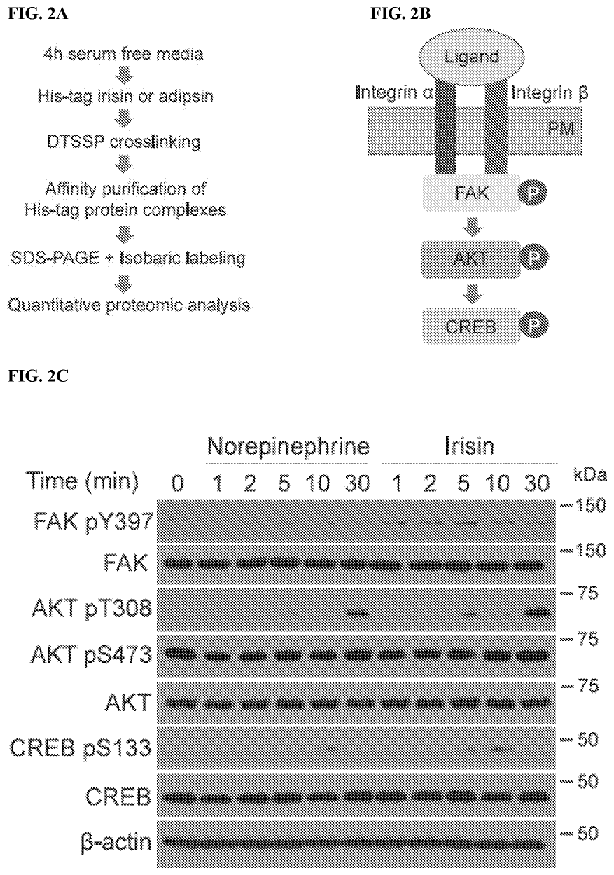

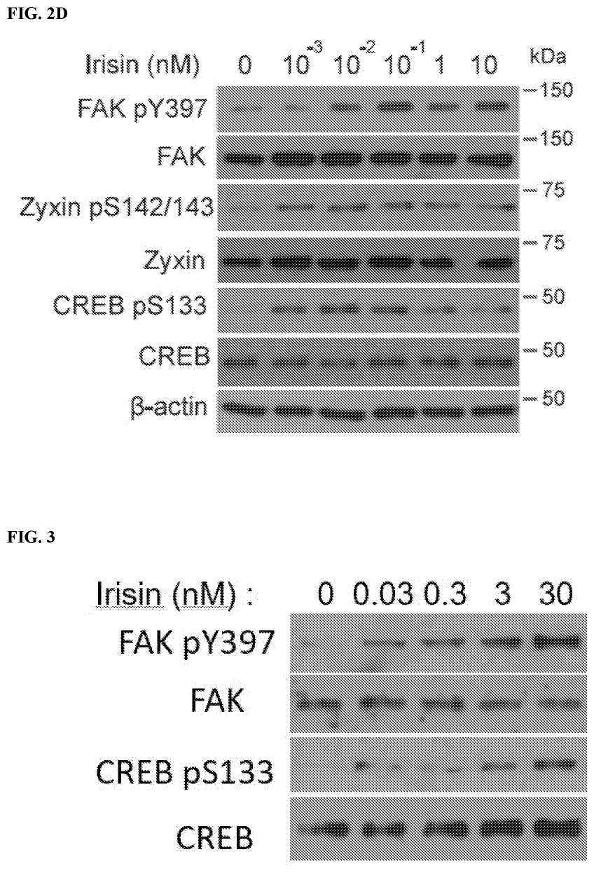

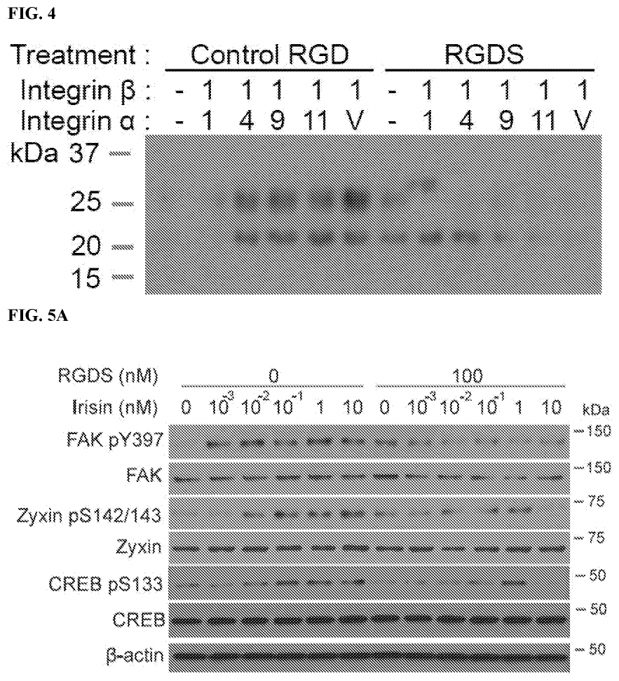

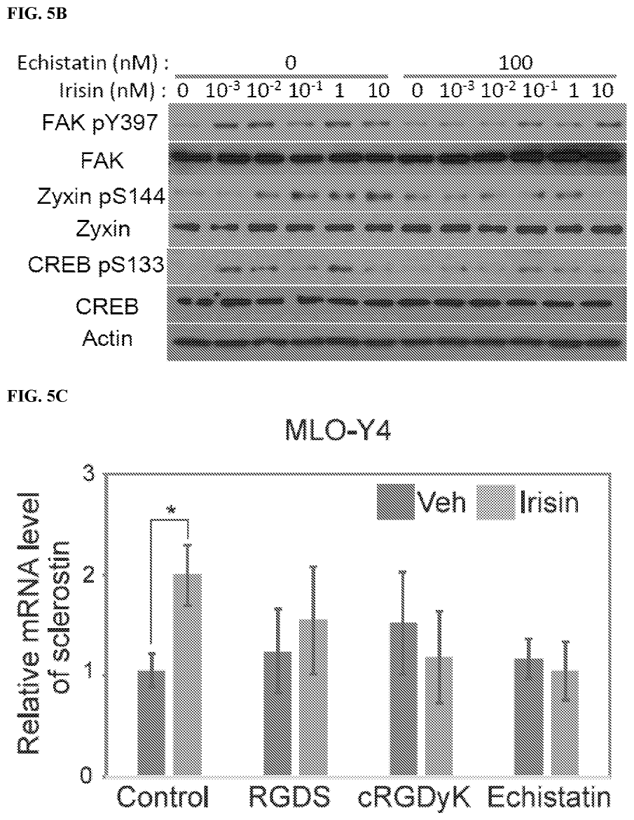

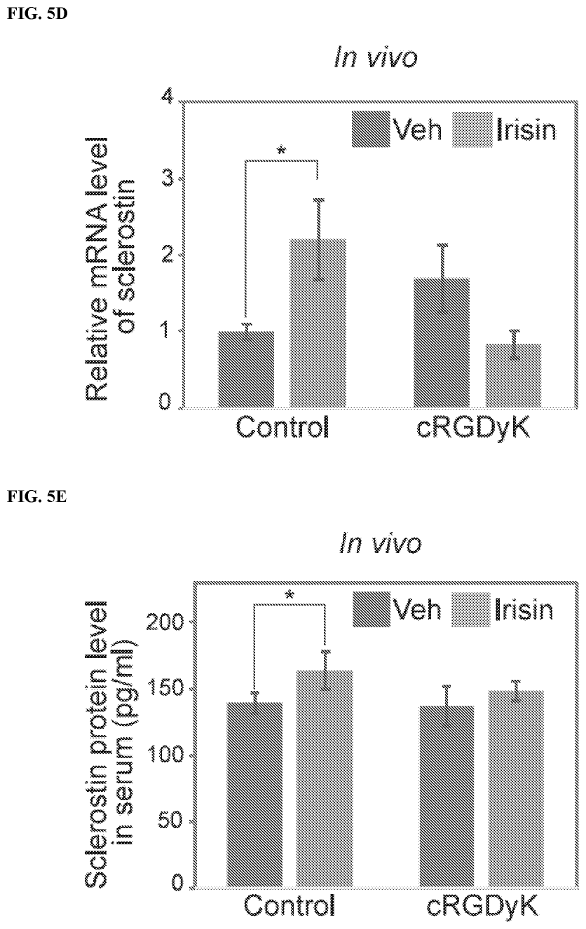

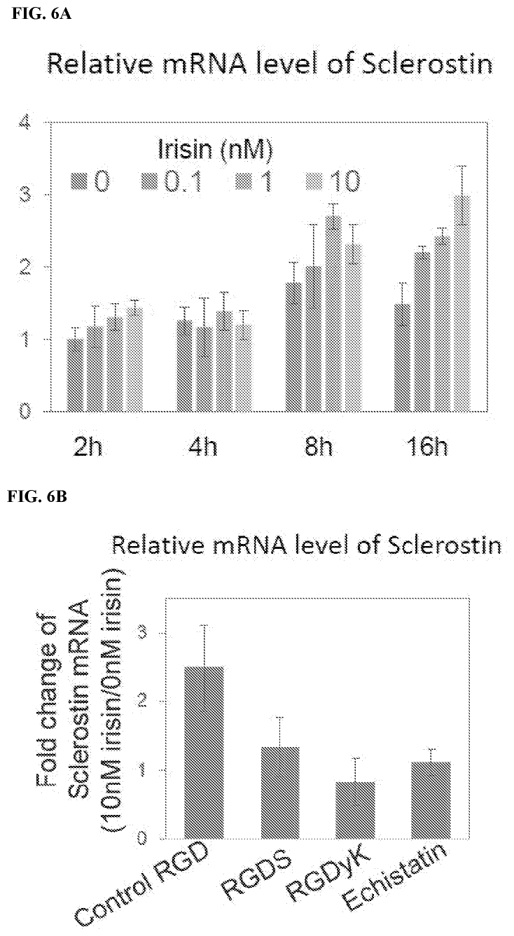

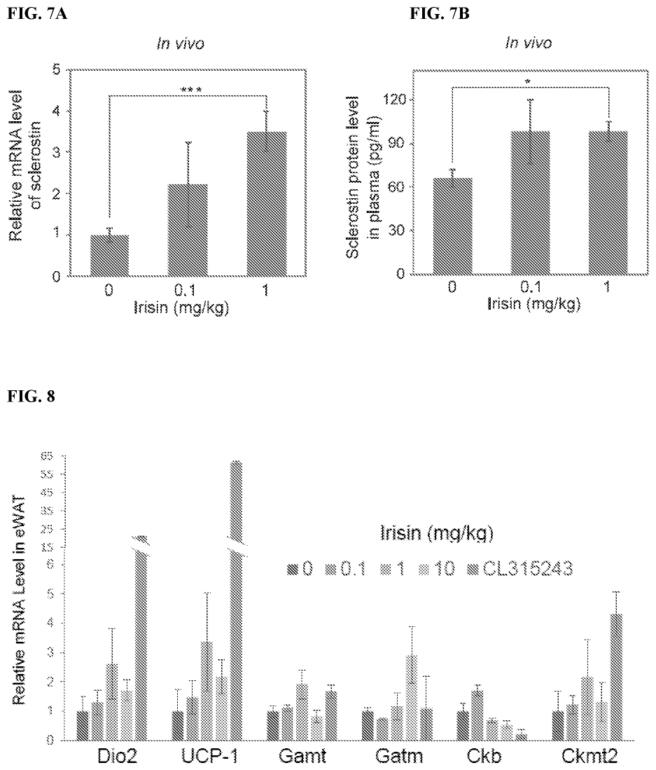

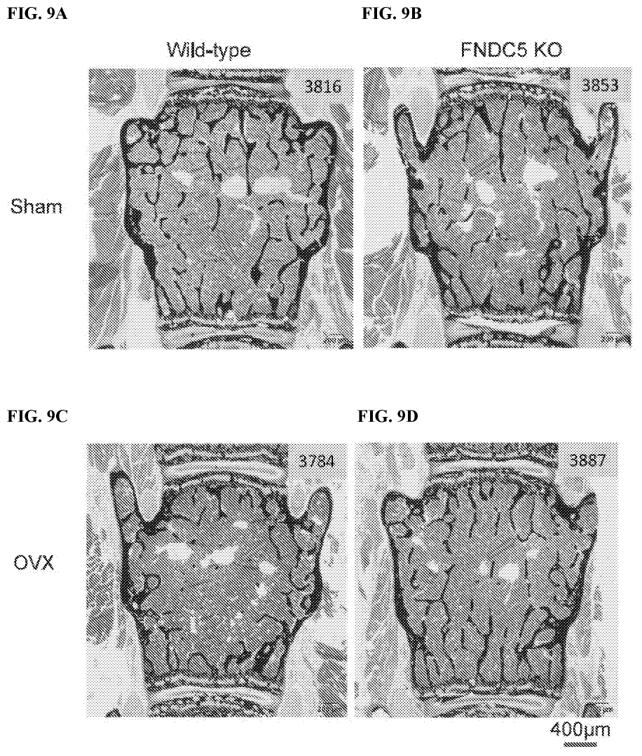

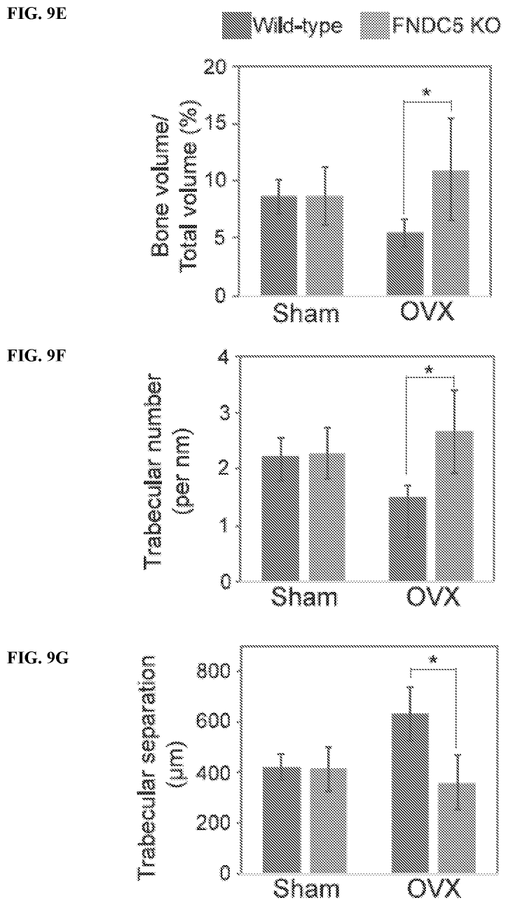

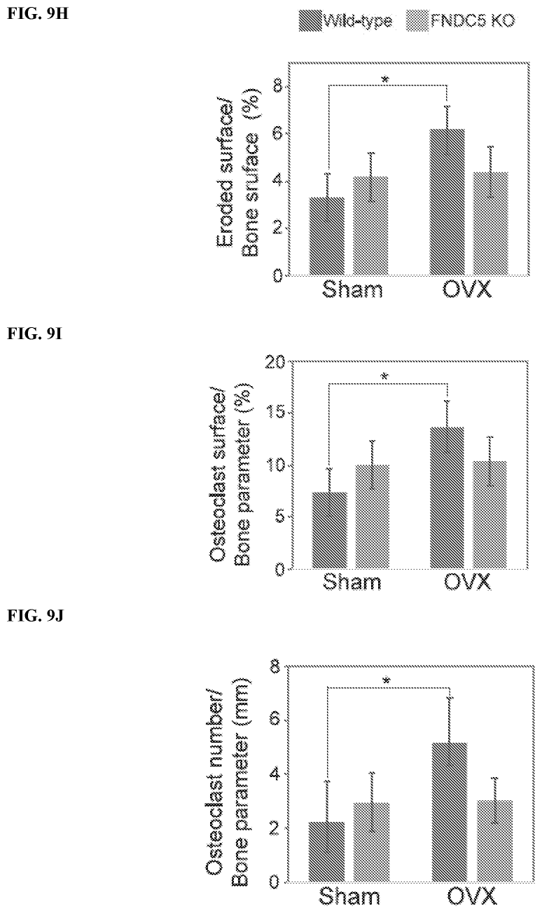

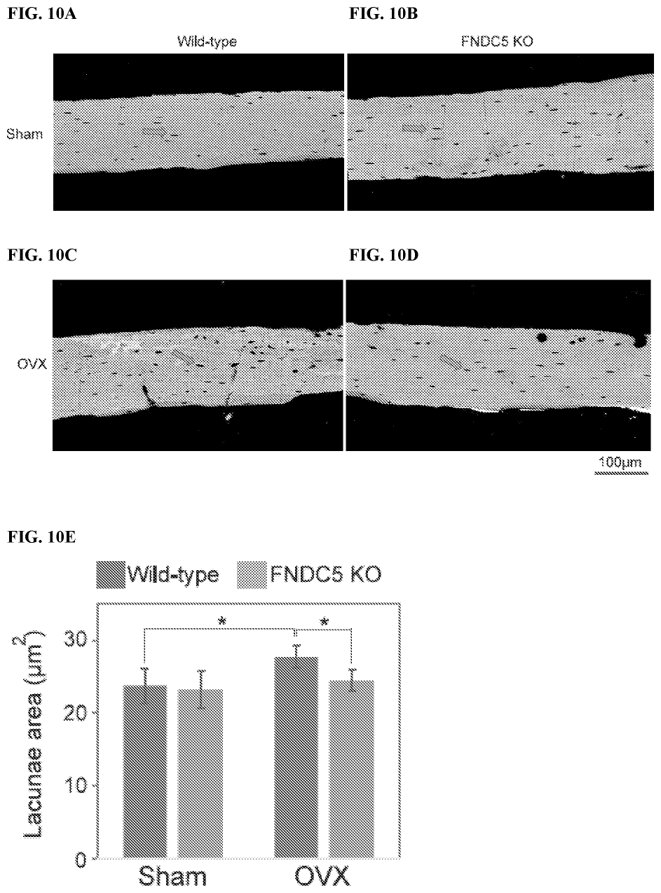

Certain materials and methods were used to generate the results described herein. For example, the data shown in FIG. 1A resulted from MLO-Y4 (an osteocyte-like cell line) cells treated with the indicated concentration of irisin and hydrogen peroxide for 4 hours. Cells were stained with Hoechst 33342 (ThermoFisher Scientific, catalog number H3570) and Eth-D1 (ThermoFisher Scientific, catalog number E1169) and analyzed to determine the percentage of cell death using ImageJ. The data shown in FIG. 2D resulted from MLO-Y4 incubated in serum free medium (FreeStyle™ 293 expression medium, ThermoFisher Scientific, catalog number 12338018) for 4 hours followed by treatment of indicated concentrations of irisin for 10 minutes. Cells were lysed to detect the indicated protein level using immunoblot analysis (Cell Signaling Technology, catalog numbers 3283S, 3285S, 8467S, 3553S, 9198S, and 9104S; Abcam, catalog number ab49900-100ul). The data shown in FIG. 3 resulted from 3T3-F442A cells incubated in serum free medium for 4 hours followed by treatment of indicated concentrations of irisin for 10 minutes. Cells were lysed by RIPA buffer to detect the indicated protein level using immunoblot analysis. The data shown in FIG. 4 were generated from 100 nM flag-tagged irisin (Enzo life sciences, catalog number ADI-908-307-3010) incubated with 5 nM of the indicated 6 his-tag integrins (R & D Systems Inc., catalog numbers 7064-AB-025, 5668-A4-050, 5438-A9-050, 6357-AB-050, and 6579-AV-025) in the presence of RGDS peptide (R & D Systems Inc., catalog number 3498/10) or its control peptide (Enzo Life Sciences, catalog Number BML-P701-0005) followed by immunoprecipitation using 6 his-tag beads (ThermoFisher Scientific, catalog number R901-01). Co-precipitated irisin was analyzed by immunoblot analysis against flag tag (Sigma Aldrich, catalog number A8592). The data shown in FIGS. 5A and 5B were generated from MLO-Y4 cells treated and analyzed as in the experiments used to generate the data shown in FIG. 2D, except that pre-treatment of 100 nM RGDS (FIG. 5A) or echistatin (R & D Systems Inc., catalog number 3202/100U) (FIG. 5B) for 10 minutes was performed before irisin treatment. The data shown in FIGS. 6A and 6B were generated from MLO-Y4 cells incubated in serum free medium for 3 hours followed by treatment of indicated time of 10 nM irisin for 16 hours. RNAs were extracted from the cells and the sclerostin mRNA level was analyzed by qPCR (FIG. 6A) or treated and analyzed as in (FIG. 6A), except that pre-treatment of 10 μM RGDS, RGDyK (Selleck, catalog number S7844), or echistatin was performed for 10 minutes before irisin treatment. The data shown in FIGS. 7A and 7B were generated from 8 week-old mice injected with the indicated dose of irisin for 6 days. Tibias were collected and treated with collagenase to obtain mRNA from the osteocyte-enriched bones. Sclerostin mRNA level was analyzed by qPCR (FIG. 7A). Serum was collected to analyze the sclerostin protein level using ELISA kit (R&D Systems Inc., catalog number MSST00) (FIG. 7B). The data shown in FIG. 8 were generated from 8 week-old mice injected with the indicated dose of irisin for 6 days. Epididymal fats were collected, RNAs were extracted from the tissues, and mRNA levels of the indicated genes were analyzed by qPCR. The data shown in FIGS. 9E and 9F were generated from ovariectomies (OVX) performed on 9 month-old wild-type mice (WT) and global FNDC5 knockout mice (FNDC KO) followed by collection of lumbar vertebra and tibia after 3 weeks. The bone histomorphometric analysis was performed in the lumbar to measure bone volume per trabecular thickness (FIG. 9E), and to count trabecular number (FIG. 9F). The data shown in FIG. 10 were generated from similar experiments to those used to generate the data shown in FIG. 9, except that bone histomorphometric analysis was performed to measure eroded surface/bone surface (FIG. 9J) and to measure lacunae area (FIG. 10E).

a. Expression and Purification of Human/Mouse Recombinant His-Tag Irisin

His-tag recombinant irisin was generated by transfection of an irisin (human/mouse)-10 his tag DNA plasmid. This protein with a C-terminal his tag was produced and purified from mammalian HEK293 cells after transient DNA transfection. The protein was purified from 250 ml conditioned media using IMAC column, followed by Superdex200 in 50 mM HEPES pH7.2, 150 mM NaCl. The protein was diluted in sterilized PBS to use in cell culture experiments and in vivo injection.

b. Cell Culture Experiments

MLO-Y4 cells were cultured as previously described (Kato et al. (1997) J. Bone Miner. Res. 12:2014-2023). The cells were seeded on type I collagen-coated 6 well plates under MEMα medium (Thermo Fisher Scientific, 12571-063), 2.5% Fetal Bovine Serum (Hyclone, SH30396.03, Lot AB217307), 2.5% calf serum (Hyclone, SH30072.03, AAL11105), penicillin-streptomycin (P/S) 100 U/ml. At 60% cell density, medium was switched to FreeStyle293 Expression medium after washing with warm PBS. After 4 hours incubation, the cells were treated with indicated doses of irisin for indicated times. For integrin inhibitor treatment, cells were treated with indicated concentration of the inhibitors for 10 minutes before irisin treatment. For antagonistic antibody treatment, cells were treated with 0.9 m/ml antagonistic antibodies against αV/β3 or αV/β5 monoclonal mouse Igg as a negative control for 10 minutes before irisin treatment. After treatments, medium was aspirated on ice and cold PBS was added to the cells. RIPA buffer for lysis was added after aspiration of cold PBS for immunoblot analysis.

c. Transient Transfection

HEK293T cells were set up for experiments at 1×10⁵ cells per well in 6 well plate. On day 2, cells were transiently transfected with the indicated plasmids with FuGENE® 6 reagent (Roche Applied Science) according to the manufacturer's protocol. After 24 hours of incubation, Freestyle 293 medium were added and the cells were incubated for 3 hours followed by treatment of indicated concentration of irisin for 5 minutes or by pre-treatment of 10 μM cyclo RGDyK for 10 minutes and treatment of 0.3 nM irisin for 5 minutes. After treatments, medium was aspirated on ice and cold PBS was added to the cells. RIPA buffer for lysis was added after aspiration of cold PBS for immunoblot analysis.

d. Primary White Adipocyte Cultures

Inguinal fat tissue from 6 weeks old mice was dissected and washed with PBS, minced and digested for 1 hour at 37° C. in PBS containing 10 mM CaCl₂, 2.4 U/ml dispase II (Roche) and 10 mg/ml collagenase D (Roche). After adding warm DMEM/F12 (1:1) with 10% FCS, digested tissue was filtered through a 70 μm cell strainer and centrifuged at 600×g for 10 minutes. Pellet was resuspended by 40 ml DMEM/F12 (1:1) with 10% FCS and filtered through a 40 μm cell strainer followed by centrifugation at 600×g for 10 minutes. Pelleted inguinal stromal vascular cells were grown to confluence and split onto type I collagen-coated coated 12 well plates. The cells were induced to differentiate by treatment with 1 μM rosiglitazone, 5 μM dexamethasone, 0.5 μM isobutyl methyl xanthine in the presence of 0, 0.5, 5 or 50 ng/ml recombinant 10 his-tag irisin protein for 2 days. After that, cells were maintained in 1 μM rosiglitazone in the presence of 0, 0.5, 5 or 50 ng/ml recombinant 10 his-tag irisin protein for 4 days with medium change every other day. mRNA levels were analyzed as described in gene expression analysis.

e. Animal Studies

Experiments were performed with sex- and age-matched global FNDC5 knockout and littermate control mice. Female mice were initially ovariectomized to deplete ovarian hormones and induce osteoporosis. Mice were sacrificed after 3 weeks of OVX at the age of 36˜38 weeks. 8 weeks old C57BL/6J wild type mice were ovariectomized and sacrificed after 2 weeks of OVX to measure irisin level in plasma. The remaining uterine fundus, cervical region and vaginal vault was removed as a whole from the mice and weighed to ensure shrinkage from the ovariectomy procedure.

C57BL/6J wild-type male mice for recombinant irisin injection were acquired from The Jackson Laboratory (000664). Mice were mock injected with sterilized PBS for at least three days. For bone studies, the mice were injected with 1 mg/kg irisin by daily intraperitoneal (IP) injection for 6 days. Plasma was collected to analyze sclerostin protein level and tibia was collected to analyze mRNA level in osteocyte-enriched bones. To get osteocyte-enriched bones, the bones were flushed with HBSS and then cut longitudinally by surgical blade in α-MEM without phenol red (Gibco, 41061-029). The bones were incubated with α-MEM containing 250 u/m collagenase (Sigma-Aldrich, C9891) for 30 minutes followed by 30 minutes incubation with 5 mM EDTA with 0.1% BSA, pH 7.4 after washing the bones with HBSS three times. The bones were incubated with α-MEM containing 250 u/m collagenase (Sigma-Aldrich, C9891) for 30 minutes additionally after washing the bones with HBSS three times. After aspiration of the medium, the osteocyte-enriched bones were homogenized by a mechanical homogenizer in cold room (4° C.) with metal beads and TRIzol for gene expression analysis. For inguinal fat, the mice were injected IP with 1 mg/kg irisin every other day for 6 days. Inguinal fats were homogenized by a mechanical homogenizer in cold room (4° C.) with metal beads and TRIzol® for gene expression analysis. For immunoblot analysis, the fats were homogenized with metal beads and 2% SDS, 150 mM NaCl, 50 mM HEPES pH 8.8, 5 mM DTT. To test the effect of cyclo RGDyK, the mice were co-injected with 1 mg/kg cyclic RGDyK or same amount of control RGD peptide. For the injection of SB273005, the compound dissolved in 5% DMSO+2% Tween 80+30% PEG 300+ddH2O.

f. Bone Histomorphometric Analysis for Trabecular Bone

Mice were subcutaneously injected with 20 mg/kg of calcein (Sigma Aldrich, St. Louis, Mo., USA) and 40 mg/kg of demeclocycline (Sigma Aldrich, St. Louis, Mo., USA) 9- and 2-day prior to the sacrifice, respectively. Lumbar vertebra (L3-L5) was harvested and immediately fixed in 70% ethanol for 3 days. The fixed bone samples were dehydrated and embedded in methylmethacrylate. Undecalcified 4-μm-thick sections were obtained using a motorized microtome (RM2255, Leica, Nussloch, Germany) and stained with Von Kossa method for showing the mineralized bone. Consecutive second section was left unstained for the analysis of fluorescence labeling and the third section was stained with 2% Toluidine Blue (pH 3.7) for the analysis of osteoblasts, osteoid, osteoclasts. The bone histomorphometric analysis was performed under 200× magnification in a 1.8 mm high×1.3 mm wide region located 400 μm away from the upper and lower growth plate using OsteoMeasure analyzing software (Osteometrics Inc., Decatur, Ga., USA). The structural parameters [bone volume (BV/TV), trabecular thickness (Tb.Th), trabecular number (Tb.N) and trabecular separation (Tb. Sp)] were obtained by taking an average from 2 different measurement of consecutive sections. The structural, dynamic and cellular parameters were calculated and expressed according to the standardized nomenclature (Dempster et al. (2013) J. Bone Miner. Res. 28:2-17).

g. Osteocyte Analysis

The residual methylmethacrylate embedded tibia sample blocks from bone histomorphometry were used for the osteocyte analysis. Blocks were trimmed and the bone surface was sequentially ground with silicon carbide sandpaper of increasing grid number (Scientific Instrument Services Inc., NJ, USA). The sample surface was then carbon coated by vacuum evaporation (Auto 306 Vacuum Coater, Boc Edwards, UK) followed by fixation on the specimen mount with aluminum conductive tape (Ted Pella Inc., CA, USA). A digital scanning electron microscope (SEM, Supra 55 VP, Zeiss, Oberkochen, Germany, Center for Nanoscale Systems in Harvard University, Cambridge, Mass.) was employed with an accelerating voltage of 20 kV, a working distance of 10 mm and 500× magnification for taking backscattered electron images of a standardized tibial midshaft area located 4.5 mm distal from the tibia-fibula junction. Images were analyzed with the Image J software (NIH, MD) for measuring osteocyte lacunae area and density.

h. Analysis of Femur Using μCT

High-resolution desktop microcomputed tomography imaging (μCT40, Scanco Medical, Brüttisellen, Switzerland), as previously reported (Spatz et al. (2013) J. Bone Miner. Res. 28:865-874) was used and trabecular and cortical bone microstructure in the distal femur and femoral diaphysis were assessed, respectively. Scans were acquired using a 10 μm³ isotropic voxel size, 70 kVP peak x-ray tube potential, 114 mAs tube current, 200 ms integration time, and were subjected to Gaussian filtration and segmentation. Image acquisition and analysis protocols adhered to the JBMR guidelines for the assessment of rodent bones by μCT (Bouxsein et al. (2010) J. Bone Miner. Res. 25:1468-4486). In the distal femur, transverse μCT slices were evaluated in a region of interest beginning 200 μm superior to the distal growth plate and extending proximally 1500 μm. The trabecular bone region was identified by semi-manually contouring the trabecular bone in the ROI with the assistance of an auto-thresholding software algorithm. Morphometric variables were computed from the binarized images. Using direct, 3D techniques, the bone volume fraction (Tb.BV/TV, %), trabecular bone mineral density (Tb.BMD, mgHA/cm3), trabecular thickness (Tb.Th, μm), trabecular number (Tb.N, mm−1), trabecular separation (Tb.Sp, μm), and connectivity density (mm−3) were assessed. Cortical bone was analyzed in 50 transverse μCT slices (ROI length=500 μm) at the femoral mid-diaphysis. The region of interest included the entire outer most edge of the cortex. Images were subjected to Gaussian filtration and segmented using a fixed threshold of 700 mgHA/cm3 to measure the following variables total cross-sectional area (Tt.Ar, mm2), cortical bone area (Ct.Ar, mm2), medullary area (Ma.Ar, mm2), bone area fraction (Ct.Ar/Tt.Ar, %), cortical tissue mineral density (Ct.TMD, mgHA/cm3), cortical thickness (Ct.Th, mm), cortical porosity (%), and the polar moment of inertia (pMOI, mm4).

i. Gene Expression Analysis

RNA was extracted from cultured cells or frozen tissues using TRIzol® (Thermo Fischer Scientific) and purified with RNeasy® mini kit (QIAGEN 74106). RNA was extracted from osteocyte-enriched tibia as described above (Qing et al. (2012) J. Bone Miner. Res. 27:1018-1029). To perform qRT-PCR analysis, normalized RNA was reverse transcribed using a high-capacity cDNA reverse-transcription kit (Applied Biosystems™). cDNA was analyzed by qRT-PCR with indicated primers. Relative mRNA levels were calculated using the comparative CT method and normalized to cyclophilin mRNA. Primer sequences used are listed in Table 3.

TABLE 3
Mouse qRT-PCR primers:

| | Forward primer | Reverse primer |
|---|---|---|
| Gene | (5′-3′) | (5′-3′) |
| Selerostin | AGCCTTCAGGAATGAT | CTTTGGCGTCATAGGG |
| | GCCAC | ATGGT |
| Ucp1 | GGTGCCTACATCGTAC | TGCTTGGTAAGCTCCT |
| | TGGC | TGGTG |
| Dio2 | CAGTGTGGTGCACGTC | TGAACCAAAGTTGACC |
| | TCCAATC | ACCAG |
| Cidea | TGC TCT TCT GTA | GCC GTG TTA AGG |
| | TCG CCC AGT | AAT CTG CTG |
| Pparg2 | GTGCCAGTTTCGATCC | GGCCAGCATCGTGTAG |
| | GTAGA | ATGA |
| Fabp4 | AAG GTG AAG AGC | TCA CGC CTT TCA |
| | ATC ATA ACCCT | TAA CAC ATT CC |
| Cyclophilin | CATCCTAAAGCATACA | TCCATGGCTTCC |
| | GGTCCTG | ACAATGTT |
| Opg | TCCGAGGACCACAATG | TGGGTTGTCCATTCAA |
| | AAC | TGATGT |

a. Immunoblot Analysis

Cells were harvested in RIPA buffer containing protease-inhibitor cocktail and phosphataseinhibitor cocktail. Whole-cell lysates were homogenized by 10 times passages through a 22G needle fitted to a 1 ml syringe. Homogenized samples were rotated gently in cold room for 20 minutes followed by 15,000×g centrifugation for 10 minutes. 10 μl supernatants were used for normalization using BCA assay and remained supernatants were mixed with 4×NuPAGE LDS sample buffer and 2.5% β-mercaptoethanol. The samples were incubated at 98° C. for 5 minutes. The samples were separated by SDS-PAGE, and transferred to Immobilon®-P membranes (Millipore). Protein levels were analyzed via western blot using indicated antibody. Inguinal fat pads were homogenized by a mechanical homogenizer in cold room (4° C.) with 800 μl of 2% SDS, 150 mM NaCl, 50 mM HEPES pH 8.8, 5 mM DTT containing proteaseinhibitor cocktail and phosphatase-inhibitor cocktail in cold room followed by incubation at 60° C. for 30 minutes. 100 μl of the homogenized samples were mixed with 300 μl methanol, 200 μl chloroform and 250 μl sterilized H2O. After centrifugation at 4000×g for 10 minutes at room temperature, upper and lower phases were removed by aspiration and interphase were washed with 1 ml cold methanol three times. After drying at 37° C., the interphase was solubilized by 8M Urea and 50 mM HEPES pH 8.5. After normalization of the protein using BCA assay, the samples were separated by SDS-PAGE, and transferred to Immobilon®-P membranes (Millipore). Protein levels were analyzed using western blot against indicated antibody.

b. Protein Protein Binding Assays

100 nM flag-tagged mammalian irisin was incubated with 5 nM of the indicated his-tag integrins in a final volume of 600 μl in 1.5 ml Protein LoBind Tubes (Eppendorf®, 022431081) for 5 minutes at room temperature under rotation. After rotation, 60 μl Ni-NTA agarose (ThermoFisher Scientific, R901-01) was applied to immunoprecipitated integrins. Precipitated integrins were detected by immunoblot analysis against his tag. Co-precipitated irisin was detected by immunoblot analysis against flag-tag.

c. Anti-Apoptosis Assay

MLO-Y4 cells were seeded in type-I collagen coated 96 well plate (3000 cells/well) in 1% FBS, 1% CS, α-MEM without phenol red (Gibco, 41061-029) on day 0. The medium was aspirated and 1% FBS, 1% CS, α-MEM without phenol red containing the indicated concentration of irisin was added to the wells. After 24 hours incubation, 0.5% FBS, 0.5% CS, α-MEM without phenol red containing the indicated concentration of irisin and 0.3 mM H₂O₂ were added and the cells were incubated for 4 hours. The cells were stained with 2 μM Ethidium Homodimer-1 (ThermoFisher Scientific, E1169) to detect dead cells. The cell images were taken using Nikon Eclipse TE300 inverted fluorescence microscope with a Photometrics® Coolsnap EZ cooled CCD camera and analyzed using ImageJ. Percentage of cell death was calculated as EthD-1 positive cells divided by the total number of cells stained with 5 μg/mL Hoechst 33342 (ThermoFisher Scientific, H3570) as a nuclear counterstain.

d. Identification of Irisin Receptor Using Quantitative Proteomics & Co-Immunoprecipitation of Candidates of Irisin Receptors

MLO-Y4 cells were seeded on 30×150 mm type-I collagen coated dishes as described in cell culture experiment. At 60% cell density, medium was switched to FreeStyle™ 293 Expression medium. After 4 hours incubation, the cells were chilled on ice for 10 minutes, followed by treatment of 10 nM his-tag irisin or his-tag adipsin for 20 minutes. The cells were then incubated with 1.5 mM DTSSP for 30 minutes on ice to do cross-linking, after washing with 15 ml cold PBS twice. The cross-linking was quenched by addition of a final concentration of 20 mM Tris-pH 7.5. The cells were then harvested and homogenized in 1 ml RIPA buffer containing proteaseinhibitor cocktailand phosphatase-inhibitor cocktail. Whole-cell lysates were homogenized by 10 times passages through a 22G needle fitted to a 3 ml syringe. Homogenized samples were rotated gently in cold room for 20 minutes followed by 15,000×g centrifugation for 10 minutes. After addition of a final concentration of 10 mM imidazole, supernatants were incubated with 100 μl Ni-NTA agarose for 1 hour. After centrifugation at 500×g for 1 minute, the supernatants were aspirated and 1 ml cold RIPA buffer containing 10 mM imidazole were added to the agarose. After 10 minutes rotation in cold room, the supernatants were aspirated and 1 ml cold RIPA buffer containing 30 mM imidazole were added to the agarose. After repeating the washing 3 times, 0.8 ml RIPA buffer containing 250 mM imidazole was added and the agarose was gently rotated in a cold room for 20 minutes. After centrifugation at 1000×g for 2 minutes, the supernatants were transferred to 1.5 ml tube and incubated with 100 μl 0.2% sodium deoxycholate and 100 μl 10% trichloroacetic acid in ice for 1 hour. After centrifugation at 12,000×g for 10 minutes at 4° C., the supernatants were removed and 1 ml cold acetone was added to the pellets followed by vortexing for 10 seconds. After one more washing with cold acetone, the pellets were dried at 37° C., and 39 μl PBS and 13 μl 4×NuPAGE LDS were added to the pellets with a final concentration of 5 mM DTT. Solubilized proteins were incubated at 65° C. for 20 minutes followed by incubation with a final concentration of 14 mM iodoacetamide for 45 minutes in the dark. 38 μl samples were loaded to 4-12% gradient SDS-PAGE for separation followed by Coomassie Blue staining. The gels were submitted to quantitative proteomics.

e. Protein Digestion and Isobaric Tag Peptide Labeling

For in-gel digestions, gels were stained with Coomassie Blue and were excised into 8 equal segments for control and irisin lanes. Gel pieces were destained and dehydrated with 100% acetonitrile, vacuumed dried, and digested in 25 mM HEPES (pH 8.5) with 500 ng sequencing grade trypsin (Promega) and incubated for an overnight at 37° C. (Shevchenko et al. (1996) Anal. Chem. 68:850-858). Digests were treated with 1% formic acid and purified using C18 Stage-Tips as previously described (Rappsilber et al. (2007) Nat. Protoc. 2:1896-1906). Peptides were eluted with 70% acetonitrile and 1% formic acid, then dried using a speedvac. Isobaric labeling of digested peptides was accomplished using 6-plex tandem mass tag (TMT) reagents (Thermo Fisher Scientific, Rockford, Ill.). Reagents, 5.0 mg, were dissolved in 252 μl acetonitrile (ACN) and 5 μl of the solution was added to the digested peptides dissolved in 25 μl of 200 mM HEPES, pH 8.5. After 1 hour at room temperature, the reaction was quenched by adding 1 μl of 5% hydroxylamine. Labeled peptides were combined and acidified prior to C18 Stage-Tips desalting.

f. Liquid Chromatography Separation and Tandem Mass Spectrometry (LC-MS/MS)

All LC-MS/MS experiments were performed on an Orbitrap Fusion™ Lumos mass spectrometer (Thermo Fisher Scientific, San Jose, Calif., USA) coupled with a Proxeon EASY-nLC™ 1200 LC pump (Thermo Fisher Scientific). Peptides were separated on a 100 μm inner diameter microcapillary column packed with 35 cm of Accucore™ C18 resin (1.8 μm, 100 Å, Thermo Fisher Scientific). Peptides were separated using a 2 hour gradient of 6-33% acetonitrile in 0.125% formic acid with a flow rate of ˜400 nL/min. Each analysis used an MS3-based TMT method as described previously (McAlister et al. (2014) Anal. Chem. 86:7150-7158. MS1 data was acquired at a mass range of m/z 350-1350, resolution 120,000, AGC target 5×105, maximum injection time 150 ms, and with a dynamic exclusion of 120 seconds for the peptide measurements in the Orbitrap™.

Data dependent MS2 spectra were acquired in the ion trap with a normalized collision energy (NCE) set at 35%, AGC target set to 2.2×104 and a maximum injection time of 120 ms. MS3 scans were acquired in the Orbitrap™ with a HCD collision energy set to 55%, AGC target set to 5.5×105, maximum injection time of 200 ms, resolution at 15,000 and with a maximum synchronous precursor selection (SPS) precursors set to 10.

g. Data Processing and Spectra Assignment

In-house developed software was used to convert mass spectrometric data (.raw files) to an mzXML format, as well as to correct monoisotopic m/z measurements. All experiments used the Mouse UniProt database (downloaded 10 Apr. 2017) where reversed protein sequences and known contaminants such as human keratins and albumin were appended. SEQUEST searches were performed using a 20 ppm precursor ion tolerance, while requiring each peptide's amino/carboxy terminus to have trypsin protease specificity and allowing up to two missed cleavages. Six-plex TMT tags on peptide N termini and lysine residues (+229.162932 Da) and carbamidomethylation of cysteine residues (+57.02146 Da) were set as static modifications while methionine oxidation (+15.99492 Da) was set as variable modification. A MS2 spectra assignment false discovery rate (FDR) of less than 1% was achieved by applying the target-decoy database search strategy (Elias and Gygi, 2007). Filtering was performed using an in-house linear discrimination analysis method to create one combined filter parameter from the following peptide ion and MS2 spectra metrics: Sequest parameters XCorr and ΔCn, peptide ion mass accuracy and charge state, peptide length and mis-cleavages. Linear discrimination scores were used to assign probabilities to each MS2 spectrum for being assigned correctly and these probabilities were further used to filter the dataset with an MS2 spectra assignment FDR of smaller than a 1% at the protein level (Huttlin et al. (2010) Cell. 143:1174-1189).

h. Determination of TMT Reporter Ion Intensities and Quantitative Data Analysis

For quantification, a 0.03 m/z window centered on the theoretical m/z value of each of the two reporter ions and the intensity of the signal closest to the theoretical m/z value was recorded. Reporter ion intensities were further de-normalized based on their ion accumulation time for each MS2 or MS3 spectrum and adjusted based on the overlap of isotopic envelopes of all reporter ions (as per manufacturer specifications). The total signal intensity across all peptides quantified was summed for each TMT channel, and all intensity values were adjusted to account for potentially sample handling variance.

i. Quantification of Irisin in Plasma Using Quantitative Proteomics & Plasma Purification

Blood was collected 2 weeks after OVX and plasma was separated by centrifugation. Plasma specimens (35 μl) were depleted of albumin and IgG using the ProteoExtract® kit and subsequently concentrated using 3 kDa molecular weight cut-off spin-filter columns (Millipore). Deglycosylation of plasma was performed using Protein Deglycosylation Mix (NEB) as per the manufacturer's denaturing protocol. Deglycosylated plasma samples were reduced with 10 mM DTT and alkylated with 50 mM iodoacetamide prior to being resolved by SDS-PAGE using 4%-12% NuPAGE Bis-Tris precast gels (Life Technologies) (Jedrychowski et al. (2015) Cell Metab. 22:734-740).

j. In-Gel Digestion

Deglycosylated murine plasma samples were reduced with 5 mM DTT and alkylated with 75 mM iodoacetamide prior to being resolved by SDS-PAGE using 4-12% Bis-Tris precast gels (Life Technologies). Gels were coomassie stained and fragments were excised and cut into smaller fragments from the 10-15 KD region. Gel pieces were destained and dehydrated with 100% acetonitrile, vacuumed dried, and 25 mM HEPES (pH 8.5) with 500 ng sequencing grade trypsin (Promega) was added for an overnight incubation at 37° C. Digests were quenched after 12 hours with 70% acetonitrile/1% formic acid, dried and desalted using in-house stage tips as previously described (Rappsilber et al. (2007) Nat. Protoc. 2:1896-1906). Peptides were eluted with 70% acetonitrile/1% formic acid, dried using a speedvac, and resuspended in 12 μl of 5% formic acid and 5% acetonitrile containing the heavy valine synthesized irisin peptides (1 femtomole).

k. Mass Spectrometry and Liquid Chromatography

Mass spectrometry data were collected using an Orbitrap Fusion™ Lumos mass spectrometer (Thermo Scientific) coupled with μHPLC (EASY-nLC™ 1200 system, Thermo Scientific). Peptides were separated onto a 75 μm inner diameter microcapillary column packed with ˜40 cm of Accucore™ C18 resin (2.6 μm, 150 Å, Thermo Fisher Scientific). For each analysis, ˜4 μl were onto the column. Peptides were separated using a 60-minute gradient of 8 to 30% acetonitrile in 0.125% formic acid with a flow rate of ˜400 nL/min.

l. Parallel Reaction Monitoring Acquisition

Parallel reaction monitoring (PRM) analyses were performed using a Q-Exactive™ mass spectrometer (Thermo Fisher Scientific). A full MS scan from 575-700 m/z at an orbitrap resolution of 120,000 (at m/z 200), AGC target 1×106 and a 1000 ms maximum injection time was performed. Full MS scans were followed by 25-50 PRM scans at 30,000 resolution (AGC target 1×106, 2000 ms maximum injection time) as triggered by a scheduled inclusion list (Tables 4-5). The PRM method employed an isolation of target ions by a 1.6 Th isolation window, fragmented with normalized collision energy (NCE) of 35. MS/MS scans were acquired with a starting mass range of 110 m/z and acquired as a profile spectrum data type. Fragment ions for all peptides were quantified using Skyline version 3.5 (Maclean et al. (2010) Bioinformatics, 26:966-968).

TABLE 4
List of heavy and light irisin peptides

| Mass [m/z] | z | Peptide Sequence |
|---|---|---|
| 604.817127 | 2 | FIQEVNTTTR (light) |
| 607.824031 | 2 | FIQEVNTTTR (heavy) |
| 605.309135 | 2 | FIQEVN [+1.0] TTTR (light) |
| 608.316039 | 2 | FIQEVN [+1.0] TTTR (heavy) |
| 621.327859 | 2 | DSPSAPVNVTVR (light) |
| 624.334763 | 2 | DSPSAPVNVTVR (heavy) |
| 621.819867 | 2 | DSPSAPVN [+1.0] VTVR (light) |
| 624.826771 | 2 | DSPSAPVN [+1.0] VTVR (heavy) |

TABLE 5
AQUA peptides used in this study (Red
bold underline is heavy amino acid)

| AQUA | | Mass (Da) | |
|---|---|---|---|
| Peptide | Sequence | Light | Heavy |
| FNDC5 34-43 | DSPSAPVNVT [symbol] R | 1240.631 | 1246.655 |
| FNDC5 79-88 | FIQE [symbol] NTTTR | 1207.609 | 1213.634 |

m. Peptide and Protein Identification

Following mass spectrometry data acquisition, raw files were converted into mzXML format and processed using a suite of software tools developed in-house for analysis of proteomics datasets. All precursors selected for MS/MS fragmentation were confirmed using algorithms to detect and correct errors in monoisotopic peak assignment and refine precursor ion mass measurements. All MS/MS spectra were then exported as individual DTA files and searched using the Sequest algorithm (Eng et al. (1994) J. Am. Soc. Mass Spectrom. 3rd 5:976-989). These spectra were searched against a database containing sequences of all human proteins reported by Uniprot (Magrane, 2011) in both forward and reversed orientations. Common contaminating protein sequences (e.g. human keratins, porcine trypsin) were included as well. The following parameters were selected to identify peptides from unenriched peptide samples: 25 ppm precursor mass tolerance; 0.02 Da product ion mass tolerance; no enzyme digestion; up to two tryptic missed cleavages; variable modifications: oxidation of methionine (+15.994915) and deamidation of asparagine (0.984016); AScore algorithm was used to quantify the confidence with which each deamidation modification could be assigned to a particular residue in each peptide (Beausoleil et al. (2006) Nat. Biotechnol. 24:1285-1292). Peptides with AScores above 13 were considered to be localized to a particular residue (p<0.05).

n. HDX/MS

Differential HDX-MS experiments were conducted as previously described with a few modifications (Chalmers et al. (2006) Anal. Chen. 78:1005-1014).

a. Peptide Identification:

Protein samples were injected for inline pepsin digestion and the resulting peptides were identified using tandem MS (MS/MS) with an Orbitrap™ mass spectrometer (Fusion Lumos, ThermoFisher). Following digestion, peptides were desalted on a C8 trap column and separated on a 1 hour linear gradient of 5-40% B (A is 0.3% formic acid and B is 0.3% formic acid 95% CH3CN). Product ion spectra were acquired in data-dependent mode with a one second duty cycle such that the most abundant ions selected for the product ion analysis by higher-energy collisional dissociation between survey scan events occurring once per second. Following MS2 acquisition, the precursor ion was excluded for 16 seconds. The resulting MS/MS data files were submitted to Mascot (Matrix Science) for peptide identification. Peptides included in the HDX analysis peptide set had a MASCOT score greater than 20 and the MS/MS spectra were verified by manual inspection. The MASCOT search was repeated against a decoy (reverse) sequence and ambiguous identifications were ruled out and not included in the HDX peptide set.

HDX-MS analysis: Apo proteins (irisin and integrin αV/β5) were analyzed at 10 μM each. For differential HDX, integrin αV/β5 (10 μM) was concentrated 3× using an Amicon® Ultra Centrifugal Filter Unit with a 50K membrane (Part #: UFC505008) and the protein complex was formed by incubating irisin (10 μM) with integrin αV/β5 (30 μM) for 1 hour at room temperature. Next, 5 μl of sample was diluted into 20 μl D2O buffer (20 mM Tris-HCl, pH 7.4, 150 mM NaCl, 2 mM DTT) and incubated for various time points (0, 10, 60, 300, 900 and 3600 s) at 4° C. The deuterium exchange was then slowed by mixing with 25 μl of cold (4° C.) 3M urea and 1% trifluoroacetic acid. Quenched samples were immediately injected into the HDX platform. Upon injection, samples were passed through an immobilized pepsin column (2 mm×2 cm) at 200 μl min⁻¹ and the digested peptides were captured on a 2 mm×1 cm C8 trap column (Agilent) and desalted. Peptides were separated across a 2.1 mm×5 cm C18 column (1.90 Hypersil Gold, ThermoFisher) with a linear gradient of 4%-40% CH3CN and 0.3% formic acid, over 5 minutes. Sample handling, protein digestion and peptide separation were conducted at 4° C. Mass spectrometric data were acquired using an Orbitrap mass spectrometer (Q Exactive, ThermoFisher). HDX analyses were performed in triplicate, with single preparations of each protein ligand complex. The intensity weighted mean m/z centroid value of each peptide envelope was calculated and subsequently converted into a percentage of deuterium incorporation. This was accomplished by determining the observed averages of the undeuterated and fully deuterated spectra and using the conventional formula described elsewhere (Zhang and Smith, 1993). Statistical significance for the differential HDX data was determined by an unpaired t-test for each time point, a procedure that was integrated into the HDX Workbench software (Pascal et al. (2012) J Am. Soc. Mass Spectrom. 23:1512-1521).

Corrections for back-exchange were made on the basis of an estimated 70% deuterium recovery, and accounting for the known 80% deuterium content of the deuterium exchange buffer.

b. Data Rendering:

The HDX data from all overlapping peptides were consolidated to individual amino acid values using a residue averaging approach. Briefly, for each residue, the deuterium incorporation values and peptide lengths from all overlapping peptides were assembled. A weighting function was applied in which shorter peptides were weighted more heavily and longer peptides were weighted less. Each of the weighted deuterium incorporation values were then averaged to produce a single value for each amino acid. The initial two residues of each peptide, as well as prolines, were omitted from the calculations. This approach is similar to that previously described (Keppel and Weis, 2015). HDX analyses were performed in triplicate, with single preparations of each purified protein/complex. Statistical significance for the differential HDX data was determined by t-test for each time point, and was integrated into the HDX Workbench software (Pascal et al. (2012) J Am. Soc. Mass Spectrom. 23:1512-1521).

o. Generation of Docking Model with ZDOCK

A model for irisin-αVβ5 was generated using homology modeling. The models for b5 and irisin were generated using Modeller (Sali & Blundell et al. (1993) J. Mol. Biol. 234:779-815) based on a model of Fibronectin-αVβ3 (PDB 4MMX). Irisin was docked to β5 using the ZDOCK server (available on the World Wide Web at zdock.umassmed.edu/) according to the guide line Pierce et al. (2014) Bioinformatics. 30:1771-1773). The resulting model that agreed with the observed HDX-MS data was used to generate the Irisin-αV/β5 model.

p. Statistical Analysis

All values in graphs are presented as mean+/−S.E.M. Two-way ANOVA for multiple comparison were used to analyze the data. Significant differences between two groups were evaluated using a two-tailed, unpaired Student's t-test as the samples groups displayed a normal distribution and comparable variance (*: p<0.05; **: p<0.01; ***: p<0.001).

TABLE 14
Key resources table

| | | |
|---|---|---|
| REAGENT of RESOURCE | SOURCE | IDENTIFIER |

Antibodies

| | | |
|---|---|---|
| Anti-Rabbit phospho-FAL (Tyr397) | Cell Signaling | Cat. # 3283S |
| | Technology | |
| Anti-Rabbit FAK | Cell Signaling | Cat. # 3285S |
| | Technology | |
| Anti-Rabbit phospho-Zyxin (Ser142/143)(D1E8) | Cell Signaling | Cat. # 8467S |
| | Technology | |
| Anti-Rabbit Zyxin | Cell Signaling | Cat. # 3553S |
| | Technology | |
| Anti-Rabbit phospho-CREB (SerI33)(87G3) | Cell Signaling | Cat. # 9198S |
| | Technology | |
| Anti-Mouse CREB (86B10) | Cell Signaling | Cat. # 9104S |
| | Technology | |
| Anti-Rabbit Vinculin | Cell Signaling | Cat. # 13901S |
| | Technology | |
| Anti-Rabbit Integrin αV (D2N5H) | Cell Signaling | Cat. # 60896S |
| | Technology | |
| Anti-Mouse Myc-Tag (9B11) | Cell Signalling | Cat. # 2276S |
| | Technology | |
| Ant-Rabbit Flag-tag | Cell Signaling | Cat. # 2368S |
| | Technology | |
| 6X His tag ® antibody (HRP) | Cell Signaling | Cat. # 2276S |
| | Technology | |
| Anti-Rabbit UCP1 | Abcam | Cat. # ab10983 |
| Beta Actin antibody [AC-15] (HRP) | Abcam | Cat. # ab-49900 |
| Anti-Rabbit Irisin | Cell Signaling | N/A |
| | Technology | |
| Mouse IgG1 Isotype Control (Clone 11711) | R&D Systems | Cat. # MAB002 |
| Integrin alpha V beta 3 antibody [27.1 (VNR-1)]- | Abcam | Cat. # ab78289- |
| 50MICROG | | 50 ug |
| anti-Integrin αVβ5, Clone: P1F6 | EMD Millipore | Cat. # |
| | | MAB1961ZMI |

Chemicals, Peptides, and Recombinant Proteins

| | | |
|---|---|---|
| 10 His-tag irisin | Lake Pharma | N/A |
| Flag-tagged mammalian irisin | Enzo life sciences | Cat. # ADI-908- |
| | | 307-3010 |
| His-tag adipsin | R&D Systems | Cat. # AF1824-SP |
| Cyclo RGDyK | Selleck | Cat. # S7844 |
| RGDS peptide | R&D Systems | Cat. # 3498/10 |
| Control RGD peptide | Enzo life sciences | Cat. # BML-P701- |
| | | 0005 |
| Echistatin | R & D Systems | Cat. # 3202100U |
| SB273005 | Selleck | Cat. # S7540 |
| PBS | Coming ™ cellgro ™ | Cat. # |
| | | MT21040CV |
| MEMα medium | Thermo Fisher | Cat. # 12571-063 |
| | Scientific | |
| α-MEM without phenol red | Gibco | Cat. # 41061-029 |
| Fetal Bovine Serum | Gemini Bio- | Cat. # 100-106 |
| | Products | |
| Fetal Bovine Serum | Hyclone | Cat. # |
| | | SH30396.03, Lot |
| | | AB217307 |
| Calf serum | Hyclone | Cat. # |
| | | SH30072.03, Lot |
| | | AAL11105 |
| FreeStyle293 Expression medium | Life Technologies | Cat. # 12338018 |
| RIPA buffer | Thermo Fisher | Cat. # 89901 |
| | Scientific | |
| Collagenase | Sigma-Aldrich | Cat. # C9891 |
| Calcein | Sigma Aldrich | Cat. # C0875-5G |
| Demeclocycline hydrochloride 90% (HPLC), | Sigma Aldrich | Cat. # D6140-1G |
| powder | | |
| HBSS | Coming | Cat. # |
| | | MT21023CV |
| TRIzol | Thermo Fischer | Cat. # 15596018 |
| | Scientific | |
| FuGENE 6 Transfection Reagent, 1 ml | Promega | Cat. # E2691 |
| Ni-NTA agarose | ThermoFisher | Cat. # R901-01 |
| | Scientific | |
| Ethidium Homodimer-I (EthD-1) | ThermoFisher | Cat. # E1169 |
| | Scientific | |
| Hoechst 33342 | ThermoFisher | Cat. # H3570 |
| | Scientific | |
| 3,3′-Dithiobissulfosuccinimidyl propionate | Thermo Fisher | Cat. # PI121578 |
| (DTSSP) 50 mg/pk | Scientific | |
| Complete, Mini, EDTA-free (Protease Inhibitor) | Roche Diagnostics | Cat. # 1836170 |
| PhosSTOP ™ | Roche Diagnostics | Cat. # PHOSS-RO |
| Recombinant Human Integrin alpha 1 beta 1 | R&D Systems | Cat. # 7064-AB- |
| Protein, CF | | 025 |
| Recombinant Human Integrin alpha 4 beta 1 | R&D Systems | Cat. # 5668-A4- |
| Protein, CF | | 050 |
| Recombinant Human Integrin alpha 9 beta 1 | R&D Systems | Cat. # 5438-A9- |
| Protein, CF | | 050 |
| Recombinant Human Integrin alpha 11 beta 1 | R&D Systems | Cat. # 6357-AB- |
| Protein, CF | | 050 |
| Recombinant Human Integrin alpha V beta 1 | R&D Systems | Cat. # 6579-AV- |
| Protein, CF | | 025 |
| Recombinant Human Integrin alpha 5 beta 1 | R&D Systems | Cat. # 3230-A5- |
| Protein, CF | | 050 |
| Recombinant Human Integrin alpha 10 beta 1 | R&D Systems | Cat. # 5895-AB- |
| Protein, CF | | 050 |
| Recombinant Human Integrin alpha 6 beta 1 | R&D Systems | Cat. # 7809-A6- |
| Protein, CF | | 050 |
| Recombinant Human Integrin alpha 3 beta 1/VLA- | R&D Systems | Cat. # 2840-A3- |
| 3 Protein, CF | | 050 |
| Recombinant Human Integrin alpha 2 beta 1 | R&D Systems | Cat. # 5698-A2- |
| Protein, CF | | 050 |
| Recombinant Human Integrin alpha V beta 5 | R&D Systems | Cat. # 2528-AV- |
| Protein, CF | | 050 |
| Recombinant Mouse Integrin alpha V beta 1 | R&D Systems | Cat. # 7705-AV- |
| Protein, CF | | 050 |
| Recombinant Mouse Integrin alpha V beta 3 | R&D Systems | Cat. # 7889-AV- |
| Protein, CF | | 050 |
| Recombinant Mouse Integrin alpha V beta 5 | R&D Systems | Cat. # 7706-AV- |
| Protein, CF | | 050 |
| Recombinant Mouse Integrin alpha 4 beta 1 | R&D Systems | Cat. # 6054-A4- |
| Protein, CF | | 050 |
| Recombinant Mouse Integrin alpha 5 beta 1 | R&D Systems | Cat. # 7728-A5- |
| Protein, CF | | 050 |
| Recombinant Mouse Integrin alpha 7 beta 1 | R&D Systems | Cat. # 7958-A7- |
| Protein, CF | | 050 |
| Recombinant Mouse Integrin alpha 9 beta 1 | R&D Systems | Cat. # 7826-A9- |
| Protein, CF | | 050 |
| Recombinant Mouse Integrin alpha 10 beta 1 | R&D Systems | Cat. # 7827-AB- |
| Protein, CF | | 050 |
| Recombinant Mouse Integrin alpha 1 beta 1 | R&D Systems | Cat. # 8188-AB- |
| Protein, CF | | 025 |
| Recombinant Mouse Integrin alpha 2 beta 1 | R&D Systems | Cat. # 7828-A2- |
| Protein, CF | | 050 |
| Human Vitronectin 50 ug Carrier Free | R&D Systems | Cat. # 2308-VN- |
| | | 050 |
| Collagenase from Clostridium histolyticum Type | Sigma Aldrich | Cat. # C9891- |
| IA, 0.5-5.0 FALGPA units/mg solid, ≥125 | | 500MG |
| CDU/mg solid | | |
| POLY (ETHYLENE GLYCOL) 300 | Thermo Fisher | Cat. # NC1022938 |
| | Scientific | |
| SuperSignal West Femto Maximum Sensitivity | Life Technologies | Cat. # 34095 |
| Luminata Crescendo Western HRP Substrate | Fisher Scientific | Cat. # |
| | | WBLUR0500 |
| L-(−)-Norephinephrine (+)-bitartrate salt | Sigma Aldrich | Cat. # A9512- |
| monohyrdate 99%, solid | | 250MG |
| PRIMOCIN 500 MG | Thermo Fisher | Cat. # NC9141851 |
| | Scientific | |

Critical Commercial Assays

| | | |
|---|---|---|
| Mouse/Rat SOST Quantikine ELISA Kit | R&D Systems | Cat. # MSST00 |
| ProteoExtract Albumin/IgG Removal Kit | EMD Millipore | Cat. # 122642- |
| | | 1KIT |
| Protein Deglycosylation Mix II | New England | Cat. # P6044S |
| | Biolabs | |
| Pierce ™ BCA Protein Assay Kit | Thermo Fisher | Cat. # 23225 |
| | Scientific | |
| cDNA reverse-transcription kit | Applied Biosystems | Cat. # 4368814 |
| RNase-Free DNase Set (50) | Qiagen | Cat. # 79254 |

Experimental Models: Cell Lines

| | |
|---|---|
| MLO-Y4 | Bonewald lab (Kato |
| | et al, 1997) |

Experimental Models: Organisms/Strains

| | | |
|---|---|---|
| C57BL/6J wild-type male mice | The Jackson | 000664 |
| | Laboratory | |
| FNDC5 KO | This study | |

Oligonucleotides

| | | |
|---|---|---|
| Tnfsf11 TaqMan ™ Gene Expression Assay | Thermo Fisher | Cat. # 4331182 |
| Mm00441906_m1 | Biosciences | |
| See Table S9 for qRT-PCR primer list | N/A | N/A |

Recombinant DNA

| | | |
|---|---|---|
| Human Integrin alpha V/CD51/ITGAV Gene ORF | Sino Biological | Cat. # HG11269- |
| cDNA clone expression plasmid, C-Myc tag | | CM |
| Human Integrin alpha 5/CD49e/ITGA5 Gene ORF | Sino Biological | Cat. # HG10366- |
| cDNA clone expression plasmid, C-Myc tag | | M-M |
| Human Integrin beta 5/ITGB5 Gene ORF cDNA | Sino Biological | Cat. # HG10779- |
| clone expression plasmid, N-Myc tag | | NM |
| Human Integrin alpha 11/ITGA11 Gene ORF | Sino Biological | Cat. # HG13017- |
| cDNA clone expression plasmid, C-Myc tag | | CM |
| Human ITGB1/Integrin beta-1/CD29 transcript | Sino Biological | Cat. # HG10587- |
| variant 1A Gene ORF cDNA clone expression | | CM |
| plasmid, C-Myc tag | | |
| Human CD61/Integrin beta 3/ITGB3 Gene ORF | Sino Biological | Cat. # HG10787- |
| cDNa clone expression plasmid, C-Myc tag | | CM |

Software and Algorithms

| | |
|---|---|
| OsteoMeasure analyzing software | Osteometrics Inc. |
| Image J software | NIH |
| Mascot | Matrix Science |
| ZDOCK server | available on the World Wide |
| | Web at zdock. |

Other

| | | |
|---|---|---|
| Motorized microtome | Leica | Cat. # RM2255 |
| Digital scanning electron microscope | Zeiss | Supra 55 VP |
| RNeasy mini kit | QIAGEN | Cat. # 74106 |
| ImmobilionP membranes | EMD Millipore | |
| 1.5 ml Protein LoBind Tubes | Eppendorf | Cat. # 022431081 |
| Amicon Ultra Centrifugal Filter Unit with a 50K | EMD Millipore | Cat. # UFC505008 |
| membrane | | |
| Amicon Ultra Centrifugal Filter Unit with a 3K | EMD Millipore | Cat. # UFC900324 |
| membrane | | |

### Example 2

### Irisin and Its Receptors: Mechanisms and Metabolic Physiology

Osteocytes are a key cell type that receives and integrates various chemical and physical signals within bone matrix. MLO-Y4 osteocytes (Kato et al. (2001) J. Bone Miner. Res. 16: 1622-1633) were first examined for the effects of various doses of irisin on H₂O₂ induced apoptotic death. This model has been used previously in studies of osteocyte function (Kato et al. (1997) J. Bone Miner. Res. 12:2014-2023; Plotkin et al. (2007) J. Biol. Chem. 282:24120-24130). As shown in FIG. 1A, irisin prevented osteocyte cell death in a dose dependent manner, at concentrations as low as 1 ng/ml (70 pM).

Based on these data and, particularly, the potency of the irisin effects, these osteocytes were used to determine the irisin receptor by chemically cross-linking his-tagged irisin to cell surface proteins and subjecting the resulting complexes to mass spectrometry (Table 2). MLO-Y4 cells were inclubated in serum free medium for 4 hours followed by treatment of 35 nM 6 his-tag irisin or his-adipsin (as a control) for 10 minutes on ice. Cells were homogenized and immunoprecipitated using 6 his-tag agarose after treatment of DTSSP cross-linker. Immunoprecipitated proteins were labeled with TMT and analyzed by mass spectrometer. The proteins with greatest enrichment with irisin, compared to a control protein (adipsin), are listed in Table 2. The only protein substantially enriched and containing the function of a bona fide signaling receptor (β1 integrin) is highlighted.

TABLE 2
Irisin can be crossed-linked to β1-integrin in osteocytes.

| | Gene | | Number of | |
|---|---|---|---|---|
| | Symbol | Description | peptides | irsin/adipsin |
| 1 | Pcdha4 | Protocadherin alpha-4 | 1 | 4.39 |
| 2 | Cd81 | CD81 antigen | 3 | 4.05 |
| 3 | Itgb1 | Integrin beta-1 | 4 | 3.44 |
| 4 | Mmgt1 | Membrane magnesium transporter 1 | 1 | 2.94 |
| 5 | Fndc3b | Fibronectin type III | 8 | 2.88 |
| | | domain-containing protein 3B | | |

(Top 5 enriched proteins with irisin versus adipsin. See also Tables 6A and 6B for full list.)

TABLE 6A
Data dissemination for Table 6B

| Columns | Description |
|---|---|
| Protein Id | Uniprot protein ID |
| Gene Symbol | gene symbol ascribed to uniprot protein ID |
| Description | protein functional annonation |
| Number of peptides | number of quantified peptides with |
| | signal_to_noise > 100 |
| adipsin | summed signal-to-noise value for TMT |
| | channel for adipsin for a given protein |
| irisin | summed signal-to-noise value for TMT |
| | channel for irisin for a given protein |
| irisin/adipsin | TMT ratio of each protein |

TABLE 6B
TMT signal-to-noise ratio (related to figure 2; list of proteins in crosslinking/co-
immunoprecipitation/mass spectrometry experiments)

| | | | Num- | | | |
|---|---|---|---|---|---|---|
| | | | ber | | | |
| | | | of | | | |
| | Gene | | pep- | | | irisin/ |
| Protein Id | Symbol | Description | tides | adipsin | irisin | adipsin |
| sp\|Q811B1\|XYLT1_MOUSE | Xylt1 | XYLT1_MOUSE Xylosyltransferase 1 | 1 | 13.0202 | 84.1019 | 6.459 |
| sp\|Q64737\|PUR2_MOUSE | Gart | PUR2_MOUSE Trifunctional purine biosynthetic protein | 2 | 29.5264 | 165.7 | 5.612 |
| | | adenosine-3 | | | | |
| sp\|Q8BGQ7\|SYAC_MOUSE | Aars | SYAC_MOUSE Alanine--tRNA ligase, cytoplasmic | 1 | 12.2224 | 67.8792 | 5.554 |
| sp\|O88689-3\|PCDA4_MOUSE | Pcdha4 | PCDA4_MOUSE Isoform 3 of Protocadherin alpha-4 | 1 | 17.1177 | 75.2281 | 4.395 |
| sp\|P97467\|AMD_MOUSE | Pam | AMD_MOUSE Peptidyl-glycine alpha-amidating | 1 | 14.6233 | 63.287 | 4.328 |
| | | monooxygenase | | | | |
| sp\|P35762\|CD81_MOUSE | Cd81 | CD81_MOUSE CD81 antigen | 3 | 105.428 | 427.394 | 4.054 |
| sp\|P28574\|MAX_MOUSE | Max | MAX_MOUSE Protein max | 1 | 26.3241 | 101.358 | 3.850 |
| sp\|Q78PY7\|SND1_MOUSE | Snd1 | SND1_MOUSE Staphylococcal nuclease domain- | 8 | 187.801 | 699.379 | 3.724 |
| | | containing protein 1 | | | | |
| sp\|Q04750\|TOP1_MOUSE | Top1 | TOP1_MOUSE DNA topoisomerase 1 | 41 | 3421.12 | 12610.9 | 3.686 |
| sp\|Q8BYC6\|TAOK3_MOUSE | Taok3 | TAOK3_MOUSE Serine/threonine-protein kinase TAO3 | 2 | 42.9041 | 149.719 | 3.490 |
| sp\|P09055\|ITB1_MOUSE | Itgb1 | ITB1_MOUSE Integrin beta-1 | 4 | 249.507 | 857.731 | 3.438 |
| sp\|B2RY56\|RBM25_MOUSE | Rbm25 | RBM25_MOUSE RNA-binding protein 25 | 7 | 270.762 | 918.102 | 3.391 |
| sp\|Q64152\|BTF3_MOUSE | Btf3 | BTF3_MOUSE Transcription factor BTF3 | 1 | 20.2487 | 67.7263 | 3.345 |
| sp\|P97310\|MCM2_MOUSE | Mcm2 | MCM2_MOUSE DNA replication licensing factor MCM2 | 19 | 872.212 | 2618.38 | 3.002 |
| sp\|Q8K273\|MMGTL_MOUSE | Mmgt1 | MMGT1_MOUSE Membrane magnesium transporter 1 | 1 | 17.4386 | 51.1993 | 2.936 |
| sp\|Q9Z0J0\|NPC2_MOUSE | Npc2 | NPC2_MOUSE Epididymal secretory protein E1 | 1 | 38.4464 | 112.274 | 2.920 |
| sp\|Q6NWW9\|FND3B_MOUSE | Fndc3b | FND3B_MOUSE Fibronectin type III domain-containing | 8 | 449.327 | 1294.806 | 2.882 |
| | | protein 3B | | | | |
| sp\|Q8BP67\|RL24_MOUSE | Rpl24 | RL24_MOUSE 60S ribosomal protein L24 | 3 | 266.119 | 762.477 | 2.865 |
| sp\|Q9Z2H5\|E41LI_MOUSE | Epb41l1 | E41L1_MOUSE Band 4.1-like protein 1 | 7 | 351.055 | 998.379 | 2.844 |
| sp\|Q6ZWY3\|RS27L_MOUSE | Rps27l | RS27L_MOUSE 40S ribosomal protein S27-like | 1 | 184.707 | 523.367 | 2.833 |
| sp\|P24788\|CD11B_MOUSE | Cdk11b | CD11B_MOUSE Cyclin-dependent kinase 11B | 5 | 373.169 | 1053.49 | 2.823 |
| sp\|P62751\|RL23A_MOUSE | Rpl23a | RL23A_MOUSE 60S ribosomal protein L23a | 4 | 617.555 | 1740.18 | 2.818 |
| sp\|P62717\|RL18A_MOUSE | Rpl18a | RL18A_MOUSE 60S ribosomal protein L18a | 5 | 617.707 | 1710.3 | 2.769 |
| sp\|Q9Z0F8\|ADA17_MOUSE | Adam17 | ADA17_MOUSE Disintegrin and metalloproteinase | 1 | 27.5718 | 75.8796 | 2.752 |
| | | domain-containing protein 17 | | | | |
| sp\|P62830\|RL23_MOUSE | Rpl23 | RL23_MOUSE 60S ribosomal protein L23 | 4 | 786.216 | 2155.69 | 2.742 |
| sp\|Q6P5F9\|XPO1_MOUSE | Xpo1 | XPO1_MOUSE Exportin-1 | 5 | 136.202 | 372.446 | 2.735 |
| sp\|O08810\|U5Sl_MOUSE | Eftud2 | U5S1_MOUSE 116 kDa U5 small nuclear | 2 | 60.8528 | 163.947 | 2.694 |
| | | ribonucleoprotein component | | | | |
| sp\|P17897\|LYZ1_MOUSE | Lyz1 | LYZ1_MOUSE Lysozyme C-1 | 1 | 18.3796 | 49.1298 | 2.673 |
| tr\|A8DUK4\|A8DUK4_MOUSE | Hbbt1 | A8DUK4_MOUSE Beta-globin | 5 | 594.828 | 1577.85 | 2.653 |
| sp\|Q9Z2D6\|MECP2_MOUSE | Mecp2 | MECP2_MOUSE Methyl-CpG-binding protein 2 | 10 | 658.105 | 1739.03 | 2.642 |
| sp\|Q9CPR4\|RL17_MOUSE | Rpl17 | RL17_MOUSE 60S ribosomal protein L17 | 9 | 750.641 | 1899.99 | 2.531 |
| sp\|Q8BIZ6\|SNIP1_MOUSE | Snip1 | SNIP1_MOUSE Smad nuclear-interacting protein 1 | 2 | 48.3057 | 122.168 | 2.529 |
| sp\|Q9CY58\|PAIRB_MOUSE | Serbp1 | PAIRB_MOUSE Plasminogen activator inhibitor 1 RNA- | 2 | 299.749 | 751.971 | 2.509 |
| | | binding protein | | | | |
| sp\|Q9WTI7\|MYOIC_MOUSE | Myo1c | MYO1C_MOUSE Unconventional myosin-Ic | 6 | 249.278 | 624.222 | 2.504 |
| sp\|P02301\|H3C_MOUSE | H3f3c | H3C_MOUSE Histone H3.3C | 3 | 675.077 | 1647.38 | 2.440 |
| sp\|A2A8Z1\|OSBL9_MOUSE | Osbpl9 | OSBL9_MOUSE Oxysterol-binding protein-related | 1 | 99.0679 | 235.331 | 2.375 |
| | | protein 9 | | | | |
| sp\|P86048\|RL10L_MOUSE | Rpl101 | RL10L_MOUSE 60S ribosomal protein L10-like | 6 | 1130.21 | 2622.86 | 2.321 |
| sp\|Q8VEK3\|HNRPU_MOUSE | Hnrnpu | HNRPU_MOUSE Heterogeneous nuclear | 1 | 99.4281 | 230.188 | 2.315 |
| | | ribonucleoprotein U | | | | |
| sp\|P53568\|CEBPG_MOUSE | Cebpg | CEBPG_MOUSE CCAAT/enhancer-binding | 2 | 558.082 | 1282.25 | 2.298 |
| | | protein gamma | | | | |
| sp\|Q8R4X3\|RBM12_MOUSE | Rbm12 | RBM12_MOUSE RNA-binding protein 12 | 5 | 309.967 | 710.869 | 2.293 |
| sp\|Q80TZ9\|RERE_MOUSE | Rere | RERE_MOUSE Arginine-glutamic acid dipeptide | 2 | 104.986 | 239.658 | 2.283 |
| | | repeats protein | | | | |
| sp\|Q9JJI8\|RL38_MOUSE | Rpl38 | RL38_MOUSE 60S ribosomal protein L38 | 2 | 224.482 | 510.009 | 2.272 |
| sp\|P57780\|ACTN4_MOUSE | Actn4 | ACTN4_MOUSE Alpha-actinin-4 | 2 | 92.5513 | 207.199 | 2.239 |
| sp\|Q9CSH3\|RRP44_MOUSE | Dis3 | RRP44_MOUSE Exosome complex exonuclease RRP44 | 3 | 89.4238 | 200.111 | 2.238 |
| tr\|E9PX68\|E9PX68_MOUSE | Slc4a1ap | E9PX68_MOUSE Protein Slc4a1ap | 1 | 22.2195 | 49.6912 | 2.236 |
| sp\|P60605\|UB2G2_MOUSE | Ube2g2 | UB2G2_MOUSE Ubiquitin-conjugating enzyme E2 G2 | 1 | 19.5968 | 43.7351 | 2.232 |
| sp\|Q8R1W8\|IMPG1_MOUSE | Impg1 | IMPG1_MOUSE Interphotoreceptor matrix proteoglycan 1 | 1 | 462.442 | 1031.78 | 2.231 |
| sp\|Q9DCD5\|TJAP1_MOUSE | Tjap1 | TJAP1_MOUSE Tight junction-associated protein 1 | 1 | 20.239 | 44.6089 | 2.204 |
| sp\|Q8VDJ3\|VIGLN_MOUSE | Hdlbp | VIGLN_MOUSE Vigilin | 4 | 130.328 | 283.367 | 2.174 |
| sp\|Q8C7X2\|EMC1_MOUSE | Emc1 | EMC1_MOUSE ER membrane protein complex subunit 1 | 16 | 1163.04 | 2525.65 | 2.172 |
| sp\|P14115\|RL27A_MOUSE | Rpl27a | RL27A_MOUSE 60S ribosomal protein L27a | 37 | 6776.95 | 14713.4 | 2.171 |
| sp\|O55128\|SAP18_MOUSE | Sap18 | SAP18_MOUSE Histone deacetylase complex subunit | 1 | 19.6072 | 42.5585 | 2.171 |
| | | SAP18 | | | | |
| sp\|Q9CZM2\|RL15_MOUSE | Rpl15 | RL15_MOUSE 60S ribosomal protein L15 | 6 | 518.529 | 1125.24 | 2.170 |
| sp\|Q99PL5\|RRBP1_MOUSE | Rrbp1 | RRBP1_MOUSE Ribosome-binding protein 1 | 3 | 106.128 | 228.495 | 2.153 |
| sp\|Q9WV85\|NDK3_MOUSE | Nme3 | NDK3_MOUSE Nucleoside diphosphate kinase 3 | 1 | 35.7928 | 76.7654 | 2.145 |
| sp\|P19253\|RL13A_MOUSE | Rpl13a | RL13A_MOUSE 60S ribosomal protein L13a | 2 | 104.356 | 223.514 | 2.142 |
| sp\|P62242\|RS8_MOUSE | Rps8 | RS8_MOUSE 40S ribosomal protein S8 | 8 | 955.926 | 2043.3 | 2.138 |
| sp\|Q8K5B2\|MCFD2_MOUSE | Mcfd2 | MCFD2_MOUSE Multiple coagulation factor deficiency | 8 | 752.846 | 1607.72 | 2.136 |
| | | protein 2 homolog | | | | |
| sp\|Q9CZX9\|EMC4_MOUSE | Emc4 | EMC4_MOUSE ER membrane protein complex subunit 4 | 1 | 53.2033 | 112.101 | 2.107 |
| sp\|Q52KE7\|CCNL1_MOUSE | Ccnl1 | CCNL1_MOUSE Cyclin-L1 | 3 | 105.691 | 222.284 | 2.103 |
| sp\|Q80WJ7\|LYRIC_MOUSE | Mtdh | LYRIC_MOUSE Protein LYRIC | 2 | 112.794 | 236.998 | 2.101 |
| sp\|O08747\|UNC5C_MOUSE | Unc5c | UNC5C_MOUSE Netrin receptor UNC5C | 3 | 109.781 | 230.609 | 2.101 |
| sp\|P51480\|CD2A1_MOUSE | Cdkn2a | CD2A1_MOUSE Cyclin-dependent kinase inhibitor 2A, | 1 | 23.0013 | 48.1565 | 2.094 |
| | | isoforms 1/2 | | | | |
| sp\|Q8CB77\|ELOA1_MOUSE | Tceb3 | ELOA1_MOUSE Transcription elongation factor B | 1 | 58.6001 | 121.827 | 2.079 |
| | | polypeptide 3 | | | | |
| sp\|Q8BM55\|TM214_MOUSE | Tmem214 | TM214_MOUSE Transmembrane protein 214 | 1 | 80.6229 | 166.576 | 2.066 |
| sp\|P27659\|RL3_MOUSE | Rpl3 | RL3_MOUSE 60S ribosomal protein L3 | 5 | 522.829 | 1078.24 | 2.062 |
| sp\|P62849\|RS24_MOUSE | Rps24 | RS24_MOUSE 40S ribosomal protein S24 | 7 | 1035.19 | 2129.9 | 2.057 |
| sp\|Q5SYH2\|TM199_MOUSE | Tmem199 | TM199_MOUSE Transmembrane protein 199 | 1 | 20.7734 | 42.6125 | 2.051 |
| sp\|Q99MR6\|SRRT_MOUSE | Srrt | SRRT_MOUSE Senate RNA effector molecule homolog | 1 | 76.9583 | 156.413 | 2.032 |
| sp\|Q9EP72\|EMC7_MOUSE | Emc7 | EMC7_MOUSE ER membrane protein complex subunit 7 | 2 | 328.111 | 664.743 | 2.026 |
| sp\|Q9CR57\|RL14_MOUSE | Rpl14 | RL14_MOUSE 60S ribosomal protein L14 | 2 | 523.501 | 1060.0.3 | 2.025 |
| sp\|P62918\|RL8_MOUSE | Rpl8 | RL8_MOUSE 60S ribosomal protein L8 | 4 | 277.599 | 557.688 | 2.009 |
| tr\|A2AUK5\|A2AUK5_MOUSE | Epb4.1l1 | A2AUK5_MOUSE Band 4.1-like protein 1 | 1 | 49.9328 | 99.7811 | 1.998 |
| sp\|P70302\|STIM1_MOUSE | Stim1 | STIM1_MOUSE Stromal interaction molecule 1 | 14 | 1659.79 | 3307.16 | 1.993 |
| sp\|Q99LE6\|ABCF2_MOUSE | Abcf2 | ABCF2_MOUSE ATP-binding cassette sub-family F | 3 | 236.622 | 471.414 | 1.992 |
| | | member 2 | | | | |
| sp\|Q9D8E6\|RL4_MOUSE | Rpl4 | RL4_MOUSE 60S ribosomal protein L4 | 3 | 292.539 | 582.734 | 1.992 |
| sp\|Q3U284\|TM231_MOUSE | Tmem231 | TM231_MOUSE Transmembrane protein 231 | 1 | 25.3926 | 50.5689 | 1.991 |
| sp\|P12970\|RL7A_MOUSE | Rpl7a | RL7A_MOUSE 60S ribosomal protein L7a | 2 | 134.184 | 267.121 | 1.991 |
| sp\|Q8BMK4\|CKAP4_MOUSE | Ckap4 | CKAP4_MOUSE Cytoskeleton-associated protein 4 | 158 | 22113.3 | 43794.9 | 1.980 |
| sp\|Q6ZWU9\|RS27_MOUSE | Rps27 | RS27_MOUSE 40S ribosomal protein S27 | 6 | 625.843 | 1238.83 | 1.979 |
| sp\|O70172\|PI42A_MOUSE | Pip4k2a | PI42A_MOUSE Phospliatidylinositol 5-phosphate | 5 | 494.436 | 971.277 | 1.964 |
| | | 4-kinase type-2 alpha | | | | |
| sp\|Q9D1R9\|RL34_MOUSE | Rpl34 | RL34_MOUSE 60S ribosomal protein L34 | 2 | 190.553 | 374.304 | 1.964 |
| sp\|P62082\|RS7_MOUSE | Rps7 | RS7_MOUSE 40S ribosomal protein S7 | 1 | 87.3402 | 171.546 | 1.964 |
| sp\|Q3UPF5\|ZCCHV_MOUSE | Zc3hav1 | ZCCHV_MOUSE Zinc finger CCCH-type antiviral | 23 | 1256.51 | 2463.77 | 1.961 |
| | | protein 1 | | | | |
| sp\|Q8VH51\|RBM39_MOUSE | Rbm39 | RBM39_MOUSE RNA-binding protein 39 | 1 | 29.2134 | 57.1712 | 1.957 |
| sp\|O08746\|MATN2_MOUSE | Matn2 | MATN2_MOUSE Matrilin-2 | 8 | 377.23 | 731.992 | 1.940 |
| sp\|Q9CW79\|GOGA1_MOUSE | Golga1 | GOGA1_MOUSE Golgin subfamily A member 1 | 3 | 217.617 | 421.49 | 1.937 |
| sp\|Q80X50\|UBP2L_MOUSE | Ubap2l | UBP2L_MOUSE Ubiquitin-associated protein 2-like | 5 | 283.669 | 548.461 | 1.933 |
| sp\|Q3THE2\|ML12B_MOUSE | Myl12b | ML12B_MOUSE Myosin regulatoiy light chain 12B | 1 | 104.924 | 202.508 | 1.930 |
| sp\|P25444\|RS2_MOUSE | Rps2 | RS2_MOUSE 40S ribosomal protein S2 | 11 | 1612.02 | 3101.67 | 1.924 |
| sp\|Q6P1H6\|ANKL2_MOUSE | Ankle2 | ANKL2_MOUSE Ankyrin repeat and LEM domain- | 7 | 401.473 | 772.347 | 1.924 |
| | | containing protein 2 | | | | |
| sp\|Q64213\|SF01_MOUSE | Sf1 | SF01_MOUSE Splicing factor 1 | 8 | 604.687 | 1162.63 | 1.923 |
| sp\|Q9D824\|FIPl_MOUSE | Fip1l1 | FIPI_MOUSE Pre-mRNA 3′-end-processing factor FIP1 | 2 | 159.067 | 304.555 | 1.915 |
| sp\|P20152\|VIME_MOUSE | Vim | VIME_MOUSE Vimentin | 10 | 665.576 | 1273.17 | 1.913 |
| sp\|Q91VR5\|DDX1_MOUSE | Ddx1 | DDX1_MOUSE ATP-dependent RNA helicase DDX1 | 5 | 482.578 | 916.624 | 1.899 |
| sp\|Q9Z2Z9\|GFPT2_MOUSE | Gfpt2 | GFPT2_MOUSE Glutamine-fructose-6-phosphate | 9 | 641.658 | 1216.44 | 1.896 |
| | | aminotransferase [isomerizing] 2 | | | | |
| sp\|P60867\|RS20_MOUSE | Rps20 | RS20_MOUSE 40S ribosomal protein S20 | 3 | 427.61 | 808.632 | 1.891 |
| sp\|Q8BG05\|ROA3_MOUSE | Hnrnpa3 | ROA3_MOUSE Heterogeneous nuclear | 2 | 60.1673 | 113.719 | 1.890 |
| | | ribonucleoprotein A3 | | | | |
| sp\|Q9DCF9\|SSRG_MOUSE | Ssr3 | SSRG_MOUSE Translocon-associated protein subunit | 2 | 314.512 | 593.031 | 1.886 |
| | | gamma | | | | |
| sp\|P62245\|RS15A_MOUSE | Rps15a | RS15A_MOUSE 40S ribosomal protein S15a | 8 | 884.844 | 1667.52 | 1.885 |
| sp\|Q5SQX6\|CYFP2_MOUSE | Cyfip2 | CYFP2_MOUSE Cytoplasmic FMR1-interacting protein 2 | 1 | 97.522 | 183.749 | 1.884 |
| sp\|Q8CAQ8\|IMMT_MOUSE | Immt | IMMT_MOUSE Mitochondrial inner membrane protein | 4 | 322.349 | 607.277 | 1.884 |
| sp\|Q9Z2G6\|SE1L1_MOUSE | Sel1l | SE1L1_MOUSE Protein sel-1 homolog 1 | 4 | 230.748 | 433.386 | 1.878 |
| sp\|Q810J8\|ZFYV1_MOUSE | Zfyve1 | ZFYV1_MOUSE Zinc finger FYVE domain-containing | 6 | 469.284 | 880.637 | 1.877 |
| | | protein 1 | | | | |
| sp\|Q8CDG3\|VCIP1_MOUSE | Vcpip1 | VCIP1_MOUSE Deubiquitinating protein VCIP135 | 1 | 61.3015 | 115.026 | 1.876 |
| sp\|Q8K310\|MATR3_MOUSE | Matr3 | MATR3_MOUSE Matrin-3 | 1 | 48.1279 | 90.1785 | 1.874 |
| sp\|Q70FJ1\|AKAP9_MOUSE | Akap9 | AKAP9_MOUSE A-kinase anchor protein 9 | 1 | 377.548 | 707.028 | 1.873 |
| sp\|Q9R0B9\|PLOD2_MOUSE | Plod2 | PLOD2_MOUSE Procollagen-lysine, 2-oxoglutarate | 39 | 4275.15 | 8004.98 | 1.872 |
| | | 5-dioxygenase 2 | | | | |
| sp\|Q9QYC7\|VKGC_MOUSE | Ggcx | VKGC_MOUSE Vitamin K-dependent gamma- | 3 | 187.073 | 348.26 | 1.862 |
| | | carboxylase | | | | |
| sp\|P97351\|RS3A_MOUSE | Rps3a | RS3A_MOUSE 40S ribosomal protein S3a | 7 | 1198.41 | 2226.82 | 1.858 |
| sp\|Q8BSY0\|ASPH_MOUSE | Asph | ASPH_MOUSE Aspartyl/asparaginyl beta-hydroxylase | 26 | 3796.02 | 7007.73 | 1.846 |
| sp\|P47857\|K6PF_MOUSE | Pfkm | K6PF_MOUSE 6-phosphofructokinase, muscle type | 5 | 385.498 | 710.273 | 1.842 |
| sp\|P62855\|RS26_MOUSE | Rps26 | RS26_MOUSE 40S ribosomal protein S26 | 3 | 534.213 | 983.928 | 1.842 |
| sp\|Q9CXW4\|RL11_MOUSE | Rpl11 | RL11_MOUSE 60S ribosomal protein L11 | 5 | 1011.18 | 1857.65 | 1.837 |
| sp\|P62281\|RS11_MOUSE | Rps11 | RS11_MOUSE 40S ribosomal protein S11 | 18 | 3318.01 | 6075.02 | 1.831 |
| sp\|P62075\|TIM13_MOUSE | Timm13 | TIM13_MOUSE Mitochondrial import inner membrane | 1 | 43.2536 | 79.0238 | 1.827 |
| | | translocase subunit Tim13 | | | | |
| sp\|Q8C6U2\|PQLC3_MOUSE | Pqlc3 | PQLC3_MOUSE PQ-loop repeat-containing protein 3 | 1 | 29.2895 | 53.3758 | 1.822 |
| sp\|Q6ZWN5\|RS9_MOUSE | Rps9 | RS9_MOUSE 40S ribosomal protein S9 | 16 | 2339.6 | 4255.79 | 1.819 |
| sp\|055187\|CBX4_MOUSE | Cbx4 | CBX4_MOUSE E3 SUMO-protein ligase CBX4 | 1 | 22.426 | 40.7207 | 1.816 |
| sp\|Q8BY71\|HAT1_MOUSE | Hat1 | HAT1_MOUSE Histone acetyltransferase type B | 1 | 40.7498 | 73.9024 | 1.814 |
| | | catalytic subunit | | | | |
| sp\|P61022\|CHP1_MOUSE | Chp1 | CHP1_MOUSE Calcincurin B homologous protein 1 | 1 | 29.0663 | 52.7096 | 1.813 |
| sp\|Q91YR7\|PRP6_MOUSE | Pipf6 | PRP6_MOUSE Pre-mRNA-proccssing factor 6 | 1 | 23.9484 | 43.3901 | 1.812 |
| sp\|Q80TN4\|DJC16_MOUSE | Dnajc16 | DJC16_MOUSE DnaJ homolog subfamily C member 16 | 2 | 137.908 | 249.844 | 1.812 |
| sp\|P83882\|RL36A_MOUSE | Rpl36a | RL36A_MOUSE 60S ribosomal protein L36a | 2 | 481.537 | 870.558 | 1.808 |
| sp\|Q7TPV4\|MBB1A_MOUSE | Mvbbp1a | MBB1A_MOUSE Myb-binding protein 1A | 1 | 26.7576 | 48.3024 | 1.805 |
| sp\|E9Q7X6\|HEG1_MOUSE | Heg1 | HEG1_MOUSE Protein HEG homolog 1 | 2 | 390.492 | 704.803 | 1.805 |
| sp\|Q8BMA6\|SRP68_MOUSE | Srp68 | SRP68_MOUSE Signal recognition particle subunit SRP68 | 1 | 31.1422 | 56.1345 | 1.803 |
| sp\|P62908\|RS3_MOUSE | Rps3 | RS3_MOUSE 40S ribosomal protein S3 | 12 | 1763.09 | 3177.45 | 1.802 |
| tr\|E9QKL6\|E9QKL6_MOUSE | Ifi204 | E9QKL6_MOUSE Interferon-activable protein 204 | 3 | 188.887 | 340.313 | 1.802 |
| sp\|O35598\|ADA10_MOUSE | Adam10 | ADA10_MOUSE Disintegrin and metalloproteinase | 3 | 229.5 | 412.382 | 1.797 |
| | | domain-containing protein 10 | | | | |
| sp\|Q8BGD5\|CPT1C_MOUSE | Cpt1c | CPT1C_MOUSE Carnitine O-palmitoyltransferase 1, | 1 | 66.4396 | 119.144 | 1.793 |
| | | brain isoform | | | | |
| tr\|Q9DBK7\|Q9DBK7_MOUSE | Uba7 | Q9DBK7_MOUSE MCG18845.isoform CRA_d | 2 | 116.957 | 209.676 | 1.793 |
| sp\|P47856\|GFPT1_MOUSE | Gfpt1 | GFPT1_MOUSE Glutamine--fructose-6-phosphate | 15 | 1736.12 | 3108.2 | 1.790 |
| | | aminotransferase [isomerizing] 1 | | | | |
| sp\|Q920B9\|SP16H_MOUSE | Supt16h | SP16H_MOUSE FACT complex subunit SPT16 | 1 | 51.4846 | 92.0665 | 1.788 |
| tr\|G5E8A0\|G5E8A0_MOUSE | Osbpl11 | G5E8A0_MOUSE Oxysterol-binding protein | 4 | 497.304 | 888.907 | 1.787 |
| sp\|P70245\|EBP_MOUSE | Ebp | EBP_MOUSE 3-beta-hydroxysteroid-Delta(8), | 2 | 218.89 | 389.381 | 1.779 |
| | | Delta(7)-isomerase | | | | |
| sp\|P63017\|HSP7C_MOUSE | Hspa8 | HSP7C_MOUSE Heat shock cognate 71 kDa protein | 46 | 6718.68 | 11916.2 | 1.774 |
| sp\|Q8K4Z5\|SF3A1_MOUSE | Sf3a1 | SF3A1_MOUSE Splicing factor 3A subunit 1 | 42 | 3714.64 | 6587.79 | 1.773 |
| sp\|Q8K2C9\|HACD3_MOUSE | ptplad1 | HACD3_MOUSE Very-long-chain (3R)-3-hydroxyacyl- | 5 | 430.933 | 762.865 | 1.770 |
| | | [acyl-carrier protein] dehydratase 3 | | | | |
| tr\|G3UYI3\|G3UYI3_MOUSE | Calu | G3UYI3_MOUSE Calumenin (Fragment) | 1 | 77.4128 | 136.812 | 1.767 |
| sp\|P12382\|K6PL_MOUSE | Pfkl | K6PL_MOUSE 6-phosphofructokinase, liver type | 11 | 1037.39 | 1833.11 | 1.767 |
| sp\|Q8C4A5\|ASXL3_MOUSE | Asxl3 | ASXL3_MOUSE Putative Polycomb group protein | 1 | 63.7757 | 112.652 | 1.766 |
| | | ASXL3 | | | | |
| sp\|Q9D3Bl\|HACD2_MOUSE | Ptplb | HACD2_MOUSE Very-long-chain (3R)-3-hydroxyacyl- | 1 | 116.195 | 204.158 | 1.757 |
| | | [acyl-carrier protein] dehydratase 2 | | | | |
| sp\|P51410\|RL9_MOUSE | Rpl9 | RL9_MOUSE 60S ribosomal protein L9 | 4 | 770.996 | 1353.18 | 1.755 |
| sp\|Q9CQW0\|EMC6_MOUSE | Eme6 | EMC6_MOUSE ER membrane protein complex subunit 6 | 2 | 166.129 | 291.42 | 1.754 |
| sp\|Q7TN98\|CPEB4_MOUSE | Cpeb4 | CPEB4_MOUSE Cytoplasmic polyadenylation element- | 1 | 102.805 | 179.855 | 1.749 |
| | | binding protein 4 | | | | |
| sp\|Q8VIJ6\|SFPQ_MOUSE | Sfpq | SFPQ_MOUSE Splicing factor, proline-and glutamine-rich | 20 | 3308.83 | 5787.91 | 1.749 |
| sp\|Q62440\|TLE1_MOUSE | Tle1 | TLE1_MOUSE Transducin-like enhancer protein 1 | 1 | 32.2907 | 56.4832 | 1.749 |
| sp\|Q99K13\|EMC3_MOUSE | Eme3 | EMC3_MOUSE ER membrane protein complex subunit 3 | 3 | 632.729 | 1103.27 | 1.744 |
| sp\|P63325\|RS10_MOUSE | Rps10 | RS10_MOUSE 40S ribosomal protein S10 | 2 | 229.559 | 400.008 | 1.743 |
| sp\|Q9WUA3\|K6PP_MOUSE | Pfkp | K6PP_MOUSE 6-phosphofructokinase type C | 1 | 129.353 | 224.803 | 1.738 |
| sp\|Q8BGJ9\|U2AF4_MOUSE | U2af1l4 | U2AF4_MOUSE Splicing factor U2AF 26 kDa subunit | 2 | 189.126 | 327.879 | 1.734 |
| sp\|Q8VE22\|RT23_MOUSE | Mrps23 | RT23_MOUSE 28S ribosomal protein S23, mitochondrial | 1 | 42.2366 | 73.2139 | 1.733 |
| sp\|Q8K3A9\|MEPCE_MOUSE | Mepce | MEPCE_MOUSE 7SK snRNA methylphosphate capping | 4 | 359.387 | 620.375 | 1.726 |
| | | enzyme | | | | |
| sp\|Q9CQF3\|CPSF5_MOUSE | Nudt21 | CPSF5_MOUSE Cleavage and polyadenylation | 2 | 254.246 | 437.824 | 1.722 |
| | | specificity factor subunit 5 | | | | |
| sp\|Q9EPK7\|XPO7_MOUSE | Xpo7 | XPO7_MOUSE Expoitin-7 | 1 | 22.8009 | 39.210.3 | 1.720 |
| sp\|P18760\|COF1_MOUSE | Cfl1 | COF1_MOUSE Cofilin-1 | 2 | 337.93 | 580.524 | 1.718 |
| sp\|Q8CJ53-3\|CIP4_MOUSE | Trip10 | CIP4_MOUSE Isoform 3 of Cde42-interacting protein 4 | 9 | 554.175 | 950.556 | 1.715 |
| sp\|O35286\|DHX15_MOUSE | Dhx15 | DHX15_MOUSE Putative pre-mRNA-splicing factor | 39 | 5671.84 | 9727.46 | 1.715 |
| | | ATP-dependent RNA helicase DHX15 | | | | |
| sp\|Q99MU3\|DSRAD_MOUSE | Adar | DSRAD_MOUSE Double-stranded RNA-specific | 5 | 360.652 | 617.421 | 1.712 |
| | | adenosine deaminase | | | | |
| sp\|Q9WVA2\|TIM8A_MOUSE | Timm8a1 | TIM8A_MOUSE Mitochondrial import inner membrane | 1 | 56.3368 | 96.4104 | 1.711 |
| | | translocase subunit Tim8 A | | | | |
| sp\|Q6l656\|DDX5_MOUSE | Ddx5 | DDX5_MOUSE Probable ATP-dependent RNA helicase | 5 | 278.066 | 475.105 | 1.709 |
| | | DDX5 | | | | |
| sp\|P09405\|NUCL_MOUSE | Ncl | NUCL_MOUSE Nucleolin | 7 | 501.372 | 856.141 | 1.708 |
| sp\|O08789\|MNT_MOUSE | Mnt | MNT_MOUSE Max-binding protein MNT | 3 | 340.832 | 579.162 | 1.699 |
| sp\|Q9CRD2\|EMC2_MOUSE | Eme2 | EMC2_MOUSE ER membrane protein complex subunit 2 | 6 | 511.411 | 868.928 | 1.699 |
| sp\|Q9QZ88\|VPS29_MOUSE | Vps29 | VPS29_MOUSE Vacuolar protein sorting-associated | 1 | 136.973 | 232.523 | 1.698 |
| | | protein 29 | | | | |
| sp\|Q7TNC4\|LC7L2_MOUSE | Luc7l2 | LC7L2_MOUSE Putative RNA-binding protein Luc7- | 11 | 1373.36 | 2315.85 | 1.686 |
| | | like 2 | | | | |
| sp\|Q9WU40\|MAN1_MOUSE | Lemd3 | MAN1_MOUSE Inner nuclear membrane protein Man1 | 1 | 22.7499 | 38.2568 | 1.682 |
| sp\|Q8K297\|GT251_MOUSE | Colgalt1 | GT251_MOUSE Procollagcn galactosyltransferase 1 | 5 | 348.06 | 585.03 | 1.681 |
| sp\|P61358\|RL27_MOUSE | Rpl27 | RL27_MOUSE 60S ribosomal protein L27 | 1 | 122.949 | 206.299 | 1.678 |
| sp\|O70443\|GNAZ_MOUSE | Gnaz | GNAZ_MOUSE Guanine nucleotide-binding protein G(z) | 1 | 177.201 | 296.883 | 1.675 |
| | | subunit alpha | | | | |
| sp\|Q3TZZ7\|ESYT2_MOUSE | Esyt2 | ESYT2_MOUSE Extended svnaptotagmin-2 | 43 | 5434.1 | 9099.06 | 1.674 |
| sp\|Q61881\|MCM7_MOUSE | Mcm7 | MCM7_MOUSE DNA replication licensing factor MCM7 | 8 | 553.884 | 925 | 1.670 |
| sp\|Q9CRB9\|CHCH3_MOUSE | Chchd3 | CHCH3_MOUSE Coiled-coil-helix-coiled-coil-helix | 1 | 45.2771 | 75.5318 | 1.668 |
| | | domain-containing protein 3, mitochondrial | | | | |
| sp\|Q60598\|SRC8_MOUSE | Cttn | SRC8_MOUSE Src substrate cortactin | 1 | 34.5385 | 57.58 | 1.667 |
| sp\|Q8C145\|S39A6_MOUSE | Slc39a6 | S39A6_MOUSE Zinc transporter ZIP6 | 4 | 294.412 | 490.596 | 1.666 |
| sp\|Q8BTV2\|CPSF7_MOUSE | Cpsf7 | CPSF7_MOUSE Cleavage and polyadenylation | 2 | 129.146 | 214.993 | 1.665 |
| | | specificity factor subunit 7 | | | | |
| sp\|Q80T69\|RSBN1_MOUSE | Rsbn1 | RSBN1_MOUSE Round spermatid basic protein 1 | 2 | 72.8055 | 121.129 | 1.664 |
| sp\|Q9DBT5\|AMPD2_MOUSE | Ampd2 | AMPD2_MOUSE AMP deaminase 2 | 5 | 382.369 | 635.674 | 1.662 |
| tr\|Q8CIE4\|Q8CIE4_MOUSE | Parp10 | Q8CIE4_MOUSE Plec1 protein | 2 | 51.1457 | 84.8486 | 1.659 |
| sp\|O55143\|AT2A2_MOUSE | Atp2a2 | AT2A2_MOUSE Sarcoplasmic/endoplasmic | 34 | 3074.48 | 5075.19 | 1.651 |
| | | reticulum calcium ATPase 2 | | | | |
| sp\|P84L04\|SRSF3_MOUSE | Srsf3 | SRSF3_MOUSE Serine/arginine-rich splicing factor 3 | 6 | 523.275 | 863.441 | 1.650 |
| sp\|Q9CQE8\|CN166_MOUSE | | CN166_MOUSE UPF0568 protein C14orf166 homolog | 2 | 302.378 | 496.272 | 1.641 |
| tr\|Q6ZWQ7\|Q6ZWQ7_MOUSE | Spcs3 | Q6ZWQ7_MOUSE Signal peptidase complex subunit 3 | 2 | 243.176 | 399.021 | 1.641 |
| sp\|O8C6M1\|UBP20_MOUSE | Usp20 | UBP20_MOUSE Ubiquitin carboxyl-terminal | 1 | 27.117 | 44.4561 | 1.639 |
| | | hydrolase 20 | | | | |
| sp\|P99027\|RLA2_MOUSE | Rplp2 | RLA2_MOUSE 60S acidic ribosomal protein P2 | 2 | 194.942 | 318.932 | 1.636 |
| sp\|O70378\|EMC8_MOUSE | Eme8 | EMC8_MOUSE ER membrane protein complex subunit 8 | 7 | 859.648 | 1405.46 | 1.635 |
| sp\|Q9CZW4\|ACSL3_MOUSE | Acsl3 | ACSL3_MOUSE Long-chain-fatty-acid--CoA ligase 3 | 3 | 248.448 | 405.824 | 1.633 |
| sp\|P10852\|4F2_MOUSE | Slc3a2 | 4F2_MOUSE 4F2 cell-surface antigen heavy chain | 1 | 38.3937 | 62.6271 | 1.631 |
| sp\|Q01405\|SC23A_MOUSE | Sec23a | SC23A_MOUSE Protein transport protein Sec23A | 14 | 1865.46 | 3034.17 | 1.626 |
| sp\|Q5DTM8\|BRE1A_MOUSE | Rnf20 | BRE1A_MOUSE E3 ubiquitin-protein ligase BRE1A | 2 | 154.101 | 250.174 | 1.623 |
| sp\|Q99J56\|DERLI_MOUSE | Derl1 | DERL1_MOUSE Derlin-1 | 1 | 24.4931 | 39.7239 | 1.622 |
| sp\|Q91VX2\|UBAP2_MOUSE | Ubap2 | UBAP2_MOUSE Ubiquirin-associated protein 2 | 7 | 555.573 | 898.508 | 1.617 |
| sp\|P67871\|CSK2B_MOUSE | Csnk2b | CSK2B_MOUSE Casein kinase II subunit beta | 2 | 247.838 | 399.066 | 1.610 |
| sp\|Q9D5T0\|ATAD1_MOUSE | Atad1 | ATAD1_MOUSE ATPase family AAA domain- | 1 | 56.5177 | 90.9506 | 1.609 |
| | | containing protein 1 | | | | |
| sp\|Q9DC23\|DJC10_MOUSE | Dnajc10 | DJC10_MOUSE DnaJ homolog subfamily C member 10 | 36 | 6392.75 | 10276.9 | 1.608 |
| sp\|P62301\|RS13_MOUSE | Rps13 | RS13_MOUSE 40S ribosomal protein S13 | 9 | 1196.88 | 1921.4 | 1.605 |
| sp\|Q9D662\|SC23B_MOUSE | Sec23b | SC23B_MOUSE Protein transport protein Sec23B | 2 | 125.143 | 200.841 | 1.605 |
| sp\|Q8CFB4\|GBP5_MOUSE | Gbp5 | GBP5_MOUSE Guanylate-binding protein 5 | 6 | 700.766 | 1124.26 | 1.604 |
| sp\|Q80XI4\|PI42B_MOUSE | Pip4k2b | PI42B_MOUSE Phosphatidylinositol 5-phosphate | 1 | 30.3359 | 48.6499 | 1.604 |
| | | 4-kinase type-2 beta | | | | |
| sp\|P25206\|MCM3_MOUSE | Mcm3 | MCM3_MOUSE DNA replication licensing factor | 8 | 988.243 | 1584.29 | 1.603 |
| | | MCM3 | | | | |
| sp\|Q8C0I1\|ADAS_MOUSE | Agps | ADAS_MOUSE Alkyldihydroxyacetonephosphate | 2 | 116.769 | 187.055 | 1.602 |
| | | synthase, peroxisomal | | | | |
| sp\|Q05CL8\|LARP7_MOUSE | Larp7 | LARP7_MOUSE La-related protein 7 | 3 | 251.044 | 402.133 | 1.602 |
| sp\|Q3TLP5\|ECHD2_MOUSE | Echde2 | ECHD2_MOUSE Enoyl-CoA hydratase domain- | 8 | 915.303 | 1464.66 | 1.600 |
| | | containing protein 2, mitochondrial | | | | |
| sp\|P97857\|ATS1_MOUSE | Adamts1 | ATS1_MOUSE A disintegrin and metalloproteinase with | 1 | 32.5033 | 52.0063 | 1.600 |
| | | thrombospondin motifs 1 | | | | |
| sp\|Q9WVD4\|CLCN5_MOUSE | Clcn5 | CLCN5_MOUSE H(+)/Cl(−) exchange transporter 5 | 2 | 264.246 | 422.739 | 1.600 |
| sp\|Q8VHE0\|SEC63_MOUSE | Sec63 | SEC63_MOUSE Translocation protein SEC63 homolog | 9 | 753.08 | 1204.6 | 1.600 |
| sp\|P13011\|ACOD2_MOUSE | Scd2 | ACOD2_MOUSE Acyl-CoA desaturase 2 | 2 | 296.031 | 472.854 | 1.597 |
| sp\|Q8BGQ4\|POMT2_MOUSE | Pomt2 | POMT2_MOUSE Protein O-mannosyl-transferase 2 | 1 | 28.1767 | 44.9129 | 1.594 |
| sp\|P49717\|MCM4_MOUSE | Mem4 | MCM4_MOUSE DNA replication licensing factor MCM4 | 5 | 350.99 | 558.824 | 1.592 |
| sp\|P67984\|RL22_MOUSE | Rpl22 | RL22_MOUSE 60S ribosomal protein L22 | 2 | 522.253 | 831.361 | 1.592 |
| sp\|Q8R4H9\|ZNT5_MOUSE | Slc30a5 | ZNT5_MOUSE Zinc transporter 5 | 14 | 3364.63 | 5355.85 | 1.592 |
| sp\|Q80UM7\|MOGS_MOUSE | Mogs | MOGS_MOUSE Mannosyl-oligosaccharide glucosidase | 11 | 1179.65 | 1875.43 | 1.590 |
| sp\|P21981\|TGM2_MOUSE | Tgm2 | TGM2_MOUSE Protein-glutaminc gamma- | 2 | 106.401 | 169.012 | 1.588 |
| | | glutamyltransferase 2 | | | | |
| sp\|P04227\|HA2Q_MOUSE | H2-Aa | HA2Q_MOUSE H-2 class II histocompatibility antigen, | 1 | 83.0723 | 131.863 | 1.587 |
| | | A-Q alpha chain (Fragment) | | | | |
| sp\|P62702\|RS4X_MOUSE | Rps4x | RS4X_MOUSE 40S ribosomal protein S4, X isoform | 15 | 3698.66 | 5869.48 | 1.587 |
| sp\|P08003\|PDIA4_MOUSE | Pdia4 | PDIA4_MOUSE Protein disulfide-isomerase A4 | 59 | 10222.7 | 16219.9 | 1.587 |
| sp\|Q9D0Z3\|TMM53_MOUSE | Tmem53 | TMM53_MOUSE Transmembrane protein 53 | 1 | 51.2604 | 81.3235 | 1.586 |
| sp\|Q922B2\|SYDC_MOUSE | Dars | SYDC_MOUSE Aspartate--tRNA ligase, cytoplasmic | 1 | 24.7852 | 39.2851 | 1.585 |
| sp\|O70230\|ZN143_MOUSE | Znf143 | ZN143_MOUSE Zinc finger protein 143 | 2 | 108.183 | 171 | 1.581 |
| sp\|P62996\|TRA2B_MOUSE | Tra2b | TRA2B_MOUSE Transformer-2 protein homolog beta | 2 | 134.611 | 212.728 | 1.580 |
| sp\|Q8BHI7\|ELOV5_MOUSE | Elovl5 | ELOV5_MOUSE Elongation of very long chain fatty | 1 | 47.1272 | 74.4343 | 1.579 |
| | | acids protein 5 | | | | |
| spO88712\|CTBP1_MOUSE | Ctbp1 | CTBP1_MOUSE C-terminal-binding protein 1 | 1 | 39.606 | 62.4419 | 1.577 |
| sp\|Q62093\|SRSF2_MOUSE | Srsf2 | SRSF2_MOUSE Serine/arginine-rich splicing factor 2 | 1 | 159.35 | 251.108 | 1.576 |
| sp\|Q8BH59\|CMC1_MOUSE | Slc25a12 | CMC1_MOUSE Calcium-binding mitochondrial | 5 | 328.159 | 516.502 | 1.574 |
| | | carrier protein Aralar1 | | | | |
| sp\|Q8BGC0\|HTSF1_MOUSE | Htatsf1 | HTSF1_MOUSE HIV Tat-specific factor 1 homolog | 10 | 436.735 | 686.233 | 1.571 |
| sp\|Q7TT37\|ELP1_MOUSE | Ikbkap | ELP1_MOUSE Elongator complex protein 1 | 1 | 68.9312 | 108.28 | 1.571 |
| tr\|D3YVL4\|D3YVL4_MOUSE | Mcfd2 | D3YVL4_MOUSE Multiple coagulation factor | 4 | 124.931 | 196.162 | 1.570 |
| | | deficiency protein 2 homolog (Fragment) | | | | |
| sp\|O70492\|SNX3_MOUSE | Snx3 | SNX3_MOUSE Sorting nexin-3 | 1 | 32.2636 | 50.5612 | 1.567 |
| sp\|P47964\|RL36_MOUSE | Rpl36 | RL36_MOUSE 60S ribosomal protein L36 | 1 | 89.3497 | 139.699 | 1.564 |
| sp\|Q9R0E2\|PLOD1_MOUSE | Plod1 | PLOD1_MOUSE Procollagen-lysine,2-oxoglutarate | 14 | 1465.12 | 2288.84 | 1.562 |
| | | 5-dioxygenase 1 | | | | |
| sp\|P23188\|FURIN_MOUSE | Furin | FURIN_MOUSE Furin | 1 | 80.3499 | 125.277 | 1.559 |
| sp\|Q9EPE9\|AT131_MOUSE | Atp13a1 | AT131_MOUSE Probable cation-transporting | 11 | 674.305 | 1051.25 | 1.559 |
| | | ATPase 13A1 | | | | |
| sp\|Q8R2U0\|SEH1_MOUSE | Seh1l | SEH1_MOUSE Nucleoporin SEH1 | 1 | 24.5172 | 38.1974 | 1.558 |
| sp\|E9Q9A9\|OAS2_MOUSE | Oas2 | OAS2_MOUSE 2′-5′-oligoadenylate synthase 2 | 2 | 138.065 | 214.864 | 1.556 |
| sp\|Q9QYE6\|GOGA5_MOUSE | Golga5 | GOGA5_MOUSE Golgin subfamily A member 5 | 3 | 148.426 | 230.836 | 1.555 |
| sp\|Q922Q9\|CHID1_MOUSE | Chid1 | CHID1_MOUSE Chitinase domain-containing protein 1 | 1 | 151.657 | 235.63 | 1.554 |
| sp\|Q68FD5\|CLH1_MOUSE | Cltc | CLH1_MOUSE Clatluin heavy chain 1 | 5 | 361.756 | 562.028 | 1.554 |
| sp\|Q9WV55\|VAPA_MOUSE | Vapa | VAPA_MOUSE Vesicle-associated membrane | 4 | 875.265 | 1358.25 | 1.552 |
| | | protein-associated protein A | | | | |
| sp\|P70295\|AUP1_MOUSE | Aup1 | AUP1_MOUSE Ancient ubiquitous protein 1 | 1 | 24.9451 | 38.7002 | 1.551 |
| sp\|O35066\|KIF3C_MOUSE | Kif3c | KIF3C_MOUSE Kinesin-like protein KIF3C | 1 | 331.386 | 514.032 | 1.551 |
| sp\|P35979\|RL12_MOUSE | Rpl12 | RL12_MOUSE 60S ribosomal protein L12 | 5 | 542.56 | 841.589 | 1.551 |
| sp\|Q6NVF9\|CPSF6_MOUSE | Cpsf6 | CPSF6_MOUSE Cleavage and polyadenylation | 6 | 531.205 | 823.057 | 1.549 |
| | | specificity factor subunit 6 | | | | |
| sp\|P61750\|ARF4_MOUSE | Arf4 | ARF4_MOUSE ADP-ribosylation factor 4 | 6 | 1033.62 | 1600.87 | 1.549 |
| sp\|P42225\|STAT1_MOUSE | Stat1 | STAT1_MOUSE Signal transducer and activator | 1 | 50.6718 | 78.4153 | 1.548 |
| | | of transcription 1 | | | | |
| sp\|P57722\|PCBP3_MOUSE | Pcbp3 | PCBP3_MOUSE Poly(rC)-binding protein 3 | 1 | 31.3564 | 48.4924 | 1.546 |
| sp\|P62264\|RS14_MOUSE | Rps14 | RS14_MOUSE 40S ribosomal protein S14 | 8 | 833.171 | 1287.71 | 1.546 |
| sp\|Q9R1C7\|PR40A_MOUSE | Prpf40a | PR40A_MOUSE Pre-mRNA-processing factor 40 | 1 | 57.7385 | 89.2333 | 1.545 |
| | | homolog A | | | | |
| sp\|Q8CG70\|P3H3_MOUSE | Leprel2 | P3H3_MOUSE Prolyl 3-hydroxylase 3 | 8 | 568.298 | 878.118 | 1.545 |
| sp\|P97461\|RS5_MOUSE | Rps5 | RS5_MOUSE 40S ribosomal protein S5 | 4 | 656.444 | 1014.3 | 1.545 |
| sp\|P61514\|RL37A_MOUSE | Rpl37a | RL37A_MOUSE 60S ribosomal protein L37a | 1 | 26.6041 | 41.0953 | 1.545 |
| sp\|Q8BGA9\|OXA1L_MOUSE | Oxa1l | OXA1L_MOUSE Mitochondrial inner membrane | 1 | 39.0934 | 60.2437 | 1.541 |
| | | protein OXA1L | | | | |
| sp\|Q9CQB5\|CISD2_MOUSE | Cisd2 | CISD2_MOUSE CDGSH iron-sulfur domain- | 4 | 584.55 | 900.314 | 1.540 |
| | | containing protein 2 | | | | |
| sp\|Q8C3F2\|F120C_MOUSE | Fam120c | F120C_MOUSE Constitutive coactivator of PPAR- | 1 | 29.6643 | 45.6769 | 1.540 |
| | | gamma-like protein 2 | | | | |
| sp\|O35218\|CPSF2_MOUSE | Cpsf2 | CPSF2_MOUSE Cleavage and polyadenylation | 1 | 31.8059 | 48.966 | 1.540 |
| | | specificity factor subunit 2 | | | | |
| sp\|Q8BVG4\|DPP9_MOUSE | Dpp9 | DPP9_MOUSE Dipeptidyl peptidase 9 | 18 | 3081.15 | 4741.73 | 1.539 |
| sp\|Q60597\|ODO1_MOUSE | Ogdh | ODO1_MOUSE 2-oxoglutarate dehydrogenase, | 38 | 2685.69 | 4128.79 | 1.537 |
| | | mitochondrial | | | | |
| sp\|O88569\|ROA2_MOUSE | Hnrnpa2b1 | ROA2_MOUSE Heterogeneous nuclear | 4 | 280.213 | 430.516 | 1.536 |
| | | ribonuclcoproteins A2/B1 | | | | |
| sp\|Q9CXG3\|PPIL4_MOUSE | Ppil4 | PPIL4_MOUSE Peptidyl-prolyl cis-trans isomerase-like 4 | 3 | 141.419 | 217.225 | 1.536 |
| sp\|Q9CW46\|RAVR1_MOUSE | Raver1 | RAVR1_MOUSE Ribonucleoprotein PTB -binding 1 | 1 | 32.8984 | 50.5256 | 1.536 |
| sp\|Q69ZH9\|RHG23_MOUSE | Arhgap23 | RHG23_MOUSE Rho GTPase-activating protein 23 | 1 | 519.561 | 797.28 | 1.535 |
| sp\|Q8BRH0\|TMTC3_MOUSE | Tmte3 | TMTC3_MOUSE Transmembrane and TPR repeat- | 1 | 26.5953 | 40.8043 | 1.534 |
| | | containing protein 3 | | | | |
| sp\|Q62318\|TIF1B_MOUSE | Trim28 | TIF1B_MOUSE Transcription intermediary factor 1-beta | 10 | 440.189 | 675.21 | 1.534 |
| sp\|P97311\|MCM6_MOUSE | Mem6 | MCM6_MOUSE DNA replication licensing factor MCM6 | 16 | 1634.26 | 2505.13 | 1.533 |
| sp\|Q6PDM2\|SRSFI_MOUSE | Srsf1 | SRSF1_MOUSE Serine/arginine-rich splicing factor 1 | 3 | 145.5 | 222.638 | 1.530 |
| sp\|Q8CHK3\|MBOA7_MOUSE | Mboat7 | MBOA7_MOUSE Lysophospholipid acyltransferase 7 | 1 | 43.6744 | 66.79 | 1.529 |
| sp\|Q8BMF4\|ODP2_MOUSE | Dlat | ODP2_MOUSE Dihydrolipoyllysine-residue acetyl | 2 | 119.329 | 182.466 | 1.529 |
| | | transferase component of pyruvate dehydrogenase | | | | |
| | | complex, mitochondrial | | | | |
| sp\|Q3TVI8\|PBIP1_MOUSE | Pbxip1 | PBIP1_MOUSE Pre-B-cell leukemia transcription | 41 | 4322.21 | 6605.71 | 1.528 |
| | | factor-interacting protein 1 | | | | |
| sp\|Q9QYI4\|DJB12_MOUSE | Dnajb12 | DJB12_MOUSE DnaJ homolog subfamily B member 12 | 3 | 261.454 | 399.379 | 1.528 |
| sp\|P47963\|RL13_MOUSE | Rpl13 | RL13_MOUSE 60S ribosomal protein L13 | 15 | 1598.54 | 2440.03 | 1.526 |
| sp\|Q60605\|MYL6_MOUSE | Myl6 | MYL6_MOUSE Myosin light polypeptide 6 | 1 | 65.7344 | 100.126 | 1.523 |
| tr\|F8VQ06\|F8VQ06_MOUSE | Ltbp3 | F8VQ06_MOUSE Latent-transforming growth factor | 32 | 3001.14 | 4566.79 | 1.522 |
| | | beta-binding protein 3 | | | | |
| sp\|P46935\|NEDD4_MOUSE | Nedd4 | NEDD4_MOUSE E3 ubiquitin-protein ligase NEDD4 | 4 | 431.767 | 656.56 | 1.521 |
| sp\|Q3TYS2\|CQ062_MOUSE | | CQ062_MOUSE Uncharacterized protein C17orf62 | 3 | 298.723 | 453.702 | 1.519 |
| | | homolog | | | | |
| sp\|Q8VDN2\|AT1A1_MOUSE | Atp1a1 | AT1A1_MOUSE Sodium/potassium-transporting | 26 | 2637.05 | 4002.79 | 1.518 |
| | | ATPase subunit alpha-1 | | | | |
| sp\|P19324\|SERPH_MOUSE | Serpinh1 | SERPH_MOUSE Seipin H1 | 31 | 5875.98 | 8915.68 | 1.517 |
| sp\|Q6VN19\|RBP10_MOUSE | Ranbp10 | RBP10_MOUSE Ran-binding protein 10 | 1 | 44.5569 | 67.4237 | 1.513 |
| sp\|O70475\|UGDH_MOUSE | Ugdh | UGDH_MOUSE UDP-glucose 6-dehydrogcnase | 26 | 3357.84 | 5079.04 | 1.513 |
| sp\|P29341\|PABP1_MOUSE | Pabpc1 | PABP1_MOUSE Polyadenylate-binding protein 1 | 1 | 34.6144 | 52.3049 | 1.511 |
| sp\|Q9DBG7\|SRPR_MOUSE | Srpr | SRPR_MOUSE Signal recognition particle receptor | 4 | 390.789 | 588.705 | 1.506 |
| | | subunit alpha | | | | |
| sp\|Q91XU3\|P142C_MOUSE | Pip4k2c | PI42C_MOUSE Phospliatidylinositol 5-phosphate | 5 | 828.515 | 1247.96 | 1.506 |
| | | 4-kinase type-2 gamma | | | | |
| sp\|Q9JKF1\|QGA1_MOUSE | Iqgap1 | IQGA1_MOUSE Ras GTPase-activating-like protein | 1 | 100.434 | 151.239 | 1.506 |
| | | IQGAP1 | | | | |
| sp\|Q8VCM8\|NCLN_MOUSE | Ncln | NCLN_MOUSE Nicalin | 4 | 283.571 | 426.853 | 1.505 |
| sp\|P83093\|STIM2_MOUSE | Stim2 | ST1M2_MOUSE Stromal interaction molecule 2 | 8 | 461.603 | 692.942 | 1.501 |
| sp\|Q810A7\|DDX42_MOUSE | Ddx42 | DDX42_MOUSE ATP-depcndent RNA helicase DDX42 | 22 | 1737.79 | 2604.89 | 1.499 |
| sp\|P61804\|DAD1_MOUSE | Dad1 | DAD1_MOUSE Dolichyl-diphosphooligosaccharide-- | 3 | 340.184 | 509.908 | 1.499 |
| | | protein glycosyltransferase subunit DAD1 | | | | |
| tr\|Q80ZX0\|Q80ZX0_MOUSE | Sec24b | Q80ZX0_MOUSE Protein Scc24b | 5 | 1026.19 | 1537.97 | 1.499 |
| sp\|P62962\|PROF1_MOUSE | Pfn1 | PROF1_MOUSE Profilin-1 | 1 | 84.6303 | 126.719 | 1.497 |
| sp\|Q3TMX7\|QSOX2_MOUSE | Qsox2 | QSOX2_MOUSE Sulfhydryl oxidase 2 | 13 | 2035.89 | 3047.82 | 1.497 |
| sp\|P62754\|RS6_MOUSE | Rps6 | RS6_MOUSE 40S ribosomal protein S6 | 14 | 2852.35 | 4259.6 | 1.493 |
| sp\|P14131\|RS16_MOUSE | Rps16 | RS16_MOUSE 40S ribosomal protein S16 | 5 | 842.123 | 1257.13 | 1.493 |
| sp\|P62267\|RS23_MOUSE | Rps23 | RS23_MOUSE 40S ribosomal protein S23 | 7 | 1479.69 | 2207.32 | 1.492 |
| sp\|Q91V98\|CD248_MOUSE | Cd248 | CD248_MOUSE Endosialin | 2 | 61.7627 | 92.0986 | 1.491 |
| sp\|P11499\|HS90B_MOUSE | Hsp90ab1 | HS90B_MOUSE Heat shock protein HSP 90-beta | 9 | 1432.38 | 2135.38 | 1.491 |
| sp\|Q9D6K9\|CERS5_MOUSE | Cers5 | CERS5_MOUSE Ceramide synthase 5 | 1 | 65.6112 | 97.7559 | 1.490 |
| sp\|Q920A5\|RISC_MOUSE | Scpep1 | RISC_MOUSE Retinoid-inducible serine carboxypeptidase | 3 | 563.401 | 839.335 | 1.490 |
| sp\|Q91WJ8\|FUBP1_MOUSE | Fubp1 | FUBP1_MOUSE Far upstream element-binding protein 1 | 1 | 75.3009 | 112.145 | 1.489 |
| tr\|D3Z2R5\|D3Z2R5_MOUSE | Sepn1 | D3Z2R5_MOUSE Protein Sepn1 | 1 | 30.2104 | 44.9805 | 1.489 |
| sp\|P62889\|RL30_MOUSE | Rpl30 | RL30_MOUSE 60S ribosomal protein L30 | 2 | 123.587 | 184.004 | 1.489 |
| sp\|Q64310\|SURF4_MOUSE | Surf4 | SURF4_MOUSE Surfeit locus protein 4 | 1 | 201.124 | 299.145 | 1.487 |
| sp\|P97464\|EXT1_MOUSE | Ext1 | EXT1_MOUSE Exostosin-1 | 2 | 180.01 | 266.484 | 1.480 |
| sp\|P61620\|S61A1_MOUSE | Sec61a1 | S61A1_MOUSE Protein transport protein Sec61 subunit | 8 | 956.643 | 1415.34 | 1.479 |
| | | alpha isoform 1 | | | | |
| sp\|Q9D9V3\|ECHD1_MOUSE | Echde1 | ECHD1_MOUSE Ethylmalonyl-CoA decarboxylase | 1 | 68.9995 | 101.901 | 1.477 |
| sp\|Q62186\|SSRD_MOUSE | Ssr4 | SSRD_MOUSE Translocon-associated protein subunit delta | 3 | 281.048 | 415.041 | 1.477 |
| sp\|Q9QZ03\|S39A1_MOUSE | Slc39a1 | S39A1_MOUSE Zinc transporter ZIP1 | 1 | 57.2988 | 84.5561 | 1.476 |
| sp\|O09005\|DEGSI_MOUSE | Degs1 | DEGS1_MOUSE Sphingolipid delta(4)-desaturase DES1 | 1 | 65.8841 | 97.0959 | 1.474 |
| sp\|P55096\|ABCD3_MOUSE | Abcd3 | ABCD3_MOUSE ATP-binding cassette sub-family | 8 | 1112.63 | 1639.09 | 1.473 |
| | | D member 3 | | | | |
| sp\|Q923A2\|SPDLY_MOUSE | Spdl1 | SPDLY_MOUSE Protein Spindly | 1 | 137.522 | 202.467 | 1.472 |
| sp\|P10853\|H2B1F_MOUSE | Hist1h2bf | H2B1F_MOUSE Histone H2B type 1-F/J/L | 13 | 2975.42 | 4378.88 | 1.472 |
| sp\|Q4VAA7\|SNX33_MOUSE | Snx33 | SNX33_MOUSE Sorting nexin-33 | 1 | 60.7951 | 89.3494 | 1.470 |
| sp\|P0C0S6\|H2AZ_MOUSE | H2afz | H2AZ_MOUSE Histone H2A.Z | 6 | 636.894 | 935.779 | 1.469 |
| sp\|Q9D7B7\|GPX8_MOUSE | Gpx8 | GPX8_MOUSE Probable glutathione peroxidase 8 | 3 | 353.45 | 518.569 | 1.467 |
| sp\|Q8BH60\|GOPC_MOUSE | Gopc | GOPC_MOUSE Golgi-associated PDZ and coiled-coil | 12 | 939.495 | 1377.94 | 1.467 |
| | | motif-containing protein | | | | |
| sp\|P84099\|RL19_MOUSE | Rpl19 | RL19_M0USE 60S ribosomal protein L19 | 8 | 864.525 | 1265.76 | 1.464 |
| sp\|Q99LF4\|RTCB_MOUSE | Rtcb | RTCB_MOUSE tRNA-splicing ligase RtcB homolog | 1 | 121.338 | 177.583 | 1.464 |
| sp\|Q9R0E1\|PLOD3_MOUSE | Plod3 | PLOD3_MOUSE Procollagen-lysine, 2-oxoglutarate | 10 | 987.129 | 1443.54 | 1.462 |
| | | 5-dioxygenase 3 | | | | |
| sp\|P26041\|MOES_MOUSE | Msn | MOES_MOUSE Moesin | 7 | 529.068 | 773.496 | 1.462 |
| sp\|Q8BUV3\|GEPH_MOUSE | Gphn | GEPH_MOUSE Gephyrin | 8 | 519.214 | 758.578 | 1.461 |
| sp\|Q6PEE2\|CTIF_MOUSE | Ctif | CTIF_MOUSE CBP80/20-dependent translation initiation | 3 | 146.769 | 213.518 | 1.455 |
| | | factor | | | | |
| sp\|Q03173\|ENAH_MOUSE | Enah | ENAH_MOUSE Protein enabled homolog | 1 | 278.846 | 405.582 | 1.455 |
| sp\|Q9DBC3\|CMTR1_MOUSE | Cmtr1 | CMTR1_MOUSE Cap-specific mRNA (nuclcoside-2′- | 1 | 56.1019 | 81.5529 | 1.454 |
| | | O-)-methyltransferase 1 | | | | |
| sp\|Q8K!L0\|CREB5_MOUSE | Creb5 | CREB5_MOUSE Cyclic AMP-responsive element- | 2 | 349.455 | 506.559 | 1.450 |
| | | binding protein 5 | | | | |
| sp\|Q8BMD8\|SCMCI_MOUSE | Slc25a24 | SCMC1_MOUSE Calcium-binding mitochondrial | 2 | 132.972 | 192.706 | 1.449 |
| | | carrier protein SCaMC-1 | | | | |
| sp\|P56395\|CYB5_MOUSE | Cyb5a | CYB5_MOUSE Cytochrome b5 | 3 | 345.867 | 501.171 | 1.449 |
| sp\|Q8VBZ3\|CLPT1_MOUSE | Clptm1 | CLPT1_MOUSE Cleft lip and palate transmembrane | 5 | 309.605 | 448.19 | 1.448 |
| | | protein 1 homolog | | | | |
| sp\|P11031\|TCP4_MOUSE | Sub1 | TCP4_MOUSE Activated RNA polymerase II | 1 | 78.0226 | 112.919 | 1.447 |
| | | transcriptional coactivator p15 | | | | |
| sp\|070309\|ITB5_MOUSE | Itgb5 | ITB5_MOUSE Integrin beta-5 | 2 | 148.382 | 214.546 | 1.446 |
| sp\|Q3U0Ml\|TPPC9_MOUSE | Trappc9 | TPPC9_MOUSE Trafficking protein particle complex | 1 | 103.129 | 148.945 | 1.444 |
| | | subunit 9 | | | | |
| sp\|P97402\|GCNT2_MOUSE | Gcnt2 | GCNT2_MOUSE N-acetyllactosaminide beta-1,6-N- | 1 | 248.97 | 359.52 | 1.444 |
| | | acetylglucosaminyl-transferase | | | | |
| sp\|Q9ZlZ0\|USO1_MOUSE | Uso1 | USO1_MOUSE General vesicular transport factor p15 | 23 | 1628 | 2350.61 | 1.444 |
| sp\|P08121\|CO3A1_MOUSE | Col3a1 | CO3A1_MOUSE Collagen alpha-1(III) chain | 26 | 2212.19 | 3192.29 | 1.443 |
| tr\|E9Q740\|E9Q740_MOUSE | Srp72 | E9Q740_MOUSE Signal recognition particle subunit SRP72 | 2 | 103.141 | 148.827 | 1.443 |
| sp\|Q9D880\|TIM50_MOUSE | Timm50 | TIM50_MOUSE Mitochondrial import inner membrane | 1 | 71.8627 | 103.519 | 1.441 |
| | | translocase subunit TIM50 | | | | |
| sp\|O70318\|E41L2_MOUSE | Epb41l2 | E41L2_MOUSE Band 4.1-like protein 2 | 7 | 499.305 | 718.6 | 1.439 |
| sp\|P62862\|RS30_MOUSE | Fau | RS30_MOUSE 40S ribosomal protein S30 | 1 | 128.306 | 184.638 | 1.439 |
| sp\|Q8BIH0\|SP130_MOUSE | Sap130 | SP130_MOUSE Histonc deacetylase complex subunit | 1 | 91.6314 | 131.725 | 1.438 |
| | | SAP 130 | | | | |
| sp\|Q9R001\|ATS5_MOUSE | Adamts5 | ATS5_MOUSE A disintegrin and metalloproteinase with | 7 | 351.988 | 505.889 | 1.437 |
| | | thrombospondin motifs 5 | | | | |
| sp\|Q61316\|HSP74_MOUSE | Hspa4 | HSP74_MOUSE Heat shock 70 kDa protein 4 | 3 | 218.303 | 313.403 | 1.436 |
| sp\|Q9Z175\|LOXL3_MOUSE | Loxl3 | LOXL3_MOUSE Lysyl oxidase homolog 3 | 74 | 10062.1 | 14442.6 | 1.435 |
| sp\|Q62407\|SPEG_MOUSE | Speg | SPEG_MOUSE Striated muscle-specific serine/threonine- | 1 | 186.347 | 266.982 | 1.433 |
| | | protein kinase | | | | |
| sp\|Q6GQT9\|NOMO1_MOUSE | Nomo1 | NOMO1_MOUSE Nodal modulator 1 | 17 | 2287.97 | 3273.88 | 1.431 |
| sp\|P97363\|SPTC2_MOUSE | Sptlc2 | SPTC2_MOUSE Serine palmitoyltransferase 2 | 1 | 36.2186 | 51.8072 | 1.430 |
| sp\|Q8BHT6\|B3GLT_MOUSE | B3galtl | B3GLT_MOUSE Beta-1,3-ghucosyltransferase | 9 | 2163.23 | 3090.27 | 1.429 |
| sp\|Q80T85\|DCAF5_MOUSE | Dcaf5 | DCAF5_MOUSE DDB1-and CUL4-associated factor 5 | 1 | 76.3429 | 108.945 | 1.427 |
| sp\|Q9CQW9\|IFM3_MOUSE | Ifitm3 | IFM3_MOUSE Interferon-induced transmembrane protein 3 | 6 | 772.295 | 1102 | 1.427 |
| sp\|Q9D0E1\|HNRPM_MOUSE | Hnrnpm | HNRPM_MOUSE Heterogeneous nuclear | 1 | 75.1792 | 106.802 | 1.421 |
| | | ribonucleoprotein M | | | | |
| sp\|O35904\|PK3CD_MOUSE | Pik3cd | PK3CD_MOUSE Phosphatidylinositol 4,5-bisphosphate | 1 | 279.936 | 397.601 | 1.420 |
| | | 3-kinase catalytic subunit delta isoform | | | | |
| sp\|P70168\|IMB1_MOUSE | Kpnb1 | IMB1_MOUSE Importin subunit beta-1 | 2 | 98.4947 | 139.88 | 1.420 |
| sp\|Q8R2Y8\|PTH2_MOUSE | Ptrh2 | PTH2_MOUSE Peptidyl-tRNA hydrolase 2, mitochondrial | 1 | 89.2262 | 126.64 | 1.419 |
| sp\|Q8VCF0\|MAVS_MOUSE | Mavs | MAVS_MOUSE Mitochondrial antiviral-signaling protein | 1 | 71.4359 | 101.299 | 1.418 |
| sp\|P33434\|MMP2_MOUSE | Mmp2 | MMP2_MOUSE 72 kDa type IV collagenase | 1 | 61.7476 | 87.542 | 1.418 |
| tr\|A1L341\|A1L341_MOUSE | Dyrk1a | A1L341_MOUSE Dual-specificity tyrosine- | 5 | 494.434 | 700.874 | 1.418 |
| | | phosphorylation-regulated kinase 1A | | | | |
| sp\|Q9JIY2\|HAKAI_MOUSE | Cbll1 | HAKAI_MOUSE E3 ubiquitin-protein ligase Hakai | 2 | 245.373 | 347.805 | 1.417 |
| sp\|Q8CJ69\|BMPER_MOUSE | Bmper | BMPER_MOUSE BMP-binding endothelial regulator | 1 | 57.1406 | 80.9236 | 1.416 |
| | | protein | | | | |
| sp\|Q8R3Q0\|SARAF_MOUSE | Tme66 | SARAF_MOUSE Store-operated calcium entry- | 1 | 70.0032 | 99.0022 | 1.414 |
| | | associated regulatory factor | | | | |
| sp\|P15864\|H12_MOUSE | Hist1h1c | H12_MOUSE Histone H1.2 | 3 | 506.263 | 715.608 | 1.414 |
| sp\|Q9Z0R9\|FADS2_MOUSE | Fads2 | FADS2_MOUSE Fatty acid desaturase 2 | 4 | 970.687 | 1371.96 | 1.413 |
| sp\|Q8BG51-3\|MIRO1_MOUSE | Rhot1 | MIRO1_MOUSE Isoform 3 of Mitochondrial Rho GTPase 1 | 9 | 817.439 | 1155.13 | 1.413 |
| sp\|Q9CY27\|TECR_MOUSE | Tecr | TECR__MOUSE Very-long-chain enoyl-CoA reductase | 12 | 1333.34 | 1882.62 | 1.412 |
| sp\|Q8BZ20\|PAR12_MOUSE | Parp12 | PAR12_MOUSE Poly [ADP-ribose] polymerase 12 | 3 | 221.145 | 312.069 | 1.411 |
| sp\|Q9D023\|MPC2_MOUSE | Mpc2 | MPC2_MOUSE Mitochondrial pyruvate carrier 2 | 2 | 274.697 | 387.158 | 1.409 |
| sp\|Q9R233\|TPSN_MOUSE | Tapbp | TPSN_MOUSE Tapasin | 12 | 825.18 | 1162.87 | 1.409 |
| sp\|Q9CR46\|SKA2_MOUSE | Ska2 | SKA2_MOUSE Spindle and kinetochore-associated | 1 | 53.8084 | 75.8284 | 1.409 |
| | | protein 2 | | | | |
| sp\|Q8BRF7\|SCFD1_MOUSE | Scfd1 | SCFD1_MOUSE Sec1 family domain-containing protein 1 | 2 | 124.108 | 174.891 | 1.409 |
| sp\|P45878\|FKBP2_MOUSE | Fkbp2 | FKBP2_MOUSE Peptidyl-prolyl cis-trans isomerase | 1 | 76.2142 | 107.381 | 1.409 |
| | | FKBP2 | | | | |
| sp\|Q921Q3\|ALG1_MOUSE | Alg1 | ALG1_MOUSE Chitobiosyldiphosphodolichol beta- | 1 | 41.9345 | 59.0265 | 1.408 |
| | | mannosyltransferase | | | | |
| sp\|Q3V1T4\|P3H1_MOUSE | Lepre1 | P3H1_MOUSE Prolyl 3-hydroxylase 1 | 6 | 482.481 | 678.686 | 1.407 |
| sp\|Q60738\|ZNT1_MOUSE | Slc30a1 | ZNT1_MOUSE Zinc transporter 1 | 25 | 2103.14 | 2956.37 | 1.406 |
| sp\|Q6NV83\|SR140_MOUSE | U2surp | SR140_MOUSE U2 snRNP-associated SURP motif- | 4 | 301.917 | 424.212 | 1.405 |
| | | containing protein | | | | |
| tr\|G3UWV6\|G3UWV6_MOUSE | Agpat1 | G3UWV6_MOUSE 1-acyl-sn-glycerol-3-phosphate | 1 | 80.3406 | 112.598 | 1.402 |
| | | acyltransferase alpha (Fragment) | | | | |
| sp\|Q62130\|PTN14_MOUSE | Ptpn14 | PIN14_MOUSE Tyrosine-protein phosphatase non- | 1 | 26.47 | 37.0683 | 1.400 |
| | | receptor type 14 | | | | |
| sp\|Q99K01-2\|PDXD1_MOUSE | Pdxde1 | PDXD1_MOUSE Isoform 2 of Pyrtdoxal-dependent | 1 | 39.8486 | 55.7489 | 1.399 |
| | | decarboxylase domain-containing protein 1 | | | | |
| sp\|P24270\|CATA_MOUSE | Cat | CATA_MOUSE Catalase | 1 | 110.097 | 153.982 | 1.399 |
| sp\|P61205\|ARF3_MOUSE | Arf3 | ARF3_MOUSE ADP-ribosylation factor 3 | 13 | 2227.48 | 3113.18 | 1.398 |
| sp\|P70428\|EXT2_MOUSE | Ext2 | EXT2_MOUSE Exostosin-2 | 8 | 726.598 | 1014.78 | 1.397 |
| sp\|Q8BLN5\|ERG7_MOUSE | Lss | ERG7_MOUSE Lanosterol synthase | 17 | 1884.28 | 2631.22 | 1.396 |
| sp\|Q922Q8\|LRC59_MOUSE | Lrrc59 | LRC59_MOUSE Leucine-rich repeat-containing | 7 | 524.738 | 731.871 | 1.395 |
| | | protein 59 | | | | |
| sp\|Q8CFXl\|G6PE_MOUSE | H6pd | G6PE_MOUSE GDH/6PGL endoplasmic bifunctional | 21 | 3322.43 | 4628.93 | 1.393 |
| | | protein | | | | |
| tr\|G5E829\|G5E829_MOUSE | Atp2b1 | G5E829_MOUSE MCG13663, isoform CRA_a | 3 | 389.435 | 542.481 | 1.393 |
| sp\|P62305\|RUXE_MOUSE | Snrpe | RUXE_MOUSE Small nuclear ribonucleoprotein E | 1 | 370.129 | 514.624 | 1.390 |
| sp\|P62869\|ELOB_MOUSE | Tceb2 | ELOB_MOUSE Transcription elongation factor B | 3 | 250.261 | 347.851 | 1.390 |
| | | polypeptide 2 | | | | |
| sp\|P62342\|SELT_MOUSE | Selt | SELT_MOUSE Selenoprotein T | 1 | 119.152 | 165.583 | 1.390 |
| sp\|P51881\|ADT2_MOUSE | Slc25a5 | ADT2__MOUSE ADP/ATP translocase 2 | 7 | 1104.24 | 1534.48 | 1.390 |
| sp\|Q99KV1\|DJB11_MOUSE | Duajb11 | DJB11_MOUSE DnaJ homolog subfamily B member 11 | 24 | 3441.89 | 4775.4 | 1.387 |
| sp\|Q3TDQ1\|STT3B_MOUSE | Stt3b | STT3B_MOUSE Dolichyl-diphosphooligosaccharide- | 24 | 5880.17 | 8155.08 | 1.387 |
| | | protein glycosyltransferase subunit STT3B | | | | |
| sp\|Q3UN04\|UBP30_MOUSE | Usp30 | UBP30_MOUSE Ubiquitin carboxyl-terminal hydrolase 30 | 7 | 368.236 | 510.101 | 1.385 |
| sp\|Q31125\|S39A7_MOUSE | Slc39a7 | S39A7_MOUSE Zinc transporter SLC39A7 | 16 | 2935.64 | 4062.16 | 1.384 |
| sp\|Q9CQR2\|RS21_MOUSE | Rps21 | RS21_MOUSE 40S ribosomal protein S21 | 1 | 60.1972 | 83.2587 | 1.383 |
| sp\|Q64282\|IFIT1_MOUSE | Ifit1 | IFIT1_MOUSE Interferon-induced protein with | 2 | 113.885 | 157.51 | 1.383 |
| | | telratricopeptide repeats 1 | | | | |
| sp\|O54774\|AP3D1_MOUSE | Ap3d1 | AP3D1_MOUSE AP-3 complex subunit delta-1 | 1 | 40.3052 | 55.6511 | 1.381 |
| sp\|Q5SF07\|IF2B2_MOUSE | Igf2bp2 | IF2B2_MOUSE Insulin-like growth factor 2 mRNA- | 18 | 2084.98 | 2877.52 | 1.380 |
| | | binding protein 2 | | | | |
| sp\|Q9EQH2\|ERAPI_MOUSE | Erap1 | ERAP1_MOUSE Endoplasmic reticulum aminopeptidase 1 | 19 | 1555.91 | 2145.91 | 1.379 |
| sp\|Q9D0R8\|LSM12_MOUSE | Lsm12 | LSM12_MOUSE Protein LSM12 homolog | 1 | 156.058 | 215.221 | 1.379 |
| sp\|O35379\|MRP1_MOUSE | Abcc1 | MRP1_MOUSE Multidrug resistance-associated protein 1 | 5 | 293.471 | 404.591 | 1.379 |
| sp\|A3KGW5\|GT253_MOUSE | Cercam | GT253_MOUSE Probable inactive glycosyltransferase | 14 | 2673.57 | 3685.44 | 1.378 |
| | | 25 family member 3 | | | | |
| sp\|Q9WV92\|E41L3_MOUSE | Epb41l3 | E41L3_MOUSE Band 4.1-like protein 3 | 9 | 583.457 | 803.101 | 1.376 |
| sp\|Q8BYU6\|TOIP2_MOUSE | Tor1aip2 | TOIP2_MOUSE Torsin-1A-interacting protein 2 | 17 | 1622.98 | 2232.22 | 1.375 |
| sp\|Q921J2\|RHEB_MOUSE | Rheb | RHEB_MOUSE GTP-binding protein Rheb | 1 | 24.294 | 33.4023 | 1.375 |
| sp\|Q9JJD0\|THA11_MOUSE | Thap11 | THA11_MOUSE THAP domain-containing protein 11 | 1 | 171.224 | 235.401 | 1.375 |
| sp\|P22892\|AP1G1_MOUSE | Ap1g1 | AP1G1_MOUSE AP-1 complex subunit gamma-1 | 1 | 54.8356 | 75.3114 | 1.373 |
| sp\|P15379\|CD44_MOUSE | Cd44 | CD44_MOUSE CD44 antigen | 4 | 162.462 | 222.91 | 1.372 |
| sp\|Q61335\|BAP31_MOUSE | Bcap31 | BAP31_MOUSE B-cell receptor-associated protein 31 | 4 | 546.984 | 749.532 | 1.370 |
| sp\|Q61508\|ECM1_MOUSE | Ecm1 | ECM1_MOUSE Extracellular matrix protein 1 | 23 | 2514.52 | 3443.01 | 1.369 |
| sp\|A2AJ15\|MA1B1_MOUSE | Man1b1 | MA1B1_MOUSE Endoplasmic reticulum mannosyl- | 3 | 142.337 | 194.787 | 1.368 |
| | | oligosaccharide 1,2-alpha-mannosidase | | | | |
| sp\|Q04857\|CO6A1_MOUSE | Col6a1 | CO6A1_MOUSE Collagen alpha-1(VI) chain | 10 | 1201.87 | 1644.51 | 1.368 |
| sp\|Q9D1M7\|FKB11_MOUSE | Fkbp11 | FKB11_MOUSE Peptidyl-prolyl cis-trans isomerase | 2 | 350.175 | 478.449 | 1.366 |
| | | FKBP11 | | | | |
| sp\|Q8BU14\|SEC62_MOUSE | Sec62 | SEC62_MOUSE Translocation protein SEC62 | 2 | 255.38 | 348.831 | 1.366 |
| sp\|P14148\|RL7_MOUSE | Rpl7 | RL7_MOUSE 60S ribosomal protein L7 | 5 | 340.455 | 464.953 | 1.366 |
| sp\|P15306\|TRBM_MOUSE | Thbd | TRBM_MOUSE Thrombomodulin | 3 | 294.175 | 401.338 | 1.364 |
| sp\|Q8VEK2\|RHBD2_MOUSE | Rhbdd2 | RHBD2_MOUSE Rhomboid domain-containing protein 2 | 1 | 34.5852 | 47.1663 | 1.364 |
| sp\|P70698\|PYRG1_MOUSE | Ctps1 | PYRG1_MOUSE CTP synthase 1 | 1 | 44.513 | 60.7046 | 1.364 |
| sp\|Q9CPZ6\|ORML3_MOUSE | Ormdl3 | ORML3_MOUSE ORM1-like protein 3 | 1 | 114.329 | 155.847 | 1.363 |
| sp\|Q8VBT0\|TMX1_MOUSE | Tmx1 | TMX1_MOUSE Thioredoxin-related transmembrane | 4 | 368.095 | 501.76 | 1.363 |
| | | protein 1 | | | | |
| sp\|P63101\|1433Z_MOUSE | Ywhaz | 1433Z_MOUSE 14-3-3 protein zeta/delta | 2 | 92.9474 | 126.674 | 1.363 |
| sp\|P11928\|OAS1A_MOUSE | Oas1a | OAS1A_MOUSE 2′-5′-oligoadenylate synthase 1A | 2 | 151.26 | 206.021 | 1.362 |
| tr\|E9Q616\|E9Q616_MOUSE | Ahnak | E9Q616_MOUSE Protein Ahnak | 4 | 764.699 | 1041.5 | 1.362 |
| sp\|P39098\|MA1A2_MOUSE | Man1a2 | MA1A2_MOUSE Mannosyl-oligosaccharide 1,2-alpha- | 3 | 342.297 | 465.723 | 1.361 |
| | | mannosidase IB | | | | |
| sp\|Q3TAS6\|EMC10_MOUSE | Eme10 | EMC10_MOUSE ER membrane protein complex subunit 10 | 2 | 92.6145 | 125.999 | 1.360 |
| sp\|Q9D1G1\|RAB1B_MOUSE | Rab1b | RAB1B_MOUSE Ras-related protein Rab-1B | 2 | 437.333 | 594.646 | 1.360 |
| sp\|P62340\|TBPL1_MOUSE | Tbpl1 | TBPL1_MOUSE TATA box-binding protein-like protein 1 | 1 | 37.0008 | 50.2561 | 1.358 |
| tr\|Q9DCE9\|Q9DCE9_MOUSE | IgtP | Q9DCE9_MOUSE Protein Igtp | 11 | 876.376 | 1190.17 | 1.358 |
| sp\|Q7TQH0-2\|ATX2L_MOUSE | Atxn21 | ATX2L_MOUSE Isofonn 2 of Ataxin-2-like protein | 16 | 1676.76 | 2275.59 | 1.357 |
| sp\|Q9CR67\|TMM33_MOUSE | Tmem33 | TMM33_MOUSE Transmembrane protein 33 | 4 | 520.374 | 705.23 | 1.355 |
| sp\|Q14C59\|TM11L_MOUSE | Tmprss | TM11L_MOUSE Transmembrane protease serine | 1 | 274.892 | 372.511 | 1.355 |
| | 11bnl | 11B-like protein | | | | |
| sp\|P31750\|AKT1_MOUSE | Akt1 | AKT1__MOUSE RAC-alpha serine/threonine- | 2 | 323.834 | 438.716 | 1.355 |
| | | protein kinase | | | | |
| sp\|Q8CFG9\|C1RB_MOUSE | C1rb | C1RB_MOUSE Complement C1r-B subcomponent | 1 | 40.3706 | 54.6396 | 1.353 |
| sp\|Q91W97\|HKDC1_MOUSE | Hkde1 | HKDC1_MOUSE Putative hexokinase HKDC1 | 1 | 137.321 | 185.83 | 1.353 |
| sp\|Q01853\|TERA_MOUSE | Vcp | TERA_MOUSE Transitional endoplasmic reticulum ATPase | 30 | 4603.83 | 6227.3 | 1.353 |
| sp\|Q8VCBl\|NDC1_MOUSE | Nde1 | NDC1_MOUSE Nucleoporin NDC1 | 4 | 182.905 | 246.746 | 1.349 |
| sp\|Q62371\|DDR2_MOUSE | Ddr2 | DDR2_MOUSE Discoidin domain-containing receptor 2 | 1 | 33.0832 | 44.6112 | 1.348 |
| sp\|Q9Z2V5\|HDAC6_MOUSE | Hdac6 | HDAC6__MOUSE Histone deacetylase 6 | 4 | 522.556 | 704.462 | 1.348 |
| sp\|P47740\|AL3A2_MOUSE | Aldh3a2 | AL3A2_MOUSE Fatty aldehyde dehydrogenase | 1 | 43.2284 | 58.2591 | 1.348 |
| sp\|Q923Q2\|STA13_MOUSE | Stard13 | STA13_MOUSE StAR-related lipid transfer protein 13 | 1 | 62.5142 | 84.1833 | 1.347 |
| tr\|S4R2K0\|S4R2K0_MOUSE | Pdf | S4R2K0_MOUSE Protein Pdf | 1 | 77.0682 | 103.779 | 1.347 |
| sp\|Q8R4U7\|LUZP1_MOUSE | Luzp1 | LUZP1_MOUSE Leucine zipper protein 1 | 1 | 207.966 | 279.987 | 1.346 |
| sp\|Q9D6J5\|NDUB8_MOUSE | Ndufb8 | NDUB8_MOUSE NADH dehydrogenase [ubiquinone] | 20 | 2366.74 | 3185.42 | 1.346 |
| | | 1 beta subcomplex subunit 8, mitochondrial | | | | |
| sp\|Q9EPU4\|CPSF1_MOUSE | Cpsf1 | CPSF1_MOUSE Cleavage and polyadenylation specificity | 1 | 69.2682 | 93.2217 | 1.346 |
| | | factor subunit 1 | | | | |
| sp\|P10630\|IF4A2_MOUSE | Eif4a2 | IF4A2_MOUSE Eukaryotic initiation factor 4A-II | 1 | 164.461 | 221.14 | 1.345 |
| sp\|Q9CRC0\|VKOR1_MOUSE | Vkorc1 | VKOR1_MOUSE Vitamin K epoxide reductase complex | 1 | 113.084 | 152.033 | 1.344 |
| | | subunit 1 | | | | |
| sp\|P46978\|STT3A_MOUSE | Stt3a | STT3A_MOUSE Dolichyl-diphosphooligosaccharide-- | 25 | 3204.91 | 4308.57 | 1.344 |
| | | protein glycosyltransferase subunit STT3A | | | | |
| sp\|O35126\|ATN1_MOUSE | Atn1 | ATN1_MOUSE Atrophin-1 | 5 | 889.577 | 1195.65 | 1.344 |
| sp\|Q8BH64\|EHD2_MOUSE | Ehd2 | EHD2_MOUSE EH domain-containing protein 2 | 5 | 319.824 | 429.796 | 1.344 |
| sp\|O88561\|S27A3_MOUSE | Slc27a3 | S27A3_MOUSE Long-chain fatty acid transport protein 3 | 3 | 262.242 | 352.411 | 1.344 |
| sp\|P58742\|AAAS_MOUSE | Aaas | AAAS_MOUSE Aladin | 2 | 123.071 | 165.349 | 1.344 |
| sp\|Q3V3Rl\|C1TM_MOUSE | Mthfd1l | C1TM_MOUSEMonofunctional C1-tetrahydrofolate | 23 | 2212.07 | 2970.4 | 1.343 |
| | | synthase, mitochondrial | | | | |
| sp\|P61979\|HNRPK_MOUSE | Hnrnpk | HNRPK_MOUSE Heterogeneous nuclear | 1 | 82.8958 | 111.167 | 1.341 |
| | | ribonucleoprotein K | | | | |
| sp\|P46460\|NSF_MOUSE | Nsf | NSF_MOUSE Vesicle-fusing ATPase | 8 | 897.47 | 1202.61 | 1.340 |
| sp\|Q9R0Q3\|TMED2_MOUSE | Tmed2 | TMED2_MOUSE Transmembrane emp24 domain- | 8 | 962.403 | 1288.63 | 1.339 |
| | | containing protein 2 | | | | |
| sp\|Q8C176\|TAF2_MOUSE | Taf2 | TAF2_MOUSE Transcription initiation factor TFIID | 2 | 112.656 | 150.263 | 1.334 |
| | | subunit 2 | | | | |
| sp\|Q62392\|PHLA1_MOUSE | Phlda1 | PHLA1_MOUSE Pleckstrin homology-like domain family | 2 | 207.706 | 276.913 | 1.333 |
| | | A member 1 | | | | |
| sp\|Q9JI75\|NQO2_MOUSE | Nqo2 | NQO2_MOUSE Ribosyldihydronicotinamide | 1 | 61.4262 | 81.8721 | 1.333 |
| | | dehydrogenase [quinone] | | | | |
| sp\|Q9QXZ0\|MACF1_MOUSE | Macf1 | MACF1_MOUSE Microtubule-actin cross-linking factor 1 | 1 | 99.9363 | 132.923 | 1.330 |
| sp\|035737\|HNRHI_MOUSE | Hnrnph1 | HNRH1_MOUSE Heterogeneous nuclear | 1 | 357.069 | 474.654 | 1.329 |
| | | ribonucleoprotein H | | | | |
| sp\|P16858\|G3P_MOUSE | Gapdh | G3P_MOUSE Glyceraldehyde-3-phosphate dehydrogenase | 7 | 934.749 | 1242.46 | 1.329 |
| sp\|Q64339\|ISG15_MOUSE | Isg15 | ISG15_MOUSE Ubiquitin-like protein ISG15 | 9 | 1297.39 | 1724.38 | 1.329 |
| sp\|P37040\|NCPR_MOUSE | Por | NCPR_MOUSE NADPH--cytochrome P450 reductase | 16 | 2320.79 | 3084.43 | 1.329 |
| sp\|Q3UN02\|LCLT1_MOUSE | Lclat1 | LCLT1_MOUSE Lysocardiolipin acyltransferase 1 | 2 | 205.69 | 273.359 | 1.329 |
| sp\|B2RXS4\|PLXB2_MOUSE | Plxnb2 | PLXB2_MOUSE Plexin-B2 | 2 | 78.3367 | 104.104 | 1.329 |
| sp\|Q920L1\|FADSI_MOUSE | Fads1 | FADS1_MOUSE Fatty acid desaturase 1 | 32 | 5574.38 | 7403.93 | 1.328 |
| sp\|P35822\|PTPRK_MOUSE | Ptprk | PTPRK_MOUSE Receptor-type tyrosine-protein | 1 | 89.1689 | 118.357 | 1.327 |
| | | phosphatase kappa | | | | |
| sp\|Q9WVL2\|STAT2_MOUSE | Stat2 | STAT2_MOUSE Signal transducer and activator of | 3 | 185.238 | 245.763 | 1.327 |
| | | transcription 2 | | | | |
| sp\|P58022\|LOXL2_MOUSE | Loxl2 | LOXL2_MOUSE Lysyl oxidase homolog 2 | 8 | 1099.55 | 1457.95 | 1.326 |
| sp\|Q3UMR5\|MCU_MOUSE | Mcu | MCU_MOUSE Calcium uniporter protein, mitochondrial | 6 | 851.15 | 1128.28 | 1.326 |
| tr\|E9PYB0\|E9PYB0_MOUSE | Ahnak2 | E9PYB0_MOUSE Protein Ahnak2 (Fragment) | 1 | 211.535 | 280.271 | 1.325 |
| sp\|Q9QY24\|ZBP1_MOUSE | Zbp1 | ZBP1_MOUSE Z-DNA-binding protein 1 | 1 | 95.6922 | 126.504 | 1.322 |
| sp\|Q80ZM8\|CRLS1_MOUSE | Crls1 | CRLS1_MOUSE Cardiolipin synthase | 1 | 31.1035 | 41.1133 | 1.322 |
| sp\|Q8C0L0\|TMX4_MOUSE | Tmx4 | TMX4_MOUSE Thioredoxin-related transmembrane | 1 | 45.6548 | 60.2878 | 1.321 |
| | | protein 4 | | | | |
| sp\|Q9JMH3\|NEUR2_MOUSE | Neu2 | NEUR2_MOUSE Sialidase-2 | 1 | 62.5922 | 82.6423 | 1.320 |
| sp\|P68040\|GBLP_MOUSE | Gnb2l1 | GBLP_MOUSE Guanine nucleotide-binding protein | 14 | 1741.41 | 2298.93 | 1.320 |
| | | subunit beta-2-like 1 | | | | |
| sp\|P20029\|GRP78_MOUSE | Hspa5 | GRP78_MOUSE 78 kDa glucose-regulated protein | 126 | 20609.5 | 27183 | 1.319 |
| sp\|Q8BFZ9\|ERLN2_MOUSE | Erlin2 | ERLN2_MOUSE Erlin-2 | 1 | 29.8083 | 39.3096 | 1.319 |
| sp\|Q8BMP6\|GCP60_MOUSE | Acbd3 | GCP60_MOUSE Golgi resident protein GCP60 | 2 | 245.296 | 323.327 | 1.318 |
| sp\|Q8R0X7\|SGPL1_MOUSE | Sgpl1 | SGPL1_MOUSE Sphingosine-1-phosphate lyase 1 | 1 | 112.302 | 147.951 | 1.317 |
| sp\|P68372\|TBB4B_MOUSE | Tubb4b | TBB4B_MOUSE Tubulin beta-4B chain | 8 | 554.775 | 730.682 | 1.317 |
| sp\|Q9EPL4\|METL9_MOUSE | Mettl9 | METL9_MOUSE Methyltransferase-like protein 9 | 4 | 627.306 | 825.945 | 1.317 |
| sp\|Q9CZD3\|SYG_MOUSE | Gars | SYG_MOUSE Glycine--tRNA ligase | 2 | 150.657 | 198.329 | 1.316 |
| sp\|Q6ZWV7\|RL35_MOUSE | Rpl35 | RL35_MOUSE 60S ribosomal protein L35 | 2 | 454.467 | 597.89 | 1.316 |
| sp\|Q91X88\|PMGT1_MOUSE | Pomgnt1 | PMGT1_MOUSE Protein O-linked-mannose beta- | 3 | 311.386 | 409.491 | 1.315 |
| | | 1,2-N-acetylglucosaminyltransferase 1 | | | | |
| sp\|Q810U5\|CCD50_MOUSE | Ccdc50 | CCD50_MOUSE Coilcd-coil domain-containing | 3 | 296.401 | 389.443 | 1.314 |
| | | protein 50 | | | | |
| sp\|Q02788\|CO6A2_MOUSE | Col6a2 | C06A2_MOUSE Collagen alpha-2(VI) chain | 6 | 690.522 | 907.264 | 1.314 |
| sp\|P02769\|ALBU_BOVIN_ | ALB | ALBU_BOVIN_contaminant Serum albumin | 76 | 13308.2 | 17481.2 | 1.314 |
| contaminant | | | | | | |
| sp\|Q8BU33\|ILVBL_MOUSE | Ilybl | ILVBL_MOUSE Acetolactate synthase-like protein | 4 | 218.39 | 286.584 | 1.312 |
| tr\|Q6ZWZ6\|Q6ZWZ6_MOUSE | Rps12 | Q6ZWZ6_MOUSE 40S ribosomal protein S12 | 3 | 465.14 | 610.246 | 1.312 |
| sp\|Q9D967\|MGDP1_MOUSE | Mdp1 | MGDP1_MOUSE Magnesium-dependent phosphatase 1 | 5 | 938.987 | 1231.22 | 1.311 |
| sp\|Q9Z210\|PX11B_MOUSE | Pex11b | PX11B_MOUSE Peroxisomal membrane protein 11B | 1 | 47.4335 | 62.1948 | 1.311 |
| sp\|Q6P4S6\|SIK3_MOUSE | Sik3 | SIK3_MOUSE Serine/threonine-protein kinase SIK3 | 3 | 185.721 | 243.484 | 1.311 |
| sp\|Q9ESU6\|BRD4_MOUSE | Brd4 | BRD4_MOUSE Bromodomain-containing protein 4 | 5 | 368.514 | 483.057 | 1.311 |
| sp\|Q8K2Z2\|PRP39_MOUSE | Prpf39 | PRP39_MOUSE Pre-mRNA-proccssing factor 39 | 1 | 87.6357 | 114.791 | 1.310 |
| sp\|P80314\|TCPB_MOUSE | Cct2 | TCPB_MOUSE T-complex protein 1 subunit beta | 1 | 117.455 | 153.832 | 1.310 |
| sp\|P06151\|LDHA_MOUSE | Ldha | LDHA_MOUSE L-lactate dehydrogenase A chain | 35 | 3790.95 | 4964.73 | 1.310 |
| sp\|Q9D6Y9\|GLGB_MOUSE | Gbe1 | GLGB_MOUSE 1,4-alpha-glucan-branching enzyme | 1 | 45.3509 | 59.3703 | 1.309 |
| tr\|A1L3P4\|A1L3P4_MOUSE | Slc9a6 | A1L3P4_MOUSE Sodium/hydrogen exchanger | 1 | 78.1096 | 102.205 | 1.308 |
| sp\|O35160\|GOSR2_MOUSE | Gosr2 | GOSR2_MOUSE Golgi SNAP receptor complex member 2 | 2 | 382.684 | 500.712 | 1.308 |
| sp\|Q9QY16\|DNJB9_MOUSE | Duajb9 | DNJB9_MOUSE DnaJ homolog subfamily B member 9 | 2 | 213.634 | 279.515 | 1.308 |
| tr\|F8VQC9\|F8VQC9_MOUSE | Slc4a7 | F8VQC9_MOUSE NBCn1-G | 20 | 2605.81 | 3406.24 | 1.307 |
| sp\|P62320\|SMD3_MOUSE | Snrpd3 | SMD3_MOUSE Small nuclear ribonuclcoprotein Sm D3 | 3 | 281.088 | 366.945 | 1.305 |
| sp\|Q91V01\|MBOA5_MOUSE | Lpcat3 | MBOA5_MOUSE Lysophospholipid acyltransferase 5 | 1 | 26.0371 | 33.9856 | 1.305 |
| sp\|Q62177\|SEM3B_MOUSE | Sema3b | SEM3B_MOUSE Semaphorin-3B | 7 | 641.568 | 836.991 | 1.305 |
| sp\|P52963\|E41LA_MOUSE | Epb41l4a | E41LA_MOUSE Band 4.1-like protein 4A | 1 | 35.5374 | 46.3374 | 1.304 |
| tr\|Q8K094\|Q8K094_MOUSE | Pvr | Q8K094_MOUSE Poliovirus receptor | 1 | 29.9661 | 39.0597 | 1.303 |
| sp\|Q99KC8\|VMASA_MOUSE | Vwa5a | VMA5A_MOUSE von Willebrand factor A domain- | 1 | 75.8405 | 98.7964 | 1.303 |
| | | containing protein 5A | | | | |
| sp\|Q99JY8\|LPP3_MOUSE | Ppap2b | LPP3_MOUSE Lipid phosphate phosphohydrolase 3 | 9 | 1157.69 | 1507.96 | 1.303 |
| sp\|Q8BTM8\|FLNA_MOUSE | Flna | FLNA_MOUSE Filamin-A | 10 | 751.716 | 977.941 | 1.301 |
| sp\|Q8BMG7\|RBGPR_MOUSE | Rab3gap2 | RBGPR_MOUSE Rab3 GTPase-activating protein | 1 | 44.2096 | 57.4679 | 1.300 |
| | | non-catalytic subunit | | | | |
| sp\|Q99K48\|NONO_MOUSE | Nono | NONO_MOUSE Non-POU domain-containing | 7 | 520.261 | 675.874 | 1.299 |
| | | octamer-binding protein | | | | |
| sp\|O70579\|PM34_MOUSE | Slc25a17 | PM34_MOUSE Peroxisomal membrane protein PMP34 | 1 | 177.441 | 230.468 | 1.299 |
| tr\|B9EJ80\|B9EJ80_MOUSE | Pdzd8 | B9EJ80_MOUSE PDZ domain containing 8 | 1 | 52.739 | 68.4242 | 1.297 |
| sp\|P52875\|TM165_MOUSE | Tmem165 | TM165_MOUSE Transmembrane protein 165 | 5 | 275.016 | 356.667 | 1.297 |
| sp\|P16381\|DDX3L_MOUSE | D1Pas1 | DDX3L_MOUSE Putative ATP-dependent RNA | 2 | 149.873 | 194.144 | 1.295 |
| | | helicase Pl10 | | | | |
| sp\|Q8K183\|PDXK_MOUSE | Pdxk | PDXK_MOUSE Pyridoxal kinase | 2 | 247.291 | 320.333 | 1.295 |
| sp\|Q8CBA2\|SLFN5_MOUSE | Slfn5 | SLFN5_MOUSE Schlafen family member 5 | 1 | 212.344 | 275.017 | 1.295 |
| sp\|O89001\|CBPD_MOUSE | Cpd | CBPD_MOUSE Caiboxypeptidase D | 5 | 693.984 | 898.447 | 1.295 |
| sp\|O09167\|RL21_MOUSE | Rpl21 | RL21_MOUSE 60S ribosomal protein L21 | 3 | 547.769 | 709.058 | 1.294 |
| sp\|Q9CQG6\|TM147_MOUSE | Tmem147 | TM147_MOUSE Transmembrane protein 147 | 1 | 52.1134 | 67.4014 | 1.293 |
| sp\|Q63961\|EGLN_MOUSE | Eng | EGLN_MOUSE Endoglin | 1 | 98.4879 | 127.324 | 1.293 |
| sp\|Q924C6\|LOXL4_MOUSE | Loxl4 | LOXL4_MOUSE Lysyl oxidase homolog 4 | 6 | 486.195 | 628.437 | 1.293 |
| sp\|Q62523\|ZYX_MOUSE | Zyx | ZYX_MOUSE Zyxin | 1 | 36.9278 | 47.6926 | 1.292 |
| sp\|Q9D6K8\|FUND2_MOUSE | Fundc2 | FUND2_MOUSE FUN14 domain-containing protein 2 | 1 | 78.5813 | 101.366 | 1.290 |
| tr\|Q3U3W2\|Q3U3W2_MOUSE | Tmem181a | Q3U3W2_MOUSE Protein Tmem181a | 1 | 34.7979 | 44.8868 | 1.290 |
| sp\|Q9CQU0\|TXD12_MOUSE | Txnde12 | TXD12_MOUSE Thioredoxin domain-containing | 5 | 324.254 | 418.063 | 1.289 |
| | | protein 12 | | | | |
| sp\|P49718\|MCM5_MOUSE | Mcm5 | MCM5_MOUSE DNA replication licensing factor MCM5 | 3 | 418.392 | 539.333 | 1.289 |
| sp\|Q91Z96\|BMP2K_MOUSE | Bmp2k | BMP2K_MOUSE BMP-2-inducible protein kinase | 1 | 67.5706 | 87.0983 | 1.289 |
| sp\|P10639\|THIO_MOUSE | Txn | THIO_MOUSE Thioredoxin | 1 | 221.823 | 285.819 | 1.289 |
| sp\|Q70E20\|SNED1_MOUSE | Sned1 | SNED1_MOUSE Sushi, nidogen and EGF-like domain- | 26 | 3749.24 | 4830.79 | 1.288 |
| | | containing protein 1 | | | | |
| sp\|O08579\|EMD_MOUSE | Emd | EMD_MOUSE Emerin | 2 | 198.907 | 256.184 | 1.288 |
| sp\|Q9Z1J3\|NFS1_MOUSE | Nfs1 | NFS1_MOUSE Cysteine desulfurase, mitochondrial | 3 | 317.325 | 408.506 | 1.287 |
| sp\|Q9ET30\|TM9S3_MOUSE | Tm9sf3 | TM9S3_MOUSE Transmembrane 9 superfamily member 3 | 5 | 741.671 | 954.252 | 1.287 |
| sp\|Q9CZT5\|VASN_MOUSE | Vasn | VASN_MOUSE Vasorin | 6 | 600.67 | 771.926 | 1.285 |
| sp\|P98154\|IDD_MOUSE | Dgcr2 | IDD_MOUSE Integral membrane protein DGCR2/IDD | 2 | 153.3 | 196.96 | 1.285 |
| sp\|P97873\|LOXL1_MOUSE | Loxl1 | LOXL1_MOUSE Lysyl oxidase homolog 1 | 6 | 397.501 | 510.51 | 1.284 |
| sp\|Q60520\|SIN3A_MOUSE | Sin3a | SIN3A_MOUSE Paired amphipathic helix protein Sin3a | 5 | 316.821 | 406.862 | 1.284 |
| sp\|O55029\|COPB2_MOUSE | Copb2 | COPB2_MOUSE Coatomer subunit beta' | 3 | 177.494 | 227.932 | 1.284 |
| sp\|Q9R1J0\|NSDHL_MOUSE | Nsdhl | NSDHL_MOUSE Sterol-4-alpha-carboxylate 3- | 1 | 57.1712 | 73.3885 | 1.284 |
| | | dehydrogenase, decarboxylating | | | | |
| sp\|Q99K01\|PDXD1_MOUSE | Pdxde1 | PDXD1_MOUSE Pyridoxal-dependent decarboxylase | 13 | 1397.45 | 1792.16 | 1.282 |
| | | domain-containing protein 1 | | | | |
| sp\|P41105\|RL28_MOUSE | Rpl28 | RL28_MOUSE 60S ribosomal protein L28 | 8 | 1331.16 | 1707.05 | 1.282 |
| sp\|035625\|AXIN1_MOUSE | Axin1 | AXIN1_MOUSE Axin-1 | 1 | 33.386 | 42.8027 | 1.282 |
| sp\|Q8BZH0\|S39AD_MOUSE | Slc39a13 | S39AD_MOUSE Zinc transporter ZIP13 | 8 | 893.672 | 1144.88 | 1.281 |
| sp\|Q5ND52\|RMTL1_MOUSE | Rnmt11 | RMTL1_MOUSE RNA methyltransferase-like protein 1 | 1 | 55.2006 | 70.7108 | 1.281 |
| sp\|Q3U9G9\|LBR_MOUSE | Lbr | LBR_MOUSE Lamin-B receptor | 2 | 115.721 | 147.963 | 1.279 |
| sp\|P35700\|PRDX1_MOUSE | Prdx1 | PRDX1_MOUSE Peroxiredoxin-1 | 5 | 330.018 | 420.928 | 1.275 |
| sp\|P07356\|ANXA2_MOUSE | Anxa2 | ANXA2_MOUSE Annexin A2 | 6 | 353.084 | 450.248 | 1.275 |
| sp\|P48962\|ADT1_MOUSE | Slc25a4 | ADT1_MOUSE ADP/ATP translocase 1 | 11 | 2215.72 | 2824.28 | 1.275 |
| sp\|Q8BV66\|IFI44_MOUSE | Ifi44 | IFI44_MOUSE Interferon-induced protein 44 | 1 | 180.136 | 229.377 | 1.273 |
| sp\|Q9QXS1\|PLEC_MOUSE | Plec | PLEC_MOUSE Plectin | 13 | 755.487 | 961.655 | 1.273 |
| sp\|P97333\|NRP1_MOUSE | Nip1 | NRP1_MOUSE Neuropilin-1 | 2 | 127.287 | 161.72 | 1.271 |
| sp\|P62743\|AP2S1_MOUSE | Ap2s1 | AP2S1_MOUSE AP-2 complex subunit sigma | 2 | 132.265 | 167.986 | 1.270 |
| sp\|Q61292\|LAMB2_MOUSE | Lamb2 | LAMB2_MOUSE Laminin subunit beta-2 | 18 | 1228.79 | 1560.47 | 1.270 |
| sp\|Q03350\|TSP2_MOUSE | Thbs2 | TSP2_MOUSE Thrombospondin-2 | 16 | 1719.04 | 2182.46 | 1.270 |
| sp\|Q9JM99\|PRG4_MOUSE | Prg4 | PRG4_MOUSE Proteoglycan 4 | 4 | 1065.2 | 1352.07 | 1.269 |
| sp\|Q8BPB5\|FBLN3_MOUSE | Efemp1 | FBLN3_MOUSE EGF-containing fibulin-like extracellular | 7 | 424.57 | 538.876 | 1.269 |
| | | matrix protein 1 | | | | |
| sp\|Q921X9\|PDIA5_MOUSE | Pdia5 | PDIA5_MOUSE Protein disulfide-isomerase A5 | 5 | 654.281 | 830.317 | 1.269 |
| tr\|Q3TML0\|Q3TML0_MOUSE | Pdia6 | Q3TML0_MOUSE Protein disulfide-isomerase A6 | 39 | 3847.17 | 4879.53 | 1.268 |
| sp\|Q8BXV2\|BRI3B_MOUSE | Bri3bp | BRI3B_MOUSE BRI3-binding protein | 5 | 354.444 | 449.429 | 1.268 |
| sp\|P61021\|RAB5B_MOUSE | Rab5b | RAB5B_MOUSE Ras-related protein Rab-5B | 2 | 77.3001 | 97.958 | 1.267 |
| sp\|Q8BTV1\|TUSC3_MOUSE | Tusc3 | TUSC3_MOUSE Tumor suppressor candidate 3 | 3 | 366.565 | 464.438 | 1.267 |
| sp\|Q9WV84\|NDKM_MOUSE | Nme4 | NDKM_MOUSE Nucleoside diphosphate kinase, | 23 | 3641.96 | 4611.75 | 1.266 |
| | | mitochondrial | | | | |
| sp\|Q9DB15\|RM12_MOUSE | Mrpl12 | RM12_MOUSE 39S ribosomal protein L12, mitochondrial | 1 | 128.398 | 162.58 | 1.266 |
| sp\|Q8JZR0\|ACSL5_MOUSE | Acsl5 | ACSL5_MOUSE Long-chain-fatty-acid-CoA ligase 5 | 1 | 96.4346 | 122.098 | 1.266 |
| sp\|Q8BGS7\|CEPT1_MOUSE | Cept1 | CEPT1_MOUSE Choline/ethanolaminephospho- | 5 | 1025.63 | 1298.18 | 1.266 |
| | | transferase 1 | | | | |
| sp\|P54822\|PUR8_MOUSE | Adsl | PUR8_MOUSE Adenylosuccinate lyase | 3 | 137.407 | 173.752 | 1.265 |
| sp\|Q9ERY9\|ERG28_MOUSE | ORF11 | ERG28_MOUSE Probable ergosterol biosynthetic | 2 | 409.333 | 517.165 | 1.263 |
| | | protein 28 | | | | |
| sp\|O08795\|GLU2B_MOUSE | Prkcsh | GLU2B_MOUSE Glucosidase 2 subunit beta | 13 | 1820.55 | 2297.27 | 1.262 |
| sp\|Q61526\|ERBB3_MOUSE | Erbb3 | ERBB3_MOUSE Receptor tyrosine-protein kinase erbB-3 | 1 | 110.254 | 139.086 | 1.262 |
| sp\|Q91XB7\|YIF1A_MOUSE | Yif1a | YIF1A_MOUSE Protein YIF1A | 1 | 59.594 | 75.1519 | 1.261 |
| sp\|O54734\|ST48_MOUSE | Ddost | OST48_MOUSE Dolichyl-diphosphooligosaccharide-- | 12 | 991.766 | 1250.68 | 1.261 |
| | | protein glycosyltransferase 48 kDa subunit | | | | |
| sp\|Q3TJZ6\|FA98A_MOUSE | Fam98a | FA98A_MOUSE Protein FAM98A | 2 | 278.163 | 350.607 | 1.260 |
| sp\|P63094\|GNAS2_MOUSE | Gnas | GNAS2_MOUSE Guanine nucleotide-binding protein G(s) | 1 | 28.6904 | 36.1299 | 1.259 |
| | | subunit alpha isoforms short | | | | |
| sp\|P68254\|1433T_MOUSE | Ywhaq | 1433T_MOUSE 14-3-3 protein thcta | 2 | 156.211 | 196.703 | 1.259 |
| sp\|P62069\|UBP46_MOUSE | Usp46 | UBP46_MOUSE Ubiquitin carboxyl-terminal hydrolase 46 | 1 | 38.2408 | 48.1307 | 1.259 |
| sp\|P51912\|AAAT_MOUSE | Slc1a5 | AAAT_MOUSE Neutral amino acid transporter B(0) | 1 | 30.3358 | 38.1659 | 1.258 |
| sp\|P55302\|AMRP_MOUSE | Lrpap1 | AMRP_MOUSE Alpha-2-macroglobulin receptor- | 5 | 401.025 | 504.187 | 1.257 |
| | | associated protein | | | | |
| sp\|P62500\|T22D1_MOUSE | Tsc22d1 | T22D1_MOUSE TSC22 domain family protein 1 | 2 | 255.02 | 320.338 | 1.256 |
| sp\|Q99J99\|THTM_MOUSE | Mpst | THTM_MOUSE 3-mercaptopyruvate sulfurtransferase | 3 | 249.614 | 313.044 | 1.254 |
| sp\|Q91YH5\|ATLA3_MOUSE | Atl3 | ATLA3_MOUSE Atlastin-3 | 6 | 681.67 | 854.335 | 1.253 |
| sp\|P08113\|ENPL_MOUSE | Hsp90b1 | ENPL_MOUSE Endoplasmin | 54 | 7927.39 | 9934.87 | 1.253 |
| tr\|E9Q4X2\|E9Q4X2_MOUSE | Uggt2 | E9Q4X2_MOUSE Protein Uggt2 | 4 | 345.856 | 433.256 | 1.253 |
| sp\|Q9Z1R2\|BAG6_MOUSE | Bag6 | BAG6_MOUSE Large proline-rich protein BAG6 | 2 | 210.613 | 263.092 | 1.249 |
| sp\|Q80VA0\|GALT7_MOUSE | Galnt7 | GALT7_MOUSE N-acetylgalactosaminyltransferase 7 | 3 | 550.677 | 687.6 | 1.249 |
| sp\|Q61576\|FKB10_MOUSE | Fkbp10 | FKB10_MOUSE Peptidyl-prolyl cis-trans isomerase | 8 | 1368.46 | 1707.51 | 1.248 |
| | | FKBP 10 | | | | |
| sp\|P56379\|68MP_MOUSE | Mp68 | 68MP_MOUSE 6.8 kDa mitochondrial proteolipid | 2 | 183.974 | 229.506 | 1.247 |
| sp\|P09242\|PPBT_MOUSE | Alpl | PPBT_MOUSE Alkaline phosphatase, tissue-nonspecific | 6 | 443.784 | 553.373 | 1.247 |
| | | isozyme | | | | |
| sp\|Q9JJG9\|NOA1_MOUSE | Noa1 | NOA1_MOUSE Nitric oxide-associated protein 1 | 2 | 221.15 | 275.521 | 1.246 |
| sp\|O35245\|PKD2_MOUSE | Pkd2 | PKD2_MOUSE Polycystin-2 | 2 | 151.92 | 189.211 | 1.245 |
| sp\|Q9DCG9\|TR112_MOUSE | Trmt112 | TR112_MOUSE tRNA methyltransferase 112 homolog | 1 | 43.8776 | 54.5719 | 1.244 |
| sp\|P63085\|MK01_MOUSE | Mapk1 | MK01_MOUSE Mitogen-activated protein kinase 1 | 2 | 218.652 | 271.887 | 1.243 |
| tr\|E9PZF0\|E9PZF0_MOUSE | Gm20390 | E9PZF0_MOUSE Nucleoside diphosphate kinase | 13 | 3037.16 | 3775.86 | 1.243 |
| sp\|O70305\|ATX2_MOUSE | Atxn2 | ATX2_MOUSE Ataxin-2 | 9 | 853.937 | 1061.6 | 1.243 |
| sp\|P97496\|SMRC1_MOUSE | Smarcc1 | SMRC1_MOUSE SWI/SNF complex subunit SMARCC1 | 6 | 695.339 | 864.145 | 1.243 |
| sp\|Q3U4G3\|XXLT1_MOUSE | Xxylt1 | XXLT1_MOUSE Xyloside xylosyltransferase 1 | 6 | 1096.34 | 1362.28 | 1.243 |
| sp\|Q8R361\|RFIP5_MOUSE | Rab11fip5 | RFIP5_MOUSE Rab11 family-interacting protein 5 | 9 | 975.821 | 1212.43 | 1.242 |
| sp\|P62858\|RS28_MOUSE | Rps28 | RS28_MOUSE 40S ribosomal protein S28 | 4 | 247.628 | 307.313 | 1.241 |
| sp\|P35492\|HUTH_MOUSE | Hal | HUTH_MOUSE Histidine ammonia-lyase | 1 | 140.984 | 174.964 | 1.241 |
| sp\|Q9WVD5\|ORNT1_MOUSE | Slc25a15 | ORNT1_MOUSE Mitochondrial ornithine transporter 1 | 1 | 36.8992 | 45.7797 | 1.241 |
| sp\|Q8K370\|ACD10_MOUSE | Acad10 | ACD10_MOUSE Acyl-CoA dehydrogenase family | 10 | 1300.26 | 1612.57 | 1.240 |
| | | member 10 | | | | |
| sp\|P70699\|LYAG_MOUSE | Gaa | LYAG_MOUSE Lysosomal alpha-glucosidase | 3 | 168.975 | 209.5 | 1.240 |
| sp\|Q8R422\|CD109_MOUSE | Cd109 | CD109_MOUSE CD109 antigen | 7 | 566.98 | 702.752 | 1.239 |
| sp\|Q3UBZ5\|MI4GD_MOUSE | Mif4gd | MI4GD_MOUSE MIF4G domain-containing protein | 1 | 59.0593 | 73.1884 | 1.239 |
| sp\|P08752\|GNAI2_MOUSE | Gnai2 | GNAI2_MOUSE Guanine nucleotide-binding protein | 2 | 102.961 | 127.59 | 1.239 |
| | | G(i) subunit alpha-2 | | | | |
| sp\|Q925I1\|ATAD3_MOUSE | Atad3 | ATAD3_MOUSE ATPase family AAA domain- | 11 | 1186.36 | 1469.14 | 1.238 |
| | | containing protein 3 | | | | |
| sp\|Q9EQQ2\|YIPF5_MOUSE | Yipf5 | YIPF5_MOUSE Protein YIPF5 | 7 | 914.419 | 1132.14 | 1.238 |
| sp\|Q9JHP7\|KDEL1_MOUSE | Kdelc1 | KDEL1_MOUSE KDEL motif-containing protein 1 | 3 | 230.431 | 285.03 | 1.237 |
| sp\|Q6PHN9\|RAB35_MOUSE | Rab35 | RAB35_MOUSE Ras-related protein Rab-35 | 1 | 31.4937 | 38.9477 | 1.237 |
| sp\|Q8BHN3\|GANAB_MOUSE | Ganab | GANAB_MOUSE Neutral alpha-glucosidase AB | 39 | 4991.13 | 6170.99 | 1.236 |
| sp\|P62900\|RL31_MOUSE | Rpl31 | RL31_MOUSE 60S nbosomal protein L31 | 5 | 1929.77 | 2385.84 | 1.236 |
| sp\|Q62425\|NDUA4_MOUSE | Ndufa4 | NDUA4_MOUSE NADH dehydrogenase [ubiquinone] 1 | 5 | 1133.63 | 1399.71 | 1.235 |
| | | alpha subcomplex subunit 4 | | | | |
| sp\|P28798\|GRN_MOUSE | Grn | GRN_MOUSE Granulins | 22 | 2858.69 | 3526.25 | 1.234 |
| sp\|Q6PB44\|PTN23_MOUSE | Ptpn23 | PTN23_MOUSE Tyrosine-protein phosphatase non- | 2 | 60.8877 | 75.0967 | 1.233 |
| | | receptor type 23 | | | | |
| sp\|Q9CQU3\|RER1_MOUSE | Rer1 | RER1_MOUSE Protein RER1 | 2 | 142.954 | 176.279 | 1.233 |
| sp\|P62259\|1433E_MOUSE | Ywhae | 1433E_MOUSE 14-3-3 protein epsilon | 2 | 165.26 | 203.558 | 1.232 |
| sp\|Q9WVJ9\|FBLN4_MOUSE | Efemp2 | FBLN4_MOUSE EGF-conlaining fibulin-like | 1 | 39.3083 | 48.3379 | 1.230 |
| | | extracellular matrix protein 2 | | | | |
| sp\|Q8C0S4\|TT21A_MOUSE | Ttc21a | TT21A_MOUSE Tetratricopcptide repeat protein 21A | 1 | 111.488 | 137.098 | 1.230 |
| sp\|Q8C3X8\|LMF2_MOUSE | Lmf2 | LMF2_MOUSE Lipase maturation factor 2 | 5 | 521.869 | 641.531 | 1.229 |
| sp\|Q8BJM5\|ZNT6_MOUSE | Slc30a6 | ZNT6_MOUSE Zinc transporter 6 | 8 | 1355.51 | 1665.89 | 1.229 |
| sp\|Q9DB43\|ZFPL1_MOUSE | Zfpl1 | ZFPL1_MOUSE Zinc finger protein-like 1 | 1 | 52.1804 | 64.0486 | 1.227 |
| sp\|Q6ZQI3\|MLEC_MOUSE | Mlec | MLEC_MOUSE Malectin | 9 | 1664.12 | 2039.86 | 1.226 |
| sp\|Q9QUJ7\|ACSL4_MOUSE | Acsl4 | ACSL4_MOUSE Long-chain-fatty-acid--CoA ligase 4 | 3 | 551.583 | 676.088 | 1.226 |
| sp\|Q8BX10\|PGAM5_MOUSE | Pgam5 | PGAM5_MOUSE Serine/threonine-protein phospliatase | 5 | 457.119 | 560.246 | 1.226 |
| | | PGAM5, mitochondrial | | | | |
| sp\|P22682\|CBL_MOUSE | Cbl | CBL_MOUSE E3 ubiquitin-protein ligase CBL | 3 | 200.948 | 246.155 | 1.225 |
| sp\|P37889\|FBLN2_MOUSE | Fbln2 | FBLN2_MOUSE Fibulin-2 | 21 | 1626.1 | 1991.35 | 1.225 |
| tr\|H3BKK0\|H3BKK0_MOUSE | Aldh18a1 | H3BKK0_MOUSE Delta-1-pyrroline-5-carboxylate | 1 | 103.401 | 126.593 | 1.224 |
| | | synthase (Fragment) | | | | |
| sp\|Q91X20\|ASH2L_MOUSE | Ash2l | ASH2L_MOUSE Set1/Ash2 histone methyltransferase | 1 | 138.149 | 169.133 | 1.224 |
| | | complex subunit ASH2 | | | | |
| sp\|Q9EPS3\|GLCE_MOUSE | Glce | GLCE_MOUSE D-glucuronyl C5-epimerase | 1 | 49.6178 | 60.7226 | 1.224 |
| sp\|P35569\|IRS1_MOUSE | Irs1 | IRS1_MOUSE Insulin receptor substrate 1 | 3 | 337.942 | 413.492 | 1.224 |
| sp\|Q8BP92\|RCN2_MOUSE | Rcn2 | RCN2_MOUSE Reticulocalbin-2 | 1 | 134.289 | 164.28 | 1.223 |
| sp\|O35900\|LSM2_MOUSE | Lsm2 | LSM2_MOUSE U6 snRNA-associated Sm-like protein | 1 | 55.8987 | 68.3808 | 1.223 |
| | | LSm2 | | | | |
| sp\|O08665\|SEM3A_MOUSE | Sema3a | SEM3A_MOUSE Semaphorin-3A | 12 | 2241.07 | 2737.67 | 1.222 |
| sp\|Q8BMS1\|ECHA_MOUSE | Hadha | ECHA_MOUSE Trifunctional enzyme subunit alpha, | 11 | 1976.38 | 2411.61 | 1.220 |
| | | mitochondrial | | | | |
| sp\|P22437\|PGH1_MOUSE | Ptgs1 | PGH1_MOUSE Prostaglandin G/H synthase 1 | 25 | 2688 | 3277.91 | 1.219 |
| sp\|O35704\|SPTC1_MOUSE | Sptlc1 | SPTC1_MOUSE Serine palmitoyltransferase 1 | 2 | 119.677 | 145.902 | 1.219 |
| sp\|P48678\|LMNA_MOUSE | Lmna | LMNA_MOUSE Prelamin-A/C | 53 | 8129.96 | 9909.74 | 1.219 |
| sp\|Q02853\|MMP11_MOUSE | Mmp11 | MMP11_MOUSE Stromelysin-3 | 1 | 65.5702 | 79.9015 | 1.219 |
| sp\|054951\|SEM6B_MOUSE | Sema6b | SEM6B_MOUSE Semaphorin-6B | 1 | 40.6804 | 49.5686 | 1.218 |
| sp\|Q9JJE7\|FADS3_MOUSE | Fads3 | FADS3_MOUSE Fatty acid desaturase 3 | 3 | 621.762 | 756.383 | 1.217 |
| sp\|Q8BU88\|RM22_MOUSE | Mrpl22 | RM22_MOUSE 39S ribosomal protein L22, mitochondrial | 2 | 187.685 | 228.242 | 1.216 |
| sp\|Q07797\|LG3BP_MOUSE | Lgals3bp | LG3BP_MOUSE Galectin-3-binding protein | 5 | 440.954 | 536.225 | 1.216 |
| sp\|Q8VDD5\|MYH9_MOUSE | Myh9 | MYH9_MOUSE Myosin-9 | 19 | 1715.4 | 2083.96 | 1.215 |
| sp\|Q8CGK3\|LONM_MOUSE | Lonp1 | LONM_MOUSE Lon protease homolog, mitochondrial | 6 | 586.365 | 711.947 | 1.214 |
| sp\|P62317\|SMD2_MOUSE | Snrpd2 | SMD2_MOUSE Small nuclear ribonuclcoprotein Sm D2 | 3 | 249.621 | 302.92 | 1.214 |
| sp\|O54991\|CNTP1_MOUSE | Cntnap1 | CNTP1_MOUSE Contactin-associated protein 1 | 2 | 136.302 | 165.265 | 1.212 |
| sp\|Q8R366\|IGSF8_MOUSE | Igsf8 | IGSF8_MOUSE Immunoglobulin superfamily member 8 | 3 | 316.849 | 383.974 | 1.212 |
| sp\|Q9ZlQ2\|ABHGA_MOUSE | Abhd16a | ABHGA_MOUSE Abhydrolase domain-containing | 3 | 367.784 | 445.641 | 1.212 |
| | | protein 16A | | | | |
| sp\|Q8BGT5\|ALAT2_MOUSE | Gpt2 | ALAT2_MOUSE Alanine aminotransferase 2 | 25 | 2956.36 | 3580.2 | 1.211 |
| sp\|Q8BH61\|F13A_MOUSE | F13a1 | F13A_MOUSE Coagulation factor XIII A chain | 1 | 69.9335 | 84.6339 | 1.210 |
| sp\|P70182\|PI51A_MOUSE | Pip5k1a | PI51A_MOUSE Phospliatidylinositol 4-phosphate | 11 | 831.14 | 1005.04 | 1.209 |
| | | 5-kinase type-1 alpha | | | | |
| sp\|Q80ZM7\|T2AG_MOUSE | Gtf2a2 | T2AG_MOUSE Transcription initiation factor IIA | 3 | 1127.34 | 1363.18 | 1.209 |
| | | subunit 2 | | | | |
| sp\|Q8R1Z9\|RN121_MOUSE | Rnf121 | RN121_MOUSE RING finger protein 121 | 2 | 139.991 | 169.1 | 1.208 |
| sp\|Q8BMS4\|COQ3_MOUSE | Coq3 | COQ3_MOUSE Hexaprenyldihydroxybenzoate | 1 | 87.844 | 106.023 | 1.207 |
| | | methyltransferase, mitochondrial | | | | |
| sp\|O35149\|ZNT4_MOUSE | Slc30a4 | ZNT4_MOUSE Zinc transporter 4 | 10 | 1183.69 | 1425.65 | 1.204 |
| sp\|O35972\|RM23_MOUSE | Mrpl23 | RM23_MOUSE 39S ribosomal protein L23, mitochondrial | 2 | 304.698 | 366.732 | 1.204 |
| sp\|P47758\|SRPRB_MOUSE | Srprb | SRPRB_MOUSE Signal recognition particle receptor | 5 | 749.435 | 899.863 | 1.201 |
| | | subunit beta | | | | |
| sp\|Q8K2B0\|SC65_MOUSE | Leprel4 | SC65_MOUSE Synaptonemal complex protein SC65 | 1 | 51.1549 | 61.332 | 1.199 |
| sp\|P55258\|RAB8A_MOUSE | Rab8a | RAB8A_MOUSE Ras-related protein Rab-8A | 5 | 1108.94 | 1326.98 | 1.197 |
| sp\|Q3TBT3\|STING_MOUSE | Tmem173 | STING_MOUSE Stimulator of interferon genes protein | 1 | 61.7107 | 73.8386 | 1.197 |
| sp\|Q64429\|CP1B1_MOUSE | Cvp1b1 | CP1B1_MOUSE Cytochrome P450 1B1 | 2 | 72.7199 | 86.9875 | 1.196 |
| sp\|Q8CIE6\|COPA_MOUSE | Copa | COPA_MOUSE Coatomer subunit alpha | 20 | 2502.81 | 2989.57 | 1.194 |
| tr\|Q8VCG1\|Q8VCG1_MOUSE | Dut | Q8VCG1_MOUSE Deoxyuridine triphosphatase, | 2 | 156.325 | 186.596 | 1.194 |
| | | isoform CRA_b | | | | |
| sp\|Q9D379\|HYEP_MOUSE | Ephx1 | HYEP_MOUSE Epoxide hydrolase 1 | 4 | 888.537 | 1060.14 | 1.193 |
| sp\|O70503\|DHB12_MOUSE | Hsd17b12 | DHB12_MOUSE Estradiol 17-beta-dehydrogenase 12 | 3 | 447.073 | 532.035 | 1.190 |
| sp\|Q9CQN1\|TRAP1_MOUSE | Trap1 | TRAP1_MOUSE Heat shock protein 75 kDa, | 22 | 3590.89 | 4271.84 | 1.190 |
| | | mitochondrial | | | | |
| sp\|Q8C1A5\|THOP1_MOUSE | Thop1 | THOP1_MOUSE Thimet oligopeptidase | 1 | 187.312 | 222.797 | 1.189 |
| sp\|P62315\|SMD1_MOUSE | Snrpd1 | SMD1_MOUSE Small nuclear ribonucleoprotein Sm D1 | 3 | 507.08 | 603.034 | 1.189 |
| sp\|Q8BLF1\|NCEH1_MOUSE | Nceh1 | NCEH1_MOUSE Neutral cholesterol ester hydrolase 1 | 1 | 44.0058 | 52.3286 | 1.189 |
| sp\|Q8K2Y7\|RM47_MOUSE | Mrpl47 | RM47_MOUSE 39S ribosomal protein L47, | 1 | 42.9905 | 51.1148 | 1.189 |
| | | mitochondrial | | | | |
| sp\|Q9JIM1\|S29A1_MOUSE | Slc29a1 | S29A1_MOUSE Equilibrative nucleoside transporter 1 | 1 | 97.6738 | 116.067 | 1.188 |
| tr\|Q9QZ18\|Q9QZ18_MOUSE | Olfr71 | Q9QZ18_MOUSE Olfactory receptor | 1 | 132.538 | 157.227 | 1.186 |
| sp\|Q64133\|AOFA_MOUSE | Maoa | AOFA_MOUSE Amine oxidase [flavin-containing] A | 1 | 66.5551 | 78.8288 | 1.184 |
| sp\|Q6DID7\|WLS_MOUSE | Wls | WLS_MOUSE Protein wntless homolog | 2 | 437.159 | 517.721 | 1.184 |
| sp\|P16045\|LEG1_MOUSE | Lgals1 | LEG1_MOUSE Galectin-1 | 4 | 678.314 | 803.208 | 1.184 |
| tr\|Q8R284\|Q8R284_MOUSE | Vnm1r84 | Q8R284_MOUSE Protein Vmn1r84 | 1 | 47.4254 | 56.1493 | 1.184 |
| sp\|P62821\|RAB1A_MOUSE | Rab1A | RAB1A_MOUSE Ras-related protein Rab-1A | 11 | 1722.69 | 2038.04 | 1.183 |
| sp\|P35279\|RAB6A_MOUSE | Rab6a | RAB6A_MOUSE Ras-related protein Rab-6A | 3 | 449.282 | 531.483 | 1.183 |
| sp\|Q4PJXl\|ODR4_MOUSE | Odr4 | ODR4_MOUSE Protein odr-4 homolog | 1 | 39.6916 | 46.9334 | 1.182 |
| sp\|088736\|DHB7_MOUSE | Hsd17b7 | DHB7_MOUSE 3-keto-steroid reductase | 3 | 137.047 | 162.038 | 1.182 |
| sp\|Q00993\|UFO_MOUSE | Axl | UFO_MOUSE Tyrosine-protein kinase receptor UFO | 2 | 96.4829 | 114.059 | 1.182 |
| tr\|B2RXX5\|B2RXX5_MOUSE | Adamts14 | B2RXX5_MOUSE A disintegrin-like and | 1 | 45.301 | 53.5512 | 1.182 |
| | | metallopeptidase (Reprolysin type) with | | | | |
| | | thrombospondin type 1 motif, 14 | | | | |
| sp\|P34884\|MIF_MOUSE | Mif | MIF_MOUSE Macrophage migration inhibitory factor | 1 | 117.17 | 138.488 | 1.182 |
| tr\|G3XA02\|G3XA02_MOUSE | Sgsh | G3XA02_MOUSE N-sulfoglucosamine sulfohydrolase | 1 | 32.4285 | 38.2702 | 1.180 |
| | | (Sulfamidase), isoform CRA_e | | | | |
| sp\|P97820\|M4K4_MOUSE | Map4k4 | M4K4_MOUSE Mitogen-activated protein kinase | 9 | 805.618 | 950.653 | 1.180 |
| | | kinase kinase kinase 4 | | | | |
| sp\|P24369\|PPIB_MOUSE | Ppib | PPIB_MOUSE Peptidyl-prolyl cis-trans isomerase B | 11 | 1544.43 | 1822.39 | 1.180 |
| tr\|D3Z2E7\|D3Z2E7_MOUSE | AI607873 | D3Z2E7_MOUSE Protein AI607873 | 1 | 73.373 | 86.5724 | 1.180 |
| sp\|Q9CRA4\|MSMO1_MOUSE | Msmo1 | MSMO1_MOUSE Methylsterol monooxy genase 1 | 9 | 1517.07 | 1789.56 | 1.180 |
| sp\|Q9QZ85\|IIGP1_MOUSE | Iigp1 | IIGP1_MOUSE Interferon-inducible GTPase 1 | 5 | 321.557 | 379.262 | 1.179 |
| sp\|054941\|SMCE1_MOUSE | Smarce1 | SMCE1_MOUSE SWI/SNF-related matrix-associated | 1 | 44.9976 | 53.0614 | 1.179 |
| | | actin-dependent regulator of chromatin | | | | |
| | | subfamily E member 1 | | | | |
| sp\|Q91VM9\|IPYR2_MOUSE | Ppa2 | IPYR2_MOUSE Inorganic pyrophosphatase 2, | 2 | 179.17 | 210.927 | 1.177 |
| | | mitochondrial | | | | |
| sp\|Q9DCC4\|P5CR3_MOUSE | Pycrl | P5CR3_MOUSE Pynoline-5-carboxy ate reductase 3 | 10 | 1418.94 | 1670.42 | 1.177 |
| sp\|P48024\|EIF1_MOUSE | Eif1 | EIF1_MOUSE Eukaryotic translation initiation factor 1 | 1 | 62.9705 | 74.11 | 1.177 |
| sp\|P15105\|GLNA_MOUSE | Glul | GLNA_MOUSE Glutamine synthetase | 1 | 89.4631 | 105.28 | 1.177 |
| sp\|Q91YW3\|DNJC3_MOUSE | Dnajc3 | DNJC3_MOUSE DnaJ homolog subfamily C member 3 | 3 | 112.68 | 132.497 | 1.176 |
| sp\|Q9CQY5\|MAGT1_MOUSE | Magt1 | MAGT1_MOUSE Magnesium transporter protein 1 | 6 | 489.32 | 574.927 | 1.175 |
| sp\|Q3U7R1\|ESYT1_MOUSE | Esyt1 | ESYT1_MOUSE Extended synaptotagmin-1 | 81 | 11841.2 | 13904.3 | 1.174 |
| sp\|Q3TDN2\|FAF2_MOUSE | Faf2 | FAF2_MOUSE FAS-associated factor 2 | 2 | 117.044 | 137.393 | 1.174 |
| sp\|Q9EQH3\|VPS35_MOUSE | Vps35 | VPS35_MOUSE Vacuolar protein sorting-associated | 5 | 698.401 | 819.809 | 1.174 |
| | | protein 35 | | | | |
| sp\|Q3U319\|BRE1B_MOUSE | Rnf40 | BRE1B_MOUSE E3 ubiquitin-protein ligase BRE1B | 3 | 157.182 | 184.434 | 1.173 |
| sp\|P01887\|B2MG_MOUSE | B2m | B2MG_MOUSE Beta-2-microglobulin | 1 | 109.123 | 127.935 | 1.172 |
| sp\|088696\|CLPP_MOUSE | Clpp | CLPP_MOUSE ATP-dependent Clp protease proteolytic | 3 | 328.162 | 384.464 | 1.172 |
| | | subunit, mitochondrial | | | | |
| sp\|P63002\|AES_MOUSE | Aes | AES_MOUSE Amino-terminal enhancer of split | 1 | 53.6217 | 62.8007 | 1.171 |
| sp\|Q99P88\|NU155_MOUSE | Nup155 | NU155_MOUSE Nuclear pore complex protein Nup155 | 1 | 50.647 | 59.2756 | 1.170 |
| sp\|Q9Z0L0\|TPBG_MOUSE | Tpbg | TPBG_MOUSE Trophoblast glycoprotein | 4 | 586.531 | 685.607 | 1.169 |
| sp\|Q99LJ6\|GPX7_MOUSE | Gpx7 | GPX7_MOUSE Glutathione peroxidase 7 | 6 | 1085.84 | 1268.36 | 1.168 |
| tr\|E9Q5M6\|E9Q5M6_MOUSE | Wdr52 | E9Q5M6_MOUSE Protein Wdr52 | 1 | 126.639 | 147.92 | 1.168 |
| sp\|Q91YQ5\|RPN1_MOUSE | Rpn1 | RPN1_MOUSE Dolichyl-diphosphooligosaccharide-- | 77 | 15238.2 | 17793.5 | 1.168 |
| | | protein glycosyltransferase subunit 1 | | | | |
| sp\|Q5RKS2\|SMIM7_MOUSE | Smim7 | SMIM7_MOUSE Small integral membrane protein 7 | 2 | 186.963 | 218.247 | 1.167 |
| sp\|Q99L04\|DHRSI_MOUSE | Dhrs1 | DHRS1_MOUSE Dehydrogenase/reductase SDR | 1 | 96.2888 | 112.37 | 1.167 |
| | | family member 1 | | | | |
| tr\|Q3UWE6\|Q3UWE6_MOUSE | Wdr20 | Q3UWE6_MOUSE MCG14935, isoform CRA_a | 2 | 142.744 | 166.545 | 1.167 |
| sp\|Q8VDL4\|ADPGK_MOUSE | Adpgk | ADPGK_MOUSE ADP-dependent glucokinase | 11 | 974.848 | 1137.31 | 1.167 |
| tr\|Q9Z1M2\|Q9Z1M2_MOUSE | Irgm2 | Q9Z1M2_MOUSE Interferon-g induced GTPase | 1 | 105.464 | 122.745 | 1.164 |
| sp\|Q6P5E4\|UGGG1_MOUSE | Uggt1 | UGGG1_MOUSE UDP-glucose:glycoprotein | 67 | 10994.1 | 12792.6 | 1.164 |
| | | glucosyltransferase 1 | | | | |
| sp\|Q9DBSl\|TMM43_MOUSE | Tmem43 | TMM43_MOUSE Transmembrane protein 43 | 11 | 1207.43 | 1404.9 | 1.164 |
| sp\|Q8R2Q4\|RRF2M_MOUSE | Gfm2 | RRF2M_MOUSE Ribosome-releasing factor 2, | 2 | 107.692 | 125.292 | 1.163 |
| | | mitochondrial | | | | |
| sp\|P61255\|RL26_MOUSE | Rpl26 | RL26_MOUSE 60S ribosomal protein L26 | 7 | 1242.47 | 1445.41 | 1.163 |
| sp\|Q9CPR5-2\|RM15MOUSE | Mrpl15 | RM15_MOUSE Isoform 2 of 39S ribosomal protein L15, | 1 | 77.1038 | 89.631 | 1.162 |
| | | mitochondrial | | | | |
| sp\|Q8QZY9\|SF3B4_MOUSE | Sf3b4 | SF3B4_MOUSE Splicing factor 3B subunit 4 | 1 | 142.796 | 165.851 | 1.161 |
| sp\|Q5F2E8\|TAOK1_MOUSE | Taok1 | TAOK1_MOUSE Serine/threonine-protein kinase TAO1 | 1 | 75.3488 | 87.3674 | 1.160 |
| sp\|P47962\|RL5_MOUSE | Rpl5 | RL5_MOUSE 60S ribosomal protein L5 | 1 | 62.7301 | 72.7117 | 1.159 |
| sp\|Q8CG19\|LTBPI_MOUSE | Ltbp1 | LTBP1_MOUSE Latent-transforming growth factor | 12 | 1065.95 | 1235.4 | 1.159 |
| | | beta-binding protein 1 | | | | |
| sp\|Q9CZX8\|RS19_MOUSE | Rps19 | RS19_MOUSE 40S ribosomal protein S19 | 4 | 708.933 | 821.593 | 1.159 |
| sp\|Q80XC3\|US6NL_MOUSE | Usp6n1 | US6NL_MOUSE USP6 N-terminal-like protein | 2 | 68.0847 | 78.9006 | 1.159 |
| sp\|E9Q555\|RN213_MOUSE | Rnf213 | RN213_MOUSE E3 ubiquitin-protein ligase RNF213 | 18 | 1458.47 | 1690.04 | 1.159 |
| sp\|Q9QXT0\|CNPY2_MOUSE | Cnpy2 | CNPY2_MOUSE Protein canopy homolog 2 | 4 | 533.024 | 617.094 | 1.158 |
| sp\|Q00612\|G6PD1_MOUSE | G6pdx | G6PD1_MOUSE Glucose-6-phosphate 1-dehydrogenase X | 28 | 5020 | 5809.34 | 1.157 |
| sp\|Q3V1L4\|5NTC_MOUSE | Nt5c2 | 5NTC_MOUSE Cytosolic purine S′-nucleotidase | 2 | 309.364 | 357.597 | 1.156 |
| sp\|Q91ZA3\|PCCA_MOUSE | Pcca | PCCA_MOUSE Propionyl-CoA carboxylase alpha | 5 | 576.376 | 666.088 | 1.156 |
| | | chain, mitochondrial | | | | |
| sp\|Q3UKC1\|TAXB1_MOUSE | Tax1bp1 | TAXB1_MOUSE Taxi-binding protein 1 homolog | 1 | 33.9741 | 39.2312 | 1.155 |
| sp\|P13808\|B3A2_MOUSE | Slc4a2 | B3A2_MOUSE Anion exchange protein 2 | 12 | 1192.5 | 1376.54 | 1.154 |
| sp\|P07901\|HS90A_MOUSE | Hsp90aa1 | HS90A_MOUSE Heat shock protein HSP 90-alpha | 22 | 3454.04 | 3987.02 | 1.154 |
| sp\|P26618\|PGFRA_MOUSE | Pdgfra | PGFRA_MOUSE Platelet-derived growth factor | 3 | 350.205 | 404.071 | 1.154 |
| | | receptor alpha | | | | |
| sp\|Q9CYN2\|SPCS2_MOUSE | Spcs2 | SPCS2_MOUSE Signal peptidase complex subunit 2 | 6 | 1030.29 | 1188.5 | 1.154 |
| sp\|Q9D710\|TMX2_MOUSE | Tmx2 | TMX2_MOUSE Thioredoxin-related transmembrane | 2 | 233.459 | 269.014 | 1.152 |
| | | protein 2 | | | | |
| sp\|Q8C3X4\|GUF1_MOUSE | Guf1 | GUF1_MOUSE Translation factor Guf1, mitochondrial | 3 | 301.777 | 347.623 | 1.152 |
| sp\|Q8CGA0\|PPM1F_MOUSE | Ppm1f | PPM1F_MOUSE Protein phosphatase 1F | 1 | 60.169 | 69.287 | 1.152 |
| sp\|Q64521\|GPDM_MOUSE | Gpd2 | GPDM_MOUSE Glycerol-3-phosphate dehydrogenase, | 3 | 173.423 | 199.431 | 1.150 |
| | | mitochondrial | | | | |
| sp\|Q01279\|EGFR_MOUSE | Egfr | EGFR_MOUSE Epidermal growth factor receptor | 23 | 3544.6 | 4072.92 | 1.149 |
| sp\|P62852\|RS25_MOUSE | Rps25 | RS25_MOUSE 40S ribosomal protein S25 | 2 | 253.233 | 290.605 | 1.148 |
| sp\|Q9CZH7\|MXRA7_MOUSE | Mxran | MXRA7_MOUSE Matrix-remodeling-associated protein 7 | 1 | 54.0513 | 62.0192 | 1.147 |
| sp\|Q8R2Q8\|BST2_MOUSE | Bst2 | BST2_MOUSE Bone marrow stromal antigen 2 | 2 | 83.577 | 95.8743 | 1.147 |
| sp\|Q91XB0\|TREX1_MOUSE | Trex1 | TREX1_MOUSE Three-prime repair exonuclease 1 | 1 | 135.835 | 155.523 | 1.145 |
| sp\|Q810S1\|MCUB_MOUSE | Ccde109b | MCUB_MOUSE Mitochondrial calcium uniporter | 4 | 436.418 | 499.386 | 1.144 |
| | | regulatory subunit MCUb | | | | |
| sp\|Q9D2C7\|BI1_MOUSE | Tmbim6 | BI1_MOUSE Bax inhibitor 1 | 1 | 103.338 | 118.238 | 1.144 |
| sp\|P12787\|COX5A_MOUSE | Cox5a | COX5A_MOUSE Cytochrome c oxidase subunit 5A, | 2 | 957.165 | 1094.68 | 1.144 |
| | | mitochondrial | | | | |
| sp\|Q9ESP1\|SDF2L_MOUSE | Sdf211 | SDF2L_MOUSE Stromal cell-derived factor 2-like | 14 | 2898.81 | 3314.65 | 1.143 |
| | | protein 1 | | | | |
| tr\|Q3TMX5\|Q3TMX5_MOUSE | Manf | Q3TMX5_MOUSE Arginine-rich, mutated in early | 4 | 586.987 | 669.928 | 1.141 |
| | | stage tumors, isoform CRA_b | | | | |
| sp\|Q6PD26\|PIGS_MOUSE | Pigs | PIGS_MOUSE GPI transamidase component PIG-S | 10 | 1056.75 | 1206.03 | 1.141 |
| tr\|G3XA59\|G3XA59_MOUSE | Lrrc32 | G3XA59_MOUSE MCG51019, isoform CRA_b | 1 | 51.8598 | 59.1549 | 1.141 |
| sp\|O35887\|CALU_MOUSE | Calu | CALU_MOUSE Calumenin | 11 | 1697.13 | 1932.04 | 1.138 |
| sp\|Q9CQV8\|1433B_MOUSE | Ywhab | 1433B_MOUSE 14-3-3 protein beta/alpha | 1 | 132.342 | 150.48 | 1.137 |
| sp\|Q8BFP9\|PDK1_MOUSE | Pdk1 | PDK1_MOUSE [Pyiuvate dehydrogenase (acetyl- | 1 | 75.5572 | 85.859 | 1.136 |
| | | transferring)] kinase isozyme 1, mitochondrial | | | | |
| sp\|Q8BKE6\|CP20A_MOUSE | Cyp20a1 | CP20A_MOUSE Cytochrome P450 20A1 | 8 | 823.396 | 935.332 | 1.136 |
| sp\|P35283\|RAB12_MOUSE | Rab12 | RAB12_MOUSE Ras-related protein Rab-12 | 1 | 63.0901 | 71.6611 | 1.136 |
| sp\|Q810B6\|ANFY1_MOUSE | Ankfy1 | ANFY1_MOUSE Ankyrin repeat and FYVE domain- | 1 | 112.736 | 127.968 | 1.135 |
| | | containing protein 1 | | | | |
| sp\|Q91V04\|TRAM1_MOUSE | Tram1 | TRAM1_MOUSE Translocating chain-associated | 2 | 139.492 | 158.073 | 1.133 |
| | | membrane protein 1 | | | | |
| sp\|Q9QZD8\|DIC_MOUSE | Slc25a10 | DIC_MOUSE Mitochondrial dicarboxylate carrier | 1 | 50.061 | 56.6778 | 1.132 |
| sp\|Q62159\|RHOC_MOUSE | Rhoc | RHOC_MOUSE Rho-related GTP-binding protein RhoC | 1 | 60.9858 | 69.0452 | 1.132 |
| sp\|P35564\|CALX_MOUSE | Canx | CALX_MOUSE Calnexin | 37 | 9066.52 | 10257.4 | 1.131 |
| sp\|Q61207\|SAP_MOUSE | Psap | SAP_MOUSE Sulfated glycoprotein 1 | 2 | 151.129 | 170.977 | 1.131 |
| tr\|A2AG36\|A2AG36_MOUSE | Pigo | A2AG36_MOUSE GPI ethanolamine phosphate | 1 | 38.9223 | 44.0285 | 1.131 |
| | | transferase 3 | | | | |
| sp\|Q9DBG6\|RPN2_MOUSE | Rpn2 | RPN2_MOUSE Dolichyl-diphosphooligosaccliaride- | 29 | 4528.71 | 5121.73 | 1.131 |
| | | protein glycosyltransferase subunit 2 | | | | |
| sp\|Q91WS0\|CISD1_MOUSE | Cisd1 | CISD1_MOUSE CDGSH iron-sulfur domain-containing | 1 | 351.35 | 397.347 | 1.131 |
| | | protein 1 | | | | |
| sp\|Q5I012\|S38AA_MOUSE | Slc38a10 | S38AA_MOUSE Putative sodium-coupled neutral amino | 3 | 282.708 | 319.714 | 1.131 |
| | | acid transporter 10 | | | | |
| sp\|P38647\|GRP75_MOUSE | Hspa9 | GRP75_MOUSE Stress-70 protein, mitochondrial | 64 | 11329.6 | 12810.2 | 1.131 |
| sp\|P15532\|NDKA_MOUSE | Nme1 | NDKA_MOUSE Nucleoside diphosphate kinase A | 39 | 9870.85 | 11160.8 | 1.131 |
| sp\|Q9Z1Q9\|SYVC_MOUSE | Vars | SYVC_MOUSE Valine--tRNA ligase | 1 | 44.3958 | 50.1269 | 1.129 |
| sp\|Q9DlQ4\|DPM3_MOUSE | Dpm3 | DPM3_MOUSE Dolichol-phosphate mannosyl- | 2 | 304.647 | 343.935 | 1.129 |
| | | transferase subunit 3 | | | | |
| sp\|Q9R0P6\|SC11A_MOUSE | Sec11a | SC11A_MOUSE Signal peptidase complex catalytic | 5 | 657.868 | 742.341 | 1.128 |
| | | subunit SEC11A | | | | |
| sp\|P17047\|LAMP2_MOUSE | Lamp2 | LAMP2_MOUSE Lysosome-associated membrane | 2 | 417.519 | 470.637 | 1.127 |
| | | glycoprotein 2 | | | | |
| sp\|Q9CXK9\|RBM33_MOUSE | Rbm33 | RBM33_MOUSE RNA-binding protein 33 | 2 | 123.18 | 138.561 | 1.125 |
| sp\|Q9ESD6\|CKLF7_MOUSE | Cmtm7 | CKLF7_MOUSE CKLF-like MARVEL transmembrane | 1 | 46.5865 | 52.4002 | 1.125 |
| | | domain-containing protein 7 | | | | |
| sp\|P05064\|ALDOA_MOUSE | Aldoa | ALDOA_MOUSE Fructose-bisphosphate aldolase A | 3 | 142.752 | 160.43 | 1.124 |
| sp\|Q3UQ84\|SYTM_MOUSE | Tars2 | SYTM_MOUSE Threonine--tRNA ligase, mitochondrial | 69 | 11933.6 | 13388.3 | 1.122 |
| sp\|Q8VEM8\|MPCP_MOUSE | Slc25a3 | MPCP_MOUSE Phosphate carrier protein, mitochondrial | 8 | 778.131 | 872.578 | 1.121 |
| sp\|Q9DCZ4\|APOO_MOUSE | Apoo | APOO_MOUSE Apolipoprotein O | 1 | 30.7286 | 34.4439 | 1.121 |
| sp\|P35293\|RAB18_MOUSE | Rab18 | RAB18_MOUSE Ras-related protein Rab-18 | 4 | 357.172 | 400.283 | 1.121 |
| sp\|O88986\|KBL_MOUSE | Gcat | KBL_MOUSE 2-amino-3-ketobutyrate coenzyme A ligase, | 5 | 387.814 | 434.516 | 1.120 |
| | | mitochondrial | | | | |
| sp\|Q8K203\|NEIL3_MOUSE | Neil3 | NEIL3_MOUSE Endonuclease 8-like 3 | 1 | 305.623 | 342.15 | 1.120 |
| sp\|Q7TMV3\|FAKD5_MOUSE | Fastkd5 | FAKD5_MOUSE FAST kinase domain-containing protein 5 | 6 | 956.246 | 1070.39 | 1.119 |
| sp\|Q8BKG3\|PTK7_MOUSE | Ptk7 | PTK7_MOUSE Inactive tyrosine-protein kinase 7 | 2 | 108.406 | 121.294 | 1.119 |
| sp\|Q78IS1\|TMED3_MOUSE | Tmed3 | TMED3_MOUSE Transmembrane cmp24 domain- | 1 | 121.926 | 136.316 | 1.118 |
| | | containing protein 3 | | | | |
| sp\|Q7TMK9\|HNRPQ_MOUSE | Syncrip | HNRPQ_MOUSE Heterogeneous nuclear | 2 | 112.492 | 125.705 | 1.117 |
| | | ribonucleoprotein Q | | | | |
| sp\|Q3U2A8\|SYVM_MOUSE | Vars2 | SYVM_MOUSE Valine--tRNA ligase, mitochondrial | 6 | 544.884 | 607.791 | 1.115 |
| sp\|Q8BH97\|RCN3_MOUSE | Rcn3 | RCN3_MOUSE Reticulocalbin-3 | 22 | 2491.69 | 2778.47 | 1.115 |
| sp\|Q8VI93\|OAS3_MOUSE | Oas3 | OAS3_MOUSE 2′-5′-oligoadenylate synthase 3 | 1 | 61.6264 | 68.6598 | 1.114 |
| sp\|Q91VE0\|S27A4_MOUSE | Slc27a4 | S27A4_MOUSE Long-chain fatty acid transport protein 4 | 2 | 127.98 | 142.507 | 1.114 |
| sp\|Q64191\|ASPG_MOUSE | Aga | ASPG_MOUSE N(4)-(beta-N-acetylglucosaminyl)- | 7 | 694.784 | 773.575 | 1.113 |
| | | L-asparaginase | | | | |
| sp\|Q8BK08\|TMM11_MOUSE | Tmem11 | TMM11_MOUSE Transmembrane protein 11, | 3 | 325.466 | 362.371 | 1.113 |
| | | mitochondrial | | | | |
| sp\|Q00899\|TYY1_MOUSE | Yy1 | TYY1_MOUSE Transcriptional repressor protein YY1 | 9 | 1205.97 | 1342.39 | 1.113 |
| sp\|P43406\|ITAV_MOUSE | Itgav | ITAV_MOUSE Integrin alpha-V | 1 | 54.4186 | 60.5738 | 1.113 |
| sp\|P36371\|TAP2_MOUSE | Tap2 | TAP2_MOUSE Antigen peptide transporter 2 | 22 | 2097.14 | 2333.37 | 1.113 |
| sp\|Q8K358\|PIGU_MOUSE | Pigu | PIGU_MOUSE Phosphatidylinositol glycan anchor | 1 | 73.3837 | 81.6282 | 1.112 |
| | | biosynthesis class U protein | | | | |
| sp\|Q3TXS7\|PSMD1_MOUSE | Psmd1 | PSMD1_MOUSE 26S proteasome non-ATPase regulatory | 2 | 62.2717 | 69.2511 | 1.112 |
| | | subunit 1 | | | | |
| sp\|Q9Z2Z6\|MCAT_MOUSE | Slc25a20 | MCAT_MOUSE Mitochondrial carnitine/acylcarnitine | 1 | 156.316 | 173.739 | 1.111 |
| | | carrier protein | | | | |
| sp\|Q9D554\|SF3A3_MOUSE | Slc25a20 | SF3A3_MOUSE Splicing factor 3A subunit 3 | 8 | 820.582 | 911.86 | 1.111 |
| sp\|Q8BIP0\|SYDM_MOUSE | Dars2 | SYDM_MOUSE Aspartate--tRNA ligase, mitochondrial | 6 | 650.527 | 722.499 | 1.111 |
| sp\|Q8K215\|LYRM4_MOUSE | Lyrm4 | LYRM4_MOUSE LYR motif-containing protein 4 | 3 | 1048.92 | 1164.69 | 1.110 |
| sp\|Q60766\|IRGM1_MOUSE | Irgm1 | IRGM1_MOUSE Immunity-related GTPase family | 4 | 422.155 | 468.646 | 1.110 |
| | | M protein 1 | | | | |
| sp\|Q9CXE7\|TMEDS_MOUSE | Tmed5 | TMED5_MOUSE Transmembrane emp24 domain- | 1 | 134.734 | 149.509 | 1.110 |
| | | containing protein 5 | | | | |
| sp\|Q9DCT5\|SDF2_MOUSE | Sdf2 | SDF2_MOUSE Stromal cell-derived factor 2 | 5 | 1253.36 | 1390.21 | 1.109 |
| sp\|Q922E6\|FAKD2_MOUSE | Fastkd2 | FAKD2_MOUSE FAST kinase domain-containing protein 2 | 1 | 50.744 | 56.246 | 1.108 |
| sp\|Q91W90\|TXND5_MOUSE | Txnde5 | TXND5_MOUSE Thioredoxin domain-containing protein 5 | 10 | 2003.6 | 2220.47 | 1.108 |
| sp\|Q640N1\|AEBP1_MOUSE | Aebp1 | AEBP1_MOUSE Adipocyte enhancer-binding protein 1 | 3 | 367.056 | 406.63 | 1.108 |
| sp\|Q7TSQ8\|PDPR_MOUSE | Pdpr | PDPR_MOUSE Pyruvate dehydrogenase phosphatase | 2 | 62.9469 | 69.7201 | 1.108 |
| | | regulatory subunit, mitochondrial | | | | |
| sp\|Q8C522\|ENDD1_MOUSE | Endod1 | ENDD1_MOUSE Endonuclease domain-containing | 1 | 57.7879 | 63.9513 | 1.107 |
| | | 1 protein | | | | |
| sp\|Q9QZE5\|COPG1_MOUSE | Copg1 | COPG1_MOUSE Coatomer subunit gamma-1 | 6 | 572.688 | 633.745 | 1.107 |
| sp\|Q9QUR8\|SEM7A_MOUSE | Sema7a | SEM7A_MOUSE Semaphorin-7A | 1 | 48.33 | 53.4748 | 1.106 |
| sp\|Q8R0S2\|IQEC1_MOUSE | Iqsec1 | IQEC1_MOUSE IQ motif and SEC7 domain-containing | 12 | 2272.6 | 2513.66 | 1.106 |
| | | protein 1 | | | | |
| sp\|Q91V61\|SFXN3_MOUSE | Sfxn3 | SFXN3_MOUSE Sideronexin-3 | 7 | 644.024 | 711.907 | 1.105 |
| sp\|Q8K199\|COXM2_MOUSE | Cme2 | COXM2_MOUSE COX assembly mitochondrial | 2 | 661.315 | 730.807 | 1.105 |
| | | protein 2 homolog | | | | |
| sp\|O70152\|DPM1_MOUSE | Dpm1 | DPM1_MOUSE Dolichol-phosphate | 5 | 420.832 | 465.015 | 1.105 |
| | | mannosyltransferase subunit 1 | | | | |
| sp\|Q80SU7\|GVIN1_MOUSE | Gvin1 | GVIN1_MOUSE Interferon-induced very large GTPase 1 | 5 | 379.374 | 419.079 | 1.105 |
| sp\|P50427\|STS_MOUSE | Sts | STS_MOUSE Steryl-sulfatase | 30 | 3970.67 | 4384.82 | 1.104 |
| sp\|P70398\|USP9X_MOUSE | Usp9x | USP9X_MOUSE Probable ubiquitin carboxyl-terminal | 16 | 997.834 | 1101.52 | 1.104 |
| | | hydrolase FAF-X | | | | |
| sp\|Q6P4S8\|INT1_MOUSE | Ints1 | INTI_MOUSE Integrator complex subunit 1 | 1 | 217.192 | 239.64 | 1.103 |
| sp\|Q9CQN7\|RM41_MOUSE | Mrp141 | RM41_MOUSE 39S ribosomal protein L41, mitochondrial | 3 | 804.585 | 886.554 | 1.102 |
| sp\|Q99NB9\|SF3B1_MOUSE | Sf3b1 | SF3B1_MOUSE Splicing factor 3B subunit 1 | 26 | 4549.92 | 5012.67 | 1.102 |
| sp\|Q3URS9\|CCD51_MOUSE | Ccdc51 | CCD51_MOUSE Coiled-coil domain-containing protein 51 | 4 | 436.156 | 480.44 | 1.102 |
| sp\|Q9D8Yl\|T126A_MOUSE | Tmem | T126A_MOUSE Transmembrane protein 126A | 1 | 99.4216 | 109.483 | 1.101 |
| | 126a | | | | | |
| sp\|P62983\|RS27A_MOUSE | Rps27a | RS27A_MOUSE Ubiquitin-40S ribosomal protein S27a | 47 | 9629.85 | 10589.5 | 1.100 |
| sp\|P83870\|PHE5A_MOUSE | Phf5a | PHF5A_MOUSE PHD finger-like domain-containing | 3 | 487.769 | 536.285 | 1.099 |
| | | protein 5A | | | | |
| sp\|Q8BHC4\|DCAKD_MOUSE | Dcakd | DCAKD_MOUSE Dcphospho-CoA kinase domain- | 2 | 153.557 | 168.829 | 1.099 |
| | | containing protein | | | | |
| sp\|Q91W2\|ATPG_MOUSE | Atp5c1 | ATPG_MOUSE ATP synthase subunit gamma, | 6 | 1059.59 | 1164.7 | 1.099 |
| | | mitochondrial | | | | |
| sp\|O55022\|PGRC1_MOUSE | Pgrmc1 | PGRC1_MOUSE Membrane-associated progesterone | 6 | 641.278 | 704.698 | 1.099 |
| | | receptor component 1 | | | | |
| sp\|Q99PM3\|TF2AA_MOUSE | Gtf2a1 | TF2AA_MOUSE Transcription initiation factor IIA | 5 | 1627.02 | 1787.44 | 1.099 |
| | | subunit 1 | | | | |
| sp\|P17742\|PPIA_MOUSE | Ppia | PPIA_MOUSE Peptidyl-prolyl cis-trans isomerase A | 13 | 2342.03 | 2572.38 | 1.098 |
| sp\|Q3U3R4\|LMF1_MOUSE | Lmf1 | LMF1_MOUSE Lipase maturation factor 1 | 1 | 62.1717 | 68.2776 | 1.098 |
| sp\|Q5SWU9\|ACACA_MOUSE | Acaca | ACACA_MOUSE Acetyl-CoA carboxylase 1 | 1 | 139.391 | 153.022 | 1.098 |
| sp\|Q9D6I9\|LURA1_MOUSE | Lurap1 | LURA1_MOUSE Leucine rich adaptor protein 1 | 1 | 96.3602 | 105.72 | 1.097 |
| sp\|Q5EG47\|AAPK1_MOUSE | Prkaa1 | AAPK1_MOUSE 5′-AMP-activated protein kinase catalytic | 7 | 571.023 | 626.341 | 1.097 |
| | | subunit alpha-1 | | | | |
| sp\|Q9JIG8\|PRAF2_MOUSE | Praf2 | PRAF2_MOUSE PRA1 family protein 2 | 2 | 281.818 | 308.718 | 1.095 |
| sp\|P21958\|TAP1_MOUSE | Tap1 | TAP1_MOUSE Antigen peptide transporter 1 | 21 | 2228.64 | 2440.98 | 1.095 |
| sp\|Q8BGV0\|SYNM_MOUSE | Nars2 | SYNM_MOUSE Probable asparagine--tRNA ligase, | 1 | 35.5726 | 38.8607 | 1.092 |
| | | mitochondrial | | | | |
| tr\|M0QWP1\|M0QWP1_MOUSE | Agrn | M0QWP1_MOUSE Agrin | 39 | 4005.33 | 4374.89 | 1.092 |
| sp\|Q6DI86\|FAKD1_MOUSE | Fastkd1 | FAKD1_MOUSE FAST kinase domain-containing protein 1 | 1 | 77.2246 | 84.3038 | 1.092 |
| sp\|P59708\|SF3B6_MOUSE | Sf3b6 | SF3B6_MOUSE Splicing factor 3B subunit 6 | 1 | 103.581 | 113.058 | 1.091 |
| sp\|P17751\|TPIS_MOUSE | Tpi1 | TPIS_MOUSE Triosephosphate isomerase | 5 | 435.671 | 475.504 | 1.091 |
| sp\|Q8BRK9\|MA2A2_MOUSE | Man2a2 | MA2A2_MOUSE Alpha-mannosidase 2x | 1 | 113.121 | 123.458 | 1.091 |
| tr\|G5E8J0\|G5E8J0_MOUSE | Noteh2 | G5E8J0_MOUSE Neurogenic locus notch homolog protein 2 | 6 | 434.001 | 473.628 | 1.091 |
| sp\|Q8R0F3\|SUMF1_MOUSE | Sumf1 | SUMF1_MOUSE Sulfatase-modifying factor 1 | 3 | 278.441 | 303.796 | 1.091 |
| sp\|Q9WTK3\|GPAA1_MOUSE | Gpaa1 | GPAA1_MOUSE Glycosylphosphatidylinositol anchor | 6 | 483.065 | 527.014 | 1.091 |
| | | attachment 1 protein | | | | |
| sp\|Q9CZU3\|SK2L2_MOUSE | Skiv2l2 | SK2L2_MOUSE Superkiller viralicidic activity 2-like 2 | 2 | 70.0752 | 76.3903 | 1.090 |
| sp\|O08749\|DLDH_MOUSE | Dld | DLDH_MOUSE Dihydrolipoyl dehydrogenase, | 8 | 1519.75 | 1655.08 | 1.089 |
| | | mitochondrial | | | | |
| sp\|P53994\|RAB2A_MOUSE | Rab2a | RAB2A_MOUSE Ras-rclated protein Rab-2A | 10 | 1977.93 | 2149.02 | 1.086 |
| sp\|P27773\|PDIA3_MOUSE | Pdia3 | PDIA3_MOUSE Protein disulfide-isomerase A3 | 81 | 12003.1 | 13020.3 | 1.085 |
| sp\|F8VPU2\|FARP1_MOUSE | Farp1 | FARP1_MOUSE FERM, RhoGEF and pleckstrin domain- | 60 | 7838.7 | 8475.56 | 1.081 |
| | | containing protein 1 | | | | |
| sp\|Q09XV5\|CHD8_MOUSE | Chd8 | CHD8_MOUSE Chromodomain-helicase-DNA-binding | 9 | 1154.43 | 1248.1 | 1.081 |
| | | protein 8 | | | | |
| sp\|P61963\|DCAF7_MOUSE | Dcaf7 | DCAF7_MOUSE DDB1-and CUL4-associated factor 7 | 1 | 32.8058 | 35.4654 | 1.081 |
| sp\|Q9CQS3\|FIBIN_MOUSE | Fibin | FIBIN_MOUSE Fin bud initiation factor homolog | 2 | 118.495 | 127.975 | 1.080 |
| sp\|Q91V41\|RAB14_MOUSE | Rab14 | RAB14_MOUSE Ras-related protein Rab-14 | 8 | 1666.47 | 1799.47 | 1.080 |
| sp\|P40142\|TKT_MOUSE | Tkt | TKT_MOUSE Transketolase | 1 | 69.9087 | 75.386 | 1.078 |
| sp\|Q9DB25\|ALG5_MOUSE | Alg5 | ALG5_MOUSE Dolichyl-phosphate beta- | 3 | 658.615 | 710.204 | 1.078 |
| | | glucosyltransferase | | | | |
| tr\|E9Q0B6\|E9Q0B6_MOUSE | Dnah6 | E9Q0B6_MOUSE Protein Dnah6 | 1 | 187.049 | 201.491 | 1.077 |
| tr\|E9QLA5\|E9QLAS_MOUSE | Inf2 | E9QLA5_MOUSE Inverted formin-2 | 10 | 1094.61 | 1178.5 | 1.077 |
| sp\|Q6PDG5\|SMRC2_MOUSE | Smarcc2 | SMRC2_MOUSE SWI/SNF complex subunit SMARCC2 | 3 | 341.423 | 367.495 | 1.076 |
| sp\|Q9R0M0\|CELR2_MOUSE | Celsr2 | CELR2_MOUSE Cadherin EGF LAG seven-pass G-type | 1 | 167.035 | 179.778 | 1.076 |
| | | receptor 2 | | | | |
| sp\|B7ZMP1\|XPP3_MOUSE | Xpnep3 | XPP3_MOUSE Probable Xaa-Pro aminopeptidase 3 | 3 | 200.514 | 215.806 | 1.076 |
| sp\|Q6PFR5\|TRA2A_MOUSE | Tra2a | TRA2A_MOUSE Transformer-2 protein homolog alpha | 2 | 215.451 | 231.826 | 1.076 |
| sp\|P62806\|H4_MOUSE | Hist1h4a | H4_MOUSE Histone H4 | 9 | 1217.65 | 1309.96 | 1.076 |
| sp\|Q80SZ7\|GBG5_MOUSE | Gng5 | GBG5_MOUSE Guanine nucleotide-binding protein | 1 | 183.111 | 196.96 | 1.076 |
| | | G(I)/G(S)/G(O) subunit gamma-5 | | | | |
| sp\|P35282\|RAB21_MOUSE | Rab21 | RAB21_MOUSE Ras-related protein Rab-21 | 3 | 367.73 | 395.498 | 1.076 |
| sp\|Q9D1I6\|RM14_MOUSE | Mrpl14 | RM14_MOUSE 39S ribosomal protein L14, mitochondrial | 4 | 313.787 | 337.208 | 1.075 |
| sp\|Q9WU42\|NCOR2_MOUSE | Ncor2 | NCOR2_MOUSE Nuclear receptor corepressor 2 | 2 | 219.692 | 235.944 | 1.074 |
| sp\|P12265\|BGLR_MOUSE | Gusb | BGLR_MOUSE Beta-glucuronidase | 6 | 837.438 | 899.278 | 1.074 |
| sp\|Q99LS3\|SERB_MOUSE | Psph | SERB_MOUSE Phosphoserine phosphatase | 5 | 584.03 | 626.979 | 1.074 |
| sp\|P05213\|TBA1B_MOUSE | Tuba1b | TBA1B_MOUSE Tubulin alpha-1B chain | 6 | 856.107 | 918.866 | 1.073 |
| sp\|Q60994\|ADIPO_MOUSE | Adipoq | ADIPO_MOUSE Adiponectin | 1 | 30.2393 | 32.4557 | 1.073 |
| sp\|P05622\|PGFRB_MOUSE | Pdgfrb | PGFRB_MOUSE Platelet-derived growth factor | 13 | 2072.53 | 2219.1 | 1.071 |
| | | receptor beta | | | | |
| sp\|Q99JX3\|GORS2_MOUSE | Gorasp2 | GORS2_MOUSE Golgi reassembly-stacking protein 2 | 1 | 32.4969 | 34.7429 | 1.069 |
| sp\|P16110\|LEG3_MOUSE | Lgals3 | LEG3_MOUSE Galectin-3 | 3 | 654.441 | 699.667 | 1.069 |
| sp\|Q9QY76\|VAPB_MOUSE | Vapb | VAPB_MOUSE Vesicle-associated membrane protein- | 2 | 509.422 | 544.035 | 1.068 |
| | | associated protein B | | | | |
| sp\|Q9D404\|OXSM_MOUSE | Oxsm | OXSM_MOUSE 3-oxoacyl-[acyl-carrier-protein] synthase, | 3 | 445.364 | 475.339 | 1.067 |
| | | mitochondrial | | | | |
| sp\|Q8K009\|AL1L2_MOUSE | Aldh1l2 | AL1L2_MOUSE Mitochondrial 10-formyltetrahydrofolate | 1 | 110.96 | 118.374 | 1.067 |
| | | dehydrogenase | | | | |
| sp\|Q8VD31\|TPSNR_MOUSE | Tapbpl | TPSNR_MOUSE Tapasin-related protein | 2 | 226.665 | 241.744 | 1.067 |
| sp\|P62774\|MTPN_MOUSE | Mtpn | MTPN_MOUSE Myotrophin | 1 | 99.7197 | 106.326 | 1.066 |
| sp\|Q9CQE1\|NPS3B_MOUSE | Nipsnap3b | NPS3B_MOUSE Protein NipSnap homolog 3B | 3 | 434.458 | 463.193 | 1.066 |
| sp\|Q02819\|NUCB1_MOUSE | Nucb1 | NUCB1_MOUSE Nuclcobindin-1 | 6 | 476.968 | 508.132 | 1.065 |
| sp\|Q9WV86\|KTNA1_MOUSE | Katna1 | KTNA1_MOUSE Katanin p60 ATPase-containing subunit A1 | 4 | 512.359 | 545.553 | 1.065 |
| sp\|Q9D8L5\|CCD91_MOUSE | Ccde91 | CCD91_MOUSE Coiled-coil domain-containing protein 91 | 4 | 507.555 | 540.084 | 1.064 |
| sp\|Q8R127\|SCPDL_MOUSE | Sccpdh | SCPDL_MOUSE Saccharopine dehydrogenase-like | 1 | 51.4619 | 54.7553 | 1.064 |
| | | oxido reductase | | | | |
| sp\|Q8VCL2\|SCO2_MOUSE | Sco2 | SCO2_MOUSE Protein SCO2 homolog, mitochondrial | 1 | 54.1642 | 57.6174 | 1.064 |
| sp\|A2ASS6\|TITIN_MOUSE | Ttn | TITIN_MOUSE Titin | 1 | 121.424 | 128.979 | 1.062 |
| sp\|O08547\|SC22B_MOUSE | Sec22b | SC22B_MOUSE Vesicle-trafficking protein SEC22b | 7 | 1632.78 | 1730.24 | 1.060 |
| sp\|Q9CPT4\|CS010MOUSE | D17W | CS010_MOUSE UPF0556 protein C19orfl0 homolog | 3 | 662.8 | 701.932 | 1.059 |
| | sul04e | | | | | |
| sp\|Q9ES97\|RTN3_MOUSE | Rtn3 | RTN3_MOUSE Reticulon-3 | 1 | 197.621 | 209.18 | 1.058 |
| sp\|P62835\|RAP1A_MOUSE | Rap1a | RAP1A_MOUSE Ras-related protein Rap-1A | 3 | 896.887 | 948.204 | 1.057 |
| sp\|Q8C129\|LCAP_MOUSE | Lnpep | LCAP_MOUSE Leucyl-cystinyl aminopeptidase | 2 | 119.239 | 125.937 | 1.056 |
| sp\|Q64008\|RA34_MOUSE | Rab34 | RAB34_MOUSE Ras-related protein Rab-34 | 3 | 315.486 | 333.142 | 1.056 |
| sp\|Q9R112\|SQRD_MOUSE | Sqrdl | SQRD_MOUSE Sulfide:quinone oxidoreductase, | 3 | 246.447 | 259.886 | 1.055 |
| | | mitochondrial | | | | |
| sp\|P01901\|HAIB_MOUSE | H2-K1 | HA1B_MOUSE H-2 class I histocompatibility antigen, | 4 | 297.442 | 313.652 | 1.054 |
| | | K-B alpha chain | | | | |
| sp\|Q91YJ5\|IF2M_MOUSE | Mtif2 | IF2M_MOUSE Translation initiation factor IF-2, | 2 | 140.219 | 147.664 | 1.053 |
| | | mitochondrial | | | | |
| sp\|Q6P2K6\|P4R3A_MOUSE | Smek1 | P4R3A_MOUSE Serine/threonine-protein phosphatase 4 | 1 | 152.952 | 161.067 | 1.053 |
| | | regulators subunit 3A | | | | |
| sp\|Q9ER38\|TOR3A_MOUSE | Tor3a | TOR3A_MOUSE Torsin-3A | 1 | 44.7951 | 47.1654 | 1.053 |
| sp\|Q9EQ06\|DHB11_MOUSE | Hsd17b11 | DHB11_MOUSE Estradiol 17-beta-dehydrogenase 11 | 4 | 318.71 | 335.167 | 1.052 |
| sp\|Q8VHX6\|FLNC_MOUSE | Flnc | FLNC_MOUSE Filamin-C | 7 | 552.044 | 580.016 | 1.051 |
| sp\|P03930\|ATP8_MOUSE | Mtat8 | ATP8_MOUSE ATP synthase protein 8 | 1 | 114.114 | 119.882 | 1.051 |
| sp\|Q99L43\|CDS2_MOUSE | Cds2 | CDS2_MOUSE Phosphatidate cytidylyltransferase 2 | 1 | 56.533 | 59.3162 | 1.049 |
| tr\|E9Q7L0\|E9Q7L0_MOUSE | Ogdh1 | E9Q7L0_MOUSE Protein Ogdhl | 1 | 59.775 | 62.6994 | 1.049 |
| sp\|Q924Z4\|CERS2_MOUSE | Cers2 | CERS2_MOUSE Ceramide synthase 2 | 4 | 516.749 | 541.977 | 1.049 |
| sp\|F6ZDS4\|TPR_MOUSE | Tpr | TPR_MOUSE Nucleoprotein TPR | 1 | 35.7834 | 37.5039 | 1.048 |
| sp\|Q8CG76\|AK72_MOUSE | Akr7a2 | ARK72_MOUSE Aflatoxin B1 aldehyde reductase member 2 | 2 | 139.923 | 146.59 | 1.048 |
| sp\|Q8BK62\|OLFL3_MOUSE | Olfml3 | OLFL3_MOUSE Olfactomedin-like protein 3 | 4 | 276.99 | 290.003 | 1.047 |
| sp\|Q9RlB9\|SLIT2_MOUSE | Slit2 | SLIT2_MOUSE Slit homolog 2 protein | 1 | 248.246 | 259.694 | 1.046 |
| sp\|O88632\|SEM3F_MOUSE | Sema3f | SEM3F_MOUSE Semaphorin-3F | 3 | 383.392 | 401.068 | 1.046 |
| sp\|Q921M3\|SF3B3_MOUSE | Sf3b3 | SF3B3_MOUSE Splicing factor 3B subunit 3 | 24 | 4296.57 | 4493.51 | 1.046 |
| sp\|Q8BWR2\|PITH1_MOUSE | Pithd1 | PITH1_MOUSE PITH domain-containing protein 1 | 4 | 238.527 | 249.11 | 1.044 |
| sp\|P35278\|RAB5C_MOUSE | Rab5c | RAB5C_MOUSE Ras-related protein Rab-5C | 8 | 1246.6 | 1299.38 | 1.042 |
| sp\|P14211\|CALR_MOUSE | Calr | CALR_MOUSE Calreticulin | 16 | 3991.22 | 4153.83 | 1.04! |
| sp\|Q8R180\|ERO1A_MOUSE | Ero1l | ERO1A_MOUSE ERO1-like protein alpha | 36 | 4343.84 | 4517.8 | 1.040 |
| sp\|Q03265\|ATA_MOUSE | Atp5a1 | ATPA_MOUSE ATP synthase subunit alpha, mitochondrial | 17 | 3006.34 | 3121.68 | 1.038 |
| sp\|Q99J47\|DRS7B_MOUSE | Dhrs7b | DRS7B_MOUSE Dehydrogenase/reductase SDR family | 3 | 296.729 | 307.542 | 1.036 |
| | | member 7B | | | | |
| sp\|Q80UU9\|PGRC2_MOUSE | Pgrme2 | PGRC2_MOUSE Membrane-associated progesterone | 13 | 1340.35 | 1387.95 | 1.036 |
| | | receptor component 2 | | | | |
| sp\|Q99J93\|IFM2_MOUSE | Ifitm2 | IFM2_MOUSE Interferon-induced transmembrane protein 2 | 5 | 670.949 | 694.605 | 1.035 |
| tr\|A2AQ53\|A2A053_MOUSE | Fbn1 | A2AQ53_MOUSE Fibrillin-1 | 8 | 671.314 | 694.897 | 1.035 |
| sp\|Q6P4T2\|U520_MOUSE | Snrnp200 | U520_MOUSE U5 small nuclear ribonucleoprotein 200 kDa | 4 | 273.11 | 282.661 | 1.035 |
| | | helicase | | | | |
| sp\|Q8BUY5\|TIDC1_MOUSE | Timmde1 | TIDC1_MOUSE Complex I assembly factor TIMMDC1, | 1 | 70.9877 | 73.4023 | 1.034 |
| | | mitochondrial | | | | |
| sp\|Ql4A16\|RUSD3_MOUSE | Rpusd3 | RUSD3_MOUSE RNA pseudouridylate synthase domain- | 1 | 140.191 | 144.946 | 1.034 |
| | | containing protein 3 | | | | |
| sp\|Q9D1Q6\|ERP44_MOUSE | Erp44 | ERP44_MOUSE Endoplasmic reticulum resident protein 44 | 73 | 15816.3 | 16339.1 | 1.033 |
| sp\|Q99KY4\|GAK_MOUSE | Gak | GAK_MOUSE Cyclin-G-associated kinase | 2 | 152.563 | 157.444 | 1.032 |
| sp\|Q8CIT9\|SBSN_MOUSE | Sbsn | SBSN_MOUSE Suprabasin | 3 | 370.967 | 382.454 | 1.031 |
| sp\|Q99JR5\|TINAL_MOUSE | Tinagl1 | TINAL_MOUSE Tubulointerstitial nephritis antigen-like | 1 | 128.82 | 132.74 | 1.030 |
| sp\|P59481\|LMA2L_MOUSE | Lman2l | LMA2L_MOUSE VIP36-like protein | 1 | 73.3098 | 75.5355 | 1.030 |
| sp\|Q9D1B9\|RM28_MOUSE | Mrpl28 | RM28_MOUSE 39S ribosomal protein L28, mitochondrial | 2 | 232.95 | 239.99 | 1.030 |
| sp\|Q61127\|NAB2_MOUSE | Nab2 | NAB2_MOUSE NGFI-A-binding protein 2 | 1 | 42.7683 | 44.0338 | 1.030 |
| sp\|P62071\|RRAS2_MOUSE | Rras2 | RRAS2_MOUSE Ras-related protein R-Ras2 | 2 | 127.598 | 131.327 | 1.029 |
| sp\|Q61578\|ADRO_MOUSE | Fdxr | ADRO_MOUSE NADPH:adrenodoxin oxidoreductase, | 14 | 2467.52 | 2536.58 | 1.028 |
| | | mitochondrial | | | | |
| sp\|Q99MN1\|SYK_MOUSE | Kars | SYK_MOUSE Lysine--tRNA ligase | 6 | 613.523 | 630.485 | 1.028 |
| sp\|Q3UPL0\|SC31A_MOUSE | Scc31a | SC31A_MOUSE Protein transport protein Sec31A | 2 | 119.718 | 123.016 | 1.028 |
| sp\|O55242\|SGMR1_MOUSE | Sigmar1 | SGMR1_MOUSE Sigma non-opioid intracellular receptor 1 | 2 | 543.476 | 557.576 | 1.026 |
| sp\|Q9CR13\|CG055_MOUSE | | CG055_MOUSE UPF0562 protein C7orf55 homolog | 1 | 206.146 | 211.121 | 1.024 |
| sp\|P01899\|HA11_MOUSE | H2-D1 | HA11_MOUSE H-2 class I histocompatibility antigen, | 11 | 1133.78 | 1160.92 | 1.024 |
| | | D-B alpha chain | | | | |
| sp\|P10126\|EF1A1_MOUSE | Eef1a1 | EF1A1_MOUSE Elongation factor 1-alpha 1 | 7 | 758.488 | 776.453 | 1.024 |
| tr\|E9Q3M9\|E9Q3M9_MOUSE | 2010 300C | E9Q3M9_MOUSE Protein 2010300C02Rik | 2 | 291.671 | 298.416 | 1.023 |
| | 02Rik | | | | | |
| sp\|P62311\|LSM3_MOUSE | Lsm3 | LSM3_MOUSE U6 snRNA-associated Sm-like protein | 1 | 43.9648 | 44.9766 | 1.023 |
| | | LSm3 | | | | |
| sp\|Q9D1K7\|CT027_MOUSE | | CT027_MOUSE UPF0687 protein C20orf27 homolog | 1 | 129.189 | 132.156 | 1.023 |
| sp\|Q06185\|ATP5I_MOUSE | Atp5i | ATP5I_MOUSE ATP synthase subunite, mitochondrial | 1 | 51.9718 | 53.1155 | 1.022 |
| sp\|O35682\|MYDM_MOUSE | Myadm | MYADM_MOUSE Myeloid-associated differentiation | 1 | 97.7058 | 99.7049 | 1.020 |
| | | marker | | | | |
| sp\|Q91XL3\|UXS1_MOUSE | Uxs1 | UXS1_MOUSE UDP-glucuronic acid decarboxylase 1 | 1 | 230.091 | 234.746 | 1.020 |
| sp\|P61027\|RAB10_MOUSE | Rab10 | RAB10_MOUSE Ras-related protein Rab-10 | 4 | 619.995 | 632.259 | 1.020 |
| sp\|Q8BWT1\|THIM_MOUSE | Acaa2 | THIM_MOUSE 3-ketoacyl-CoA thiolase, mitochondrial | 1 | 69.585 | 70.9337 | 1.019 |
| sp\|Q91ZW2\|OFUT1_MOUSE | Pofut1 | OFUT1_MOUSE GDP-fucose protein O-fucosyltransferase 1 | 1 | 112.381 | 114.475 | 1.019 |
| sp\|P14206\|RSSA_MOUSE | Rpsa | RSSA_MOUSE 40S ribosomal protein SA | 5 | 371.192 | 377.83 | 1.018 |
| sp\|P28301\|LYOX_MOUSE | Lox | LYOX_MOUSE Protein-lysine 6-oxidase | 26 | 3069.07 | 3122.75 | 1.017 |
| sp\|Q9CQF9\|PCYOX_MOUSE | Pcyox1 | PCYOX_MOUSE Prenylcysteine oxidase | 4 | 498.987 | 507.085 | 1.016 |
| sp\|Q9WUM5\|SUCA_MOUSE | Suclg1 | SUCA_MOUSE Succinyl-CoA ligase [ADP/GDP-forming] | 8 | 1880.63 | 1908.56 | 1.015 |
| | | subunit alpha, mitochondrial | | | | |
| sp\|Q69ZP3-2\|PNKD_MOUSE | Pnkd | PNKD_MOUSE Isoform 2 of Probable hydrolase PNKD | 2 | 205.09 | 207.934 | 1.014 |
| sp\|Q78IK2\|USMG5_MOUSE | Usmg5 | USMG5_MOUSE Up-regulated during skeletal muscle | 1 | 72.4926 | 73.4812 | 1.014 |
| | | growth protein 5 | | | | |
| sp\|Q9D7J9\|ECHD3_MOUSE | Echdc3 | ECHD3_MOUSE Enoyl-CoA hydratase domain-containing | 1 | 402.256 | 407.676 | 1.013 |
| | | protein 3, mitochondrial | | | | |
| sp\|Q62179\|SEM4B_MOUSE | Sema4b | SEM4B_MOUSE Semaphorin-4B | 1 | 61.6067 | 62.4332 | 1.013 |
| sp\|Q99PU8-3\|DHX30_MOUSE | Dhx30 | DHX30_MOUSE Isoform 3 of Putative ATP-dependent | 37 | 5940.11 | 6011.96 | 1.012 |
| | | RNA helicase DHX30 | | | | |
| sp\|P70193\|LRIG1_MOUSE | Lrig1 | LRIG1_MOUSE Leucine-rich repeats and immunoglobulin- | 5 | 656.185 | 663.783 | 1.012 |
| | | like domains protein 1 | | | | |
| tr\|E9PWQ3\|E9PWO3_MOUSE | Col6a3 | E9PWQ3_MOUSE Protein Col6a3 | 38 | 4656.95 | 4710.64 | 1.012 |
| sp\|Q61147\|CERU_MOUSE | Cp | CERU_MOUSE Ceruloplasmin | 19 | 1755.01 | 1775.16 | 1.011 |
| sp\|Q9JHI7\|EXOS9_MOUSE | Exosc9 | EXOS9_MOUSE Exosome complex component RRP45 | 1 | 33.4173 | 33.8005 | 1.011 |
| sp\|Q80TN7\|NAV3_MOUSE | Nav3 | NAV3_MOUSE Neuron navigator 3 | 5 | 2151.48 | 2175.69 | 1.011 |
| sp\|Q3UW53\|NIBAN_MOUSE | Fam129a | NIBAN_MOUSE Protein Niban | 7 | 563.894 | 570.113 | 1.011 |
| sp\|P62270\|RS18_MOUSE | Rps18 | RS18_MOUSE 40S ribosomal protein S18 | 9 | 1250.89 | 1264.24 | 1.011 |
| tr\|G3X972\|G3X972_MOUSE | Sec24c | G3X972_MOUSE Protein Sec24c | 5 | 357.951 | 361.637 | 1.010 |
| sp\|Q14CH7\|SYAM_MOUSE | Aars2 | SYAM_MOUSE Alanine--tRNA ligase, mitochondrial | 3 | 156.172 | 157.624 | 1.009 |
| sp\|P70206\|PLXA1_MOUSE | Plxna1 | PLXA1_MOUSE Plexin-A1 | 24 | 2283.64 | 2303.69 | 1.009 |
| sp\|Q9CZP5\|BCS1_MOUSE | Bcs1l | BCS1_MOUSE Mitochondrial chaperone BCS1 | 5 | 1587.82 | 1601.43 | 1.009 |
| sp\|Q9CQJ8\|NDUB9_MOUSE | Ndufb9 | NDUB9_MOUSE NADH dehydrogenase [ubiquinone] 1 | 19 | 3023.96 | 3049.05 | 1.008 |
| | | beta subcomplex subunit 9 | | | | |
| tr\|D3YVW2\|D3YVW2_MOUSE | Golim4 | D3YVW2_MOUSE Golgi integral membrane protein 4 | 70 | 10772.8 | 10861.5 | 1.008 |
| sp\|B1AR13\|CISD3_MOUSE | Cisd3 | CISD3_MOUSE CDGSH iron-sulfur domain-containing | 5 | 1868.28 | 1882.21 | 1.007 |
| | | protein 3, mitochondrial | | | | |
| sp\|Q921L3\|TMCO1_MOUSE | Tmco1 | TMCO1_MOUSE Transmembrane and coiled-coil | 2 | 88.3051 | 88.9579 | 1.007 |
| | | domain-containing protein 1 | | | | |
| sp\|P09103\|PDIA1_MOUSE | P4hb | PDIA1_MOUSE Protein disulfide-isomerase | 90 | 16288 | 16403.9 | 1.007 |
| sp\|Q501Pl\|FBLN7_MOUSE | Fbln7 | FBLN7_MOUSE Fibulin-7 | 2 | 310.985 | 313.041 | 1.007 |
| sp\|Q8BYB9\|PGLT1_MOUSE | Poglut1 | PGLT1_MOUSE Protein O-glucosyltransferase 1 | 1 | 120.933 | 121.672 | 1.006 |
| sp\|Q8BXQ2\|PIGT_MOUSE | Pigt | PIGT_MOUSE GPI transamidase component PIG-T | 8 | 1594.68 | 1601.47 | 1.004 |
| sp\|Q6PA06\|ATLA2_MOUSE | Atl2 | ATLA2_MOUSE Atlastin-2 | 1 | 31.2987 | 31.4074 | 1.003 |
| sp\|Q8K248\|HPDL_MOUSE | Hpdl | HPDL_MOUSE 4-hydroxyphenylpyruvate dioxygenase-like | 5 | 445.667 | 447.059 | 1.003 |
| | | protein | | | | |
| sp\|Q8K3J1\|NDUS8_MOUSE | Ndufs8 | NDUS8_MOUSE NADH dehydrogenase [ubiquinone] iron- | 13 | 2282.19 | 2289.18 | 1.003 |
| | | sulfur protein 8, mitochondrial | | | | |
| sp\|P38060\|HMGCL_MOUSE | Hmgc1 | HMGCL_MOUSE Hydroxymethylglutaryl-CoA lyase, | 3 | 343.231 | 344.281 | 1.003 |
| | | mitochondrial | | | | |
| sp\|P17809\|GTRl_MOUSE | Slc2a1 | GTR1_MOUSE Solute carrier family 2, facilitated glucose | 2 | 277.355 | 278.092 | 1.003 |
| | | transporter member 1 | | | | |
| sp\|P18572\|BASI_MOUSE | Bsg | BASI_MOUSE Basigin | 1 | 32.5389 | 32.6241 | 1.003 |
| sp\|O88322\|NID2_MOUSE | Nid2 | NID2_MOUSE Nidogen-2 | 10 | 1086.44 | 1088.96 | 1.002 |
| sp\|Q9DCN2\|NB5R3_MOUSE | Cyb5r3 | NB5R3_MOUSE NADH-cytochrome b5 reductase 3 | 8 | 1781.57 | 1785.64 | 1.002 |
| sp\|Q9D924\|ISCA1_MOUSE | Isca1 | ISCA1_MOUSE Iron-sulfur cluster assembly 1 homolog, | 2 | 155.686 | 155.917 | 1.001 |
| | | mitochondrial | | | | |
| sp\|P03903\|NU4LM_MOUSE | Mtnd4l | NU4LM_MOUSE NADH-ubiquinone oxidoreductase | 2 | 209.285 | 209.43 | 1.001 |
| | | chain 4L | | | | |
| sp\|Q923D4\|SF3B5_MOUSE | Sf3b5 | SF3B5_MOUSE Splicing factor 3B subunit 5 | 1 | 98.291 | 98.065 | 0.998 |
| sp\|P61982\|1433G_MOUSE | Ywhag | 1433G_MOUSE 14-3-3 protein gamma | 5 | 1243.49 | 1239.69 | 0.997 |
| sp\|E9PZM4\|CHD2_MOUSE | Chd2 | CHD2_MOUSE Chromodomain-helicase-DNA-binding | 2 | 176.696 | 176.083 | 0.997 |
| | | protein 2 | | | | |
| sp\|P63276\|RS17_MOUSE | Rps17 | RS17_MOUSE 40S ribosomal protein S17 | 1 | 147.354 | 146.833 | 0.996 |
| sp\|Q8R5A6\|TB2A_MOUSE | Tbc1d22a | TB22A_MOUSE TBC1 domain family member 22A | 2 | 96.7152 | 96.3283 | 0.996 |
| tr\|Q1AN92\|Q1AN92_MOUSE | Gm5150 | Q1AN92_MOUSE Protein Gm5150 | 1 | 39.0905 | 38.9156 | 0.996 |
| sp\|Q9CQ65\|MTAP_MOUSE | Mtap | MTAP_MOUSE S-methyl-5′-thioadenosine phosphorylase | 4 | 429.417 | 427.229 | 0.995 |
| tr\|Q6XPS7\|Q6XPS7_MOUSE | Tha1 | Q6XPS7_MOUSE L-threonine aldolase | 1 | 189.93 | 188.923 | 0.995 |
| sp\|Q9DBV4\|MXRA8_MOUSE | Mxra8 | MXRA8_MOUSE Matrix-remodeling-associated protein 8 | 63 | 11357.4 | 11295.9 | 0.995 |
| sp\|Q9QZ23\|NFU1_MOUSE | Nfu1 | NFU1_MOUSE NFU1 iron-sulfur cluster scaffold homolog, | 2 | 204.588 | 203.31 | 0.994 |
| | | mitochondrial | | | | |
| sp\|Q9D0F3\|LMAN1_MOUSE | Lman1 | LMAN1_MOUSE Protein ERGIC-53 | 168 | 29181.7 | 28983.1 | 0.993 |
| sp\|Q80X90\|FLNB_MOUSE | Flnb | FLNB_MOUSE Filamin-B | 6 | 565.781 | 561.438 | 0.992 |
| sp\|B2RWS6\|EP300_MOUSE | Ep300 | EP300_MOUSE Histone acetyltransferase p300 | 5 | 685.87 | 679.853 | 0.991 |
| sp\|P17918\|PCNA_MOUSE | Pcna | PCNA_MOUSE Proliferating cell nuclear antigen | 3 | 359.187 | 356.016 | 0.991 |
| sp\|Q8C5H8\|NAKD2_MOUSE | Nadk2 | NAKD2_MOUSE NAD kinase 2, mitochondrial | 24 | 3085.19 | 3056.11 | 0.991 |
| sp\|Q99KI0\|ACON_MOUSE | Aco2 | ACON_MOUSE Aconitate hydratase, mitochondrial | 25 | 3456.2 | 3421.78 | 0.990 |
| sp\|Q9CR21\|ACPM_MOUSE | Ndufab1 | ACPM_MOUSE Acyl earner protein, mitochondrial | 6 | 1420.88 | 1403.95 | 0.988 |
| sp\|Q9JKR6\|HYOU1_MOUSE | Hyou1 | HYOU1_MOUSE Hypoxia up-regulated protein 1 | 26 | 4536.67 | 4482.09 | 0.988 |
| sp\|P47968\|RPIA_MOUSE | Rpia | RPIA_MOUSE Ribose-5-phosphate isomerase | 5 | 656.423 | 648.092 | 0.987 |
| sp\|Q99JY0\|ECHB_MOUSE | Hadhb | ECHB_MOUSE Trifunctional enzyme subunit beta, | 3 | 275.727 | 272.068 | 0.987 |
| | | mitochondrial | | | | |
| sp\|P36552\|HEM6_MOUSE | Cpox | HEM6_MOUSE Oxygen-dependent coproporphyrinogen-III | 13 | 1716.87 | 1691.78 | 0.985 |
| | | oxidase, mitochondrial | | | | |
| sp\|O09159\|MA2B1_MOUSE | Man2b1 | MA2B1_MOUSE Lysosomal alpha-mannosidase | 8 | 638.186 | 628.096 | 0.984 |
| sp\|Q9CX13\|CNIH4_MOUSE | Cnih4 | CNIH4_MOUSE Protein cornichon homolog 4 | 3 | 126.949 | 124.913 | 0.984 |
| sp\|Q7TPM3\|TRI17_MOUSE | Trim17 | TRI17_MOUSE E3 ubiquitin-protein ligase TRIM17 | 1 | 189.679 | 186.62 | 0.984 |
| sp\|P39447\|ZO1_MOUSE | Tjp1 | ZO1_MOUSE Tight junction protein ZO-1 | 2 | 86.4191 | 85.0038 | 0.984 |
| sp\|Q9CQ89\|CUTA_MOUSE | Cuta | CUTA_MOUSE Protein CutA | 3 | 520.493 | 511.867 | 0.983 |
| sp\|Q9WVG6\|CARM1_MOUSE | Carm1 | CARM1_MOUSE Histone-arginine methyltransferase | 6 | 1103.53 | 1084.67 | 0.983 |
| | | CARM1 | | | | |
| sp\|Q99LY9\|NDUS5_MOUSE | Ndnfs5 | NDUS5_MOUSE NADH dehydrogenase [ubiquinone] | 6 | 1372.95 | 1348.66 | 0.982 |
| | | iron-sulfur protein 5 | | | | |
| sp\|P11276\|FINC_MOUSE | Fn1 | FINC_MOUSE Fibronectin | 61 | 8760.41 | 8599.17 | 0.982 |
| sp\|Q8R1A4\|DOCK7_MOUSE | Dock7 | DOCK7_MOUSE Dedicator of cytokinesis protein 7 | 1 | 148.829 | 146.064 | 0.981 |
| sp\|Q6P5F6\|S39AA_MOUSE | Slc39a10 | S39AA_MOUSE Zinc transporter ZIP10 | 5 | 363.123 | 356.374 | 0.981 |
| sp\|P62897\|CYC_MOUSE | Cycs | CYC_MOUSE Cytochrome c, somatic | 4 | 746.177 | 731.51 | 0.980 |
| sp\|P62274\|RS29_MOUSE | Rps29 | RS29_MOUSE 40S ribosomal protein S29 | 1 | 298.677 | 292.74 | 0.980 |
| sp\|Q99JR1\|SFXN1_MOUSE | Sfxn1 | SFXN1_MOUSE Sideroflexin-1 | 6 | 507.527 | 497.374 | 0.980 |
| sp\|P10518\|HEM2_MOUSE | Alad | HEM2_MOUSE Delta-aminolevulinic acid dehydratase | 8 | 626.883 | 614.006 | 0.979 |
| sp\|Q9JIF7\|COPB_MOUSE | Copb1 | COPB_MOUSE Coatomer subunit beta | 7 | 909.896 | 889.676 | 0.978 |
| sp\|P80318\|TCPG_MOUSE | Cct3 | TCPG_MOUSE T-complex protein 1 subunit gamma | 9 | 927.727 | 907.018 | 0.978 |
| sp\|Q9DC71\|RT15_MOUSE | Mrps15 | RT15_MOUSE 28S ribosomal protein S15, mitochondrial | 1 | 178.19 | 174.138 | 0.977 |
| sp\|P57784\|RU2A_MOUSE | Snrpa1 | RU2A_MOUSE U2 small nuclear ribonucleoprotein A′ | 3 | 354.554 | 346.206 | 0.976 |
| sp\|Q9CXY9\|GPI8_MOUSE | Pigk | GPI8_MOUSE GPI-anchor transamidase | 1 | 260.671 | 254.526 | 0.976 |
| sp\|P97470\|PP4C_MOUSE | Ppp4c | PP4C_MOUSE Serine/threonine-protein phosphatase 4 | 1 | 35.3666 | 34.5305 | 0.976 |
| | | catalytic subunit | | | | |
| sp\|Q9JKN2\|ZNT7_MOUSE | Slc30a7 | ZNT7_MOUSE Zinc transporter 7 | 36 | 6759.25 | 6592.14 | 0.975 |
| sp\|Q9D1D4\|TMEDA_MOUSE | Tmed10 | TMEDA_MOUSE Transmembrane cmp24 domain- | 7 | 1065.18 | 1038.35 | 0.975 |
| | | containing protein 10 | | | | |
| sp\|Q921V5\|MGAT2_MOUSE | Mgat2 | MGAT2_MOUSE Alpha-1,6-mannosyl-glycoprotein 2- | 2 | 652.383 | 635.939 | 0.975 |
| | | beta-N-acetylglucosaminyltransferase | | | | |
| sp\|Q9Z110\|P5CS_MOUSE | Aldh18a1 | P5CS_MOUSE Delta-1-pyrroline-5-carboxylate synthase | 206 | 45807 | 44643.7 | 0.975 |
| sp\|P14901\|HMOX1_MOUSE | Hmox1 | HMOX1_MOUSE Heme oxygenase 1 | 5 | 1067.26 | 1040.07 | 0.975 |
| sp\|Q8K0D5\|EFGM_MOUSE | Gfm1 | EFGM_MOUSE Elongation factor G, mitochondrial | 7 | 1107.78 | 1079.18 | 0.974 |
| sp\|P10493\|NID1_MOUSE | Nid1 | NID1_MOUSE Nidogen-1 | 10 | 857.801 | 834.751 | 0.973 |
| sp\|P62827\|RAN_MOUSE | Ran | RAN_MOUSE GTP-binding nuclear protein Ran | 3 | 616.951 | 600.134 | 0.973 |
| sp\|Q8VHY0\|CSPG4_MOUSE | Cspg4 | CSPG4_MOUSE Chondroitin sulfate proteoglycan 4 | 1 | 66.9039 | 64.9836 | 0.971 |
| sp\|P00405\|COX2_MOUSE | Mtco2 | COX2_MOUSE Cytochrome c oxidase subunit 2 | 6 | 1756.05 | 1704.84 | 0.971 |
| sp\|O88876\|DHRS3_MOUSE | Dhrs3 | DHRS3_MOUSE Short-chain dehydrogenase/reductase 3 | 1 | 56.9175 | 55.2447 | 0.971 |
| tr\|O88325\|O88325_MOUSE | Naglu | O88325_MOUSE Alpha-N-acetylglucosaminidase | 7 | 886.514 | 860.438 | 0.971 |
| tr\|A2AFQ2\|A2AFQ2_MOUSE | Hsd1Tb10 | A2AFQ2_MOUSE 3-hydroxyacyl-CoA dehydrogenase | 4 | 285.414 | 276.75 | 0.970 |
| | | type-2 | | | | |
| sp\|Q91ZX7\|LRP1_MOUSE | Lrp1 | LRP1_MOUSE Prolow-density lipoprotein receptor- | 265 | 53336.7 | 51612.8 | 0.968 |
| | | related protein 1 | | | | |
| sp\|Q9CWV0\|MASU1_MOUSE | Malsu1 | MASU1_MOUSE Mitochondrial assembly of ribosomal | 3 | 352.636 | 341.185 | 0.968 |
| | | large subunit protein 1 | | | | |
| sp\|Q91VA6\|PDIP2_MOUSE | Poldip2 | PDIP2_MOUSE Polymerase delta-interacting protein 2 | 4 | 485.243 | 469.274 | 0.967 |
| sp\|Q3U2U7\|MET17_MOUSE | Mettl17 | MET17_MOUSE Methyltransferase-like protein 17, | 3 | 389.307 | 375.953 | 0.966 |
| | | mitochondrial | | | | |
| sp\|Q9CXZl\|NDUS4_MOUSE | Ndnfs4 | NDUS4_MOUSE NADH dehydrogenase [ubiquinone] | 12 | 2894.37 | 2789.34 | 0.964 |
| | | iron-sulfur protein 4, mitochondrial | | | | |
| sp\|P26039\|TLN1_MOUSE | Tln1 | TLN1_MOUSE Talin-1 | 5 | 391.99 | 377.741 | 0.964 |
| sp\|Q3V009\|TMED1_MOUSE | Tmed1 | TMED1_MOUSE Transmembrane emp24 domain- | 1 | 97.1165 | 93.5057 | 0.963 |
| | | containing protein 1 | | | | |
| sp\|Q9CR89\|ERG12_MOUSE | Ergic2 | ERGI2_MOUSE Endoplasmic reticulum-Golgi | 25 | 3838.77 | 3689.73 | 0.961 |
| | | intermediate compartment protein 2 | | | | |
| sp\|Q8K411\|PREP_MOUSE | Pitrm1 | PREP_MOUSE Presequence protease, mitochondrial | 134 | 19935.5 | 19133.9 | 0.960 |
| sp\|P99028\|QCR6_MOUSE | Uqcrh | QCR6_MOUSE Cytochrome b-c1 complex subunit | 5 | 629.677 | 604.217 | 0.960 |
| | | 6, mitochondrial | | | | |
| tr\|G5E924\|G5E924_MOUSE | Hnrnpl | G5E924_MOUSE Heterogeneous nuclear | 18 | 3422.63 | 3281.15 | 0.959 |
| | | ribonucleoprotein L (Fragment) | | | | |
| sp\|Q9DAS9\|GBG12_MOUSE | Gng12 | GBG12_MOUSE Guanine nucleotide-binding protein | 1 | 100.843 | 96.6516 | 0.958 |
| | | G(I)/G(S)/G(O) subunit gamma-12 | | | | |
| sp\|Q8VDP6\|CDIPT_MOUSE | Cdipt | CDIPT_MOUSE CDP-diacylglycerol--inositol 3- | 1 | 80.0057 | 76.601 | 0.957 |
| | | phosphatidyltransferase | | | | |
| sp\|P61211\|ARL1_MOUSE | Arl1 | ARL1_MOUSE ADP-ribosylation factor-like protein 1 | 6 | 493.393 | 472.231 | 0.957 |
| tr\|A2A5V2\|A2A5V2_MOUSE | Sh3bP1 | A2A5V2_MOUSE SH3 domain-binding protein 1 | 1 | 221.725 | 212.135 | 0.957 |
| sp\|Q91VD9\|NDUS1_MOUSE | Ndnfs1 | NDUS1_MOUSE NADH-ubiquinone oxidoreductase | 158 | 27066.2 | 25852.8 | 0.955 |
| | | 75 kDa subunit, mitochondrial | | | | |
| sp\|Q9CWH6\|PSA7L_MOUSE | Psma8 | PSA7L_MOUSE Proteasome subunit alpha type-7-like | 1 | 48.4075 | 46.1758 | 0.954 |
| sp\|Q8BFR5\|EFTU_MOUSE | Tufm | EFTU_MOUSE Elongation factor Tu, mitochondrial | 49 | 10592.2 | 10103.4 | 0.954 |
| sp\|Q9CZ42\|NNRD_MOUSE | Carkd | NNRD_MOUSE ATP-dependent (S)-NAD(P)H- | 40 | 6548.55 | 6244.63 | 0.954 |
| | | hydrate dehydratase | | | | |
| sp\|P54071\|IDHP_MOUSE | Idh2 | IDHP_MOUSE Isocitrate dehydrogenase [NADP], | 11 | 1760.04 | 1677.2 | 0.953 |
| | | mitochondrial | | | | |
| sp\|Q9CQD1\|RAB5A_MOUSE | Rab5a | RAB5A_MOUSE Ras-related protein Rab-5A | 4 | 217.113 | 206.752 | 0.952 |
| sp\|P21460\|CYTC_MOUSE | Cst3 | CYTC_MOUSE Cystatin-C | 1 | 324.684 | 309.081 | 0.952 |
| sp\|Q9D823\|RL37_MOUSE | Rpl37 | RL37_MOUSE 60S ribosomal protein L37 | 1 | 133.801 | 127.221 | 0.951 |
| sp\|Q9CQ75\|NDUA2_MOUSE | Ndufa2 | NDUA2_MOUSE NADH dehydrogenase [ubiquinone] | 10 | 1879.73 | 1787.24 | 0.951 |
| | | 1 alpha subcomplex subunit 2 | | | | |
| sp\|Q80TN5\|ZDH17_MOUSE | Zdhhc17 | ZDH17_MOUSE Palmitoyltransferase ZDHHC17 | 1 | 101.819 | 96.8062 | 0.951 |
| sp\|Q9Z247\|EKBP9_MOUSE | Fkbp9 | FKBP9_MOUSE Peptidyl-prolyl cis-trans isomerase FKBP9 | 7 | 1120.99 | 1065.09 | 0.950 |
| sp\|Q99M01\|SYFM_MOUSE | Fars2 | SYFM_MOUSE Phenylalanine--tRNA ligase, mitochondrial | 3 | 376.023 | 356.972 | 0.949 |
| sp\|Q9CQZ5\|NDUA6_MOUSE | Ndufa6 | NDUA6_MOUSE NADH dehydrogenase [ubiquinone] | 8 | 2095.47 | 1989.25 | 0.949 |
| | | 1 alpha subcomplex subunit 6 | | | | |
| sp\|P52503\|NDUS6_MOUSE | Ndufs6 | NDUS6_MOUSE NADH dehydrogenase [ubiquinone] | 10 | 2756.57 | 2616.12 | 0.949 |
| | | iron-sulfur protein 6, mitochondrial | | | | |
| sp\|Q9CPQ3\|TOM22_MOUSE | Tomm22 | TOM22_MOUSE Mitochondrial import receptor subunit | 2 | 112.572 | 106.679 | 0.948 |
| | | TOM22 homolog | | | | |
| sp\|Q9CQV5\|RT24_MOUSE | Mrps24 | RT24_MOUSE 28S ribosomal protein S24, mitochondrial | 2 | 423.643 | 401.457 | 0.948 |
| sp\|Q8BFU1\|S3A9_MOUSE | Slc39a9 | S39A9_MOUSE Zinc transporter ZIP9 | 1 | 204.545 | 193.814 | 0.948 |
| sp\|Q61937\|NPM_MOUSE | Npm1 | NPM_MOUSE Nucleophosmin | 3 | 426.233 | 403.696 | 0.947 |
| sp\|P02666\|CASB_BOVIN_ | CSN2 | CASB_BOVIN_contaminant Beta-casein | 17 | 4182.23 | 3960.8 | 0.947 |
| contaminant | | | | | | |
| sp\|P63024\|VAMP3_MOUSE | Vamp3 | VAMP3_MOUSE Vesicle-associated membrane protein 3 | 1 | 48.344 | 45.7587 | 0.947 |
| sp\|Q9D281\|NXP20_MOUSE | Fam114a1 | NXP20_MOUSE Protein Noxp20 | 1 | 34.5183 | 32.6542 | 0.946 |
| sp\|Q64735\|CR1L_MOUSE | Cr1l | CR1L_MOUSE Complement component receptor | 2 | 158.22 | 149.537 | 0.945 |
| | | 1-like protein | | | | |
| sp\|Q8BIJ6\|SYIM_MOUSE | Iars2 | SYIM_MOUSE Isoleucine--tRNA ligase, mitochondrial | 6 | 672.603 | 635.55 | 0.945 |
| sp\|O88396\|GRPE2_MOUSE | Grpsl2 | GRPE2_MOUSE GrpE protein homolog 2, mitochondrial | 3 | 304.793 | 287.995 | 0.945 |
| sp\|Q9CPX7\|RT16_MOUSE | Mrps16 | RT16_MOUSE 28S ribosomal protein S16, mitochondrial | 2 | 417.878 | 394.653 | 0.944 |
| sp\|Q8BYM8\|SYCM_MOUSE | Cars2 | SYCM_MOUSE Probable cysteine-tRNA ligase, | 31 | 4961.96 | 4682.55 | 0.944 |
| | | mitochondrial | | | | |
| sp\|Q60715\|P4HA1_MOUSE | P4ha1 | P4HA1_MOUSE Prolyl 4-hydroxylase subunit alpha-1 | 14 | 2481.56 | 2339.82 | 0.943 |
| sp\|Q924T2\|RT02_MOUSE | Mrps2 | RT02_MOUSE 28S ribosomal protein S2, mitochondrial | 1 | 56.0968 | 52.8725 | 0.943 |
| sp\|P35980\|RL18_MOUSE | Rpl18 | RL18_MOUSE 60S ribosomal protein L18 | 3 | 584.324 | 550.388 | 0.942 |
| sp\|Q9DCC8\|TOM20_MOUSE | Tomm20 | TOM20_MOUSE Mitochondrial import receptor subunit | 2 | 474.662 | 446.715 | 0.941 |
| | | TOM20 homolog | | | | |
| sp\|P51150\|RAB7A_MOUSE | Rab7a | RAB7A_MOUSE Ras-related protein Rab-7a | 9 | 1334.58 | 1255.87 | 0.941 |
| tr\|A2AIX1\|A2AIXI_MOUSE | Sec16a | A2AIX1_MOUSE Protein Sec16a | 1 | 58.3297 | 54.7342 | 0.938 |
| sp\|Q8BYR1\|TYW4_MOUSE | Lcmt2 | TYW4_MOUSE tRNA wybutosine-synthesizing protein 4 | 1 | 176.941 | 165.949 | 0.938 |
| sp\|P97300\|NPTN_MOUSE | Nptn | NPTN_MOUSE Neuroplastin | 1 | 114.336 | 107.058 | 0.936 |
| sp\|Q6X7S9\|EID2_MOUSE | Eid2 | EID2_MOUSE EP300-interacting inhibitor of | 1 | 257.785 | 241.36 | 0.936 |
| | | differentiation 2 | | | | |
| sp\|Q91VK4\|ITM2C_MOUSE | Itm2c | ITM2C_MOUSE Integral membrane protein 2C | 2 | 105.589 | 98.7142 | 0.935 |
| sp\|O70252\|HMOX2_MOUSE | Hmox2 | HMOX2_MOUSE Heme oxygenase 2 | 1 | 119.354 | 111.536 | 0.934 |
| tr\|Q6NXL1\|Q6NXLI_MOUSE | Sec24d | Q6NXL1_MOUSE Protein Sec24d | 4 | 290.035 | 271.036 | 0.934 |
| sp\|Q8BKC5\|IPO5_MOUSE | Ipo5 | IPO5_MOUSE Impoitin-5 | 4 | 185.81 | 173.601 | 0.934 |
| sp\|Q9CR60\|GOT1B_MOUSE | Got1b | GOT1B_MOUSE Vesicle transport protein GOT1B | 1 | 61.8544 | 57.7658 | 0.934 |
| sp\|P19536\|COX5B_MOUSE | Cox5b | COX5B_MOUSE Cytochrome c oxidase subunit 5B, | 4 | 1128.74 | 1053.9 | 0.934 |
| | | mitochondrial | | | | |
| sp\|Q3UHB1\|NT5D3_MOUSE | Nt5dc3 | NT5D3_MOUSE 5′-nucleotidase domain-containing | 75 | 13196.7 | 12319.2 | 0.934 |
| | | protein 3 | | | | |
| sp\|Q91V16\|LYRM5_MOUSE | Lyrm5 | LYRM5_MOUSE LYR motif-containing protein 5 | 3 | 565.131 | 527.527 | 0.933 |
| sp\|P70404\|IDHG1_MOUSE | Idh3g | IDHG1_MOUSE Isocitrate dehydrogenase [NAD] subunit | 31 | 5865.52 | 5468.31 | 0.932 |
| | | gamma 1, mitochondrial | | | | |
| sp\|P27046\|MA2A1_MOUSE | Man2a1 | MA2A1_MOUSE Alpha-mannosidase 2 | 48 | 8123.85 | 7571.99 | 0.932 |
| sp\|Q9WUD1\|CHIP_MOUSE | Stub1 | CHIP_MOUSE STIP1 homolog and U box-containing | 3 | 211.253 | 196.523 | 0.930 |
| | | protein 1 | | | | |
| sp\|O55028\|BCKD_MOUSE | Bckdk | BCKD_MOUSE [3-methyl-2-oxobutanoate dehydrogenase | 15 | 2482.17 | 2308.47 | 0.930 |
| | | [lipoaniide]] kinase, mitochondrial | | | | |
| sp\|O08553\|DPYL2_MOUSE | Dpysl2 | DPYL2_MOUSE Dihydropyrimidinase-related protein 2 | 2 | 273.159 | 253.529 | 0.928 |
| sp\|P35288\|RAB23_MOUSE | Rab23 | RAB23_MOUSE Ras-related protein Rab-23 | 1 | 51.4396 | 47.7401 | 0.928 |
| sp\|Q9CQS8\|SC6IB_MOUSE | Sec61b | SC61B_MOUSE Protein transport protein Sec61 | 1 | 136.796 | 126.919 | 0.928 |
| | | subunit beta | | | | |
| sp\|Q8VE38\|OXND1_MOUSE | Oxnad1 | OXND1_MOUSE Oxidoreductase NAD-binding domain- | 5 | 844.171 | 783.163 | 0.928 |
| | | containing protein 1 | | | | |
| sp\|A2AJ88\|PLPL7_MOUSE | Pnpla7 | PLPL7_MOUSE Patatin-like phospholipase domain- | 1 | 326.286 | 302.487 | 0.927 |
| | | containing protein 7 | | | | |
| sp\|Q8CGE7\|TCRG1_MOUSE | Tcerg4 | TCRG1_MOUSE Transcription elongation regulator 1 | 1 | 250.319 | 231.697 | 0.926 |
| sp\|Q9EP69\|SAC1_MOUSE | Sacm1l | SAC1_MOUSE Phosphatidylinositide phosphatase SAC1 | 3 | 284.326 | 263.128 | 0.925 |
| sp\|Q9CQQ7\|AT5FI_MOUSE | Atp5f1 | AT5F1_MOUSE ATP synthase F(0) complex subunit B1, | 1 | 125.481 | 116.109 | 0.925 |
| | | mitochondrial | | | | |
| sp\|Q3TCN2\|PLBL2_MOUSE | Plbd2 | PLBL2_MOUSE Putative phospholipase B-like 2 | 2 | 109.218 | 101.009 | 0.925 |
| sp\|Q9D7R2\|PMEPA_MOUSE | Pmepa1 | PMEPA_MOUSE Transmembrane prostate androgen- | 1 | 67.4421 | 62.3685 | 0.925 |
| | | induced protein | | | | |
| sp\|P62307\|RUXF_MOUSE | Snrpf | RUXF_MOUSE Small nuclear ribonucleoprotein F | 1 | 51.4124 | 47.5257 | 0.924 |
| sp\|Q9DCJ5\|NDUA8_MOUSE | Ndufa8 | NDUA8_MOUSE NADH dehydrogenase [ubiquinone] 1 | 14 | 5765.04 | 5327.39 | 0.924 |
| | | alpha subcomplex subunit 8 | | | | |
| tr\|E9QK04\|E9QK04_MOUSE | Neo1 | E9QK04_MOUSE Neogenin | 12 | 1950.14 | 1797.03 | 0.921 |
| sp\|Q922W5\|P5CR1_MOUSE | Pvcr1 | P5CR1_MOUSE Pyrroline-5-carboxylate reductase 1, | 13 | 2406.04 | 2216.54 | 0.921 |
| | | mitochondrial | | | | |
| sp\|Q9CX30\|YIFB_MOUSE | Yif1b | YIF1B_MOUSE Protein YIF1B | 2 | 170.597 | 157.147 | 0.921 |
| tr\|G3X8R0\|G3X8R0_MOUSE | Reep5 | G3X8R0_MOUSE Receptor accessory protein 5, isoform | 3 | 582.219 | 536.25 | 0.921 |
| | | CRA_a | | | | |
| sp\|O35231\|KIFC3_MOUSE | Kifc3 | KIFC3_MOUSE Kinesin-like protein KIFC3 | 1 | 166.223 | 153.053 | 0.921 |
| sp\|Q9IYT0\|NDUV1_MOUSE | Ndufv1 | NDUV1_MOUSE NADH dehydrogenase [ubiquinone] | 53 | 8791.6 | 8089.02 | 0.920 |
| | | flavoprotein 1, mitochondrial | | | | |
| sp\|Q923X4\|GLRX2_MOUSE | Glrx2 | GLRX2_MOUSE Glutaredoxin-2, mitochondrial | 3 | 621.51 | 571.754 | 0.920 |
| sp\|Q99MR8\|MCCA_MOUSE | Mccc1 | MCCA_MOUSE Methylcrotonoyl-CoA carboxylase | 107 | 20917 | 19239 | 0.920 |
| | | subunit alpha, mitochondrial | | | | |
| sp\|Q8VHI3\|OFUT2_MOUSE | Pofut2 | OFUT2_MOUSE GDP-fucosc protein O- | 1 | 96.261 | 88.4303 | 0.919 |
| | | fucosyltransferase 2 | | | | |
| sp\|O70435\|PSA3_MOUSE | Psma3 | PSA3_MOUSE Proteasome subunit alpha type-3 | 1 | 79.8914 | 73.3687 | 0.918 |
| sp\|Q9R045\|ANGL2_MOUSE | Angptl2 | ANGL2_MOUSE Angiopoietin-related protein 2 | 35 | 4757.75 | 4365.89 | 0.918 |
| sp\|Q9CQX2\|CYB5B_MOUSE | Cyb5b | CYB5B_MOUSE Cytochrome b5 type B | 4 | 731.944 | 670.698 | 0.916 |
| tr\|D3Z4W5\|D3Z4W5_MOUSE | 1700 | D3Z4W5_MOUSE Protein 1700074P13Rik (Fragment) | 4 | 1036.3 | 949.226 | 0.916 |
| | 074P13Rik | | | | | |
| sp\|Q9D8V7\|SC11C_MOUSE | Sec11c | SC11C_MOUSE Signal peptidase complex catalytic | 2 | 338.616 | 310.009 | 0.916 |
| | | subunit SEC11C | | | | |
| sp\|P03911\|NU4M_MOUSE | Mtnd4 | NU4M_MOUSE NADH-ubiquinone oxidoreductase | 13 | 4217.35 | 3857.08 | 0.915 |
| | | chain 4 | | | | |
| sp\|Q62351\|TFR1_MOUSE | Tfrc | TFR1_MOUSE Transferrin receptor protein 1 | 3 | 167.012 | 152.481 | 0.913 |
| sp\|Q922Q4\|P5CR2_MOUSE | Pycr2 | P5CR2_MOUSE Pyrroline-5-carboxylate reductase 2 | 40 | 7088.09 | 6469.15 | 0.913 |
| sp\|Q8K211\|COPT1_MOUSE | Slc31a1 | COPT1_MOUSE High affinity copper uptake protein 1 | 13 | 3012.42 | 2746.51 | 0.912 |
| sp\|Q5XKN4\|JAGN1_MOUSE | Jagn1 | JAGN1_MOUSE Protein jagunal homolog 1 | 1 | 246.353 | 224.5 | 0.911 |
| sp\|P10605\|CATB_MOUSE | Ctsb | CATB_MOUSE Cathepsin B | 8 | 657.116 | 598.743 | 0.911 |
| sp\|Q07113\|MPRI_MOUSE | Igf2r | MPRI_MOUSE Cation-indepcndent mannose-6- | 60 | 12080.4 | 11003.9 | 0.911 |
| | | phosphate receptor | | | | |
| sp\|Q75N73\|S39AE_MOUSE | Slc39a14 | S39AE_MOUSE Zinc transporter ZIP14 | 25 | 5756.87 | 5240.79 | 0.910 |
| sp\|Q8K2B3\|SDHA_MOUSE | Sdha | SDHA_MOUSE Succinate dehydrogenase [ubiquinone] | 10 | 1388.24 | 1263.44 | 0.910 |
| | | flavoprotein subunit, mitochondrial | | | | |
| sp\|Q9JJ06\|C1GLT_MOUSE | C1galt1 | C1GLT_MOUSE Glycoprotein-N-acetylgalactosamine | 25 | 4020.77 | 3658.18 | 0.910 |
| | | 3-beta-galactosyltransferase 1 | | | | |
| sp\|Q9D6R2\|IDH3A_MOUSE | Idh3a | IDH3A_MOUSE Isocitrate dehydrogenase [NAD] subunit | 56 | 9754.68 | 8863 | 0.909 |
| | | alpha, mitochondrial | | | | |
| sp\|Q99LD4\|CSN1_MOUSE | Gps1 | CSN1_MOUSE COP9 signalosome complex subunit 1 | 1 | 277.558 | 252.168 | 0.909 |
| sp\|Q8VCH8\|UBXN4_MOUSE | Ubxn4 | UBXN4_MOUSE UBX domain-containing protein 4 | 3 | 191.957 | 174.376 | 0.908 |
| sp\|Q91ZN5\|S35B2_MOUSE | Slc35b2 | S35B2_MOUSE Adenosine 3′-phospho 5′-phosphosulfate | 2 | 131.417 | 119.279 | 0.908 |
| | | transporter 1 | | | | |
| sp\|Q8BQ47\|CNPY4_MOUSE | Cnpy4 | CNPY4_MOUSE Protein canopy homolog 4 | 1 | 123.137 | 111.718 | 0.907 |
| sp\|P52480\|KPYM_MOUSE | Pkm | KPYM_MOUSE Pyruvate kinase PKM | 3 | 582.912 | 528.694 | 0.907 |
| sp\|Q9D172\|ES1_MOUSE | D10Jhu81e | ES1_MOUSE ES1 protein homolog, mitochondrial | 14 | 4494.13 | 4065.6 | 0.905 |
| sp\|O88630\|GOSR1_MOUSE | Gosr1 | GOSR1_MOUSE Golgi SNAP receptor complex member 1 | 1 | 54.0344 | 48.8184 | 0.903 |
| sp\|Q3TIU4\|PDE12_MOUSE | Pde12 | PDE12_MOUSE 2′,5′-phosphodiesterase 12 | 2 | 170.822 | 154.308 | 0.903 |
| sp\|Q64449\|MRC2_MOUSE | Mrc2 | MRC2_MOUSE C-type mannose receptor 2 | 226 | 45871.8 | 41405.9 | 0.903 |
| tr\|Q6A099\|Q6A099_MOUSE | Gbf1 | Q6A099_MOUSE MKIAA0248 protein (Fragment) | 13 | 1524.22 | 1375.7 | 0.903 |
| sp\|P84096\|RHOG_MOUSE | Rhog | RHOG_MOUSE Rho-related GTP-binding protein RhoG | 10 | 1872.05 | 1688.43 | 0.902 |
| sp\|P02662\|CASA1_BOVIN_ | CSN1S1 | CASA1_BOVIN contaminant Alpha-S1-casein | 44 | 7718.03 | 6955.14 | 0.901 |
| contaminant | | | | | | |
| sp\|Q91ZE0\|TMLH_MOUSE | Tmlhe | TMLH_MOUSE Trimethyllysine dioxygenase, | 73 | 13396.8 | 12072.2 | 0.901 |
| | | mitochondrial | | | | |
| sp\|P45952\|ACADM_MOUSE | Acadm | ACADM_MOUSE Medium-chain specific acyl-CoA | 6 | 1240.45 | 1117.25 | 0.901 |
| | | dehydrogenase, mitochondrial | | | | |
| sp\|P35486\|ODPA_MOUSE | Pdha1 | ODPA_MOUSE Pyruvate dehydrogenase E1 | 1 | 79.3274 | 71.4386 | 0.901 |
| | | component subunit alpha, somatic form, mitochondrial | | | | |
| sp\|Q91WD5\|NDUS2_MOUSE | Ndufs2 | NDUS2_MOUSE NADH dehydrogenase [ubiquinone] | 41 | 6512.93 | 5851.23 | 0.898 |
| | | iron-sulfur protein 2, mitochondrial | | | | |
| sp\|Q9CY73\|RM44_MOUSE | Mrpl44 | RM44_MOUSE 39S ribosomal protein L44, mitochondrial | 1 | 45.49 | 40.8471 | 0.898 |
| sp\|Q99KB8\|GLO2_MOUSE | Hagh | GLO2_MOUSE Hydroxyacylglutathione hydrolase, | 1 | 66.0535 | 59.2726 | 0.897 |
| | | mitochondrial | | | | |
| sp\|P19096\|FAS_MOUSE | Fasn | FAS_MOUSE Fatty acid synthase | 6 | 957.819 | 859.132 | 0.897 |
| sp\|Q9JMD0\|ZN207_MOUSE | Znf207 | ZN207_MOUSE BUB3-interacting and GLEBS motif- | 2 | 127.971 | 114.757 | 0.897 |
| | | containing protein ZNF207 | | | | |
| sp\|Q9CY50\|SSRA_MOUSE | Ssr1 | SSRA_MOUSE Translocon-associated protein subunit alpha | 2 | 549.35 | 492.283 | 0.896 |
| lr\|Q91VA7\|Q91VA7_MOUSE | Idh3b | Q91VA7_MOUSE Isocitrate dehydrogenase 3 (NAD+) beta | 31 | 6816.85 | 6106.47 | 0.896 |
| tr\|Q91X76\|Q91X76_MOUSE | Nt5de2 | Q91X76_MOUSE 5′-nuclcotidase domain containing 2 | 114 | 30643.7 | 27413.3 | 0.895 |
| sp\|Q6NZC7\|S23IP_MOUSE | Sec23ip | S23IP_MOUSE SEC23-interacting protein | 7 | 8090.94 | 7226.31 | 0.893 |
| sp\|Q9D8B4\|NDUAB_MOUSE | Ndnfa11 | NDUAB_MOUSE NADH dehydrogenase [ubiquinone] 1 | 1 | 119.461 | 106.532 | 0.892 |
| | | alpha subcomplex subunit 11 | | | | |
| sp\|Q9JLZ3\|AUHM_MOUSE | Auh | AUHM_MOUSE Methylglutaconyl-CoA hydratase, | 34 | 7743.09 | 6904.77 | 0.892 |
| | | mitochondrial | | | | |
| sp\|P03888\|NU1M_MOUSE | Mtnd1 | NU1M_MOUSE NADH-ubiquinone oxidoreductase chain 1 | 15 | 2056.16 | 1833.45 | 0.892 |
| sp\|Q9CQE7\|ERGI3_MOUSE | Ergic3 | ERGI3_MOUSE Endoplasmic reticulum-Golgi intermediate | 34 | 5158.2 | 4598.38 | 0.891 |
| | | compartment protein 3 | | | | |
| sp\|Q9D273\|MMAB_MOUSE | Mmab | MMAB_MOUSE Cob(I)yrinic acid a,c-diamide | 1 | 75.5876 | 67.3464 | 0.891 |
| | | adenosyltransferase, mitochondrial | | | | |
| sp\|O0911I\|NDUBB_MOUSE | Ndufb11 | NDUBB_MOUSE NADH dehydrogenase [ubiquinone] 1 | 24 | 5785.34 | 5154.03 | 0.891 |
| | | beta subcomplex subunit 11, mitochondrial | | | | |
| sp\|Q9DBU0\|TM9S1_MOUSE | Tm9sf1 | TM9S1_MOUSE Transmembrane 9 superfamily member 1 | 5 | 565.588 | 503.397 | 0.890 |
| sp\|Q69ZS0\|PZRN3_MOUSE | Pdzrn3 | PZRN3_MOUSE E3 ubiquitin-protein ligase PDZRN3 | 1 | 195.295 | 173.684 | 0.889 |
| sp\|Q99JT6\|TLCD1_MOUSE | Tlcd1 | TLCD1_MOUSE Calfacilitin | 1 | 103.279 | 91.8323 | 0.889 |
| sp\|P29416\|HEXA_MOUSE | Hexa | HEXA_MOUSE Beta-hexosaminidase subunit alpha | 4 | 409.104 | 363.587 | 0.889 |
| sp\|Q9CQA3\|SDHB_MOUSE | Sdhb | SDHB_MOUSE Succinate dehydrogenase [ubiquinone] | 2 | 312.49 | 277.418 | 0.888 |
| | | iron-sulfur subunit, mitochondrial | | | | |
| sp\|Q8BMF3\|MAON_MOUSE | Me3 | MAON_MOUSE NADP-dependent malic enzyme, | 9 | 2303.1 | 2044.57 | 0.888 |
| | | mitochondrial | | | | |
| sp\|Q9D0M3\|CY1_MOUSE | Cyc1 | CY1_MOUSE Cytochrome c1, heme protein, mitochondrial | 9 | 1910.29 | 1694.74 | 0.887 |
| sp\|P51569\|AGAL_MOUSE | Gla | AGAL_MOUSE Alpha-galactosidase A | 3 | 243.992 | 216.434 | 0.887 |
| tr\|G3X975\|G3X975_MOUSE | Cars2 | G3X975_MOUSE MCG11180, isoform CRA_a | 1 | 50.8561 | 44.9998 | 0.885 |
| sp\|P45481\|CBP_MOUSE | Crebbp | CBP_MOUSE CREB-binding protein | 3 | 255.918 | 226.381 | 0.885 |
| sp\|P26443\|DHE3_MOUSE | Glud1 | DHE3_MOUSE Glutamate dehydrogenase 1, mitochondrial | 233 | 61092.4 | 54022.2 | 0.884 |
| sp\|Q99PV0\|PRP8_MOUSE | Prpf8 | PRP8_MOUSE Pre-mRNA-processing-splicing factor 8 | 1 | 112.972 | 99.8095 | 0.883 |
| sp\|P97321\|SEPR_MOUSE | Fap | SEPR_MOUSE Seprase | 2 | 104.564 | 92.3737 | 0.883 |
| sp\|Q6PB66\|LPPRC_MOUSE | Lrpprc | LPPRC_MOUSE Leucine-rich PPR motif-containing | 2 | 296.458 | 261.83 | 0.883 |
| | | protein, mitochondrial | | | | |
| sp\|P51660\|DHB4_MOUSE | Hsd17b4 | DHB4_MOUSE Peroxisomal multifunctional enzyme type 2 | 1 | 37.1501 | 32.7994 | 0.883 |
| sp\|Q99JR6\|NMNA3_MOUSE | Nmnat3 | NMNA3_MOUSE Nicotinamide mononucleotide | 1 | 95.6087 | 84.3444 | 0.882 |
| | | adenylyltransferase 3 | | | | |
| sp\|P37913\|DNLI1_MOUSE | Lig1 | DNLI1_MOUSE DNA ligase 1 | 1 | 172.154 | 151.67 | 0.881 |
| sp\|Q9CR61\|NDUB7_MOUSE | Ndufb7 | NDUB7_MOUSE NADH dehydrogenase [ubiquinone] | 18 | 2947.44 | 2593.3 | 0.880 |
| | | 1 beta subcomplex subunit 7 | | | | |
| sp\|P60710\|ACTB_MOUSE | Actb | ACTB_MOUSE Actin, cytoplasmic 1 | 20 | 2570.69 | 2261.25 | 0.880 |
| sp\|Q9CWX2\|CIA30_MOUSE | Ndufaf1 | CIA30_MOUSE Complex I intermediate-associated protein | 3 | 526.681 | 463.116 | 0.879 |
| | | 30, mitochondrial | | | | |
| sp\|Q99KFl\|TMED9_MOUSE | Tmed9 | TMED9_MOUSE Transmembrane emp24 domain- | 4 | 1005.3 | 883.516 | 0.879 |
| | | containing protein 9 | | | | |
| sp\|Q9DCS9\|NDUBA_MOUSE | Ndufb10 | NDUBA_MOUSENADH dehydrogenase [ubiquinone] 1 | 14 | 3353.74 | 2946.69 | 0.879 |
| | | beta subcomplex subunit 10 | | | | |
| sp\|P06802\|ENPP1_MOUSE | Enpp1 | ENPP1_MOUSE Ectonucleotide pyrophosphatase/ | 1 | 54.5165 | 47.8701 | 0.878 |
| | | phosphodiesterase family member 1 | | | | |
| sp\|P27048\|RSMB_MOUSE | Snrpb | RSMB_MOUSE Small nuclear ribonucleoprotein- | 1 | 257.396 | 225.982 | 0.878 |
| | | associated protein B | | | | |
| sp\|P47791\|GSHR_MOUSE | Gsr | GSHR_MOUSE Glutathione reductase, mitochondrial | 2 | 132.82 | 116.322 | 0.876 |
| sp\|Q922B1\|MACD1_MOUSE | Macrod1 | MACD1_MOUSE O-acetyl-ADP-ribose deacetylase | 1 | 33.7787 | 29.5769 | 0.876 |
| | | MACROD1 | | | | |
| sp\|P97471\|SMAD4_MOUSE | Smad4 | SMAD4_MOUSE Mothers against decapentaplegic | 7 | 1012.96 | 886.576 | 0.875 |
| | | homolog 4 | | | | |
| sp\|Q99L27\|GMPR2_MOUSE | Gmpr2 | GMPR2_MOUSE GMP reductase 2 | 2 | 306.802 | 268.341 | 0.875 |
| sp\|Q9ERS2\|NDUAD_MOUSE | Ndufa13 | NDUAD_MOUSE NADH dehydrogenase [ubiquinone] 1 | 14 | 3432.89 | 2998.17 | 0.873 |
| | | alpha subcomplex subunit 13 | | | | |
| sp\|Q6PDQ2\|CHD4_MOUSE | Chd4 | CHD4_MOUSE Chromodomain-helicase-DNA- | 3 | 205.107 | 178.871 | 0.872 |
| | | binding protein 4 | | | | |
| sp\|A2AJA9\|CI172_MOUSE | Gm996 | CI172_MOUSE Uncharacterized protein C9orf172 | 3 | 5618.85 | 4898.74 | 0.872 |
| | | homolog | | | | |
| sp\|Q9EPL2\|CSTN1_MOUSE | Clstn1 | CSTN1_MOUSE Calsyntenin-1 | 1 | 58.0602 | 50.6126 | 0.872 |
| sp\|Q8CC88\|VWA8_MOUSE | Vwa8 | VWA8_MOUSE von Willebrand factor A domain- | 67 | 9717.52 | 8468.47 | 0.871 |
| | | containing protein 8 | | | | |
| sp\|Q9R0X4\|ACOT9_MOUSE | Acot9 | ACOT9_MOUSE Acyl-coenzyme A thioesterase 9, | 131 | 26985.1 | 23465.6 | 0.870 |
| | | mitochondrial | | | | |
| sp\|Q5UAK0\|MIER1_MOUSE | Mier1 | MIER1_MOUSE Mesoderm induction early response | 1 | 290.86 | 252.413 | 0.868 |
| | | protein 1 | | | | |
| sp\|Q9CQI7\|RU2B_MOUSE | Snrpb2 | RU2B_MOUSE U2 small nuclear ribonucleoprotein B″ | 1 | 35.262 | 30.5855 | 0.867 |
| tr\|F2Z4A3\|F2Z4A3_MOUSE | Fat1 | F2Z4A3_MOUSE Protein Fat1 | 1 | 59.4697 | 51.3557 | 0.864 |
| sp\|Q9DC69\|NDUA9_MOUSE | Ndtufa9 | NDUA9_MOUSE NADH dehydrogenase [ubiquinone] 1 | 38 | 9120.53 | 7869.12 | 0.863 |
| | | alpha subcomplex subunit 9, mitochondrial | | | | |
| sp\|Q61102\|ABCB7_MOUSE | Abcbn | ABCB7_MOUSE ATP-binding cassette sub-family B | 57 | 11816.4 | 10180.4 | 0.862 |
| | | member 7, mitochondrial | | | | |
| sp\|O35129\|PHB2_MOUSE | Phb2 | PHB2_MOUSE Prohibitin-2 | 14 | 2040.12 | 1755.83 | 0.861 |
| sp\|O54782\|MA2B2_MOUSE | Man2b2 | MA2B2_MOUSE Epididymis-specific alpha-mannosidase | 5 | 1020.06 | 877.704 | 0.860 |
| sp\|Q8BYL4\|SYYM_MOUSE | Yars2 | SYYM_MOUSE Tyrosine--tRNA ligase, mitochondrial | 14 | 2419.71 | 2079.76 | 0.860 |
| sp\|Q99LC3\|NDUAA_MOUSE | Ndufa10 | NDUAA_MOUSE NADH dehydrogenase [ubiquinone] | 1 | 56.3977 | 48.467 | 0.859 |
| | | 1 alpha subcomplex subunit 10, mitochondrial | | | | |
| sp\|Q9DCA2\|RT11_MOUSE | Mrps11 | RT11_MOUSE 28S ribosomal protein S11, mitochondrial | 3 | 513.612 | 441.183 | 0.859 |
| sp\|P04925\|PR10_MOUSE | Prnp | PRIO_MOUSE Major prion protein | 2 | 123.911 | 106.413 | 0.859 |
| sp\|P03893\|NU2M_MOUSE | Mtnd2 | NU2M_MOUSE NADH-ubiquinone oxidoreductase chain 2 | 10 | 1161.44 | 995.33 | 0.857 |
| tr\|F6QRE9\|F6QRE9_MOUSE | BC007180 | F6QRE9_MOUSE Protein BC007180 (Fragment) | 1 | 219.243 | 187.869 | 0.857 |
| sp\|O88822\|SC5D_MOUSE | Sc5d | SC5D_MOUSE Lathosterol oxidase | 1 | 374.422 | 320.664 | 0.856 |
| sp\|Q9CQC7\|NDUB4_MOUSE | Ndufb4 | NDUB4_MOUSE NADH dehydrogenase [ubiquinone] 1 | | 4029.17 | 3448.83 | 0.856 |
| | | beta subcomplex subunit 4 | | | | |
| sp\|P19783\|COX41_MOUSE | Cox4i1 | COX41_MOUSE Cytochrome c oxidase subunit 4 | 8 | 940.154 | 804.255 | 0.855 |
| | | isoform 1, mitochondrial | | | | |
| sp\|Q9D6M3\|GHC1_MOUSE | Slc25a22 | GHC1_MOUSE Mitochondrial glutamate carrier 1 | 2 | 277.169 | 236.957 | 0.855 |
| sp\|Q9D8X0\|MANBL_MOUSE | Manbal | MANBL_MOUSE Protein MANBAL | 1 | 67.1857 | 57.4341 | 0.855 |
| sp\|Q99MB1\|TLR3_MOUSE | Tlr3 | TLR3_MOUSE Toll-like receptor 3 | 34 | 8269.37 | 7064.53 | 0.854 |
| sp\|P56135\|ATPK_MOUSE | Atp5j2 | ATPK_MOUSE ATP synthase subunit f, mitochondrial | 1 | 106.302 | 90.7336 | 0.854 |
| sp\|P54818\|GALC_MOUSE | Galc | GALC_MOUSE Galactocerebrosidase | 19 | 2299.26 | 1957.96 | 0.852 |
| sp\|Q6ZQM8\|UD17C_MOUSE | Ugt1a7c | UD17C_MOUSE UDP-glucuronosyltransferase 1-7C | 1 | 38.2723 | 32.5856 | 0.851 |
| sp\|Q6P3Y9\|PONL1_MOUSE | Podul1 | PONL1_MOUSE Podocan-like protein 1 | 1 | 56.2432 | 47.8598 | 0.851 |
| sp\|O08807\|PRDX4_MOUSE | Prdx4 | PRDX4_MOU SE Peroxiredoxin-4 | 23 | 6548.82 | 5557.27 | 0.849 |
| sp\|P02469\|LAMB1_MOUSE | Lamb1 | LAMB1_MOUSE Laminin subunit beta-1 | 14 | 1957.43 | 1659.55 | 0.848 |
| sp\|Q9CQ62\|DECR_MOUSE | Decr1 | DECR_MOUSE 2,4-dienoyl-CoA reductase, mitochondrial | 1 | 59.9429 | 50.8072 | 0.848 |
| tr\|G5E897\|G5E897_MOUSE | Kdelc2 | G5E897_MOUSE KDEL (Lys-Asp-Glu-Leu) containing 2, | 1 | 88.0689 | 74.5534 | 0.847 |
| | | isoform CRA_b | | | | |
| tr\|D3YZZ5\|D3YZZ5_MOUSE | Tmed7 | D3YZZ5_MOUSE Protein Tmed7 | 4 | 714.843 | 604.62 | 0.846 |
| sp\|P23492\|PNPH_MOUSE | Pnp | PNPH_MOUSE Purine nucleoside phosphorylase | 2 | 118.335 | 99.9126 | 0.844 |
| sp\|Q8K385\|FRRS1_MOUSE | FRRS1 | FRRS1_MOUSE Ferric-chelate reductase 1 | 2 | 239.932 | 202.293 | 0.843 |
| sp\|Q9JM62\|REEP6_MOUSE | Reep6 | REEP6_MOUSE Receptor expression-enhancing protein 6 | 1 | 116.05 | 97.7979 | 0.843 |
| sp\|Q9D6J6\|NDUV2_MOUSE | Ndnfv2 | NDUV2_MOUSE NADH dehydrogenase [ubiquinone] | 22 | 4599.55 | 3875.71 | 0.843 |
| | | flavoprotein 2, mitochondrial | | | | |
| sp\|P10923\|OSTP_MOUSE | Spp1 | OSTP_MOUSE Osteopontin | 24 | 4758.36 | 3997.7 | 0.840 |
| sp\|Q99M71\|EPDR1_MOUSE | Epdr1 | EPDR1_MOUSE Mammalian ependymin-related protein 1 | 2 | 218.304 | 183.274 | 0.840 |
| sp\|Q9D517\|PLCC_MOUSE | Agpat3 | PLCC_MOUSE 1-acyl-sn-glycerol-3-phosphate | 2 | 241.309 | 201.973 | 0.837 |
| | | acyltransferase gamma | | | | |
| sp\|P67778\|PHB_MOUSE | Phb | PHB_MOUSE Prohibitin | 9 | 1944.97 | 1627.52 | 0.837 |
| sp\|Q9D270\|ZDH21_MOUSE | Zdhhc21 | ZDH21_MOUSE Probable palmitoyltransferase ZDHHC21 | 1 | 64.0315 | 53.4932 | 0.835 |
| sp\|Q99N94\|RM09_MOUSE | Mrpl9 | RM09_MOUSE 39S ribosomal protein L9, mitochondrial | 2 | 183.009 | 152.789 | 0.835 |
| sp\|Q8VDV8\|MITD1_MOUSE | Mitd1 | MITD1_MOUSE MIT domain-containing protein 1 | 7 | 1439.39 | 1198.16 | 0.832 |
| sp\|P56392\|CX7A1_MOUSE | Cox7a1 | CX7A1_MOUSE Cytochrome c oxidase subunit 7A1, | 1 | 89.3695 | 74.3154 | 0.832 |
| | | mitochondrial | | | | |
| sp\|P99029\|PRDX5_MOUSE | Prdx5 | PRDX5_MOUSE Peroxiredoxin-5, mitochondrial | 2 | 384.352 | 319.592 | 0.832 |
| sp\|Q3UBX0\|TM109_MOUSE | Tmem109 | TM109_MOUSE Transmembrane protein 109 | 4 | 369.445 | 306.942 | 0.831 |
| sp\|Q8BJZ4\|RT35_MOUSE | Mrps35 | RT35_MOUSE 28S ribosomal protein S35, mitochondrial | 2 | 154.231 | 127.95 | 0.830 |
| tr\|Q504M2\|Q504M2_MOUSE | Pdp2 | Q504M2_MOUSE MCG53395 | 5 | 543.49 | 450.483 | 0.829 |
| sp\|Q9WUU7\|CATZ_MOUSE | Ctsz | CATZ_MOUSE CathepsinZ | 14 | 2463.74 | 2041.39 | 0.829 |
| sp\|Q61425\|HCDH_MOUSE | Hadh | HCDH_MOUSE Hydroxyacyl-coenzyme A | 1 | 151.174 | 125.217 | 0.828 |
| | | dehydrogenase, mitochondrial | | | | |
| sp\|Q8K1R3\|PNPT1_MOUSE | Pnpt1 | PNPT1_MOUSE Polyribonucleotide nucleotidyl- | 10 | 1760.94 | 1458.46 | 0.828 |
| | | transferase 1, mitochondrial | | | | |
| sp\|Q9D3D9\|ATPD_MOUSE | Atp5d | ATPD_MOUSE ATP synthase subunit delta, | 1 | 164.56 | 136.241 | 0.828 |
| | | mitochondrial | | | | |
| tr\|G3UVU2\|G3UVU2_MOUSE | Sf3a2 | G3UVU2_MOUSE Splicing factor 3A subunit 2 | 4 | 465.827 | 384.879 | 0.826 |
| sp\|Q9ZlP5\|CD320_MOUSE | Cd320 | CD320_MOUSE CD320 antigen | 1 | 81.4048 | 67.122 | 0.825 |
| sp\|Q91V92\|ACLY_MOUSE | Acly | ACLY_MOUSE ATP-citrate synthase | 1 | 138.677 | 114.341 | 0.825 |
| sp\|Q9D2L1\|ARSK_MOUSE | Arsk | ARSK_MOUSE Arylsulfatase K | 2 | 326.657 | 269.306 | 0.824 |
| sp\|Q7TMF3\|NDUAC_MOUSE | Ndufa12 | NDUAC_MOUSE NADH dehydrogenase [ubiquinone] 1 | 18 | 3541.29 | 2912.54 | 0.822 |
| | | alpha subcomplex subunit 12 | | | | |
| sp\|Q9DCT2\|NDUS3_MOUSE | Ndnfs3 | NDUS3_MOUSE NADH dehydrogenase [ubiquinone] | 16 | 4345.78 | 3570.64 | 0.822 |
| | | iron-sulfur protein 3, mitochondrial | | | | |
| sp\|Q8R2R6\|MTG1_MOUSE | Mtg1 | MTG1_MOUSE Mitochondrial ribosome-associated | 1 | 97.4572 | 79.721 | 0.818 |
| | | GTPase 1 | | | | |
| sp\|Q640P4\|GL8D2_MOUSE | GH8d2 | GL8D2_MOUSE Glycosyltransferase 8 domain- | 1 | 119.224 | 97.5003 | 0.818 |
| | | containing protein 2 | | | | |
| sp\|P27808\|MGAT1_MOUSE | Mgat1 | MGAT1_MOUSE Alpha-1,3-mannosyl-glycoprotein | 1 | 109.721 | 89.6655 | 0.817 |
| | | 2-beta-N-acetylglucosaminyltransferase | | | | |
| sp\|P46638\|RB11B_MOUSE | Rab11b | RB11B_MOUSE Ras-related protein Rab-11B | 7 | 1413.2 | 1154.63 | 0.817 |
| sp\|Q9WTP7\|KAD3_MOUSE | Ak3 | KAD3_MOUSE GTP:AMP phosphotransferase AK3, | 2 | 217.349 | 177.535 | 0.817 |
| | | mitochondrial | | | | |
| sp\|Q9QYF1\|RDH11_MOUSE | Rdh11 | RDH11_MOUSE Retinol dehydrogenase 11 | 3 | 412.673 | 336.868 | 0.816 |
| sp\|Q8BHE8\|CB047_MOUSE | | CB047_MOUSE Uncharacterized protein C2orf47 | 1 | 33.3458 | 27.2168 | 0.816 |
| | | homolog, mitochondrial | | | | |
| sp\|Q64364\|CD2A2_MOUSE | Cdkn2a | CD2A2_MOUSE Cyclin-dependent kinase inhibitor | 3 | 506.082 | 411.595 | 0.813 |
| | | 2A, isoform 3 | | | | |
| sp\|P09528\|FR1H_MOUSE | Fth1 | FRIH_MOUSE Ferritin heavy chain | 6 | 1710 | 1390.12 | 0.813 |
| sp\|Q9D7P6\|ISCU_MOUSE | Iscu | ISCU_MOUSE Iron-sulfur cluster assembly enzyme | 1 | 133.344 | 108.295 | 0.812 |
| | | ISCU, mitochondrial | | | | |
| sp\|O35683\|NDUA1_MOUSE | Ndufa1 | NDUA1_MOUSE NADH dehydrogenase [ubiquinone] 1 | 2 | 303.666 | 246.406 | 0.811 |
| | | alpha subcomplex subunit 1 | | | | |
| sp\|P02663\|CASA2_BOVIN_ | CSN1S2 | CASA2_BOVIN_contaminant Alpha-S2-casein | 23 | 5596.11 | 4536.98 | 0.811 |
| contaminant | | | | | | |
| sp\|P14069\|S10A6_MOUSE | S100a6 | S10A6_MOUSE Protein S100-A6 | 3 | 455.712 | 368.639 | 0.809 |
| sp\|P51174\|ACADL_MOUSE | Acadl | ACADL_MOUSE Long-chain specific acyl-CoA | 4 | 682.87 | 551.439 | 0.808 |
| | | dehydrogenase, mitochondrial | | | | |
| sp\|Q9Z2W0\|DNPEP_MOUSE | Dnpep | DNPEP_MOUSE Aspartyl aminopeptidase | 1 | 67.244 | 54.2195 | 0.806 |
| sp\|P03921\|NU5M_MOUSE | Mtnd5 | NU5M_MOUSE NADH-ubiquinone oxidoreductase | 13 | 2333.81 | 1880.98 | 0.806 |
| | | chain 5 | | | | |
| sp\|P29391\|FRIL1_MOUSE | Ftl1 | FRIL1_MOUSE Ferritin light chain 1 | 22 | 5113.78 | 4118.51 | 0.805 |
| sp\|Q8C1F4\|CGAT2_MOUSE | Csgalnact2 | CGAT2_MOUSE Chondroitin sulfate N- | 2 | 123.073 | 98.9329 | 0.804 |
| | | acetylgalactosaminyltransferase 2 | | | | |
| sp\|Q8VC19\|HEM1_MOUSE | Alas1 | HEM1_MOUSE 5-aminolevulinate synthase, nonspecific, | 2 | 137.975 | 110.489 | 0.801 |
| | | mitochondrial | | | | |
| sp\|Q9CQ91\|NDUA3_MOUSE | Ndufa3 | NDUA3_MOUSE NADH dehydrogenase [ubiquinone] 1 | 6 | 1744.83 | 1396.57 | 0.800 |
| | | alpha subcomplex subunit 3 | | | | |
| tr\|F8VQJ3\|F8VQJ3_MOUSE | Lamc1 | F8VQJ3_MOUSE Laminin subunit gamma-1 | 10 | 1039.32 | 831.624 | 0.800 |
| sp\|Q99JH8\|ERD21_MOUSE | Kdelr1 | ERD21_MOUSE ER lumen protein-retaining receptor 1 | 5 | 1172.26 | 937.879 | 0.800 |
| tr\|E9PZ16\|E9PZ16_MOUSE | Hspg2 | E9PZ16_MOUSE Basement membrane-specific | 20 | 3706.43 | 2960.71 | 0.799 |
| | | heparan sulfate proteoglycan core protein | | | | |
| sp\|Q8K0Z7\|TACO1_MOUSE | Taco1 | TACO1_MOUSE Translational activator of cytochrome | 3 | 264.201 | 210.865 | 0.798 |
| | | c oxidase 1 | | | | |
| sp\|Q8R326\|PSPC1_MOUSE | Pspc1 | PSPC1_MOUSE Paraspeckle component 1 | 5 | 793.386 | 632.255 | 0.797 |
| sp\|Q3ULD5\|MCCB_MOUSE | Mccc2 | MCCB_MOUSE Methylcrotonoyl-CoA carboxylase beta | 67 | 9550.55 | 7600.64 | 0.796 |
| | | chain, mitochondrial | | | | |
| sp\|Q8BH86\|CN159_MOUSE | | CN159_MOUSE UPF0317 protein C 14orf159 homolog, | 1 | 72.1095 | 57.3648 | 0.796 |
| | | mitochondrial | | | | |
| sp\|P84084\|ARF5_MOUSE | Arf5 | ARF5_MOUSE ADP-ribosylation factor 5 | 5 | 1101.19 | 871.843 | 0.792 |
| sp\|Q924H5\|RA51C_MOUSE | Rad51c | RA51C_MOUSE DNA repair protein RADS 1 homolog 3 | 1 | 485.288 | 383.994 | 0.791 |
| sp\|Q05793\|PGBM_MOUSE | Hspg2 | PGBM_MOUSE Basement membrane-specific | 111 | 18062.6 | 14283.3 | 0.791 |
| | | heparan sulfate proteoglycan core protein | | | | |
| sp\|O88844\|IDHC_MOUSE | Idh1 | IDHC_MOUSE Isocitrate dehydrogenase [NADP] | 1 | 80.3501 | 63.5039 | 0.790 |
| | | cytoplasmic | | | | |
| sp\|Q8R1V4\|TMED4_MOUSE | Tmed4 | TMED4_MOUSE Transmembrane emp24 domain- | 4 | 390.838 | 308.794 | 0.790 |
| | | containing protein 4 | | | | |
| tr\|Q8R5L1\|Q8R5L1_MOUSE | C1qbp | Q8R5L1_MOUSE Complement component 1 Q | 1 | 104.013 | 82.0335 | 0.789 |
| | | subcomponent-binding protein, mitochondrial | | | | |
| sp\|P22315\|HEMH_MOUSE | Fech | HEMH_MOUSE Ferrochelatase, mitochondrial | 2 | 167.704 | 131.76 | 0.786 |
| sp\|Q9D2G2\|ODO2_MOUSE | Dlst | ODO2_MOUSE Dihydrolipoyllysine-residue | 3 | 607.42 | 477.067 | 0.785 |
| | | succinyltransferase component of 2-oxoglutarate | | | | |
| | | dehydrogenase complex, mitochondrial | | | | |
| sp\|Q9WTS2\|FUT8_MOUSE | Fut8 | FUT8_MOUSE Alpha-(1,6)-fucosyltransferase | 2 | 87.6965 | 68.6201 | 0.782 |
| sp\|P42125\|ECI1_MOUSE | Eci1 | ECI1_MOUSE Enoyl-CoA delta isonterase 1, | 2 | 242.874 | 189.957 | 0.782 |
| | | mitochondrial | | | | |
| sp\|P02754\|LACB_BOVIN_ | LGB | LACB_BOVIN_contaminant Beta-lactoglobulin | 30 | 8076 | 6297.19 | 0.780 |
| contaminant | | | | | | |
| sp\|Q5RKZ7\|MOCS1_MOUSE | Mocs1 | MOCS1_MOUSE Molybdenum cofactor biosynthesis | 7 | 1537.11 | 1196.75 | 0.779 |
| | | protein 1 | | | | |
| sp\|P36536\|SAR1A_MOUSE | Sar1a | SAR1A_MOUSE GTP-binding protein SAR1a | 2 | 259.316 | 201.481 | 0.777 |
| tr\|E9Q512\|E9Q512_MOUSE | Trip11 | E9Q512_MOUSE Protein Trip11 | 1 | 66.9213 | 51.8997 | 0.776 |
| sp\|Q60930\|VDAC2_MOUSE | Vdac2 | VDAC2_MOUSE Voltage-dependent anion-selective | 2 | 98.3343 | 76.241 | 0.775 |
| | | channel protein 2 | | | | |
| sp\|Q3UIU2\|NDUB6_MOUSE | Ndufb6 | NDUB6_MOUSE NADH dehydrogenase [ubiquinone] | 7 | 2930.26 | 2263.62 | 0.772 |
| | | 1 beta subcomplex subunit 6 | | | | |
| tr\|Q3UJB0\|Q3UJB0_MOUSE | Sf3b2 | Q3UJB0_MOUSE Protein Sf3b2 | 23 | 2654.7 | 2050.02 | 0.772 |
| sp\|P50429\|ARSB_MOUSE | Arsb | ARSB_MOUSE Arylsulfatase B | 43 | 10191.7 | 7867.36 | 0.772 |
| sp\|Q8BK30\|NDUV3_MOUSE | Ndnfv3 | NDUV3_MOUSE NADH dehydrogenase [ubiquinone] | 3 | 485.797 | 374.832 | 0.772 |
| | | flavoprotein 3, mitochondrial | | | | |
| sp\|Q9CPP6\|NDUA5_MOUSE | Ndufa5 | NDUA5_MOUSE NADH dehydrogenase [ubiquinone] | 7 | 3021.94 | 2331.02 | 0.771 |
| | | 1 alpha subcomplex subunit 5 | | | | |
| sp\|A2AIL4\|NDUF6_MOUSE | Ndufaf6 | NDUF6_MOUSE NADH dehydrogenase (ubiquinone) | 11 | 3009.6 | 2320.24 | 0.771 |
| | | complex 1, assembly factor 6 | | | | |
| sp\|O35405\|PLD3_MOUSE | Pld3 | PLD3_MOUSE Phospholipase D3 | 52 | 11156.2 | 8598.51 | 0.771 |
| sp\|P11214\|TPA_MOUSE | Plat | TPA_MOUSE Tissue-type plasminogen activator | 2 | 297.113 | 228.832 | 0.770 |
| sp\|Q8VCW4\|UN93B_MOUSE | Unc9b1 | UN93B_MOUSE Protein unc-93 homolog B1 | 15 | 2460.83 | 1891.63 | 0.769 |
| sp\|Q99N84\|RT18B_MOUSE | Mrps18b | RT18B_MOUSE 28S ribosomal protein S18b, | 1 | 120.792 | 92.7458 | 0.768 |
| | | mitochondrial | | | | |
| tr\|Q9QUK9\|Q9QUK9_MOUSE | Try5 | Q9QUK9_MOUSE MCG15083 | 13 | 1941.72 | 1490.52 | 0.768 |
| sp\|Q922H2\|PDK3_MOUSE | Pdk3 | PDK3_MOUSE [Pyruvate dehydrogenase (acetyl- | 1 | 116.329 | 89.2477 | 0.767 |
| | | transferring)] kinase isozyme 3, mitochondrial | | | | |
| sp\|Q8BH95\|ECHM_MOUSE | Echs1 | ECHM_MOUSE Enoyl-CoA hydratase, mitochondrial | 8 | 1497.91 | 1149.07 | 0.767 |
| sp\|P03899\|NU3M_MOUSE | Mtnd3 | NU3M_MOUSE NADH-ubiqninone oxidoreductase chain 3 | 9 | 1193.73 | 914.324 | 0.766 |
| sp\|Q9CZU6\|CISY_MOUSE | Cs | CISY_MOUSE Citrate synthase, mitochondrial | 1 | 578.518 | 442.416 | 0.765 |
| sp\|Q80SW1\|SAHH2_MOUSE | Ahcyl1 | SAHH2_MOUSE Putative adenosylhomocysteinase 2 | 1 | 73.301 | 55.9065 | 0.763 |
| sp\|P47738\|ALDH2_MOUSE | Aldh2 | ALDH2_MOUSE Aldehyde dehydrogenase, mitochondrial | 9 | 1270.66 | 966.318 | 0.760 |
| sp\|Q91XC8\|DAP1_MOUSE | Dap | DAP1_MOUSE Death-associated protein 1 | 1 | 436.685 | 331.431 | 0.759 |
| sp\|Q9WVQ1\|MAGI2_MOUSE | Magi2 | MAGI2_MOUSE Membrane-associated guanylate kinase, | 1 | 60.5005 | 45.9037 | 0.759 |
| | | WW and PDZ domain-containing protein 2 | | | | |
| sp\|Q9QUL3\|PA2GE_MOUSE | Pla2g2e | PA2GE_MOUSE Group IIE secretory phospholipase A2 | 1 | 68.4027 | 51.8909 | 0.759 |
| sp\|Q9CQ54\|NDUC2_MOUSE | Ndufe2 | NDUC2_MOUSE NADH dehydrogenase [ubiquinone] 1 | 18 | 6597.66 | 4996.41 | 0.757 |
| | | subunit C2 | | | | |
| sp\|O89017\|LGMN_MOUSE | Lgmn | LGMN_MOUSE Legumain | 53 | 9247.38 | 6998.84 | 0.757 |
| sp\|Q61543\|GSLG1_MOUSE | Glg1 | GSLG1_MOUSE Golgi apparatus protein 1 | 44 | 9544.09 | 7216.14 | 0.756 |
| sp\|P81117\|NUCB2_MOUSE | Nucb2 | NUCB2_MOUSE Nucleobindin-2 | 17 | 4519.97 | 3415.12 | 0.756 |
| sp\|Q9ER00\|STX2_MOUSE | Stx12 | STX12_MOUSE Syntaxin-12 | 1 | 76.4151 | 57.5961 | 0.754 |
| sp\|Q3U186\|SYRM_MOUSE | Rars2 | SYRM_MOUSE Probable arginine--tRNA ligase, | 4 | 385.545 | 289.749 | 0.752 |
| | | mitochondrial | | | | |
| sp\|Q64433\|CH10_MOUSE | Hspe1 | CH10_MOUSE 10 kDa heat shock protein, mitochondrial | 4 | 1094.61 | 821.627 | 0.751 |
| sp\|Q05920\|PYC_MOUSE | Pc | PYC_MOUSE Pyruvate carboxylase, mitochondrial | 130 | 29371.7 | 22044.5 | 0.751 |
| sp\|Q8BP01\|VMAC_MOUSE | Vmac | VMAC_MOUSE Vimentin-type intermediate filament- | 4 | 692.479 | 518.326 | 0.749 |
| | | associated coiled-coil protein | | | | |
| sp\|Q99KE1\|MAOM_MOUSE | Me2 | MAOM_MOUSE NAD-dependent malic enzyme, | 82 | 19569 | 14603.6 | 0.746 |
| | | mitochondrial | | | | |
| sp\|Q99N89\|RM43_MOUSE | Mrpl43 | RM43_MOUSE 39S ribosomal protein L43, mitochondrial | 1 | 69.0903 | 51.4802 | 0.745 |
| sp\|Q8CGC7\|SYEP_MOUSE | Eprs | SYEP_MOUSE Bifunctional glutaniate/proline--tRNA ligase | 2 | 943.289 | 702.066 | 0.744 |
| sp\|P63154\|CRNL1_MOUSE | Cmkl1 | CRNL1_MOUSE Crooked neck-like protein 1 | 3 | 534.826 | 397.888 | 0.744 |
| sp\|Q9DCM0\|ETHE1_MOUSE | Ethe1 | ETHE1_MOUSE Persulfide dioxygenase ETHE1, | 5 | 697.36 | 517.832 | 0.743 |
| | | mitochondrial | | | | |
| sp\|P11881\|ITPR1_MOUSE | Itpr1 | ITPR1_MOUSE Inositol 1,4,5-trisphosphate receptor type 1 | 1 | 118.639 | 88.0656 | 0.742 |
| tr\|E9PWT2\|E9PWT2_MOUSE | Zfp229 | E9PWT2_MOUSE Protein Zfp229 | 1 | 385.038 | 285.558 | 0.742 |
| sp\|P48377\|RFX1_MOUSE | Rfx1 | RFX1_MOUSE MHC class II regulatory factor RFX1 | 3 | 179.08 | 132.791 | 0.742 |
| sp\|Q91VU0\|FAM3C_MOUSE | Fam3c | FAM3C_MOUSE Protein FAM3C | 2 | 431.462 | 319.834 | 0.741 |
| sp\|Q9JKW0\|AR6P1_MOUSE | Arl6ip1 | AR6P1_MOUSE ADP-ribosylation factor-like protein 6- | 2 | 301.619 | 223.131 | 0.740 |
| | | interacting protein 1 | | | | |
| sp\|Q8BP22\|F92A1_MOUSE | Fam92a1 | F92A1_MOUSE Protein FAM92A1 | 1 | 313.623 | 231.524 | 0.738 |
| sp\|P54116\|STOM_MOUSE | Stom | STOM_MOUSE Erythrocyte band 7 integral membrane | 8 | 765.769 | 564.332 | 0.737 |
| | | protein | | | | |
| sp\|O89051\|ITM2B_MOUSE | Itm2b | ITM2B_MOUSE Integral membrane protein 2B | 2 | 232.411 | 171.236 | 0.737 |
| sp\|Q8R2G6\|CCD80_MOUSE | Ccdc80 | CCD80_MOUSE Coiled-coil domain-containing protein 80 | 1 | 42.0849 | 30.9838 | 0.736 |
| sp\|P97287\|MCL1_MOUSE | Mcl1 | MCL1_MOUSE Induced myeloid leukemia cell | 1 | 35.5196 | 25.9144 | 0.730 |
| | | differentiation protein Mcl-1 homolog | | | | |
| sp\|P20060\|HEXB_MOUSE | Hexb | HEXB_MOUSE Beta-hexosaminidase subunit beta | 1 | 167.543 | 122.166 | 0.729 |
| sp\|Q80VP2\|SPAT7_MOUSE | Spata7 | SPAT7_MOUSE Spermatogenesis-associated protein | 4 | 984.399 | 717.312 | 0.729 |
| | | 7 homolog | | | | |
| sp\|Q9DBH5\|LMAN2_MOUSE | Lman2 | LMAN2_MOUSE Vesicular integral-membrane protein | 9 | 986.81 | 717.802 | 0.727 |
| | | VIP36 | | | | |
| sp\|Q9CYL5\|GAPR1_MOUSE | Glipr2 | GAPR1_MOUSE Golgi-associated plant pathogenesis- | 2 | 114.51 | 83.1831 | 0.726 |
| | | related protein 1 | | | | |
| sp\|A2BH40\|ARI1A_MOUSE | Arid1a | ARI1A_MOUSE AT-rich interactive domain-containing | 2 | 98.0565 | 71.0532 | 0.725 |
| | | protein 1A | | | | |
| sp\|Q9DB20\|ATPO_MOUSE | Atp50 | ATPO_MOUSE ATP synthase subunit O, mitochondrial | 4 | 485.026 | 351.028 | 0.724 |
| sp\|P63001\|RAC1_MOUSE | Rac1 | RAC1_MOUSE Ras-related C3 botulinum toxin substrate 1 | 6 | 1195.12 | 863.421 | 0.722 |
| sp\|P48771\|CX7A2_MOUSE | Cox7a2 | CX7A2_MOUSE Cytochrome c oxidase subunit 7A2, | 3 | 403.62 | 291.482 | 0.722 |
| | | mitochondrial | | | | |
| sp\|Q3TKT4\|SMCA4_MOUSE | Smarca4 | SMCA4_MOUSE Transcription activator BRG1 | 1 | 58.5914 | 42.304 | 0.722 |
| sp\|P56391\|CX6B1_MOUSE | Cox6b1 | CX6B1_MOUSE Cytochrome c oxidase subunit 6B1 | 1 | 265.586 | 191.638 | 0.722 |
| sp\|Q9JIK9\|RT34_MOUSE | Mrps34 | RT34_MOUSE 28S ribosomal protein S34, mitochondrial | 1 | 220.479 | 158.827 | 0.720 |
| sp\|Q8VEA4\|MIA40_MOUSE | Chchd4 | MIA40_MOUSE Mitochondrial intermembrane space import | 2 | 468.253 | 336.954 | 0.720 |
| | | and assembly protein 40 | | | | |
| sp\|P61924\|COPZ1_MOUSE | Copz1 | COPZ1_MOUSE Coatomer subunit zeta-1 | 2 | 268.434 | 192.617 | 0.718 |
| sp\|P53395\|ODB2_MOUSE | Dbt | ODB2_MOUSE Lipoamide acyltransferase component | 16 | 2746.66 | 1969.14 | 0.717 |
| | | of branched-chain alpha-keto acid dehydrogenase | | | | |
| | | complex, mitochondrial | | | | |
| sp\|P00761\|TRYP_PIG_ | | TRYP_PIG_contaminant Trypsin | 182 | 36651.9 | 26242.8 | 0.716 |
| contaminant | | | | | | |
| tr\|Q3U3J1\|Q3U3J1_MOUSE | Bckdha | Q3U3J1_MOUSE 2-oxoisovalerate dehydrogenase | 3 | 760.852 | 542.62 | 0.713 |
| | | subunit alpha, mitochondrial | | | | |
| sp\|P97821\|CATC_MOUSE | Ctsc | CATC_MOUSE Dipeptidyl peptidase 1 | 23 | 2777.71 | 1980.48 | 0.713 |
| sp\|Q61001\|LAMA5_MOUSE | Lama5 | LAMA5_MOUSE Laminin subunit alpha-5 | 11 | 1132.25 | 805.923 | 0.712 |
| sp\|Q9JI18\|LRP1B_MOUSE | Lrp1b | LRP1B_MOUSE Low-density lipoprotein receptor- | 1 | 388.621 | 275.781 | 0.710 |
| | | related protein 1B | | | | |
| sp\|Q9D6U8\|F162A_MOUSE | Fam162a | F162A_MOUSE Protein FAM162A | 2 | 385.912 | 273.333 | 0.708 |
| sp\|P56542\|DNS2A_MOUSE | Dnase2 | DNS2A_MOUSE Deoxyribonuclease-2-alpha | 3 | 485.203 | 342.966 | 0.707 |
| sp\|Q9JMG2\|C1GLC_MOUSE | C1galt1c1 | C1GLC_MOUSE C1GALT1-specific chaperone 1 | 28 | 8073.3 | 5689.91 | 0.705 |
| sp\|Q6P2L7\|CASC4_MOUSE | Casc4 | CASC4_MOUSE Protein CASC4 | 6 | 829.476 | 584.265 | 0.704 |
| tr\|Q9CZN7\|Q9CZN7_MOUSE | Shmt2 | Q9CZN7_MOUSE Serine hydroxymethyltransferase | 12 | 1682.84 | 1183.05 | 0.703 |
| sp\|Q9CQZ6\|NDUB3_MOUSE | Ndnfb3 | NDUB3_MOUSE NADH dehydrogenase [ubiquinone] 1 | 2 | 352.714 | 247.379 | 0.701 |
| | | beta subcomplex subunit 3 | | | | |
| sp\|Q9DC70\|NDUS7_MOUSE | Ndufs7 | NDUS7_MOUSE NADH dehydrogenase [ubiquinone] | 16 | 5408.45 | 3790.99 | 0.701 |
| | | iron-sulfur protein 7, mitochondrial | | | | |
| sp\|Q5XJY5\|COPD_MOUSE | Arcn1 | COPD_MOUSE Coatomer subunit delta | 2 | 126.355 | 88.5577 | 0.701 |
| sp\|Q9CZ13\|QCR1_MOUSE | Uqcrc1 | QCR1_MOUSE Cytochrome b-c1 complex subunit 1, | 1 | 38.2718 | 26.7355 | 0.699 |
| | | mitochondrial | | | | |
| sp\|Q91VT4\|CBR4_MOUSE | Cbr4 | CBR4_MOUSE Carbonyl reductase family member 4 | 1 | 166.95 | 116.418 | 0.697 |
| sp\|Q8CGZ0\|CHERP_MOUSE | Cherp | CHERP_MOUSE Calcium homeostasis endoplasmic | 1 | 248.394 | 173.057 | 0.697 |
| | | reticulum protein | | | | |
| sp\|P24638\|PPAL_MOUSE | Acp2 | PPAL_MOUSE Lysosomal acid phosphatase | 25 | 5429.02 | 3770.7 | 0.695 |
| sp\|Q9DB77\|QCR2_MOUSE | Uqcrc2 | QCR2_MOUSE Cytochrome b-c1 complex subunit 2, | 1 | 125.618 | 87.1025 | 0.693 |
| | | mitochondrial | | | | |
| sp\|Q9CY62\|RN181_MOUSE | Rnf181 | RN181_MOUSE E3 ubiquitin-protein ligase RNF181 | 5 | 1158.87 | 802.825 | 0.693 |
| sp\|Q9CQE3\|RT17_MOUSE | Mrps17 | RT17_MOUSE 28S ribosomal protein S17, mitochondrial | 1 | 62.0447 | 42.8025 | 0.690 |
| sp\|Q9CPU9\|COPT2_MOUSE | SlC31a2 | COPT2_MOUSE Probable low affinity copper uptake | 4 | 550.125 | 379.129 | 0.689 |
| | | protein 2 | | | | |
| sp\|Q9D8T7\|SLIRP_MOUSE | Slirp | SLIRP_MOUSE SRA stem-loop-interacting RNA-binding | 2 | 194.247 | 133.821 | 0.689 |
| | | protein, mitochondrial | | | | |
| sp\|Q99M87\|DNJA3_MOUSE | Dnaja3 | DNJA3_MOUSE DnaJ homolog subfamily A member 3, | 1 | 343.712 | 236.234 | 0.687 |
| | | mitochondrial | | | | |
| sp\|P56382\|ATP5E_MOUSE | Atp5e | ATP5E_MOUSE ATP synthase subunit epsilon, | 1 | 199.349 | 136.921 | 0.687 |
| | | mitochondrial | | | | |
| sp\|P56480\|ATPB_MOUSE | Atp5b | ATPB_MOUSE ATP synthase subunit beta, mitochondrial | 14 | 1072.13 | 729.676 | 0.681 |
| sp\|O09106\|HDC1_MOUSE | Hdac1 | HDAC1_MOUSE Histone deacetylase 1 | 1 | 71.1035 | 48.3112 | 0.679 |
| sp\|Q9D1L0\|CHCH2_MOUSE | Chchd2 | CHCH2_MOUSE Coiled-coil-helix-coiled-coil-helix | 3 | 173.062 | 117.398 | 0.678 |
| | | domain-containing protein 2, mitochondrial | | | | |
| sp\|Q9Z0L8\|GGH_MOUSE | Ggh | GGH_MOUSE Gamma-glutamyl hydrolase | 5 | 1066.15 | 721.018 | 0.676 |
| sp\|Q9ERQ3\|ZN704_MOUSE | Znf704 | ZN704_MOUSE Zinc finger protein 704 | 1 | 248.328 | 167.434 | 0.674 |
| sp\|Q9ERN0\|SCAM2_MOUSE | Scamp2 | SCAM2_MOUSE Secretory carrier-associated membrane | 1 | 46.5977 | 31.3833 | 0.673 |
| | | protein 2 | | | | |
| sp\|PO5202\|AATM_MOUSE | Got2 | AATM_MOUSE Aspartate aminotransferase, | 3 | 178.916 | 120.354 | 0.673 |
| | | mitochondrial | | | | |
| sp\|Q9Z1P6\|NDUA7_MOUSE | Ndnfa7 | NDUA7_MOUSE NADH dehydrogenase [ubiquinone] 1 | 11 | 5552 | 3730.38 | 0.672 |
| | | alpha subcomplex subunit 7 | | | | |
| sp\|D3Z7P3-2\|GLSK_MOUSE | Gls | GLSK_MOUSE Isoform 2 of Glutaminase kidney isoform, | 3 | 340.175 | 226.803 | 0.667 |
| | | mitochondrial | | | | |
| sp\|P17665\|COX7C_MOUSE | Cox7c | COX7C_MOUSE Cytochrome c oxidase subunit 7C, | 2 | 423.669 | 282.394 | 0.667 |
| | | mitochondrial | | | | |
| sp\|Q9D358\|PPAC_MOUSE | Acp1 | PPAC_MOUSE Low molecular weight phosphotyrosine | 1 | 69.016 | 45.9346 | 0.666 |
| | | protein phosphatase | | | | |
| sp\|P29758\|OAT_MOUSE | Oat | OAT_MOUSE Ornithine aminotransferase, mitochondrial | 2 | 238.013 | 157.701 | 0.663 |
| sp\|Q62087\|PON3_MOUSE | Pon3 | PON3_MOUSE Serum paraoxonase/lactonase 3 | 3 | 390.676 | 258.227 | 0.661 |
| sp\|Q6PB93\|GALT2_MOUSE | Galnt2 | GALT2_MOUSE Polypeptide N-acetylgalactosaminyl- | 9 | 1435.55 | 945.7 | 0.659 |
| | | transferase 2 | | | | |
| sp\|Q99LI2\|CLCC1_MOUSE | Clcc1 | CLCC1_MOUSE Chloride channel CLIC-like protein 1 | 5 | 658.761 | 433.622 | 0.658 |
| sp\|035454\|CLCN6_MOUSE | Clcn6 | CLCN6_MOUSE Chloride transport protein 6 | 1 | 83.462 | 54.8969 | 0.658 |
| sp\|Q9CXR1\|DHRS7_MOUSE | Dhrs7 | DHRS7_MOUSE Dehydrogenase/reductase SDR | 2 | 238.39 | 156.564 | 0.657 |
| | | family member 7 | | | | |
| sp\|P62204\|CALM_MOUSE | Calm1 | CALM_MOUSE Calmodulin | 3 | 236.413 | 154.839 | 0.655 |
| sp\|P0C6F1\|DYH2_MOUSE | Dnah2 | DYH2_MOUSE Dynein heavy chain 2, axonemal | 1 | 463.375 | 302.917 | 0.654 |
| sp\|Q9DC16\|ERG11_MOUSE | Ergic1 | ERGI1_MOUSE Endoplasmic reticulum-Golgi | 9 | 1600.19 | 1043.85 | 0.652 |
| | | intermediate compartment protein 1 | | | | |
| sp\|Q9CQH3\|NDUBS_MOUSE | Ndnfb5 | NDUB5_MOUSE NADH dehydrogenase [ubiquinone] | 9 | 3932.45 | 2564.2 | 0.652 |
| | | 1 beta subcomplex subunit 5, mitochondrial | | | | |
| sp\|P20108\|PRDX3_MOUSE | Prdx3 | PRDX3_MOUSE Thioredoxin-dependent peroxide | 11 | 4005.2 | 2611.31 | 0.652 |
| | | reductase, mitochondrial | | | | |
| tr\|Q3U422\|Q3U422_MOUSE | Ndufv3 | Q3U422_MOUSE NADH dehydrogenase [ubiquinone] | 9 | 1299.72 | 846.227 | 0.651 |
| | | flavoprotein 3, mitochondrial | | | | |
| sp\|Q8BYW9\|EOGT_MOUSE | Eogt | EOGT_MOUSE EGF domain-specific O-linked N- | 14 | 2007.56 | 1304.32 | 0.650 |
| | | acetylglucosamine transferase | | | | |
| sp\|Q14C51\|PTCD3_MOUSE | Ptcd3 | PTCD3_MOUSE Pentatricopeptide repeat domain- | 1 | 42.0293 | 27.2118 | 0.647 |
| | | containing protein 3, mitochondrial | | | | |
| sp\|P14847\|CRP_MOUSE | Crp | CRP_MOUSE C-reactive protein | 1 | 246.276 | 158.532 | 0.644 |
| sp\|Q8VCW8\|ACSF2_MOUSE | Acsf2 | ACSF2_MOUSE Acyl-CoA synthetase family member | 4 | 564.264 | 361.942 | 0.641 |
| | | 2, mitochondrial | | | | |
| sp\|O08912\|GALT1_MOUSE | Galnt1 | GALT1_MOUSE Polypeptide N-acetylgalactosaminyl- | 1 | 75.5893 | 48.4796 | 0.641 |
| | | transferase 1 | | | | |
| sp\|C6KI89\|CTSG2_MOUSE | Catsperg2 | CTSG2_MOUSE Cation channel sperm-associated | 1 | 91.9472 | 58.75 | 0.639 |
| | | protein subunit gamma 2 | | | | |
| sp\|P50544\|ACADV_MOUSE | Acadvl | ACADV_MOUSE Very long-chain specific acyl-CoA | 10 | 1401.75 | 890.786 | 0.635 |
| | | dehydrogenase, mitochondrial | | | | |
| sp\|P06800\|PTPRC_MOUSE | Ptprc | PTPRC_MOUSE Receptor-type lyrosine-protein | 1 | 146.128 | 92.4332 | 0.633 |
| | | phosphatase C | | | | |
| sp\|P01942\|HBA_MOUSE | Hba | HBA_MOUSE Hemoglobin subunit alpha | 2 | 333.836 | 209.009 | 0.626 |
| sp\|O35375\|NRP2_MOUSE | Nrp2 | NRP2_MOUSE Neuropilin-2 | 1 | 50.5976 | 31.6314 | 0.625 |
| sp\|P62874\|GBB1_MOUSE | Gnb1 | GBB1_MOUSE Guanine nucleotide-binding protein | 3 | 403.822 | 252.335 | 0.625 |
| | | G(I)/G(S)/G(T) subunit beta-1 | | | | |
| sp\|Q91YM4\|TBRG4_MOUSE | Tbrg4 | TBRG4_MOUSE Protein TBRG4 | 7 | 1508.16 | 936.45 | 0.621 |
| sp\|Q8BH04\|PCKGM_MOUSE | Pck2 | PCKGM_MOUSE Phosphoenolpyruvate carboxykinase | 9 | 1486.71 | 908.986 | 0.611 |
| | | mitochondrial | | | | |
| tr\|Q9CPN9\|Q9CPN9_MOUSE | 2210 | Q9CPN9_MOUSE Protein 2210010C04Rik | 14 | 6248.49 | 3814.07 | 0.610 |
| | 010C04 | | | | | |
| | Rik | | | | | |
| sp\|P58252\|EF2_MOUSE | Eef2 | EF2_MOUSE Elongation factor 2 | 3 | 474.539 | 288.142 | 0.607 |
| sp\|Q61171\|PRDX2_MOUSE | Prdx2 | PRDX2_MOUSE Peroxiredoxin-2 | 3 | 385.868 | 233.929 | 0.606 |
| sp\|Q80ZS3\|RT26_MOUSE | Mrps26 | RT26_MOUSE 28S ribosomal protein S26, mitochondrial | 1 | 310.505 | 188.225 | 0.606 |
| sp\|P62911\|RL32_MOUSE | Rpl32 | RL32_MOUSE 60S ribosomal protein L32 | 3 | 454.686 | 274.825 | 0.604 |
| sp\|P56383\|AT5G2_MOUSE | Atp5g2 | AT5G2_MOUSE ATP synthase F(0) complex subunit C2, | 1 | 510.505 | 307.799 | 0.603 |
| | | mitochondrial | | | | |
| sp\|Q9Z0Xl\|AIFM1_MOUSE | Aif1 | AIFM1_MOUSE Apoptosis-inducing factor 1, mitochondrial | 12 | 1688.68 | 1009.71 | 0.598 |
| sp\|Q9CQ69\|QCR_MOUSE | Uqcrq | QCR8_MOUSE Cytochrome b-c1 complex subunit 8 | 1 | 191.543 | 114.105 | 0.596 |
| sp\|Q9CQW2\|ARL8B_MOUSE | Arl8b | ARL8B_MOUSE ADP-ribosylation factor-like protein 8B | 1 | 97.9312 | 58.0836 | 0.593 |
| sp\|Q9D0S9\|HINT2_MOUSE | Hint2 | HINT2_MOUSE Histidine triad nucleotide-binding protein | 1 | 49.5112 | 29.3209 | 0.592 |
| | | 2, mitochondrial | | | | |
| sp\|Q8C3X2\|CC90B_MOUSE | Ccdc90b | CC90B_MOUSE Coilcd-coil domain-containing protein | 10 | 2244.88 | 1324.9 | 0.590 |
| | | 90B, mitochondrial | | | | |
| sp\|Q80X85\|RT07_MOUSE | Mrps7 | RT07_MOUSE 28S ribosomal protein S7, mitochondrial | 1 | 67.462 | 39.7676 | 0.589 |
| sp\|Q99L13\|3HIDH_MOUSE | Hibadh | 3HIDH_MOUSE 3-hydroxyisobutyrate dehydrogenase, | 1 | 47.6572 | 28.0257 | 0.588 |
| | | mitochondrial | | | | |
| sp\|Q9R1Q7\|PLP2_MOUSE | Plp2 | PLP2_MOUSE Proteolipid protein 2 | 2 | 382.528 | 224.023 | 0.586 |
| sp\|Q9Z138\|ROR2_MOUSE | Ror2 | ROR2_MOUSE Tyrosine-protein kinase transmembrane | 1 | 67.6684 | 39.5932 | 0.585 |
| | | receptor ROR2 | | | | |
| sp\|Q9CXD6\|MCUR1_MOUSE | Mcur1 | MCUR1_MOUSE Mitochondrial calcium uniporter | 6 | 1631.69 | 951.207 | 0.583 |
| | | regulator 1 | | | | |
| sp\|070404\|VAMP8_MOUSE | Vamp8 | VAMP8_MOUSE Vesicle-associated membrane protein 8 | 2 | 300.176 | 169.775 | 0.566 |
| sp\|Q9CYR0\|SSBP_MOUSE | Ssbp1 | SSBP_MOUSE Single-stranded DNA-binding protein, | 2 | 432.759 | 244.754 | 0.566 |
| | | mitochondrial | | | | |
| sp\|P62880\|GBB2_MOUSE | Gnb2 | GBB2_MOUSE Guanine nucleotide-binding protein | 3 | 294.957 | 165.95 | 0.563 |
| | | G(I)/G(S)/G(T) subunit beta-2 | | | | |
| sp\|Q921N7\|TMM70_MOUSE | Tmem70 | TMM70_MOUSE Transmembrane protein 70, | 1 | 88.4078 | 49.6598 | 0.562 |
| | | mitochondrial | | | | |
| tr\|Q792Z1\|Q792Z1_MOUSE | Try10 | Q792Z1_MOUSE MCG140784 | 12 | 1833.98 | 1030.02 | 0.562 |
| sp\|Q99LC5\|ETFA_MOUSE | Etfa | ETFA_MOUSE Electron transfer flavoprotein subunit | 1 | 80.1458 | 44.9816 | 0.561 |
| | | alpha, mitochondrial | | | | |
| sp\|Q9JHS4\|CLPX_MOUSE | Clpx | CLPX_MOUSE ATP-dependent Clp protease ATP- | 9 | 1517.78 | 851.066 | 0.561 |
| | | binding subunit clpX-like, mitochondrial | | | | |
| sp\|Q9DCW4\|ETFB_MOUSE | Etfb | ETFB_MOUSE Election transfer flavoprotein subunit beta | 4 | 730.275 | 409.026 | 0.560 |
| sp\|Q66GT5\|PTPM1_MOUSE | Ptpmt1 | PTPM1_MOUSE Phosphatidylglycerophosphatase and | 1 | 92.3664 | 51.6065 | 0.559 |
| | | protein-tyrosine phosphatase 1 | | | | |
| sp\|P61161\|ARP2_MOUSE | Actr2 | ARP2_MOUSE Actin-related protein 2 | 1 | 153.626 | 84.7644 | 0.552 |
| sp\|Q9WTP6\|KAD2_MOUSE | Ak2 | KAD2_MOUSE Adenylate kinase 2, mitochondrial | 2 | 113.461 | 61.9776 | 0.546 |
| sp\|P52825\|CPT2_MOUSE | Cpt2 | CPT2_MOUSE Carnitine O-palmitoyltransferase 2, | 2 | 177.54 | 96.8301 | 0.545 |
| | | mitochondrial | | | | |
| sp\|P97360\|ETV6_MOUSE | Etv6 | ETV6_MOUSE Transcription factor ETV6 | 1 | 137.108 | 74.6472 | 0.544 |
| sp\|Q8BJQ9\|CGAY1_MOUSE | Csgalnact1 | CGAT1_MOUSE Chondroitin sulfate N- | 14 | 2426.62 | 1300.88 | 0.536 |
| | | acetylgalactosaminyltransferase 1 | | | | |
| sp\|P59999\|ARPC4_MOUSE | Atpc4 | ARPC4_MOUSE Actin-related protein 2/3 complex | 2 | 138.296 | 74.075 | 0.536 |
| | | subunit 4 | | | | |
| sp\|Q921G7\|ETFD_MOUSE | Etfdh | ETFD_MOUSE Electron transfer flavoprotein- | 1 | 57.6477 | 30.6918 | 0.532 |
| | | ubiquinone oxidoreductase, mitochondrial | | | | |
| sp\|P41216\|ACSL1_MOUSE | Acsl1 | ACSL1_MOUSE Long-chain-fatty-acid-CoA ligase 1 | 3 | 361.33 | 192.15 | 0.532 |
| sp\|Q6ZPE2\|MTMRS_MOUSE | Sbf1 | MTMR5_MOUSE Myotubularin-related protein 5 | 1 | 71.1304 | 37.3228 | 0.525 |
| sp\|Q8R1I1\|QCR9_MOUSE | Uqcr10 | QCR9_MOUSE Cytochrome b-c1 complex subunit 9 | 1 | 243.198 | 125.477 | 0.516 |
| sp\|O35143\|ATIF1_MOUSE | Atpif1 | ATIF1_MOUSE ATPase inhibitor, mitochondrial | 1 | 454.155 | 229.372 | 0.505 |
| sp\|Q8JZN5\|ACAD9_MOUSE | Acad9 | ACAD9_MOUSE Acyl-CoA dehydrogenase family | 1 | 127.125 | 61.8219 | 0.486 |
| | | member 9, mitochondrial | | | | |
| sp\|Q8R1J9\|TOR2A_MOUSE | Tor2a | TOR2A_MOUSE Torsin-2A | 3 | 696.28 | 338.383 | 0.486 |
| sp\|B1AVY7\|KI16B_MOUSE | Kif16b | KI16B_MOUSE Kinesin-like protein KIF16B | 2 | 1117.61 | 528.649 | 0.473 |
| sp\|P63038\|CH60_MOUSE | Hspd1 | CH60_MOUSE 60 kDa heat shock protein, mitochondrial | 28 | 4263.9 | 2002.08 | 0.470 |
| tr\|K7N641\|K7N641_MOUSE | Olfr694 | K7N641_MOUSE Protein Olfr694 | 2 | 816.778 | 377.66 | 0.462 |
| sp\|P09671\|SODM_MOUSE | Sod2 | SODM_MOUSE Superoxide dismutase [Mn], mitochondrial | 3 | 1031.93 | 472.133 | 0.458 |
| sp\|Q8BFT2\|HAUS4_MOUSE | Haus4 | HAUS4_MOUSE HAUS augmin-like complex subunit 4 | 1 | 518.102 | 230.984 | 0.446 |
| sp\|P22752\|H2A1_MOUSE | Hist1h2ab | H2A1_MOUSE Histone H2A type 1 | 2 | 273.934 | 119.978 | 0.438 |
| sp\|Q8VHN7\|GPR98_MOUSE | Gpr98 | GPR98_MOUSE G-protein coupled receptor 98 | 3 | 618.49 | 249.388 | 0.403 |
| sp\|Q9Z2I9\|SUCB1_MOUSE | Sucla2 | SUCB1_MOUSE Succinyl-CoA ligase [ADP-forming] | 1 | 50.8753 | 19.8331 | 0.390 |
| | | subunit beta, mitochondrial | | | | |
| sp\|Q8VCI5\|PEX19_MOUSE | Pex19 | PEX19_MOUSE Peroxisomal biogenesis factor 19 | 1 | 60.4801 | 23.3696 | 0.386 |
| sp\|Q3U1J4\|DDB1_MOUSE | Ddb1 | DDB1_MOUSE DNA damage-binding protein 1 | 3 | 426.44 | 149.943 | 0.352 |
| sp\|Q8K221\|ARFP2_MOUSE | Arfip2 | ARFP2_MOUSE Arfaptin-2 | 2 | 1092.57 | 377.903 | 0.346 |
| sp\|Q9D051\|ODPB_MOUSE | Pdhb | ODPB_MOUSE Pyruvate dehydrogenase E1 component | 2 | 365.712 | 123.1 | 0.337 |
| | | subunit beta, mitochondrial | | | | |
| sp\|P08249\|MDHM_MOUSE | Mdh2 | MDHM_MOUSE Malate dehydrogenase, mitochondrial | 12 | 2557.03 | 858.89 | 0.336 |
| sp\|P16675\|PPGB_MOUSE | Ctsa | PPGB_MOUSE Lysosomal protective protein | 1 | 357.95 | 111.377 | 0.311 |
| sp\|P62737\|ACTA_MOUSE | Acta2 | ACTA_MOUSE Actin, aortic smooth muscle | 3 | 361.157 | 107.601 | 0.298 |
| sp\|Q9CY64\|BIEA_MOUSE | Blvra | BIEA_MOUSE Biliverdin reductase A | 1 | 302.865 | 86.0306 | 0.284 |
| sp\|P62960\|YBOX1_MOUSE | Ybx1 | YBOX1_MOUSE Nuclease-sensitive element- | 2 | 219.172 | 60.6256 | 0.277 |
| | | binding protein 1 | | | | |
| sp\|Q62028\|PLA2R_MOUSE | Pla2r1 | PLA2R_MOUSE Secretory phospholipase A2 receptor | 1 | 519.664 | 142.778 | 0.275 |
| sp\|Q3U2Pl\|SC24A_MOUSE | Sec24a | SC24A_MOUSE Protein transport protein Sec24A | 1 | 118.999 | 29.3088 | 0.246 |
| sp\|Q8BGW2-2\|WBP1L_MOUSE | Wbp1l | WBP1L_MOUSE Isoform 2 of WW domain binding | 1 | 131.148 | 27.0371 | 0.206 |
| | | protein 1-like | | | | |
| sp\|Q99KR7\|PPIF_MOUSE | Ppif | PPIF_MOUSE Peptidyl-prolyl cis-trans isomerase F, | 1 | 249.839 | 49.7858 | 0.199 |
| | | mitochondrial | | | | |
| sp\|O88492\|PLIN4_MOUSE | Plin4 | PLIN4_MOUSE Perilipin-4 | 1 | 111.989 | 18.9196 | 0.169 |
| sp\|Q08189\|TGM3_MOUSE | Tgm3 | TGM3_MOUSE Protein-glutamine gamma- | 1 | 274.388 | 45.8379 | 0.167 |
| | | glutamyltransferase E | | | | |
| sp\|Q9D125\|RT25_MOUSE | Mrps25 | RT25_MOUSE 28S ribosomal protein S25, mitochondrial | 1 | 84.7592 | 10.7103 | 0.126 |
| sp\|P60843\|IF4A1_MOUSE | Eif4a1 | IF4A1_MOUSE Eukaryotic initiation factor 4A-I | 1 | 136.49 | 17.0483 | 0.125 |
| sp\|Q6ZQ93\|UBP34_MOUSE | Usp34 | UBP34_MOUSE Ubiquitin carboxyl-terminal hydrolase 34 | | 1044.49 | 98.0506 | 0.094 |
| sp\|P97350\|PKP1_MOUSE | Pkp1 | PKP1_MOUSE Plakophilin-1 | 1 | 98.2846 | 8.01455 | 0.082 |
| sp\|P59242\|CING_MOUSE | Cgn | CING_MOUSE Cingulin | 1 | 200.289 | 16.0255 | 0.080 |
| sp\|P39654\|LOX15_MOUSE | Alox15 | LOX15_MOUSE Arachidonate 15-lipoxygenase | 1 | 141.336 | 9.02818 | 0.064 |
| sp\|P48997\|INVO_MOUSE | Ivl | INVO_MOUSE Involucrin | 1 | 222.079 | 13.3857 | 0.060 |
| sp\|Q791V5\|MTCH2_MOUSE | Mteh2 | MTCH2_MOUSE Mitochondrial carrier homolog 2 | 1 | 101.527 | 5.76631 | 0.057 |
| sp\|P04117\|FABP4_MOUSE | Fabp4 | FABP4_MOUSE Fatty acid-binding protein, adipocyte | | 746.54 | 40.1352 | 0.054 |
| tr\|E9Q0C6\|E9Q0C6_MOUSE | Gm14569 | E9Q0C6_MOUSE Protein Gm14569 | 1 | 224.172 | 8.64662 | 0.039 |
| sp\|P55292\|DSC2_MOUSE | Dsc2 | DSC2_MOUSE Desmocollin-2 | 1 | 93.7977 | 3.51005 | 0.037 |
| sp\|E9Q557\|DESP_MOUSE | Dsp | DESP_MOUSE Desmoplakin | 1 | 79.7679 | 2.70673 | 0.034 |
| sp\|Q66L42\|M3K10_MOUSE | Map3k10 | M3K10_MOUSE Mitogen-activated protein kinase kinase | 1 | 109.366 | 3.45453 | 0.032 |
| | | kinase 10 | | | | |
| sp\|P70388\|RAD50_MOUSE | Rad50 | RAD50_MOUSE DNA repair protein RAD50 | 1 | 422.246 | 10.8209 | 0.026 |
| sp\|P56567\|CYTA_MOUSE | Csta | CYTA_MOUSE Cystatin-A | 1 | 160.416 | 2.13161 | 0.013 |
| sp\|Q9D3P1\|TCHL1_MOUSE | Tchhl1 | TCHL1_MOUSE Trichohyalin-like protein 1 | | 1810.14 | 23.5307 | 0.013 |
| sp\|P16460\|ASSY_MOUSE | Ass1 | ASSY_MOUSE Argininosuccinate synthase | 1 | 285.885 | 3.2933 | 0.012 |
| tr\|E9Q9D8\|E9Q9D8_MOUSE | Ankrd35 | E9Q9D8_MOUSE Protein Ankrd35 | 1 | 314.798 | 3.32001 | 0.011 |
| tr\|E9PW83\|E9PW83_MOUSE | Fam184a | E9PW83_MOUSE Protein Fam184a | 1 | 877.972 | 8.10219 | 0.009 |
| sp\|G3X9C2\|FBX50_MOUSE | Nccrp1 | FBX50_MOUSE F-box only protein 50 | 1 | 113.564 | 0 | 0.000 |

Signal transduction pathways were then examined with various doses of irisin in the osteocytes with special reference to well-known targets of the integrins: pFAK, pZyxin and pCREB. Signal transduction pathways downstream of integrins have been associated with anti-apoptotic actions in these osteocytes (Plotkin et al. (2007) J. Biol. Chem. 282:24120-24130). The very low doses of irisin (10 pM) stimulated FAK phosphorylation (pFAK) (FIG. 2D). This analysis of signaling was extended to primary murine inguinal adipose cells as shown in FIG. 3. Again, phosphorylation of FAK and CREB were observed at relatively low doses of irisin (30 pM).

To examine whether this irisin signaling was due to integrin binding, a variety of different integrin pairs commercially available as soluble protein complexes from R&D Systems were used. The classical integrin competitive inhibitory peptide RGDS or the non-binding control RGD peptide were also used (FIG. 4).

RGDS inhibits the binding of many integrin ligands even when they do not contain a RGDS motif (Kobayashi et al. (2017) Cancers (Basel) 9(7)). In fact, the crystal structure of irisin contains a loop very analogous to the RGD-containing loop in fibronectin, although no RGD sequence is present in irisin. As shown in FIG. 4, the RGDS peptide inhibited much of the irisin binding to these integrins, compared to the control RGD peptide (GRADSP, G in RGD is switched to A). Irisin produced in mammalian cells, as shown here, ran as two bands that both result from glycosylation of the 12 kD polypeptide.

These results led to an examination of the effects of integrin inhibitors on irisin signaling within cells. As shown in FIG. 5A-FIG. 5B, most irisin signaling in osteocytes was inhibited by either RGDS peptide (FIG. 5A) or a second integrin inhibitor, echistatin (FIG. 5B). Echistatin is a natural integrin inhibitor isolated from viper venom (Atkinson et al. (1994) Int. J. Pept. Protein Res. 43:563-572).

One of the best characterized products secreted by osteocytes is sclerostin. This hormone is made specifically by osteocytes, stimulates osteoclasts and bone breakdown, and is known to be increased with exercise (Bonewald (2017) Endocrinol. Metab. Clin. North Am. 46:1-18; Pickering et al. (2017) Calcif. Tissue Int. 101:170-173). As shown in FIG. 6A-FIG. 6B, sclerostin mRNA was increased in osteocytes treated in culture with various doses of irisin. Furthermore, this irisin mRNA induction was sensitive to 3 integrin inhibitors: RGDS peptide, RGDyK circular peptide and echistatin.

Irisin or vehicle was also intraperitoneally injected into wild type C57/Bl6 mice, once a day for 6 days. Bones and blood were then harvested from these mice. As shown in FIG. 7A, irisin stimulated sclerostin mRNA in these bone preparations at 0.1 and 1.0 mg/kg. Furthermore, there was also a significant increase in circulating sclerostin (FIG. 7B).

Adipose cell-selective gene expression were also examined in these irisin-injected mice. As shown in FIG. 8, irisin injections increased expression of mRNAs for genes of the classical thermogenic pathway, such as UCP1 and DIO 2. These treatments also increased expression of genes of the futile creatine cycle, including GATM (first step of creatine synthesis) and two creatine kinases, CKMT2 and CKB. It has been recently shown the importance of adipose GATM and the creatine cycle in energy expenditure in mice (Kazak et al. (2015) Cell 163:643-655; Kazak et al. (2017) Cell Metab. 26:660-671).

Finally, FNDC5 knockout (KO) mice were made (FIG. 9E-FIG. 9F). The experiments shown were performed with whole body KOs. The effects of loss of FNDC5/irisin on osteoporosis in mice were examined via ovariectomy. This is the most widely used model of experimental osteoporosis. A nearly complete protection against bone loss in the FNDC5 KO mice was observed, as determined by bone mineral volume/total volume and trabecular thickness (FIG. 9E) and number (FIG. 9F).

Osteocytes play an important role in bone remodeling. Based on FIG. 9E-FIG. 9F, osteocyte function, including eroded bone surfaces and lacunae, was specifically examined. Lacunae are the spaces wherein the osteocytes reside. As shown in FIG. 9J and FIG. 10E, both parameters indicated reduced osteocyte function in the FNDC5 KOs.

Taken together, these data are consistent with a model whereby the osteocytes are stimulated by irisin to survive and secrete bone mobilizing hormones, especially sclerostin. When this happens intermittently, like with exercise, or via occasional irisin injection, bone remodeling and bone improvement occurs (Colaianni et al. (2015) Proc. Natl. Acad. Sci. U.S.A. 112:12157-12162). However, the chronic loss of irisin/FNDC5 clearly is negative toward osteocyte degradative function and is very protective of bone, as demonstrated herein in the context of the ovariectiomy model.

### Example 3

### Irisin Treatment Induces the Expression of Sclerostin in Osteocytes for Bone Remodeling

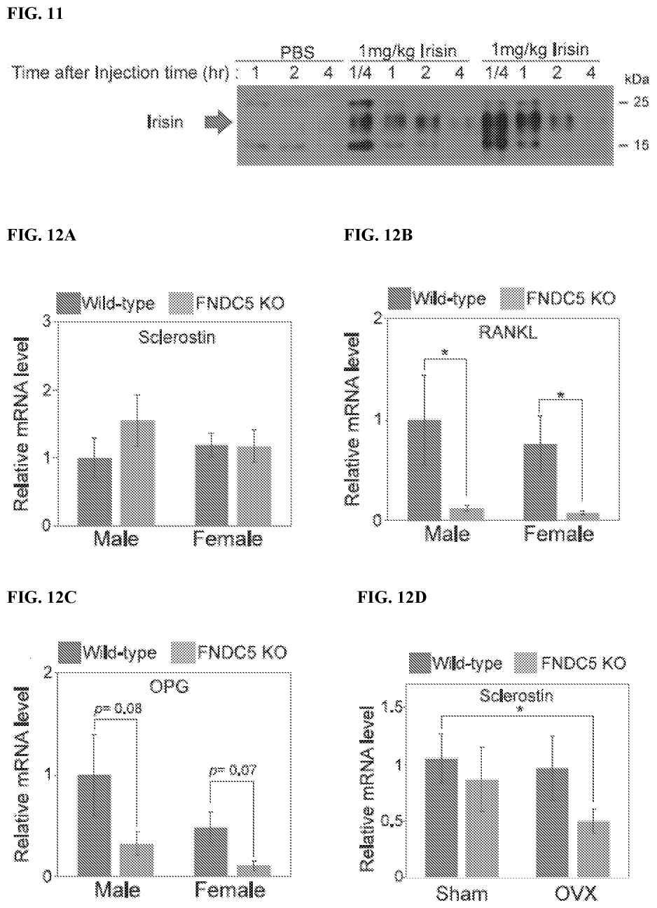

The following examples further comfirm the Example 2 described above. To study the functional roles of irisin in osteocytes, the MLO-Y4 (osteocyte-like) cell line was used (Kato et al. (1997) J. Bone Miner. Res. 12:2014-2023). Osteocytes are lost with aging and their death is thought to be an important component in the pathogenesis of age-related osteoporosis. Treatment with hydrogen peroxide has been previously used in these osteocyte-like cells as an assay for apoptotic death (Kitase et al. (2018) Cell Rep. 22:1531-1544). Therefore, MLO-Y4 cells were treated with irisin in the presence of hydrogen peroxide at amounts sufficient to induce apoptosis (FIG. 1A). Irisin treatment reduced hydrogen peroxide-induced apoptosis at concentrations of 1-500 ng/ml. Importantly, these effects were seen within the physiological concentration found in human plasma (3-5 ng/ml) (Jedrychowski et al. (2015) Cell Metab. 22:734-740) (FIG. 1A). Since exercise also raises the levels of plasma sclerostin, a specific product of osteocytes that causes bone resorption and initiates bone remodeling, expression of this hormone with irisin treatments was also examined. Irisin raised the mRNA level of sclerostin in the osteocyte cultures in a dose-dependent manner (FIG. 1B). To examine the regulation by irisin in vivo, recombinant irisin protein was injected daily into mice for 6 days (see methods). As shown in FIGS. 1C and D, these injections raised the sclerostin mRNA level in osteocyte-enriched bones, as well as the protein level in plasma even though the half-life of recombinant irisin in vivo is less than an hour (FIG. 11). These results demonstrate that irisin can protect osteocytes against apoptosis in culture and induce the expression of sclerostin, a key regulator of bone remodeling, in vivo.

### Example 4

### Deletion of FNDC5 Prevents Ovariectomy-Induced Trabecular Bone Loss by Inactivating Osteocytic Osteolysis and Osteoclastic Bone Resorption

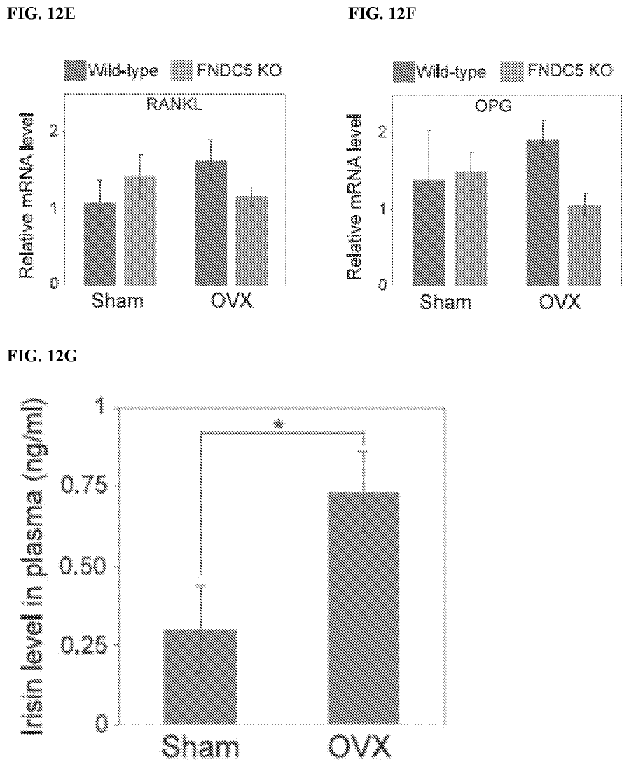

To investigate if irisin plays a role in the endogenous processes of normal bone resorption and remodeling, the femur in mice null for FNDC5 (the precursor of irisin) and littermate wild type mice were first analyzed at 5 months of age (see methods). FNDC5 null mice had significantly lower level of RANKL mRNA in whole bones both in male and female while OPG was not significantly different (FIGS. 12B and 12C). RANKL is a key factor in osteoclast activation, so the microarchitecture of bones were also analyzed. FNDC5 null mice had significantly higher femoral trabecular bone mass and greater connectivity density than wild type mice (Table 7), which is consistent with lower bone resorption and reduced expression of RANKL; on the other hand, there were no differences in cortical bone indices (Table 7). In male mice, there were no differences in bone mass, either in the cortical or trabecular compartment (Table 7).

TABLE 7
Femoral Trabecular and Cortical Bone Microstructure.

| | Female | | Male | |
|---|---|---|---|---|
| | WT | FNDC5 KO | WT | FNDC5 KO |
| | (N = 8) | (n = 7) | (n = 6) | (n = 7) |
| Age (wks) | 22.8 ± 0.7 | 21.8 ± 0.9 | 21.3 ± 1.0 | 21.6 ± 0.9 |
| Femur length | 15.6 ± 0.16 | 15.8 ± 0.1 | 15.3 ± 0.1 | 15.3 ± 0.1 |
| (mm) | | | | |
| Body weight | 23.5 ± 1.2 | 23.1 ± 1.1 | 29.4 ± 1.5 | 29.4 ± 1.7 |
| (g) | | | | |

Distal femur trabecular bone

| | | | | |
|---|---|---|---|---|
| Tb.BC/TV | 2.6 ± 0.4 | 4.1 ± 0.4** | 11.0 ± 2.4 | 10.5 ± 1.79 |
| (%) | | | | |
| Tb.DMB | 115 ± 4.5 | 136 ± 4.4** | 196 ± 22 | 195 ± 16 |
| (mgHA/cm3) | | | | |
| Tb.BS/VC | 71.0 ± 3.0 | 66.6 ± 2.0 | 59.5 ± 5.6 | 56.3 ± 3.8 |
| (mm2/mm3) | | | | |
| Tb.ConnD | 5.2 ± 2.1 | 15.2 ± 2.3** | 80.9 ± 18.6 | 66.2 ± 14.6 |
| (1/mm3) | | | | |
| SMI | 3.69 ± 0.11 | 3.26 ± 0.08** | 2.39 ± 0.31 | 2.49 ± 0.23 |
| Tb.N (1/mm) | 2.47 ± 0.13 | 2.72 ± 0.10 | 3.97 ± 0.16 | 3.80 ± 0.20 |
| Tb.Th (μm) | 44 ± 2 | 44 ± 1 | 48 ± 3 | 50 ± 2 |
| Tb.Sp (μm) | 413 ± 19 | 372 ± 14* | 248 ± 12 | 261 ± 17 |

Fermoral diaphysis cortical bone

| | | | | |
|---|---|---|---|---|
| Tt.Ar (cm2) | 1.63 ± 0.07 | 1.73 ± 0.04 | 2.21 ± 0.07 | 2.05 ± 0.15 |
| Ct.Ar (cm2) | 0.82 ± 0.03 | 0.85 ± 0.02 | 0.87 ± 0.05 | 0.85 ± 0.03 |
| Ma.Ar (cm2) | 0.80 ± 0.04 | 0.88 ± 0.03 | 1.34 ± 0.07 | 1.20 ± 0.12 |
| Ct.Ar/Tt.Ar | 50.7 ± 1.0 | 49.3 ± 0.8 | 39.3 ± 1.8 | 42.2 ± 1.7 |
| (%) | | | | |
| Ct.Th (μm) | 206 ± 5 | 207 ± 4 | 171 ± 8 | 181 ± 5 |
| Ct. TMD | 1251 ± 5 | 1247 ± 4 | 1179 ± 10 | 1197 ± 10 |
| (mgHA/cm3) | | | | |
| Ct.Po (%) | 0.69 ± 0.04 | 0.73 ± 0.02 | 1.12 ± 0.20 | 0.96 ± 0.10 |
| pMOI | 0.34 ± 0.03 | 0.37 ± 0.01 | 0.52 ± 0.03 | 0.47 ± 0.05 |

Data are mean ± SD.
*p < 0.05 vs WT,
**p < 0.01 vs WT;
# p < 0.10 vs WT

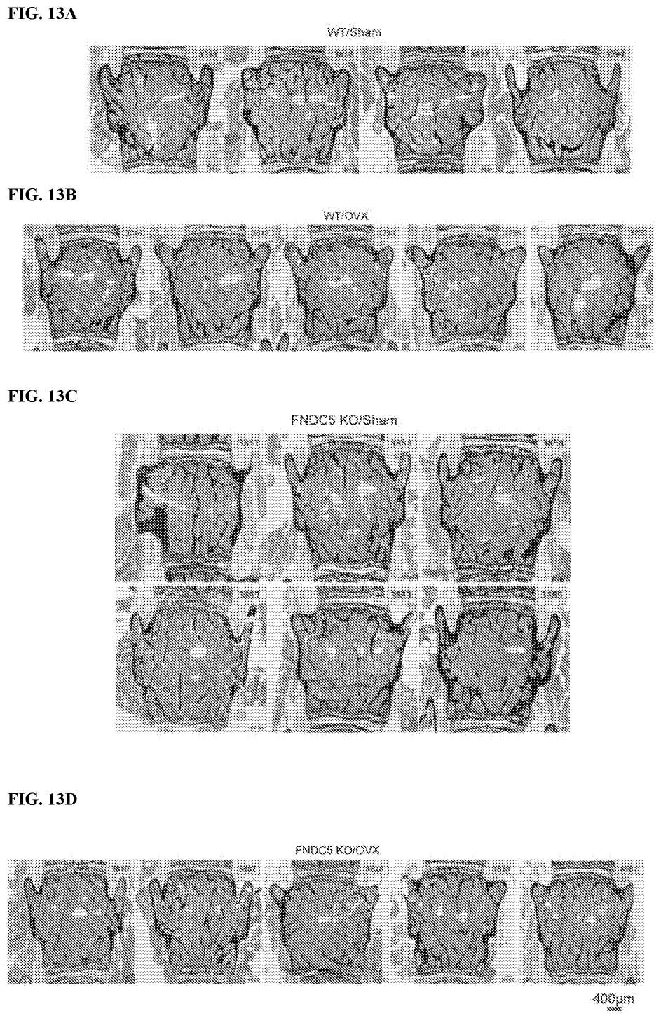

To further investigate the role of irisin in bone resorption, particularly in this pathological context, ovariectomy (OVX) (Idris, 2012) was performed in mice null for FNDC5 and their littermate controls. Ovariectomy increased bone resorption and caused bone loss in wild-type mice, compared to the sham operated group (FIGS. 9A-9D, and FIG. 13). This was apparent by the ratio of bone volume to total bone volume, trabecular number and the separation between trabeculae in the lumbar vertebrae (FIGS. 9E-G, and Tables 8-9). However, FNDC5 null mice were strikingly resistant to OVX-induced trabecular bone loss (FIG. 9A-D, FIG. 13). The maintenance of bone mass in the absence of estrogen in FNDC5 null mice was principally due to marked reduction in bone resorption (FIGS. 9H-J, and Tables 8-9). Consistent with the lack of resorption in the OVX'd null mice, whole bone RANKL mRNA remained unchanged (FIG. 12E). On the other hand, there were no differences in osteoblast number or bone formation rate for the OVX'd FNDC5 null mice compared to OVX'd wild-type mice (Tables 8-9). To ascertain the mechanism responsible for the absence of bone loss and lack of change in RANKL with estrogen deficiency in the FNDC5 KO mice, cortical bone was compared histologically from both controls and null mice after OVX. In the FNDC5 null mice, there was a striking lack of osteocytic osteolysis and lacunae enlargement (FIG. 10A-E and Tables 10-11) compared to OVX'd control mice, whose cortical bone was characterized by marked enlargement in osteocytic lacunae due to enhanced osteocytic osteolysis (FIG. 10A-E and Tables 10-11). Taken together, these data indicate that FNDC5/irisin is required for ovariectomy-induced osteolysis and strongly indicate that endogenous FNDC5/irisin induces bone resorption, at least partly through its actions on osteocytes.

TABLE 8
Bone histomorphometric analysis of Von Kossa stained lumbar vertebra from
wild-type mice or FNDC5/irisin knockout mice after OVX.

| | WT | WT | FNDC KO | FNDC KO |
|---|---|---|---|---|
| | Sham | OVX | Sham | OVX |
| Parameters | (n = 4) | (n = 5) | (n = 6) | (n = 5) |
| BV/TV(%) | 8.65 ± 1.44 | 5.48 ± 1.24 | 8.70 ± 2.58 | 11.0 ± 4.48 # |
| Tb.Th (um) | 38.7 ± 2.98 | 35.7 ± 3.47 | 37.8 ± 6.05 | 38.0 ± 5.69 |
| Th.N (/mm) | 2.23 ± 0.33 | 1.52 ± 0.21 | 2.29 ± 0.45 | 2.67 ± 0.73 # |
| Th.Sp (mu) | 423 ± 51.3 | 633 ± 106 * | 415 ± 89.4 | 360 ± 108 # |
| MAR (um/day) | 1.11 ± 0.12 | 1.12 ± 0.09 | 1.14 ± 0.16 | 1.11 ± 0.19 |
| MS/BS (%) | 40.2 ± 2.25 | 46.2 ± 1.95 * | 47.8 ± 2.69 * | 44.7 ± 4.13 |
| BFR/BV (%/day) | 2.44 ± 0.42 | 2.99 ± 0.48 | 3.09 ± 0.72 | 2.70 ± 0.50 |
| BFR/BS (um³/um²/day) | 0.451 ± 0.07 | 0.52 ± 0.05 | 0.55 ± 0.09 | 0.50 ± 0.09 |
| BFR/TV (%/day) | 0.19 ± 0.04 | 0.16 ± 0.02 | 0.25 ± 0.04 | 0.26 ± 0.09 # |
| N.Ob/B.Pm (/mm) | 6.37 ± 2.29 | 10.2 ± 3.34 | 6.27 ± 1.66 | 9.77 ± 1.66 |
| N.Ob/T.Ar (/mm²) | 28.7 ± 7.66 | 30.8 ± 12.7 | 27.6 ± 4.94 | 52.8 ± 14.0 *#$ |
| Ob.S/B.Pm (%) | 7.58 ± 2.82 | 11.5 ± 3.88 | 7.65 ± 1.91 | 11.3 ± 1.54 |
| OS/BS (%) | 10.5 ± 2.32 | 16.8 ± 3.45 | 14.0 ± 5.41 | 12.8 ± 2.67 |
| O.Th (um) | 1.93 ± 0.34 | 2.19 ± 0.25 | 2.18 ± 0.17 | 2.18 ± 0.25 |
| N.Oc/B.Pm (/mm) | 3.31 ± 1.02 | 6.20 ± 0.96* | 4.16 ± 1.01 | 4.39 ± 1.07 |
| N.Oc/T.Ar (/mm2) | 15.5 ± 5.01 | 18.3 ± 3.66 | 19.4 ± 7.47 | 24.4 ± 10.2 |
| Oc.S/B.Pm (%) | 7.41 ± 2.23 | 13.6 ± 2.51 * | 10.0 ± 2.32 | 10.4 ± 2.29 |
| ES/BS (%) | 2.22 ± 1.53 | 5.16 ± 1.68 * | 2.96 ± 1.10 | 3.02 ± 0.85 |

Data are mean ± SD.
* p < 0.05 vs WT-Sham Group.
# p < 0.05 vs WT-OVX Group.
$ p < 0.05 vs FNDC KO-Sham group.

TABLE 9
Two-way ANOVA of table 8

| | Two-way ANOVA | | |
|---|---|---|---|
| | | | Interaction |
| | WT | Sham | between |
| | vs | vs | FNDC KO |
| Parameters | FNDC KO | OVX | and OVX |
| BT/TV (%) | p = 0.0435 * | p = 0.7328 | p = 0.0467 * |
| Tb.Th (um) | p = 0.7437 | p = 0.5486 | p = 0.4682 |
| Tb.N(/mm) | p = 0.0134 * | p = 0.4610 | p = 0.0227 * |
| Tb.Sp (um) | p = 0.0042 ** | p = 0.0831 | p = 0.0062 ** |
| MAR (um/day) | p = 0.8958 | p = 0.8985 | p = 0.7907 |
| MS/BS (%) | p = 0.0316 * | p = 0.2581 | p = 0.0030 ** |
| BFR/BC (%/day) | p = 0.4946 | p = 0.7570 | p = 0.0803 |
| BFR/BS (um³/um²/day) | p = 0.2920 | p = 0.7364 | p = 0.1132 |
| BFR/TV (%/day) | p = 0.0053 ** | p = 0.7799 | p = 0.4567 |
| N.Ob/B.Pm (/mm) | p = 0.8053 | p = 0.0029 ** | p = 0.8818 |
| N.Ob/T.Ar (/mm²) | p = 0.0405 * | p = 0.0102 * | p = 0.0248 * |
| Ob.S/B.Pm (%) | p = 0.9365 | p = 0.0060 ** | p = 0.8980 |
| OS/BS (%) | p = 0.8765 | p = 0.1653 | p = 0.0481 * |
| O.Th (um) | p = 0.2926 | p = 0.2645 | p = 0.2688 |
| N.Oc/B.Pm (/mm) | p = 0.3112 | p = 0.0037 ** | p = 0.0105 * |
| N.Oc/T.Ar (/mm²) | p = 0.1422 | p = 0.2412 | p = 0.7280 |
| Oc.S/B.Pm (%) | p = 0.8054 | p = 0.0069 ** | p = 0.0155 * |
| ES/BS (%) | p = 0.2510 | p = 0.0216 * | p = 0.0267 * |

Two-way ANOVA was performed with p < 0.05 considered significant for statistical analysis by using online application ANOVA4 (http://www.hju.ac.jp/~kiriki/anova4/).
*; p < 0.05,
**; p < 0.01

TABLE 10
Osteocyte analysis to measure lacunae area of vertebra from wild-type mice or
FNDC5/irisin knockout mice after OVX using backscatter scanning electron microscopy.

| | WT | WT | FNDC KO | FMK KO |
|---|---|---|---|---|
| | Sham | OVX | Sham | OVX |
| Osteocyte Pineers | (n = 4) | (n = 5) | (n = 6) | (n = 5) |
| Lacunae Area (um²) | 23.8 ± 2.40 | 27.8 ± 1.59 * | 23.3 ± 2.53 | 24.5 ± 1.48 # |
| Lacunae Density (*10⁻⁴/um²) | 5.93 ± 0.88 | 5.12 ± 0.49 | 6.49 ± 1.02 | 5.90 ± 0.60 |

Data are mean ± SD.
* p < 0.05 vs WT-Sham Group.
# p < 0.05 vs WT-OVX Group.

TABLE 11
Two-way ANOVA of table 10

| | Two-way ANOVA | | |
|---|---|---|---|
| | WT | Sham | |
| | vs | vs | Ineraction between |
| Osteocyte Parameters | FNDC KO | OVX | FNDC KO and OVX |
| Lacunae Area | p = 0.0496 * | p = 0.0120 * | p = 0.2173 |
| Lacunae Density | p = 0.1490 | p = 0.1266 | p = 0.4426 |

Two-way ANOVA was performed with p < 0.05 considered significant for statistical analysis by using online application ANOVA4 (available on the World Wide Web at hju.ac.jp/~kiriki/anova4/).
*; p < 0.05.

In light of these data, it was determined whether ovariectomy changed irisin levels. OVX was performed in 8 weeks old wild-type mice; irisin was measured in plasma 2 weeks after OVX using quantitative Mass Spectrometry by the AQUA method (Jedrychowski et at (2015) Cell Metab. 22:734-740). Control (sham operated) mice had 0.3 ng/ml of irisin in plasma, while the OVX mice had 2.4 fold more (FIG. 12G). Interestingly, this is 10 fold less than healthy young human males (Jedrychowski et al. (2015) Cell Metab. 22:734-740).

### Example 5

### Quantitative Proteomic Analysis Identified Integrin β1 as a Candidate for the Irisin Receptor and Irisin Treatment Triggers Integrin-Like Signaling

The irisin receptor has not been identified. Since the data described herein showed that MLO-Y4 osteocytes directly respond to low concentration of irisin, these cells were used to identify its receptor. Irisin with a his-tag or an identically tagged control protein (adipsin) were first incubated with intact cell surfaces at 4° C. A chemical cross-linker was then added and incubated with cells, and the ligands were re-purified with (presumptive) cellular proteins covalently attached. The cross-links were then reversed and the products were subjected to quantitative Mass Spectrometry (FIG. 2A). This quantitative proteomic analysis, using isobaric tagging, revealed five cell surface proteins as potential receptor candidates for irisin (Table 1 and Tables 6A and 6B). Among them, only integrin β1 is known to bind protein ligands and to trigger downstream signaling. Integrin β1 (like all β-integrins) binds β-integrins to form obligate heterodimers. These heterodimers, upon ligand binding, usually trigger canonical signaling by phosphorylation of focal adhesion kinase (FAK), AKT, and cAMP response element-binding protein (CREB) (Giancotti & Ruoslahti (1999) Science 285:1028-1032; Schaller et al. (1994) Mol. Cell Biol. 14:1680-1688; D'amico et al. (2000) J. Biol. Chem. 275:32649-32657) (FIG. 2B). In response to ligand binding to many integrins, FAK is auto-phosphorylated on tyrosine 397 and then downstream signaling follows (Giancotti & Ruoslahti (1999) Science 285:1028-1032). MLO-Y4 cells were treated with irisin at 10 nM or norepinephrine at the same concentration (as a positive control for phosphorylation of CREB); irisin treatment caused phosphorylation of FAK in 1 minute and the signal decreased after 10 minutes (FIG. 2C). AKT was phosphorylated on threonine 308 while phosphorylation of serine at amino acid 473 was not induced. Additionally, CREB was phosphorylated after 5 mins with irisin and as expected, norepinephrine also did this (FIG. 2C). The dose response of these signaling events was then examined. Treatment of these osteocytes with irisin doses as low as 10 pM induced the phosphorylation of FAK (FIG. 2D). Zyxin, another downstream protein of the integrin signaling pathway (Brancaccio et al. (2006) Cardiovasc. Res. 70:422-433), was phosphorylated potently as well (FIG. 2D). These data show that irisin stimulates a very potent pathway of integrin-like signaling.

### Example 6

### Irisin Binds Directly to Integrin Complexes Through an RGD-Analogous Motif of Irisin and Well-Known Ligand-Binding Motifs Within Integrin αV/β5

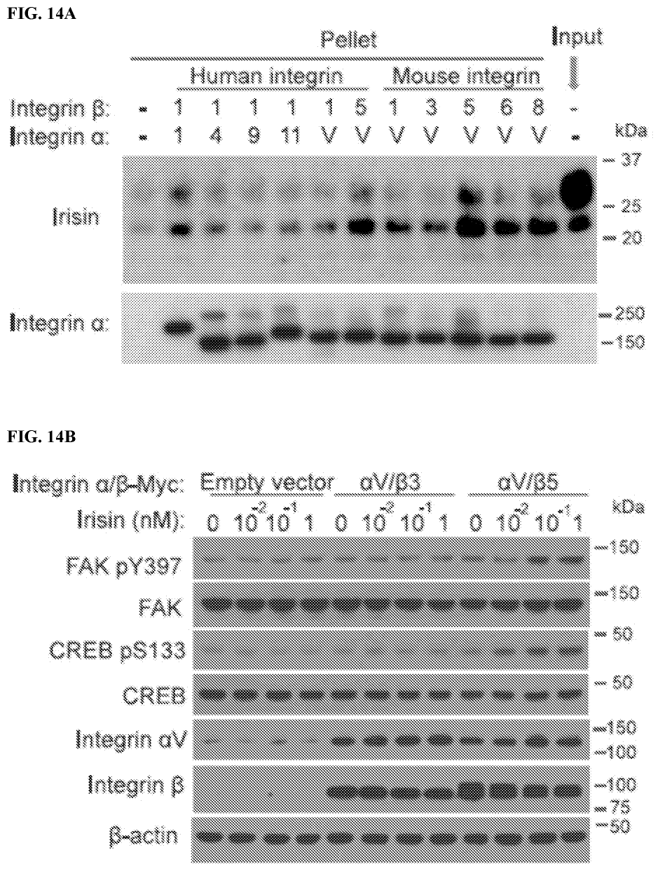

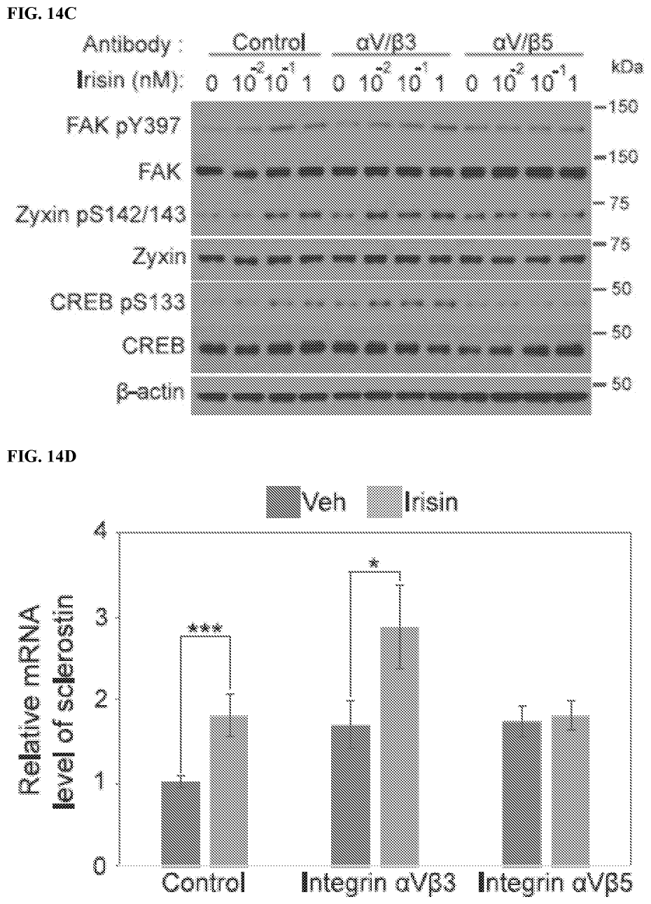

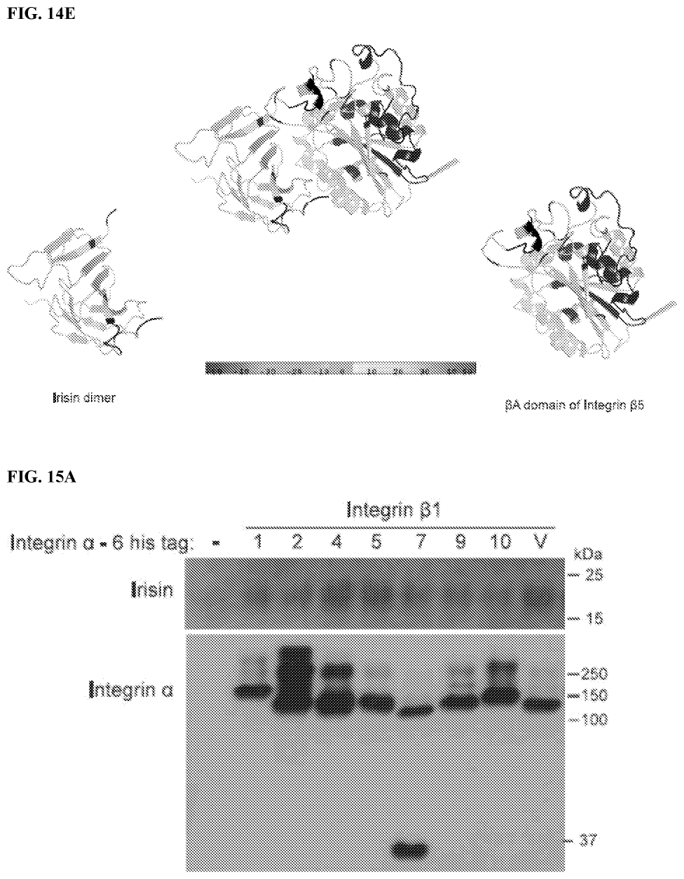

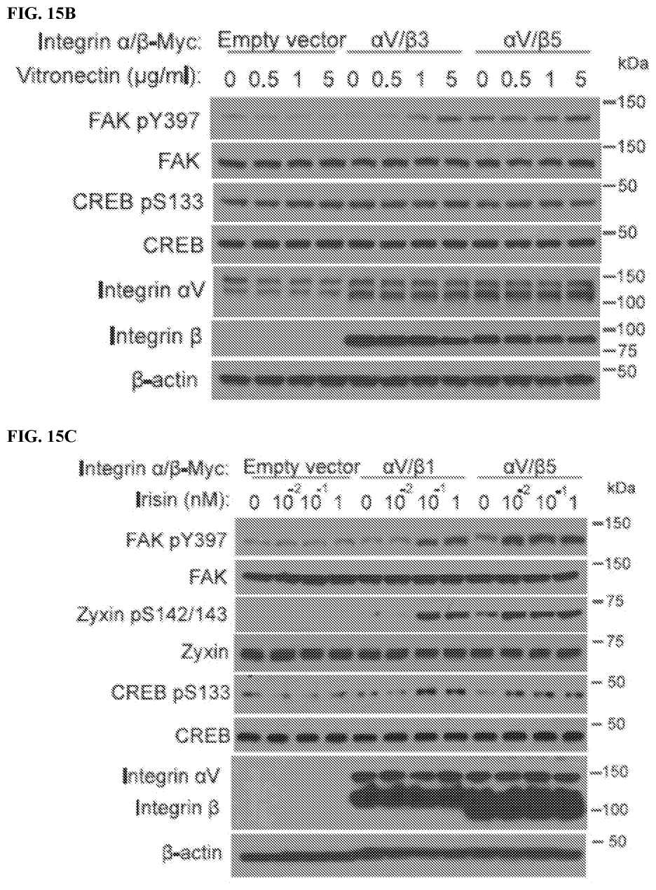

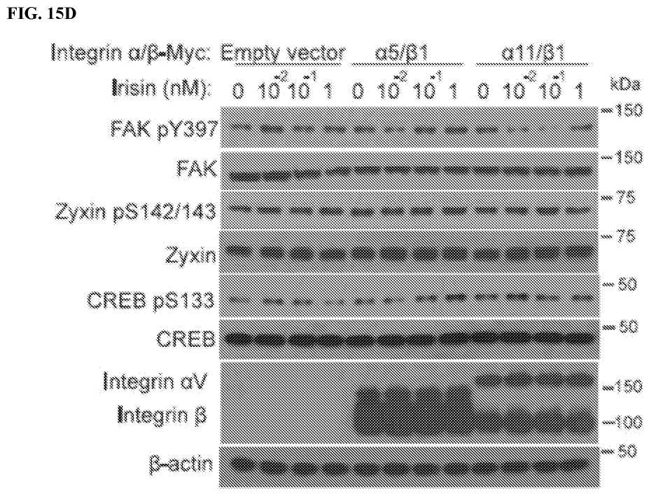

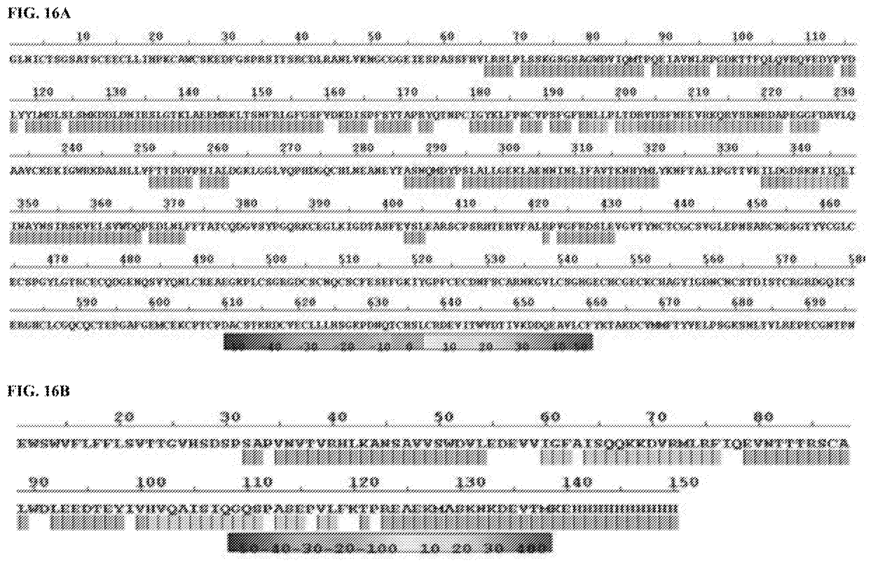

To determine whether irisin binds directly to integrins, a binding assay was performed using purified recombinant irisin and many integrin complexes that were commercially available (FIG. 14A). Most integrin complexes showed relatively weak binding to irisin (FIG. 15A). In particular several of the β1-containing complexes showed binding to irisin above the background (FIG. 14A). However, αV/β5 integrin, both murine and human, showed by far the highest extent of binding. Using quantitative proteomics using mass spectrometry (spectral counting method), expression of multiple integrins was analyzed in MLO-Y4 that bind to irisin. Integrin αV is the most abundant integrin protein in MLO-Y4 cells, followed by integrin β1, integrin α5, integrin β5 and integrin β3 (Table 12). Minor amounts of integrin β6 and integrin β8 were also observed. Therefore, integrin αV/β1, integrin αV/β3, integrin αV/β5 and integrin α5/β1 were mainly focused on in cell culture experiments.

TABLE 12
Relative integrin distribution in MLO-Y4 cells

| | | | Number of | Combined signal- |
|---|---|---|---|---|
| | | | total tryptic | to-noise intensity |
| | Gene symbol | Description | peptides | for all peptides |
| 1 | Itgav | Integrin αV | 78 | 16547 |
| 2 | Itgb1 | Integrin β1 | 53 | 15191.6 |
| 3 | Itga5 | Integrin α5 | 51 | 10278.1 |
| 4 | Itgb5 | Integrin β5 | 40 | 7620.64 |
| 5 | Itgb3 | Integrin β3 | 22 | 5272.88 |
| 6 | Itga1 | Integrin α1 | 15 | 4108.27 |
| 7 | Itga3 | Integrin α3 | 14 | 2288.18 |
| 8 | Itgb7 | Integrin β7 | 14 | 2264.64 |
| 9 | Itga2 | Integrin α2 | 5 | 936.865 |
| 10 | Itga6 | Integrin α6 | 6 | 929.863 |

Gain of function experiments were next performed, using ectopic expression of integrin subunits in cultured HEK293T cells. These cells showed little basal signaling in response to irisin; cells with forced expression of integrin αV/β5 but not of integrin αV/β3 showed an enhanced level of phosphorylation of FAK upon irisin treatment (FIG. 14B). As a positive control, the cells were treated with vitronectin, a ligand for integrin αV family, in the presence of integrin αV/β3 or integrin αV/β5. Vitronectin treatment induced phosphorylation of FAK in both, indicating that the integrins are active forms (FIG. 15B). In addition to integrin αV/β5, irisin treatment increased FAK phosphorylation after forced expression of the integrin αV/β1 (FIG. 15C). However, cells with forced expression of an empty vector, integrin α5/β1, or integrin α11/β1 showed little phosphorylation of FAK above background upon irisin treatment (FIG. 15D).

The response of these cells to irisin was also tested in a loss of function format, namely in the presence of antagonistic antibodies against integrin αV/β3 or integrin αV/β5. MLO-Y4 cells were treated with control mouse monoclonal Igg, or antagonistic antibodies against integrin αV/β3 or integrin αV/β5 before irisin treatment. It was observed that anti-integrin αV/β5 completely blocked the irisin-mediated phosphorylation of FAK, Zyxin and CREB, while control Igg or the anti-integrin αV/β3 did not block signaling (FIG. 14C). the same pattern in the irisin-mediated sclerostin gene expression was also observed (FIG. 14D). These results, taken together, indicate that integrin αV/β5 has both the highest affinity for irisin and is required for the cellular response to irisin; certain other integrins such as αV/β1 also have a significant affinity and response. Importantly, the well-known integrin αV/β3 complex does not trigger a response to irisin in this osteocyte-like cell line.

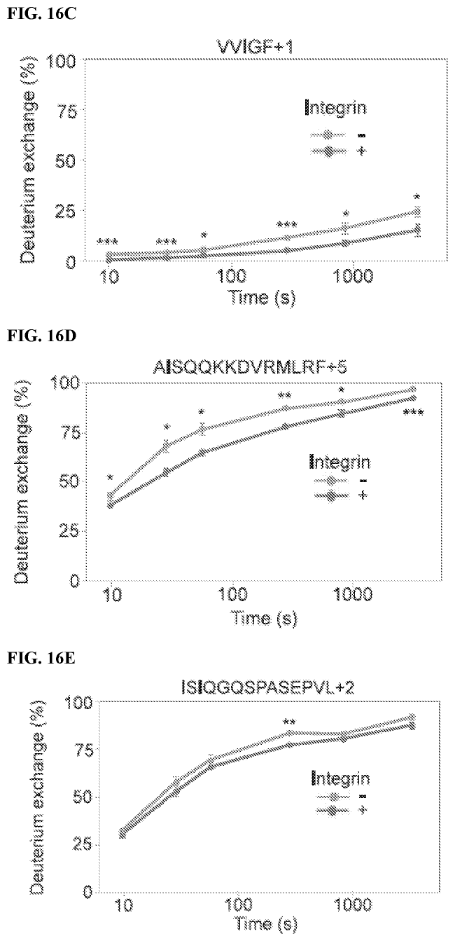

To confirm a direct interaction between irisin and integrin αV/β5 and to help identify which domains in both irisin and αV/β5 integrin participate in this binding event, differential hydrogen-deuterium exchange linked to Mass Spectrometry (HDX/MS) was used. HDX-MS measures deuterium incorporation of peptides via exchange of backbone amide hydrogens which is sensitive to hydrogen bonding and solvent accessibility. If the protein-protein interaction occurred, a reduction of solvent exchange would be expected in the regions of the protein driving the interaction. The experiment was performed as a differential comparing integrin αV/β5±saturating irisin and irisin±saturating integrin αV/β5. HDX/MS identified putative binding regions in the βA domain of integrin β5 which are stabilized (reduction in solvent exchange) when irisin is bound (FIG. 16A). Interestingly, these regions or motifs in integrin β35 have been previously reported to interact with ligands such as fibronectin, osteopontin and vitronectin (Marinelli et al. (2004) J. Med. Chem. 47:4166-4177, Humphries et al. (2006) J. Cell Sci. 119:3901-3903, Van Agthoven et al. (2014) Nat. Struct. Mol. Biol. 21:383-388, Hu et al. (1995) J. Biol. Chem. 270:26232-26238; Smith et al. (1990) J. Biol. Chem. 265:11008-11013). HDX/MS also identified a putative integrin-binding region of irisin at amino acids 60-76 and 101˜118 (FIG. 16B). Interestingly, this region of irisin is proximal to that which has been indicated as a candidate for receptor binding site based on crystal structural similarity with fibronectin (Schumacher et al. (2013) J Bio. Chem. 288: 33738-33744). Moreover, the three-dimensional structure of the proximal motif (amino acid 55-57) is very similar to the well-known “RGD” motif in fibronectin, even though irisin does not have the key amino acid primary sequence(RGD) except for aspartic acid (XXD) (Schumacher et al. (2013) J Bio. Chem. 288: 33738-33744). Likely the direct interaction of this loop motif with integrin further stabilizes the proximal region of irisin leading to reduced solvent exchange (FIG. 16C). The direct interaction of other identified motifs with integrin also has same pattern as well (FIG. 16D-E). These results demonstrate that irisin directly binds integrin αV/β5 and the regions within each protein that are protected from solvent exchange allow the generation of a working model of its three-dimensional interaction (FIG. 14E). Further studies will need to be performed to refine this model.

### Example 7

### Other Integrin Inhibitors Prevent Irisin-Induced Signaling and Sclerostin Expression

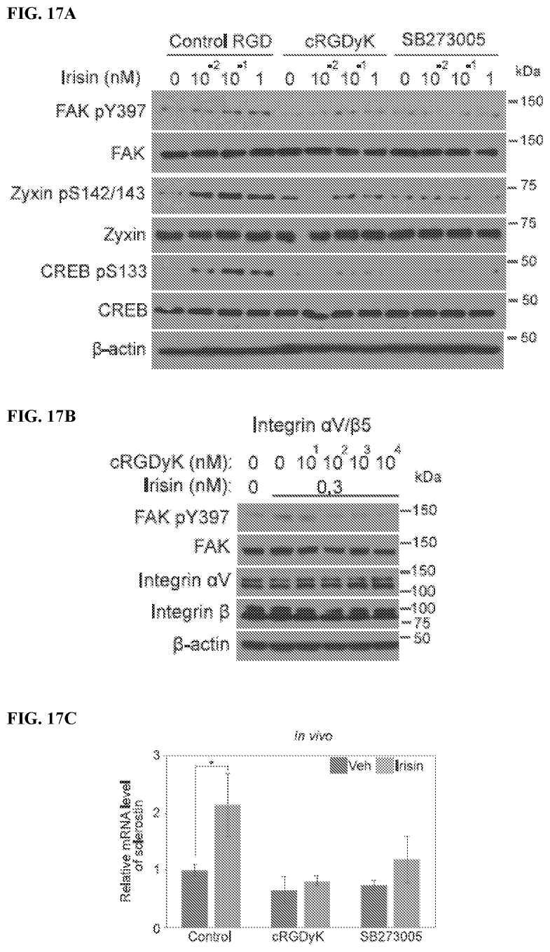

Certain peptides with an RGD motif are well-known inhibitors that prevent integrin-ligand binding and function (Plow et al. (1987) Blood 70:110-115; Plow et al. (2000) J. Biol. Chem. 275:21785-21788). While irisin does not contain an RGD sequence, irisin has a loop that has close structural similarity with certain RGD motifs (Schumacher et al. (2013) J Bio. Chem. 288: 33738-33744) and this loop is used by irisin to bind to integrin αV/β5 (FIG. 14E). Therefore, it was tested whether RGD inhibitory peptides block the interaction between integrins and irisin. As shown in FIG. 5A, the RGDS peptide, which is a commercially available form of the RGD peptide, dramatically suppressed irisin-induced phosphorylation of FAK, Zyxin, and CREB (FIG. 5A). To test whether the αV integrins are major components for FAK signaling in the osteocytes, cells were treated with echistatin, an inhibitor known to affect primarily integrin αV complexes (Kumar et al. (1997) 283:843-853). Echistatin also effectively prevented irisin signaling (FIG. 5B). In addition, irisin-induced signaling was tested with other specific inhibitors for integrin αV, such as cyclo RGDyK and SB273005 (Chen et al. (2004) Bioconjug. Chem. 15:41-49; Dechantsreiter et al. (1999) J. Med. Chem. 42:3033-3040; Miller et al. (2000) J. Med. Chem. 43:22-26; Lark et al. (2001) J. Bone Miner. Res. 16:319-327; Yu et al. (2014) Biomaterials 35:1667-1675). These inhibitors all block irisin-induced signaling (FIG. 17A).

It was also tested whether cyclo RGDyK blocked the irisin-integrin αV/β5 signaling in a dose-dependent manner. After forced expression of integrin αV/β5 in HEK293T cells, cyclo RGDyK was co-treated with irisin. Immunoblot data showed that 10 nM cyclo RGDyK prevented phosphorylation of FAK significantly and 100 nM cyclo RGDyK blocked the phosphorylation completely, indicating that IC50 is 10˜50 nM in the presence of irisin (FIG. 17B). These observations were then extended to the level of gene expression: MLO-Y4 cells were treated with irisin in the presence of a negative control RGD peptide, RGD peptide or cyclo RGDyK and echistatin (FIG. 5C). In the presence of control RGD peptide, irisin raised the mRNA level of sclerostin, while these inhibitors all prevented sclerostin induction. The irisin peptide was also injected, in combination with control RGD peptide or cyclo RGDyK, an integrin inhibitor that is widely used for in vivo studies (Chen et al. (2004) Bioconjug. Chem. 15:41-49; Guo et al. (2014) J. Nanosci. Nanotechnol. 14:4858-4864) (FIGS. 17D-E). Cyclo RGDyK prevented the irisin-induced gene expression of sclerostin in osteocyte-enriched bones, as well as the protein level in plasma. Additionally, SB273005, which has a higher affinity to integrin αV/β5 than integrin αV/β3, was also employed. As shown in FIG. 17C, SB273005 significantly prevented the irisin-induced gene expression in vivo. These results together strongly indicate that irisin acts on integrin αV family and integrin αV/β5 is particularly important in the functions of irisin on osteocyte cells.

### Example 8

### Integrins Mediates the Irisin-Induced Thermogenic Gene Program

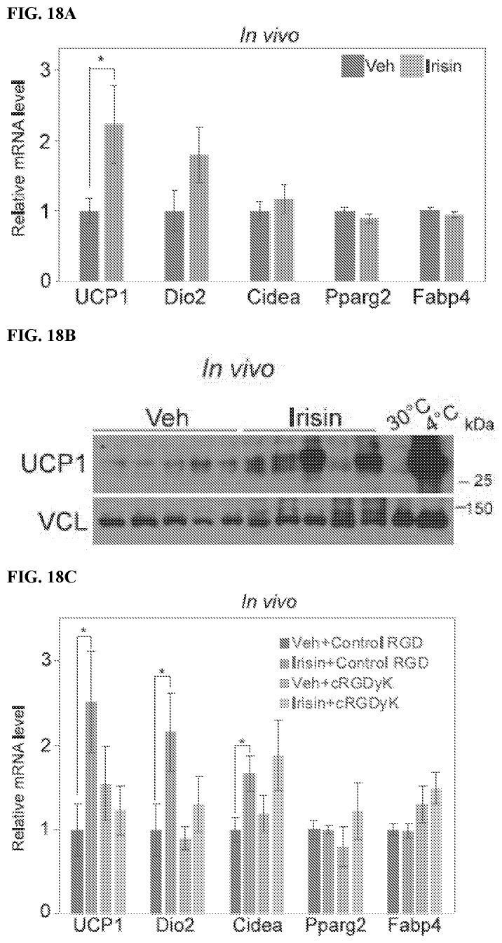

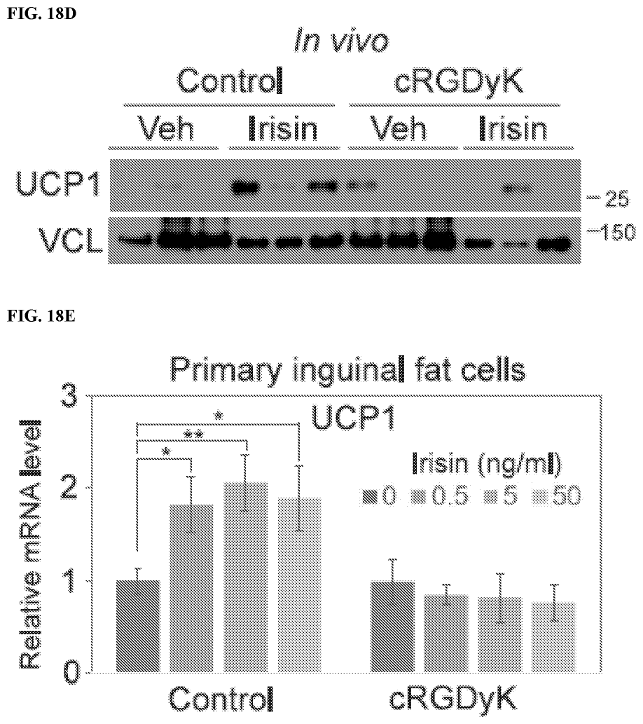

It has been shown that irisin raised the expression of Ucp1 and other thermogenic genes in fat cells (Bostrom et al. (2012) Nature 481:463-468; Lee et al. (2014) Cell Metab. 19:302-309; Huh et al. (2014) Lnt. J. Obes. {Lond} 38:1538-1544). Furthermore, thermogenic gene expression was also elevated when FNDC5 was expressed from the liver with adenoviral vectors and irisin was released in the circulation (Bostrom et al. (2012) Nature 481:463-468). To examine whether recombinant irisin induced the thermogenic gene expression in vivo, recombinant irisin was injected into wild-type mice for one week; irisin treatment increased the mRNA level of Ucp1 more than 2-fold (FIG. 18A). The protein level in whole tissue, as detected by western blots, was also increased by the irisin injections (FIG. 18B). To test whether integrins mediate these effects, the irisin peptide was injected with control RGD peptide or cyclo RGDyK. As shown in FIGS. 18C and 18D, cyclo RGDyK blocked the irisin-induced gene expression of Ucp1 and Dio2 as well as the induction of the protein level of Ucp1. It was also observed that recombinant irisin treatment increased the gene expression of Ucp1 in primary inguinal fat cells (FIG. 18E). Proteomic data showed that in primary inguinal fat cells, integrin β1 is the most abundant followed by integrin β6, integrin α1, integrin β5, and integrin αV. Integrin β3 wasn't detectable in these cells (Table 13). Cyclo RGDyK treatment prevented irisin-induced gene expression (FIG. 18E), indicating that irisin also works on fat cells directly via integrin αV family. Thus, integrin αV complexes also act as receptors for irisin in fat tissue, and mediate the irisin-induced thermogenic gene program.

TABLE 13
Relative integrin distribution in primary inguinal fat cells

| | | | Number of | Combined signal- |
|---|---|---|---|---|
| | | | total tryptic | to-noise intensity |
| | Gene symbol | Description | peptides | for all peptides |
| 1 | Itgb1 | Integrin β1 | 74 | 684481000 |
| 2 | Itga6 | Integrin α6 | 26 | 29851600 |
| 3 | Itga1 | Integrin α1 | 21 | 25502700 |
| 4 | Itgb5 | Integrin β5 | 16 | 10736200 |
| 5 | Itgav | Integrin αV | 15 | 36770300 |
| 6 | Itga5 | Integrin α5 | 8 | 20603000 |
| 7 | Itga11 | Integrin α11 | 8 | 17566500 |
| 8 | Itga8 | Integrin α8 | 2 | 425599 |
| 9 | Itga2 | Integrin α2 | 1 | 117451 |

Since its discovery in 2012, irisin has been reported to have various functions in many organs (Polyzos et al. (2018) Endocrine 59:260-274; Perakakis et al. (2017) Nat. Rev. Endocrinol. 13:324-337). These effects are related mainly to known benefits of exercise, such as strengthening bones, increasing energy expenditure and improving cognition (Colaianni et al. (2015) Proc. Natl. Acad Sci. U.S.A. 112:12157-12162; Colaianni et al. (2017) Sci. Rep. 7:2811; Bostrom et al. (2012) Nature 481:463-468; Zhang et al. (2017) Bone Res. 5:16056; Lee et al. (2014) Cell Metab. 19:302-309; Wrann et al. (2013) Cell Metab. 18:649-659). However, the mechanisms underlying these benefits were unclear, in large measure because the irisin receptor(s) had not been identified. The irisin receptor was described herein as a subset of integrin complexes. Importantly, this conclusion is drawn from several independent lines of evidence. First, the quantitative proteomic analysis showed that irisin binds to osteocyte cells in a way that allows chemical cross-linking to integrin β1. Second, proteinprotein binding assay using purified irisin and integrin complexes showed that irisin binds to several integrin complexes, including α1/β1 integrin; however, integrin αVβ5 has the highest apparent affinity in these experiments. Third, HDX/MS also demonstrated that irisin binds to integrin αV/β5 and this analysis allowed mapping of binding motifs on both irisin and the integrin complex. Fourth, irisin activates signaling characteristic of integrin receptors. One of the main features of integrin signaling is the Y397 phosphorylation of FAK upon ligand binding; irisin treatment of osteocytes raised the phosphorylation level of FAK within one minute. Irisin is also incredibly potent in that 10 pM irisin triggers this phosphorylation and other phosphorylation events known to occur with integrin signaling. Fifth, ectopic expression of αV/β1 or αV/β5 in cultured HEK293T cells showed that irisin can trigger elevated integrin signaling compared to cells transfected with empty vectors. Lastly, it is notable that well-characterized integrin inhibitors or an antagonistic antibody directed against αV/β5 suppressed nearly all irisin-mediated signaling and its downstream gene expression. Taken together, these data prove that a subset of integrins, especially those involving αV integrin, are functional irisin receptors, at least in osteocytes and fat tissues.

The αV family of integrins has previously been reported to contribute to bone remodeling (Thi et al. (2013) Proc. Natl. Acad. Sci. U.S.A. 110:21012-21017; Duong et al. (2000) Matrix Biol. 19:97-105; Duong & Rodan (1998) Front Biosci. 3:D757-768). Interactions of the αV family of integrins with extracellular matrix proteins such as osteopontin and vitronectin lead to adhesion of osteoclasts to the bone surface followed by bone resorption (Flores et al. (1992) Exp. Cell Res. 201:526-530; Horton et al. (1991) Exp. Cell Res. 195:368-375; Duong et al. (2000) Matrix Biol. 19:97-105; Duong & Rodan (1998) Front Biosci. 3:D757-768). HDX/MS experiment determined herein that regions proximal to the RGD like loop of irisin is involved in the interaction with integrin αV/β5. Interestingly, this loop (amino acids 55 to 57), was predicted as a potential receptor binding loop based on the structural similarity with an RGD-sequence containing loop in fibronectin (Schumacher et al. (2013) J Bio. Chem. 288: 33738-33744). In addition, within integrin β5 subunit, the HDX/MS method identified putative binding motifs in the βA domain, which are also reported as the interaction site for RGD-containing ligands (Marinelli et al. (2004) J. Med. Chem. 47:4166-4177, Van Agthoven et al. (2014) Nat. Struct. Mol. Biol. 21:383-388). Based on these data, the ability of RGD-mimetics to block both irisin-induced signaling and irisin-induced gene expression (FIGS. 5 and 18) is understandable from a mechanistic perspective.

The studies described herein reveal for the first time that osteocytes are direct targets of irisin, acting via the integrin αV family. Osteocytes use both mechanical and chemical sensing to maintain bone homeostasis (Bonewald et al. (2011) J. Bone Miner. Res. 26:229-238) by directly controlling skeletal remodeling. In respect to the bone resorption component of skeletal remodeling, osteocytes regulate osteoclasts in two ways: First, by directly secreting RANKL, the most potent inducer of osteoclastogenesis, and second, by secreting sclerostin, an inhibitor of bone formation that also suppresses osteoprotogerin (OPG) a decoy receptor for RANKL. In the most common animal model of osteoporosis, OVX, the loss of estrogen triggers RANKL production and suppresses OPG, leading to greater RANKL bioactivity, increased bone resorption and ultimately bone loss (Komori et al. (2015) Eur. J. Pharmacol. 759:287-294). Histologically, this is manifested by greater numbers of osteoclasts on the bone surface and enhanced osteocytic osteolysis (Almeida et al. (2017) Physiol. Rev. 97:135-187). In experiments described herein, deletion of FDNC5 suppressed bone resorption, by blocking the increase in osteoclast number and eroded surfaces, thereby preventing bone loss after OVX. Furthermore, deficiency of FNDC5 inhibited OVX-induced perilacunar enlargement a manifestation of osteocytic osteolysis, indicating that the phenotype is at least mediated partly through an inactivation of osteocyte function(s), as well as through inhibition of osteoclast number and function. In addition, it was demonstrated that sclerostin was directly induced by irisin in vitro and in vivo. Of course, irisin can have additional effects on other bone cells in the remodeling unit, as demonstrated by (Colaianni et al. (2014) Tnt. J. Endocrinol. 2014:902186).

The data described herein and previous results from others (Colaianni et al. (2015) Proc. Natl. Acad. Sci. U.S.A. 112:12157-12162, Colaianni et al. (2017) Sci. Rep. 7:2811) indicate that irisin can be a useful target for the treatment of osteoporosis. Although irisin targets bone resorption, intermittent treatment with irisin has been shown to improve bone density and strength. Considered within the light of the data described herein, this may seem counter-intuitive. However, a comparable example of a peptide that both stimulates resorption and is anabolic when administered intermittently, is parathyroid hormone (i.e., PTH). Chronically high PTH levels drive bone resorption to maintain eucalcemia. Moreover, Kohrt et al recently demonstrated that during an acute bout of physical activity, serum calcium rapidly decreased and this drived a secondary increase in PTH. Yet it has been well established that intermittent PTH treatment is anabolic to the skeleton, at least over the first twelve months of therapy (Dempster et al. (2001) J Bone Miner. Res. 16:1846-1853; Lane et al. (1998) J. Clin. Invest. 102:1627-1633). Therefore, irisin can both target bone resorption but also act on remodeling in a favorable manner with intermittent pulse dosing. On the other hand, the striking data that OVX induced osteoporosis is entirely prevented in the FNDC5 KO mice, indicates another more conventional therapeutic approach: inhibition/neutralization of irisin or its receptors, the αV integrins.

Ucp1 and Dio2 are key proteins contributing to mitochondrial proton leak and thermogenesis in adipose tissues. It was shown herein that treatment of mice with recombinant irisin protein raised the expression of Ucp1 and Dio2 in subcutaneous (inguinal) adipose tissues, despite the very short half-life of irisin in vivo. Importantly, irisin's effects on these thermogenic genes are also sensitive to simultaneous administration of the αV integrin inhibitor. This indicates the generality of the integrins, especially the αV integrins, as irisin receptors.

The identification of the irisin receptors as integrins in osteocytes and thermogenic fat indiates that the αV family of integrins complexes can be the major irisin receptors in all tissues. However, it is important to note that nothing presented here rules out the possibility of other receptors for irisin within the integrin family or even outside of the integrins. Importantly, the identification of an irisin receptor and its signaling systems can be very useful as both a quality control for irisin preparations and for the development of irisin inhibitors. Healthy humans have levels of circulating irisin in the 3-5 ng/ml range and they are, on average, increased with exercise (Jedrychowski et al. (2015) Cell Metab. 22:734-740). As shown herein, these are the levels of irisin that are quite sufficient to activate irisin receptors. Exercise brings well-known improvements in mood and cognition and there are already data indicating that irisin can mediate some of these effects in the brain (Wrann et al. (2013) Cell Metab. 18:649-659).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of preventing and/or treating a subject afflicted with bone loss conditions, comprising administering to the subject a therapeutically effective amount of an agent that decreases the amount and/or activity of irisin.

2. The method of claim 1, wherein the agent binds to irisin, or to an irisin receptor in osteocytes, and blocks the binding of irisin to the irisin receptor.

3. The method of claim 2, wherein the irisin receptor is an integrin which comprises alpha V subunit, optionally wherein the irisin receptor is alpha V beta 5 (αVβ5)-integrin or αVβ1-integrin.

4. The method of any one of claim 1-3, wherein the agent is a small molecule inhibitor, peptide or peptidomimetic inhibitor, aptamer, antibody, or intrabody.

5. The method of claim 4, wherein the agent comprises an antibody and/or intrabody, or an antigen binding fragment thereof, which specifically binds to irisin or the irisin receptor in osteocytes.

6. The method of claim 5, wherein the agent comprises an antibody and/or intrabody, or an antigen binding fragment thereof, which specifically binds to irisin.

7. The method of claim 5 or 6, wherein the antibody and/or intrabody, or antigen binding fragment thereof, is murine, chimeric, humanized, composite, or human.

8. The method of any one of claims 5-7, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

9. The method of any one of claims 1-8, wherein the agent binds to amino acids 60-76 and/or 101-118 of irisin, or to amino acids 162-174, 196-202, 208-227, and/or 340-346 of integrin β5.

10. The method of any one of claims 1-9, wherein the agent is a RGD inhibitory peptide.

11. The method of any one of claims 1-10, wherein the agent is RGDS peptide.

12. The method of claim 1, wherein the agent is a specific inhibitor for integrin αV.

13. The method of claim 12, wherein the agent is selected from the group consisting of echistatin, cyclo RGDyK and SB273005.

14. The method of claim 1, wherein the agent decreases the copy number and/or amount of FNDC5, the precursor of irisin, or irisin.

15. The method of claim 14, wherein the agent is a small molecule inhibitor, CRISPR guide RNA (gRNA), RNA interfering agent, antisense oligonucleotide, peptide or peptidomimetic inhibitor, aptamer, antibody, or intrabody.

16. The method of claim 15, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

17. The method of claim 15, wherein the agent comprises an antibody and/or intrabody, or an antigen binding fragment thereof, which specifically binds to FNDC5.

18. The method of claim 17, wherein the antibody and/or intrabody, or antigen binding fragment thereof, is murine, chimeric, humanized, composite, or human.

19. The method of claim 17 or 18, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

20. The method of claim 1, wherein the agent inhibits the cleavage of FNDC5 into irisin.

21. The method of claim 20, wherein the agent decreases the copy number, amount and/or activity of the protease that cleaves FNDC5.

22. The method of claim 20 or 21, wherein the agent is a small molecule inhibitor, CRISPR guide RNA (gRNA), RNA interfering agent, antisense oligonucleotide, peptide or peptidomimetic inhibitor, aptamer, antibody, or intrabody.

23. The method of claim 22, wherein the RNA interfering agent is a small interfering RNA (siRNA), CRISPR RNA (crRNA), CRISPR guide RNA (gRNA), a small hairpin RNA (shRNA), a microRNA (miRNA), or a piwi-interacting RNA (piRNA).

24. The method of claim 22, wherein the agent is a protease inhibitor.

25. The method of claim 24, wherein the protease inhibitor is a DPP4 inhibitor.

26. The method of claim 22, wherein the agent comprises an antibody and/or intrabody, or an antigen binding fragment thereof, which specifically binds to the protease that cleaves FNDC5.

27. The method of claim 26, wherein the antibody and/or intrabody, or antigen binding fragment thereof, is murine, chimeric, humanized, composite, or human.

28. The method of claim 26 or 27, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

29. A method of preventing and/or treating a subject afflicted with bone loss conditions, comprising administering to the subject a therapeutically effective amount of a biologically inactive or inhibitory irisin mutant that binds to the irisin receptor in osteocytes.

30. The method of claim 29, wherein the irisin receptor is an integrin which comprises alpha V subunit, optionally wherein the irisin receptor is alpha V beta 5 (αVβ5)-integrin or αVβ1-integrin.

31. The method of claim 29 or 30, wherein the irisin mutant is recombinant or synthetic.

32. The method of any one of claims 1-31, wherein the agent reduces the irisin-induced signaling.

33. The method of any one of claims 1-32, wherein the agent reduces the phosphorylation of FAK, Zyxin, AKT, and/or CREB.

34. The method of any one of claims 1-33, wherein the agent reduces the level of sclerostin and/or RANKL.

35. The method of any one of claims 1-34, wherein the agent prevents OVX-induced bone resorption and/or bone loss.

36. The method of any one of claims 1-35, wherein the agent prevents OVX-induced decrease in the ratio of bone volume to total bone volume, OVX-induced decrease in travecular number, OVX-induced separation between trabeculae in the lumbar vertebrae, OVX-induced increase in osteoclast number and eroded surfaces, and/or OVX-induced perilacunar enlargement.

37. The method of any one of claims 1-36, wherein the agent reduces osteocyte degradative function.

38. The method of any one of claims 1-37, wherein the agent prevents trabecular bone loss, osteoclastic bone resorption, and/or osteocytic osteolysis.

39. The method of any one of claims 1-38, further comprising administering one or more agents that reduce bone mineral density loss.

40. The method of claim 39, wherein the one or more agents that reduce bone mineral density loss are selected from the group consisting of calcium supplements, estrogen, calcitonin, estradiol, diphosphonates, vitamin D3 and/or metabolites thereof, and parathyroid hormone (PTH) and/or deritaves or fragments thereof.

41. A method of assessing the efficacy of an agent for treating bone loss conditions in a subject, comprising:
a) detecting in a subject sample at a first point in time the amount and/or acvitity of irisin;
b) repeating step a) during at least one subsequent point in time after administration of the agent; and
c) comparing the amount detected in steps a) and b), wherein the absence of, or a significant decrease in amount and/or activity of irisin in the subsequent sample as compared to the amount and/or activity of irisin in the sample at the first point in time, indicates that the agent treats bone loss in the subject.

42. The method of claim 41, wherein between the first point in time and the subsequent point in time, the subject has undergone treatment, completed treatment, and/or is in remission for the bone loss conditions.

43. The method of claim 41 or 42, wherein the first and/or at least one subsequent sample is selected from the group consisting of ex vivo and in vivo samples.

44. The method of any one of claims 41-43, wherein the first and/or at least one subsequent sample is obtained from an animal model of the bone loss condition.

45. The method of any one of claims 41-44, wherein the first and/or at least one subsequent sample is a portion of a single sample or pooled samples obtained from the subject.

46. The method of any one of claims 41-45, wherein the sample comprises cells, serum, and/or bone tissue obtained from the subject.

47. The method of any one of claims 41-46, further comprising determining osteocyte function, level of sclerostin and/or RANKL, activation of targets of the irisin receptor, bone mineral volume/total volume, trabecular thickness, trabecular number, eroded bone surface, osteoclast surface, osteoclast number, the separation between trabeculae in the lumbar vertebrae, osteocytic osteolysis, lacunae enlargement, and/or lacunae area.

48. The method of any one of claims 1-47, wherein the agent is administered in a pharmaceutically acceptable formulation.

49. The method of any one of claims 1-48, wherein the subject is an animal model of bone loss conditions, optionally wherein the animal model is a mouse model.

50. The method of any one of claims 1-49, wherein the subject is a mammal.

51. The method of claim 50, wherein the mammal is a mouse or a human.

52. The method of claim 51, wherein the mammal is a human.

53. The method of any one of claims 1-52, wherein the bone loss condition is selected from the group consisting of osteopenia, osteoporosis, and cancer.

54. The method of claim 53, wherein the cancer is multiple myeloma or breast cancer.

55. A cell-based assay for screening for a biologically inactive or inhibitory irisin mutant that binds to the irisin receptor in osteocytes, comprising:
a) contacting osteocytes with an irisin mutant;
b) detecting binding of the test irisin mutant to the isrin receptor; and
c) determining the effect of the test irisin mutant on (1) activitation of downstream targets of the irisin receptor; (2) expression level of scleostin and/or RANKL; and/or (3) H₂O₂-induced osteocyte cell death.

56. The cell-based assay of claim 54, wherein the step of contacting occurs in vivo, ex vivo, or in vitro.

57. The cell-based assay of claim 55 or 56, wherein the irisin receptor is an integrin which comprises alpha V subunit, optionally wherein the irisin receptor is alpha V beta 5 (αVβ5)-integrin or αVβ1-integrin.

58. The cell-based assay of any one of claims 55-57, wherein the downstream targets of the irisin receptor comprise pFAK, pZyxin, pAKT, and/or pCREB.

59. The cell-based assay of any one of claims 55-58, further comprising determining a reduction in the degradative function of the osteocyte cells.


SEQUENCE LISTING (51 sequences)

SEQ ID NO: 1 - DNA, 630 bp, Mus musculus
atgcccccagggccgtgcgcctggccgccccgcgccgcgctccgcctgtggctaggctgcgtctgcttcgcgctggtgcaggcggacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggagatggggaggaaccagcagctgcgaacgggggaggtgctgatcattgttgtggtcctcttcatgtgggcaggtgttatagctctcttctgccgccagtatgatatcatcaaggacaacgagcccaataacaacaaggagaaaaccaagagcgcatcagaaaccagcacaccggagcatcagggtgggggtctcctccgcagcaagatatga

SEQ ID NO: 2 - protein, 209 aa, Mus musculus
MPPGPCAWPPRAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 3 - DNA, 462 bp, Homo sapiens
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaaggtgagggcaagacctgggcctgggtgggccaccctgtgcctcatgctctggtaa

SEQ ID NO: 4 - protein, 153 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKVRARPGPGWATLCLMLW

SEQ ID NO: 5 - DNA, 639 bp, Homo sapiens
atacaccccgggtcgccgagcgcctggccgccccgcgcccgcgccgcgctccgcctgtggctgggctgcgtctgcttcgcgctggtgcaggcggacagtccctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaagatatga

SEQ ID NO: 6 - protein, 212 aa, Homo sapiens
MHPGSPSAWPPRARAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 7 - DNA, 546 bp, Homo sapiens
atacaccccgggtcgccgagcgcctggccgccccgcgcccgcgccgcgctccgcctgtggctgggctgcgtctgcttcgcgctggtgcaggcggacagtccctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcattgaagcgtga

SEQ ID NO: 8 - protein, 181 aa, Homo sapiens
MHPGSPSAWPPRARAALRLWLGCVCFALVQADSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIEA

SEQ ID NO: 9 - DNA, 414 bp, Homo sapiens
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcatcaaggacaatgaacccaataacaacaaggaaaaaaccaagagtgcatcagaaaccagcacaccagagcaccagggcggggggcttctccgcagcaagatatga

SEQ ID NO: 10 - protein, 137 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIKDNEPNNNKEKTKSASETSTPEHQGGGLLRSKI

SEQ ID NO: 11 - DNA, 321 bp, Homo sapiens
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagagatggggaggaaccaacagctgcggacaggcgaggtgctgatcatcgtcgtggtcctgttcatgtgggcaggtgtcattgccctcttctgccgccagtatgacatcattgaagcgtga

SEQ ID NO: 12 - protein, 106 aa, Homo sapiens
MLRFIQEVNTTTRSCALWDLEEDTEYIVHVQAISIQGQSPASEPVLFKTPREAEKMASKNKDEVTMKEMGRNQQLRTGEVLIIVVVLFMWAGVIALFCRQYDIIEA

SEQ ID NO: 13 - DNA, 663 bp, Gallus gallus
atggagcccttcctgggctgcaccggcgccgcgctcctgctctgcttcagctacgccggtctgcggccggtggaggcagacagcccttcggctccggtcaatgtcacagtcaaacacctgaaggccaactcagctgtagtgacttgggacgttctggaggatgaagttgtcattggatttgccatttcccagcagaagaaggacgtgcggatgctgcgcttcatccaggaggtgaacaccaccacccgctcctgtgccctctgggacctagaggaggacactgagtacattgtgcatgtccaggccatcagcatccaaggccagagccctgccagtgagccagtcctcttcaagacccccagggaagctgagaaactggcttctaaaaataaagatgaggtgacaatgaaggagatggcgaagaaaaaccaacagctgcgcgcaggggaaatactcatcattgtggtggtgttgtttatgtgggcaggggtgatcgccctgttctgcaggcagtacgacatcatcaaagacaacgagccgaacaacagcaaggagaaagccaagagcgcctcagagaacagcaccccygagcaccagggtggggggctgctccgcagcaagttcccaaaaaacaaaccctcagtgaacatcattgaggcataa

SEQ ID NO: 14 - protein, 220 aa, Gallus gallus
MEPFLGCTGAALLLCFSYAGLRPVEADSPSAPVNVTVKHLKANSAVVTWDVLEDEVVIGFAISQQKKDVRMLRFIQEVNTTTRSCALWDLEEDTEYIVHVQAISIQGQSPASEPVLFKTPREAEKLASKNKDEVTMKEMAKKNQQLRAGEILIIVVVLFMWAGVIALFCRQYDIIKDNEPNNSKEKAKSASENSTPEHQGGGLLRSKFPKNKPSVNIIEA

SEQ ID NO: 15 - DNA, 1203 bp, Gallus gallus
atggagaagaacagggacggccgcggcccccctggtgtccatctggggatggagaaggaagatgatttagagcccggtgacacgccggggctgcgcgaagccctggtggcgagatgtcaccgctgccgcgcacccgccgggggtctcaccgggacgggccccgtttgctccttccggcgatggggagcggtccgggccgagggctcccggtcccgcctgggggaaactgaggcagacggcggggccgggcggggcgggggccgagccgcccccgggccgggggagggaccggagcggggctgcccagcgctgcagcgggcggagccggggctcggcggggccgcctcccggccgagccgagccgaaccgagccgcgctgccgagggccgccgagcccgcagccgcccccggccgaaccgggcggccccgccggttccgggccccggagctctccgcggtgctgaacggcgccgccgcgcccgcgggacgccggccccggagcggctcggccccggcgcggcgcggcgggccgcggggggatggagcccttcctgggctgcaccggcgccgcgctcctgctctgctttcagctacgccggtctgcggccggtggaggcagacagcccttcggctccggtcaatgtcacagtcaaacacctgaaggccaactcagctgtagtgacttgggacgttctggaggatgaagttgtcattggatttgccatttcccagcagaagaaggacgtgcggatgctgcgcttcatccaggaggtgaacaccaccacccgctcctgtgccctctgggacctagaggaggacactgagtacattgtgcatgtccaggccatcagcatccaaggccagagccctgccagtgagccagtcctcttcaagacccccagggaagctgagaaactggcttctaaaaataaagatgaggtgacaatgaaggagatggcgaagaaaaaccaacagctgcgcgcaggggaaatactcatcattgtggtggtgttgtttatgtgggcaggggtgatcgccctgttctgcaggcagtacgacatcatcaaagacaacgagccgaacaacagcaaggagaaagccaagagcgcctcagagaacagcacccccgagcaccagggtggggggctgctccgcagcaagttcccaaaaaacaaaccctcagtgaacatcattgaggcataa

SEQ ID NO: 16 - protein, 400 aa, Gallus gallus
MEKNRDGRGPPGVHLGMEKEDDLEPGDTPGLREALVARCHRCRAPAGGLTGTGPVCSFRRWGAVRAEGSRSRLGETEADGGAGRGGGRAAPGPGEGPERGCPALQRAEPGLGGAASRPSRAEPSRAAEGRRARSRPRPNRAAPPVPGPGALRGAERRRRARGTPAPERLGPGAARRAAGGWSPSWAAPAPRSCSAFSYAGLRPVEADSPSAPVNVTVKHLKANSAVVTWDVLEDEVVIGFAISQQKKDVRMLRFIQEVNTTTRSCALWDLEEDTEYIVHVQAISIQGQSPASEPVLFKTPREAEKLASKNKDEVTMKEMAKKNQQLRAGEILIIVVVLFMWAGVIALFCRQYDIIKDNEPNNSKEKAKSASENSTPEHQGGGLLRSKFPKNKPSVNIIEA

SEQ ID NO: 17 - DNA, 555 bp, Danio rerio
atgagttcttacagtttggcagctccagtgaatgtgtccatcagggatctgaagagcagctcagccgtggtgacatgggacacgccagacggagagccagtcatcggcttcgccatcacacaacagaagaaagatgtccgcatgctgcgctttattcaagaagtgaacaccaccacgcggagctgtgcattgtgggatctggaagctgatacggattacattgtgcacgttcagtctatcagcatcagcggggcgagtcctgttagtgaagctgtgcacttcaagaccccgacagaagttgaaacacaggcctccaagaacaaagacgaggtgacgatggaggaggtcgggccgaacgctcagctcagggccggagagttcatcattattgtggtggtcctcatcatgtgggcaggtgtgatcgcactattctgccgtcagtatgacatcattaaagacaacgaaccaaacaataacaaggataaagccaagaactcgtctgaatgcagcactccagagcacacgtcaggtggcctgctgcgcagtaaggtataa

SEQ ID NO: 18 - protein, 184 aa, Danio rerio
MSSYSLAAPVNVSIRDLKSSSAVVTWDTPDGEPVIGFAITQQKKDVRMLRFIQEVNTTTRSCALWDLEADTDYIVHVQSISISGASPVSEAVHFKTPTEVETQASKNKDEVTMEEVGPNAQLRAGEFIIIVVVLIMWAGVIALFCRQYDIIKDNEPNNNKDKAKNSSECSTPEHTSGGLLRSKV

SEQ ID NO: 19 - DNA, 630 bp, Rattus norvegicus
atgcccccagggccgtgcgcctggccgccccgcgccgctctccggctgtggctgggctgcgtgtgcttcgcgctggtgcaggcggacagcccctcggcccccgtgaacgtaaccgtcaggcacctcaaggccaactcggcagtggtcagctgggacgtcctggaggacgaggttgtcatcggctttgccatctctcagcagaagaaggatgtgaggatgctgcgcttcattcaggaggtgaacaccaccacccgatcctgcgctctctgggacctggaggaggacacagagtatatcgtccacgtgcaggccatctccatccagggccagagcccagccagtgagcccgtgctcttcaagaccccacgtgaggccgagaagatggcctctaagaacaaagatgaggtgaccatgaaggagatggggaggaaccagcagctgcggacgggcgaggtgctgatcatcgtcgtggtcctcttcatgtgggcaggtgtcatagctctcttctgccgccagtatgacatcatcaaggacaacgagcccaataacaacaaggaaaaaaccaagagtgcatcagagaccagcaccccagagcaccagggtgggggtctcctccgaagcaagatatga

SEQ ID NO: 20 - protein, 209 aa, Rattus norvegicus; identical to SEQ ID NO: 2

SEQ ID NO: 21 - DNA, 336 bp, Mus musculus
gacagcccctcagcccctgtgaacgtgaccgtccggcacctcaaggccaactctgccgtggtcagctgggatgtcctggaggatgaagtggtcattggctttgccatctctcagcagaagaaggatgtgcggatgctccggttcattcaggaggtgaacaccaccacccggtcctgcgctctctgggacctggaggaggacacagaatatatcgtccatgtgcaggccatctccatccagggacagagcccagccagtgagcctgtgctcttcaagaccccacgcgaggctgaaaagatggcctcaaagaacaaagatgaggtgaccatgaaggag

SEQ ID NO: 22 - protein, 112 aa, Mus musculus
DSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKNKDEVTMKE

SEQ ID NO: 23 - DNA, 204 bp, Homo sapiens
atgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagag

SEQ ID NO: 24 - protein, 68 aa, Homo sapiens
MLRFIQEVNTTTRSCALWDLEEDTEYIVHVQAISIQGQSPASEPVLFKTPREAEKMASKNKDEVTMKE

SEQ ID NO: 25 - DNA, 336 bp, Homo sapiens
gacagtccctcagccccagtgaacgtcaccgtcaggcacctcaaggccaactctgcagtggtgagctgggatgttctggaggatgaggttgtcatcggatttgccatctcccagcagaagaaggatgtgcggatgctgcgcttcatccaggaggtgaacaccaccacccgctcatgtgccctctgggacctggaggaggatacggagtacatagtccacgtgcaggccatctccattcagggccagagcccagccagcgagcctgtgctcttcaagaccccgcgtgaggctgagaagatggcctccaagaacaaagatgaggtaaccatgaaagag

SEQ ID NO: 26 - protein, 112 aa, Homo sapiens; identical to SEQ ID NO: 22

SEQ ID NO: 27 - DNA, 336 bp, Homo sapiens; identical to SEQ ID NO: 25

SEQ ID NO: 28 - protein, 112 aa, Homo sapiens; identical to SEQ ID NO: 22

SEQ ID NO: 29 - DNA, 21 bp, Artificial Sequence
  /note=“Description of Artificial Sequence Synthetic primer sequence”
agccttcaggaatgatgccac

SEQ ID NO: 30 - DNA, 21 bp, Artificial Sequence
  /note=“Description of Artificial Sequence Synthetic primer sequence”
ctttggcgtcatagggatggt

SEQ ID NO: 31 - DNA, 20 bp, Artificial Sequence
  /note=“Description of Artificial Sequence Synthetic primer sequence”
ggtgcctacatcgtactggc

SEQ ID NO: 32 - DNA, 21 bp, Artificial Sequence
  /note=“Description of Artificial Sequence Synthetic primer sequence”
tgcttggtaagctccttggtg

SEQ ID NO: 33 - DNA, 23 bp, Artificial Sequence
  /note=“Description of Artificial Sequence Synthetic primer sequence”
cagtgtggtgcacgtctccaatc

SEQ ID NO: 34 - DNA, 21 bp, Artificial Sequence
  /note=“Description of Artificial Sequence Synthetic primer sequence”
tgaaccaaagttgaccaccag

SEQ ID NO: 35 - DNA, 21 bp, Artificial Sequence
  /note=“Description of Artificial Sequence Synthetic primer sequence”
tgctcttctgtatcgcccagt

SEQ ID NO: 36 - DNA, 21 bp, Artificial Sequence
  /note=“Description of Artificial Sequence Synthetic primer sequence”
gccgtgttaaggaatctgctg

SEQ ID NO: 37 - DNA, 21 bp, Artificial Sequence
  /note=“Description of Artificial Sequence Synthetic primer sequence”
gtgccagtttcgatccgtaga

SEQ ID NO: 38 - DNA, 20 bp, Artificial Sequence
  /note=“Description of Artificial Sequence Synthetic primer sequence”
ggccagcatcgtgtagatga

SEQ ID NO: 39 - DNA, 23 bp, Artificial Sequence
  /note=“Description of Artificial Sequence Synthetic primer sequence”
aaggtgaagagcatcataaccct

SEQ ID NO: 40 - DNA, 23 bp, Artificial Sequence
  /note=“Description of Artificial Sequence Synthetic primer sequence”
tcacgcctttcataacacattcc

SEQ ID NO: 41 - DNA, 23 bp, Artificial Sequence
  /note=“Description of Artificial Sequence Synthetic primer sequence”
catcctaaagcatacaggtcctg

SEQ ID NO: 42 - DNA, 20 bp, Artificial Sequence
  /note=“Description of Artificial Sequence Synthetic primer sequence”
tccatggcttccacaatgtt

SEQ ID NO: 43 - DNA, 19 bp, Artificial Sequence
  /note=“Description of Artificial Sequence Synthetic primer sequence”
tccgaggaccacaatgaac

SEQ ID NO: 44 - DNA, 22 bp, Artificial Sequence
  /note=“Description of Artificial Sequence Synthetic primer sequence”
tgggttgtccattcaatgatgt

SEQ ID NO: 45 - protein, 10 aa, Mus musculus
FIQEVNTTTR

SEQ ID NO: 46 - protein, 12 aa, Mus musculus
DSPSAPVNVTVR

SEQ ID NO: 47 - protein, 696 aa, Artificial Sequence
  /note=“Description of Artificial Sequence Integrin beta 5 polypeptide sequence”
GLNICTSGSATSCEECLLIHPKCAWCSKEDFGSPRSITSRCDLRANLVKNGCGGEIESPASSFHVLRSLPLSSKGSGSAGWDVIQMTPQEIAVNLRPGDKTTFQLQVRQVEDYPVDLYYLMDLSLSMKDDLDNIRSLGTKLAEEMRKLTSNFRLGFGSFVDKDISPFSYTAPRYQTNPCIGYKLFPNCVPSFGFRHLLPLTDRVDSFNEEVRKQRVSRNRDAPEGGFDAVLQAAVCKEKIGWRKDALHLLVFTTDDVPHIALDGKLGGLVQPHDGQCHLNEANEYTASNQMDYPSLALLGEKLAENNINLIFAVTKNHYMLYKNFTALIPGTTVEILDGDSKNIIQLIINAYNSIRSKVELSVWDQPEDLNLFFTATCQDGVSYPGQRKCEGLKIGDTASFEVSLEARSCPSRHTEHVFALRPVGFRDSLEVGVTYNCTCGCSVGLEPNSARCNGSGTYVCGLCECSPGYLGTRCECQDGENQSVYQNLCREAEGKPLCSGRGDCSCNQCSCFESEFGKIYGPFCECDNFSCARNKGVLCSGHGECHCGECKCHAGYIGDNCNCSTDISTCRGRDGQICSERGHCLCGQCQCTEPGAFGEMCEKCPTCPDACSTKRDCVECLLLHSGKPDNQTCHSLCRDEVITWVDTIVKDDQEAVLCFYKTAKDCVMMFTYVELPSGKSNLTVLREPECGNTPN

SEQ ID NO: 48 - protein, 141 aa, Artificial Sequence
  /note=“Description of Artificial Sequence Irisin polypeptide sequence”
EWSWVFLFFLSVTTGVHSDSPSAPVNVTVRHLKANSAVVSWDVLEDEVVIGFAISQQKKDVRMLRFIQEVNTTTRSCALWDLEEDTEYIVHVQAISIQGQSPASEPVLFKTPREAEKMASKANKDEVTMKEHHHHHHHHHH

SEQ ID NO: 49 - protein, 5 aa, Artificial Sequence
  /note=“Description of Artificial Sequence Irisin peptide sequence”
VVIGF

SEQ ID NO: 50 - protein, 14 aa, Artificial Sequence
  /note=“Description of Artificial Sequence Irisin peptide sequence”
AISQQKKDVRMLRF

SEQ ID NO: 51 - protein, 14 aa, Artificial Sequence
  /note=“Description of Artificial Sequence Irisin peptide sequence”
ISIQGQSPASEPVL